(19) United States
(12) Patent Application Publication
(10) Pub. No.: US 2005/0170344 A1
(43) Pub. Date: Aug. 4, 2005

(54) Chds as modifiers of the p53 pathway and methods of use

(76) Inventors: Lori Friedman, San Carlos, CA (US); Gregory D. Plowman, San Carlos, CA (US); Marcia Belvin, Albany, CA (US); Helen Francis-Lang, San Francisco, CA (US); Danxi Li, Zionsville, IN (US); Roel P Funke, Brisbane, CA (US); Mario N. Lioubin, San Mateo, CA (US)

Correspondence Address: PATENT DEPT;EXELIXIS, INC., 170 HARBOR WAY, P.O. BOX 511, SOUTH SAN FRANCISCO, CA, 94083-0511, US

(21) Appl. No.: 10/479,874

(22) PCT Filed: Jun. 3, 2002
(86) PCT No.: PCT/US02/17466

Related U.S. Application Data
(60) Provisional application No. 60/296,076, filed on Jun. 5, 2001.
(60) Provisional application No. 60/328,605, filed on Oct. 10, 2001.
(60) Provisional application No. 60/338,733, filed on Oct. 22, 2001.
(60) Provisional application No. 60/357,253, filed on Feb. 15, 2002.
(60) Provisional application No. 60/357,600, filed on Feb. 15, 2002.

(51) Int. Cl. C12Q 1/68; G01N 33/574

(52) U.S. Cl. 435/6; 435/7.23

(57) ABSTRACT

Human CHD genes are identified as modulators of the p53 pathway, and thus are therapeutic targets for disorders associated with defective p53 function. Methods for identifying modulators of p53, comprising screening for agents that modulate the activity of CHD are provided.

### REFERENCE TO RELATED APPLICATIONS

This application claims priority to U.S. provisional patent applications 60/296,076 filed Jun. 5, 2001, 60/328,605 filed Oct. 10, 2001, 60/338,733 filed Oct. 22, 2001, 60/357,253 filed Feb. 15, 2002, and 60/357,600 filed Feb. 15, 2002. The contents of the prior applications are hereby incorporated in their entirety.

### BACKGROUND OF THE INVENTION

The p53 gene is mutated in over 50 different types of human cancers, including familial and spontaneous cancers, and is believed to be the most commonly mutated gene in human cancer (Zambetti and Levine, FASEB (1993) 7:855-865; Hollstein, et al., Nucleic Acids Res. (1994) 22:3551-3555). Greater than 90% of mutations in the p53 gene are missense mutations that alter a single amino acid that inactivates p53 function. Aberrant forms of human p53 are associated with poor prognosis, more aggressive tumors, metastasis, and short survival rates (Mitsudomi et al., Clin Cancer Res 2000 October; 6(10):4055-63; Koshland, Science (1993) 262:1953).

The human p53 protein normally functions as a central integrator of signals including DNA damage, hypoxia, nucleotide deprivation, and oncogene activation (Prives, Cell (1998) 95:5-8). In response to these signals, p53 protein levels are greatly increased with the result that the accumulated p53 activates cell cycle arrest or apoptosis depending on the nature and strength of these signals. Indeed, multiple lines of experimental evidence have pointed to a key role for p53 as a tumor suppressor (Levine, Cell (1997) 88:323-331). For example, homozygous p53 “knockout” mice are developmentally normal but exhibit nearly 100% incidence of neoplasia in the first year of life (Donehower et al., Nature (1992) 356:215-221).

The biochemical mechanisms and pathways through which p53 functions in normal and cancerous cells are not fully understood, but one clearly important aspect of p53 function is its activity as a gene-specific transcriptional activator. Among the genes with known p53-response elements are several with well-characterized roles in either regulation of the cell cycle or apoptosis, including GADD45, p21/Waf1/Cip1, cyclin G, Bax, IGF-BP3, and MDM2 (Levine, Cell (1997) 88:323-331).

The murine gene ‘chromodomain helicase DNA-binding protein-1’ (Chd1) was isolated by in a search for proteins that bind a DNA promoter element (Delmas, V. et al. (1993) Proc. Nat. Acad. Sci. 90: 2414-2418). The presence of chromo (chromatin organization modifier) domains and an SNF2-related helicase/ATPase domain led to speculation that this gene regulates chromatin structure or gene transcription (Woodage, T. et al. (1997) Proc. Nat. Acad. Sci. 94: 11472-11477). Since this discovery, several more related genes have been found, yielding a total of 12 highly conserved CHD genes from diverse organisms from yeast to mammals. The least conserved region is in the C-terminal part of the proteins, a region with DNA-binding activity in mouse Chd1 (Woodage, T. et al. (1997) supra). Alteration of gene expression by CHD genes may occur by changing chromatin structure, which could influence access of the transcriptional apparatus to its chromosomal DNA template (Woodage, T. et al. (1997) supra).

Human Chromodomain helicase DNA binding protein 1 (CHD1) contains DNA-binding chromo (chromatin organization modifier) domains and an SNF2-related helicase/ATPase domain. It may play role in establishment or maintenance of chromatin structure (Delmas et al. (1993) supra).

Chromodomain helicase DNA binding protein 2 (CHD2) is also a putative ATP dependent helicase, which may be involved in regulation of transcription, modification of chromatin structure, and DNA repair (Woodage, T. et al. (1997) supra).

Anti-Mi2 autoantibody is associated with dermatomyositis and is found in sera of 20% of dermatomyositis patients. Mi2 antigen consists of at least 8 components (Seelig, H. et al. (1995) Arthritis Rheum 38: 1389-1399). A partial cDNA encoding Mi2-alpha, or CHD3, was found by screening various expression libraries with anti-Mi2 patient sera (Ge, Q. et al. (1995) J. Clin. Invest 96:1730-1737). The predicted, partial protein contains 4 potential zinc finger domains. Mi2-alpha and Mi2-beta (CHD4) proteins react with most or all dermatomyositis patient anti-Mi2 sera (Seelig, H. et al. (1996) Arthritis Rheum. 39: 1769-1771). While these proteins are distinct, they have stretches of identical sequence that could result in shared epitopes and contain subunit of the nucleosome remodeling and histone deacetylase (NURD) complex; an autoantigen involved in dermatomyositis (Zhang, Y. et al. (1998) Cell 95: 279-289).

CHD4 belongs to the SNF2/RAD54 family of nuclear helicases. The central portion of Mi2-beta contains the 7 motifs, including a DEAD/H box, that are characteristic of helicases as well as a putative chromatin-binding region and multiple potential nuclear targeting signals, N-glycosylation sites, N-myristoylation sites, and phosphorylation sites (Seelig, H. et al. (1996) supra).

Patients with dermatomyositis tend to have a high rate of malignancy. CHD4 exists in a complex containing histone deacetylase and nucleosome remodeling activities, suggesting that it plays a role in chromatin reorganization in cancer metastasis (Zhang, Y. et al. (1999) Genes Dev. 13: 1924-1935).

KIAA1416 is a protein containing two SNF2 related N-terminal domains and a helicase conserved C-terminal domain. It contains a region of moderate similarity to a region of chromodomain helicase DNA binding protein 4 (CHD4) which may regulate chromatin structure (Nagase, T. et al (2000) DNA Res; 7(1): 65-73).

KIAA1564 is a protein containing a helicase conserved C-terminal domain and a region of low similarity to a region of chromodomain helicase DNA binding protein 1 (mouse Chd1), which binds to DNA (Nagase, T. et al. (2000) DNA Res; 7(4): 273-81.

The ability to manipulate the genomes of model organisms such as Drosophila provides a powerful means to analyze biochemical processes that, due to significant evolutionary conservation, have direct relevance to more complex vertebrate organisms. Due to a high level of gene and pathway conservation, the strong similarity of cellular processes, and the functional conservation of genes between these model organisms and mammals, identification of the involvement of novel genes in particular pathways and their functions in such model organisms can directly contribute to the understanding of the correlative pathways and methods of modulating them in mammals (see, for example, Mechler B M et al., 1985 EMBO J 4:1551-1557; Gateff E. 1982 Adv. Cancer Res. 37: 33-74; Watson K L., et al., 1994 J Cell Sci. 18: 19-33; Miklos G L, and Rubin G M. 1996 Cell 86:521-529; Wassarman D A, et al., 1995 Curr Opin Gen Dev 5: 44-50; and Booth D R. 1999 Cancer Metastasis Rev. 18: 261-284). For example, a genetic screen can be carried out in an invertebrate model organism having underexpression (e.g. knockout) or overexpression of a gene (referred to as a “genetic entry point”) that yields a visible phenotype. Additional genes are mutated in a random or targeted manner. When a gene mutation changes the original phenotype caused by the mutation in the genetic entry-point, the gene is identified as a “modifier” involved in the same or overlapping pathway as the genetic entry point. When the genetic entry point is an ortholog of a human gene implicated in a disease pathway, such as p53, modifier genes can be identified that may be attractive candidate targets for novel therapeutics.

All references cited herein, including sequence information in referenced Genbank identifier numbers and website references, are incorporated herein in their entireties.

### SUMMARY OF THE INVENTION

We have discovered genes that modify the p53 pathway in Drosophila, and identified their human orthologs, hereinafter referred to as CHD. The invention provides methods for utilizing these p53 modifier genes and polypeptides to identify candidate therapeutic agents that can be used in the treatment of disorders associated with defective p53 function. Preferred CHD-modulating agents specifically bind to CHD polypeptides and restore p53 function. Other preferred CHD-modulating agents are nucleic acid modulators such as antisense oligomers and RNAi that repress CHD gene expression or product activity by, for example, binding to and inhibiting the respective nucleic acid (i.e. DNA or mRNA).

CHD-specific modulating agents may be evaluated by any convenient in vitro or in vivo assay for molecular interaction with a CHD polypeptide or nucleic acid. In one embodiment, candidate p53 modulating agents are tested with an assay system comprising a CHD polypeptide or nucleic acid. Candidate agents that produce a change in the activity of the assay system relative to controls are identified as candidate p53 modulating agents. The assay system may be cell-based or cell-free. CHD-modulating agents include CHD related proteins (e.g. dominant negative mutants, and biotherapeutics); CHD-specific antibodies; CHD-specific antisense oligomers and other nucleic acid modulators; and chemical agents that specifically bind CHD or compete with CHD binding target. In one specific embodiment, a small molecule modulator is identified using a helicase assay. In specific embodiments, the screening assay system is selected from a binding assay, an apoptosis assay, a cell proliferation assay, an angiogenesis assay, and a hypoxic induction assay.

In another embodiment, candidate p53 pathway modulating agents are further tested using a second assay system that detects changes in the p53 pathway, such as angiogenic, apoptotic, or cell proliferation changes produced by the originally identified candidate agent or an agent derived from the original agent. The second assay system may use cultured cells or non-human animals. In specific embodiments, the secondary assay system uses non-human animals, including animals predetermined to have a disease or disorder implicating the p53 pathway, such as an angiogenic, apoptotic, or cell proliferation disorder (e.g. cancer).

The invention further provides methods for modulating the p53 pathway in a mammalian cell by contacting the mammalian cell with an agent that specifically binds a CHD polypeptide or nucleic acid. The agent may be a small molecule modulator, a nucleic acid modulator, or an antibody and may be administered to a mammalian animal predetermined to have a pathology associated the p53 pathway.

### DETAILED DESCRIPTION OF THE INVENTION

Genetic screens were designed to identify modifiers of the p53 pathway in Drosophila in which p53 was overexpressed in the wing (Ollmann M, et al., Cell 2000 101: 91-101). The kismet gene was identified as a modifier of the p53 pathway. Accordingly, vertebrate orthologs of these modifiers, and preferably the human orthologs, Chromodomain DNA binding (CHD) genes (i.e., nucleic acids and polypeptides) are attractive drug targets for the treatment of pathologies associated with a defective p53 signaling pathway, such as cancer.

In vitro and in vivo methods of assessing CHD function are provided herein. Modulation of the CHD or their respective binding partners is useful for understanding the association of the p53 pathway and its members in normal and disease conditions and for developing diagnostics and therapeutic modalities for p53 related pathologies. CHD-modulating agents that act by inhibiting or enhancing CHD expression, directly or indirectly, for example, by affecting a CHD function such as enzymatic (e.g., catalytic) or binding activity, can be identified using methods provided herein. CHD modulating agents are useful in diagnosis, therapy and pharmaceutical development.

Nucleic Acids and Polypeptides of the Invention

Sequences related to CHD nucleic acids and polypeptides that can be used in the invention are disclosed in Genbank (referenced by Genbank identifier (GI) number) as GI#s 11417245 (SEQ ID NO: 1), 4557446 (SEQ ID NO:2), 4557448 (SEQ ID NO:3), 4557450 (SEQ ID NO:4), 17442584 (SEQ ID NO:5), 1107695 (SEQ ID NO:6), 4557452 (SEQ ID NO:7), 2224556 (SEQ ID NO:8), 11345538 (SEQ ID NO:9), 14770875 (SEQ ID NO:10), 7243050 (SEQ ID NO:11), 7243212 (SEQ ID NO:12), 17448489 (SEQ ID NO:14), 18583433 (SEQ ID NO:17), 10047192 (SEQ ID NO:18), and 16552097 (SEQ ID NO: 19) for nucleic acid, and GI#s 11417246 (SEQ ID NO:20), 4557447 (SEQ ID NO:21), 3182950 (SEQ ID NO:22), 4557449 (SEQ ID NO:23), 4557451 (SEQ ID NO:24), 17442585 (SEQ ID NO:25), 5921744 (SEQ ID NO:26), 4557453 (SEQ ID NO:27), 2224557 (SEQ ID NO:28), 11345539 (SEQ ID NO:29), 19421557 (SEQ ID NO:30), 7243213 (SEQ ID NO:31), and 18583434 (SEQ ID NO:32) for polypeptides. Additionally, novel nucleic acid sequences of SEQ ID NOs:13, 15, and 16 can also be used in the invention.

CHDs are chromodomain helicase binding proteins with chromo, helicase, or SNF2 N terminal domains. The term “CHD polypeptide” refers to a full-length CHD protein or a functionally active fragment or derivative thereof. A “functionally active” CHD fragment or derivative exhibits one or more functional activities associated with a full-length, wild-type CHD protein, such as antigenic or immunogenic activity, enzymatic activity, ability to bind natural cellular substrates, etc. The functional activity of CHD proteins, derivatives and fragments can be assayed by various methods known to one skilled in the art (Current Protocols in Protein Science (1998) Coligan et al., eds., John Wiley & Sons, Inc., Somerset, New Jersey) and as further discussed below. For purposes herein, functionally active fragments also include those fragments that comprise one or more structural domains of a CHD, such as a helicase domain or a binding domain. Protein domains can be identified using the PFAM program (Bateman A., et al., Nucleic Acids Res, 1999, 27:260-2; http://pfam.wustl.edu). For example, the SNF2N domains of CHDs from GI#s 4557447 (SEQ ID NO:21), 4557449 (SEQ ID NO:23), 4557451 (SEQ ID NO:24), 17442585 (SEQ ID NO:25), 4557453 (SEQ ID NO:27), 19421557 (SEQ ID NO:30), and 7243213 (SEQ ID NO:31), are located respectively at approximately amino acid residues 484-765, 487-768, 739-1035, 126-422, 729-1025, 462-749, and 209496 (PFAM 00176). Further, the helicase conserved C-terminal domains of CHDs from GI#s 4557447 (SEQ ID NO:21), 4557449 (SEQ ID NO:23), 4557451 (SEQ ID NO:24), 17442585 (SEQ ID NO:25), 4557453 (SEQ ID NO:27), 19421557 (SEQ ID NO:30), 7243213 (SEQ ID NO:31), and 18583434 (SEQ ID NO:32), are located respectively at approximately amino acid residues 828-902, 831-905, 1100-1174, 487-561, 1090-1164, 821-895, 568-642, and 930167. Still further, the chromatin organization modifier (CHROMO) domain (PFAM00385) of various CHDs are located approximately at amino acids 292-338 and 386-429 for GI#4557447 (SEQ ID NO:21), 281-327 and 375-433 for GI#4557449 (SEQ ID NO:23), 628-670 for GI#4557451 (SEQ ID NO:24), 12-54 for GI#17442585 (SEQ ID NO:25), 619-661 for GI#4557453 (SEQ ID NO:27), 37416 for GI#19421557 (SEQ ID NO:30), and 35-84 and 117-162 for GI#7243213 (SEQ ID NO:31). Methods for obtaining CHD polypeptides are also further described below. In some embodiments, preferred fragments are functionally active, domain-containing fragments comprising at least 25 contiguous amino acids, preferably at least 50, more preferably 75, and most preferably at least 100 contiguous amino acids of any one of SEQ ID NOs:20 through 32 (a CHD). In further preferred embodiments, the fragment comprises the entire helicase (functionally active) domain.

The term “CHD nucleic acid” refers to a DNA or RNA molecule that encodes a CHD polypeptide. Preferably, the CHD polypeptide or nucleic acid or fragment thereof is from a human, but can also be an ortholog, or derivative thereof with at least 70% sequence identity, preferably at least 80%, more preferably 85%, still more preferably 90%, and most preferably at least 95% sequence identity with CHD. Normally, orthologs in different species retain the same function, due to presence of one or more protein motifs and/or 3-dimensional structures. Orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95:5849-5856; Huynen M A et al., Genome Research (2000) 10:1204-1210). Programs for multiple sequence alignment, such as CLUSTAL Thompson J D et al, 1994, Nucleic Acids Res 22:4673-4680) may be used to highlight conserved regions and/or residues of orthologous proteins and to generate phylogenetic trees. In a phylogenetic tree representing multiple homologous sequences from diverse species (e.g., retrieved through BLAST analysis), orthologous sequences from two species generally appear closest on the tree with respect to all other sequences from these two species. Structural threading or other analysis of protein folding (e.g., using software by ProCeryon, Biosciences, Salzburg, Austria) may also identify potential orthologs. In evolution, when a gene duplication event follows speciation, a single gene in one species, such as Drosophila, may correspond to multiple genes (paralogs) in another, such as human. As used herein, the term “orthologs” encompasses paralogs. As used herein, “percent (%) sequence identity” with respect to a subject sequence, or a specified portion of a subject sequence, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. (1997) 215:403-410; http://blast.wustl.edu/blast/README.html) with all the search parameters set to default values. The HSP S and HSP S2 parameters are dynamic values and are established by the program itself depending upon the composition of the particular sequence and composition of the particular database against which the sequence of interest is being searched. A % identity value is determined by the number of matching identical nucleotides or amino acids divided by the sequence length for which the percent identity is being reported. “Percent (%) amino acid sequence similarity” is determined by doing the same calculation as for determining % amino acid sequence identity, but including conservative amino acid substitutions in addition to identical amino acids in the computation.

A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids are arginine, lysine and histidine; interchangeable acidic amino acids are aspartic acid and glutamic acid; and interchangeable small amino acids are alanine, serine, threonine, cysteine and glycine.

Alternatively, an alignment for nucleic acid sequences is provided by the local homology algorithm of Smith and Waterman (Smith and Waterman, 1981, Advances in Applied Mathematics 2:482-489; database: European Bioinformatics Institute http://www.ebi.ac.uk/MPsrch/; Smith and Waterman, 1981, J. of Molec. Biol., 147:195-197; Nicholas et al., 1998, “A Tutorial on Searching Sequence Databases and Sequence Scoring Methods” (www.psc.edu) and references cited therein.; W. R. Pearson, 1991, Genomics 11:635-650). This algorithm can be applied to amino acid sequences by using the scoring matrix developed by Dayhoff (Dayhoff: Atlas of Protein Sequences and Structure, M. O. Dayhoff ed., 5 suppl. 3:353-358, National Biomedical Research Foundation, Washington, D.C., USA), and normalized by Gribskov (Gribskov 1986 Nucl. Acids Res. 14(6):6745-6763). The Smith-Waterman algorithm may be employed where default parameters are used for scoring (for example, gap open penalty of 12, gap extension penalty of two). From the data generated, the “Match” value reflects “sequence identity.”

Derivative nucleic acid molecules of the subject nucleic acid molecules include sequences that hybridize to the nucleic acid sequence of any of SEQ ID NOs: 1 through 19. The stringency of hybridization can be controlled by temperature, ionic strength, pH, and the presence of denaturing agents such as formamide during hybridization and washing. Conditions routinely used are set out in readily available procedure texts (e.g., Current Protocol in Molecular Biology, Vol. 1, Chap. 2.10, John Wiley & Sons, Publishers (1994); Sambrook et al., Molecular Cloning, Cold Spring Harbor (1989)). In some embodiments, a nucleic acid molecule of the invention is capable of hybridizing to a nucleic acid molecule containing the nucleotide sequence of any one of SEQ ID NOs: 1 through 19 under stringent hybridization conditions that comprise: prehybridization of filters containing nucleic acid for 8 hours to overnight at 65° C. in a solution comprising 6× single strength citrate (SSC) (1×SSC is 0.15 M NaCl, 0.015 M Na citrate; pH 7.0), 5× Denhardt's solution, 0.05% sodium pyrophosphate and 100 μg/ml herring sperm DNA; hybridization for 18-20 hours at 65° C. in a solution containing 6×SSC, 1× Denhardt's solution, 100 μg/ml yeast tRNA and 0.05% sodium pyrophosphate; and washing of filters at 65° C. for 1h in a solution containing 0.2×SSC and 0.1% SDS (sodium dodecyl sulfate).

In other embodiments, moderately stringent hybridization conditions are used that comprise: pretreatment of filters containing nucleic acid for 6 h at 40° C. in a solution containing 35% formamide, 5×SSC, 50 mM Tris-HCl (pH7.5), 5mM EDTA, 0.1% PVP, 0.1% Ficoll, 1% BSA, and 500 μg/ml denatured salmon sperm DNA; hybridization for 18-20 h at 40° C. in a solution containing 35% formamide, 5×SSC, 50 mM Tris-HCl (pH7.5), 5 mM EDTA, 0.02% PVP, 0.02% Ficoll, 0.2% BSA, 100 μg/ml salmon sperm DNA, and 10% (wt/vol) dextran sulfate; followed by washing twice for 1 hour at 55° C. in a solution containing 2×SSC and 0.1% SDS.

Alternatively, low stringency conditions can be used that comprise: incubation for 8 hours to overnight at 37° C. in a solution comprising 20% formamide, 5×SSC, 50 mM sodium phosphate (pH 7.6), 5× Denhardt's solution, 10% dextran sulfate, and 20 μg/ml denatured sheared salmon sperm DNA; hybridization in the same buffer for 18 to 20 hours; and washing of filters in 1×SSC at about 37° C. for 1 hour.

Isolation, Production, Expression, and Mis-Expression of CHD Nucleic Acids and Polypeptides

CHD nucleic acids and polypeptides, useful for identifying and testing agents that modulate CHD function and for other applications related to the involvement of CHD in the p53 pathway. CHD nucleic acids and derivatives and orthologs thereof may be obtained using any available method. For instance, techniques for isolating cDNA or genomic DNA sequences of interest by screening DNA libraries or by using polymerase chain reaction (PCR) are well known in the art. In general, the particular use for the protein will dictate the particulars of expression, production, and purification methods. For instance, production of proteins for use in screening for modulating agents may require methods that preserve specific biological activities of these proteins, whereas production of proteins for antibody generation may require structural integrity of particular epitopes. Expression of proteins to be purified for screening or antibody production may require the addition of specific tags (e.g., generation of fusion proteins). Overexpression of a CHD protein for assays used to assess CHD function, such as involvement in cell cycle regulation or hypoxic response, may require expression in eukaryotic cell lines capable of these cellular activities. Techniques for the expression, production, and purification of proteins are well known in the art; any suitable means therefore may be used (e.g., Higgins S J and Hames B D (eds.) Protein Expression: A Practical Approach, Oxford University Press Inc., New York 1999; Stanbury P F et al., Principles of Fermentation Technology, 2^{nd} edition, Elsevier Science, New York, 1995; Doonan S (ed.) Protein Purification Protocols, Humana Press, New Jersey, 1996; Coligan J E et al, Current Protocols in Protein Science (eds.), 1999, John Wiley & Sons, New York). In particular embodiments, recombinant CHD is expressed in a cell line known to have defective p53 function (e.g. SAOS-2 osteoblasts, H1299 lung cancer cells, C33A and HT3 cervical cancer cells, HT-29 and DLD-1 colon cancer cells, among others, available from American Type Culture Collection (ATCC), Manassas, Va.). The recombinant cells are used in cell-based screening assay systems of the invention, as described further below.

The nucleotide sequence encoding a CHD polypeptide can be inserted into any appropriate expression vector. The necessary transcriptional and translational signals, including promoter/enhancer element, can derive from the native CHD gene and/or its flanking regions or can be heterologous. A variety of host-vector expression systems may be utilized, such as mammalian cell systems infected with virus (e.g. vaccinia virus, adenovirus, etc.); insect cell systems infected with virus (e.g. baculovirus); microorganisms such as yeast containing yeast vectors, or bacteria transformed with bacteriophage, plasmid, or cosmid DNA. A host cell strain that modulates the expression of, modifies, and/or specifically processes the gene product may be used.

To detect expression of the CHD gene product, the expression vector can comprise a promoter operably linked to a CHD gene nucleic acid, one or more origins of replication, and, one or more selectable markers (e.g. thymidine kinase activity, resistance to antibiotics, etc.). Alternatively, recombinant expression vectors can be identified by assaying for the expression of the CHD gene product based on the physical or functional properties of the CHD protein in in vitro assay systems (e.g. immunoassays).

The CHD protein, fragment, or derivative may be optionally expressed as a fusion, or chimeric protein product (i.e. it is joined via a peptide bond to a heterologous protein sequence of a different protein), for example to facilitate purification or detection. A chimeric product can be made by ligating the appropriate nucleic acid sequences encoding the desired amino acid sequences to each other using standard methods and expressing the chimeric product. A chimeric product may also be made by protein synthetic techniques, e.g. by use of a peptide synthesizer (Hunkapiller et al., Nature (1984) 310:105-111).

Once a recombinant cell that expresses the CHD gene sequence is identified, the gene product can be isolated and purified using standard methods (e.g. ion exchange, affinity, and gel exclusion chromatography; centrifugation; differential solubility; electrophoresis, cite purification reference). Alternatively, native CHD proteins can be purified from natural sources, by standard methods (e.g. immunoaffinity purification). Once a protein is obtained, it may be quantified and its activity measured by appropriate methods, such as immunoassay, bioassay, or other measurements of physical properties, such as crystallography.

The methods of this invention may also use cells that have been engineered for altered expression (mis-expression) of CHD or other genes associated with the p53 pathway. As used herein, mis-expression encompasses ectopic expression, over-expression, under-expression, and non-expression (e.g. by gene knock-out or blocking expression that would otherwise normally occur).

Genetically Modified Animals

Animal models that have been genetically modified to alter CHD expression may be used in in vivo assays to test for activity of a candidate p53 modulating agent, or to further assess the role of CHD in a p53 pathway process such as apoptosis or cell proliferation. Preferably, the altered CHD expression results in a detectable phenotype, such as decreased or increased levels of cell proliferation, angiogenesis, or apoptosis compared to control animals having normal CHD expression. The genetically modified animal may additionally have altered p53 expression (e.g. p53 knockout). Preferred genetically modified animals are mammals such as primates, rodents (preferably mice), cows, horses, goats, sheep, pigs, dogs and cats. Preferred non-mammalian species include zebrafish, C. elegans, and Drosophila. Preferred genetically modified animals are transgenic animals having a heterologous nucleic acid sequence present as an extrachromosomal element in a portion of its cells, i.e. mosaic animals (see, for example, techniques described by Jakobovits, 1994, Curr. Biol. 4:761-763.) or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal.

Methods of making transgenic animals are well-known in the art (for transgenic mice see Brinster et al., Proc. Nat. Acad. Sci. USA 82:4438-4442 (1985), U.S. Pat. Nos. 4,736,866 and 4,870,009, both by Leder et al., U.S. Pat. No. 4,873,191 by Wagner et al., and Hogan, B., Manipulating the Mouse Embryo, Cold Spring Harbor Laboratory Press, Cold Spring Harbor, N.Y., (1986); for particle bombardment see U.S. Pat. No. 4,945,050, by Sandford et al.; for transgenic Drosophila see Rubin and Spradling, Science (1982) 218:348-53 and U.S. Pat. No. 4,670,388; for transgenic insects see Berghammer A. J. et al., A Universal Marker for Transgenic Insects (1999) Nature 402:370-371; for transgenic Zebrafish see Lin S., Transgenic Zebrafish, Methods Mol Biol. (2000);136:375-3830); for microinjection procedures for fish, amphibian eggs and birds see Houdebine and Chourrout, Experientia (1991) 47:897-905; for transgenic rats see Hammer et al., Cell (1990) 63:1099-1112; and for culturing of embryonic stem (ES) cells and the subsequent production of transgenic animals by the introduction of DNA into ES cells using methods such as electroporation, calcium phosphate/DNA precipitation and direct injection see, e.g., Teratocarcinomas and Embryonic Stem Cells, A Practical Approach, E. J. Robertson, ed., IRL Press (1987)). Clones of the nonhuman transgenic animals can be produced according to available methods (see Wilmut, I. et al. (1997) Nature 385:810-813; and PCT International Publication Nos. WO 97/07668 and WO 97/07669).

In one embodiment, the transgenic animal is a “knock-out” animal having a heterozygous or homozygous alteration in the sequence of an endogenous CHD gene that results in a decrease of CHD function, preferably such that CHD expression is undetectable or insignificant. Knock-out animals are typically generated by homologous recombination with a vector comprising a transgene having at least a portion of the gene to be knocked out. Typically a deletion, addition or substitution has been introduced into the transgene to functionally disrupt it. The transgene can be a human gene (e.g., from a human genomic clone) but more preferably is an ortholog of the human gene derived from the transgenic host species. For example, a mouse CHD gene is used to construct a homologous recombination vector suitable for altering an endogenous CHD gene in the mouse genome. Detailed methodologies for homologous recombination in mice are available (see Capecchi, Science (1989) 244:1288-1292; Joyner et al., Nature (1989) 338:153-156). Procedures for the production of non-rodent transgenic mammals and other animals are also available (Houdebine and Chourrout, supra; Pursel et al., Science (1989) 244:1281-1288; Simms et al., Bio/Technology (1988) 6:179-183). In a preferred embodiment, knock-out animals, such as mice harboring a knockout of a specific gene, may be used to produce antibodies against the human counterpart of the gene that has been knocked out (Claesson MH et al., (1994) Scan J Immunol 40:257-264; Declerck PJ et al., (1995) J Biol Chem. 270:8397-400).

In another embodiment, the transgenic animal is a “knock-in” animal having an alteration in its genome that results in altered expression (e.g., increased (including ectopic) or decreased expression) of the CHD gene, e.g., by introduction of additional copies of CHD, or by operatively inserting a regulatory sequence that provides for altered expression of an endogenous copy of the CHD gene. Such regulatory sequences include inducible, tissue-specific, and constitutive promoters and enhancer elements. The knock-in can be homozygous or heterozygous.

Transgenic nonhuman animals can also be produced that contain selected systems allowing for regulated expression of the transgene. One example of such a system that may be produced is the cre/loxP recombinase system of bacteriophage P1 (Lakso et al., PNAS (1992) 89:6232-6236; U.S. Pat. No. 4,959,317). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase. Another example of a recombinase system is the FLP recombinase system of Saccharomyces cerevisiae (O'Gorman et al. (1991) Science 251:1351-1355; U.S. Pat. No. 5,654,182). In a preferred embodiment, both Cre-LoxP and Flp-Frt are used in the same system to regulate expression of the transgene, and for sequential deletion of vector sequences in the same cell (Sun X et al (2000) Nat Genet 25:83-6).

The genetically modified animals can be used in genetic studies to further elucidate the p53 pathway, as animal models of disease and disorders implicating defective p53 function, and for in vivo testing of candidate therapeutic agents, such as those identified in screens described below. The candidate therapeutic agents are administered to a genetically modified animal having altered CHD function and phenotypic changes are compared with appropriate control animals such as genetically modified animals that receive placebo treatment, and/or animals with unaltered CHD expression that receive candidate therapeutic agent.

In addition to the above-described genetically modified animals having altered CHD function, animal models having defective p53 function (and otherwise normal CHD function), can be used in the methods of the present invention. For example, a p53 knockout mouse can be used to assess, in vivo, the activity of a candidate p53 modulating agent identified in one of the in vitro assays described below. p53 knockout mice are described in the literature (Jacks et al., Nature 2001;410:1111-1116, 1043-1044; Donehower et al., supra). Preferably, the candidate p53 modulating agent when administered to a model system with cells defective in p53 function, produces a detectable phenotypic change in the model system indicating that the p53 function is restored, i.e., the cells exhibit normal cell cycle progression.

Modulating Agents

The invention provides methods to identify agents that interact with and/or modulate the function of CHD and/or the p53 pathway. Such agents are useful in a variety of diagnostic and therapeutic applications associated with the p53 pathway, as well as in further analysis of the CHD protein and its contribution to the p53 pathway. Accordingly, the invention also provides methods for modulating the p53 pathway comprising the step of specifically modulating CHD activity by administering a CHD-interacting or -modulating agent.

In a preferred embodiment, CHD-modulating agents inhibit or enhance CHD activity or otherwise affect normal CHD function, including transcription, protein expression, protein localization, and cellular or extra-cellular activity. In a further preferred embodiment, the candidate p53 pathway—modulating agent specifically modulates the function of the CHD. The phrases “specific modulating agent”, “specifically modulates”, etc., are used herein to refer to modulating agents that directly bind to the CHD polypeptide or nucleic acid, and preferably inhibit, enhance, or otherwise alter, the function of the CHD. The term also encompasses modulating agents that alter the interaction of the CHD with a binding partner or substrate (e.g. by binding to a binding partner of a CHD, or to a protein/binding partner complex, and inhibiting function).

Preferred CHD-modulating agents include small molecule compounds; CHD-interacting proteins, including antibodies and other biotherapeutics; and nucleic acid modulators such as antisense and RNA inhibitors. The modulating agents may be formulated in pharmaceutical compositions, for example, as compositions that may comprise other active ingredients, as in combination therapy, and/or suitable carriers or excipients. Techniques for formulation and administration of the compounds may be found in “Remington's Pharmaceutical Sciences” Mack Publishing Co., Easton, Pa., 19^{th} edition.

Small Molecule Modulators

Small molecules, are often preferred to modulate function of proteins with enzymatic function, and/or containing protein interaction domains. Chemical agents, referred to in the art as “small molecule” compounds are typically organic, non-peptide molecules, having a molecular weight less than 10,000, preferably less than 5,000, more preferably less than 1,000, and most preferably less than 500. This class of modulators includes chemically synthesized molecules, for instance, compounds from combinatorial chemical libraries. Synthetic compounds may be rationally designed or identified based on known or inferred properties of the CHD protein or may be identified by screening compound libraries. Alternative appropriate modulators of this class are natural products, particularly secondary metabolites from organisms such as plants or fungi, which can also be identified by screening compound libraries for CHD-modulating activity. Methods for generating and obtaining compounds are well known in the art (Schreiber S L, Science (2000) 151: 1964-1969; Radmann J and Gunther J, Science (2000) 151:1947-1948).

Small molecule modulators identified from screening assays, as described below, can be used as lead compounds from which candidate clinical compounds may be designed, optimized, and synthesized. Such clinical compounds may have utility in treating pathologies associated with the p53 pathway. The activity of candidate small molecule modulating agents may be improved several-fold through iterative secondary functional validation, as further described below, structure determination, and candidate modulator modification and testing. Additionally, candidate clinical compounds are generated with specific regard to clinical and pharmacological properties. For example, the reagents may be derivatized and re-screened using in vitro and in vivo assays to optimize activity and minimize toxicity for pharmaceutical development.

Protein Modulators

Specific CHD-interacting proteins are useful in a variety of diagnostic and therapeutic applications related to the p53 pathway and related disorders, as well as in validation assays for other CHD-modulating agents. In a preferred embodiment, CHD-interacting proteins affect normal CHD function, including transcription, protein expression, protein localization, and cellular or extra-cellular activity. In another embodiment, CHD-interacting proteins are useful in detecting and providing information about the function of CHD proteins, as is relevant to p53 related disorders, such as cancer (e.g., for diagnostic means).

A CHD-interacting protein may be endogenous, i.e. one that naturally interacts genetically or biochemically with a CHD, such as a member of the CHD pathway that modulates CHD expression, localization, and/or activity. CHD-modulators include dominant negative forms of CHD-interacting proteins and of CHD proteins themselves. Yeast two-hybrid and variant screens offer preferred methods for identifying endogenous CHD-interacting proteins (Finley, R. L. et al. (1996) in DNA Cloning-Expression Systems: A Practical Approach, eds. Glover D. & Hames B. D (Oxford University Press, Oxford, England), pp. 169-203; Fashema S F et al., Gene (2000) 250:1-14; Drees B L Curr Opin Chem Biol (1999) 3:64-70; Vidal M and Legrain P Nucleic Acids Res (1999) 27:919-29; and U.S. Pat. No. 5,928,868). Mass spectrometry is an alternative preferred method for the elucidation of protein complexes (reviewed in, e.g., Pandley A and Mann M, Nature (2000) 405:837-846; Yates J R 3^{rd}, Trends Genet (2000) 16:5-8).

A CHD-interacting protein may be an exogenous protein, such as a CHD-specific antibody or a T-cell antigen receptor (see, e.g., Harlow and Lane (1988) Antibodies, A Laboratory Manual, Cold Spring Harbor Laboratory; Harlow and Lane (1999) Using antibodies: a laboratory manual. Cold Spring Harbor, N.Y.: Cold Spring Harbor Laboratory Press). CHD antibodies are further discussed below.

In preferred embodiments, a CHD-interacting protein specifically binds a CHD protein. In alternative preferred embodiments, a CHD-modulating agent binds a CHD substrate, binding partner, or cofactor.

Antibodies

In another embodiment, the protein modulator is a CHD specific antibody agonist or antagonist. The antibodies have therapeutic and diagnostic utilities, and can be used in screening assays to identify CHD modulators. The antibodies can also be used in dissecting the portions of the CHD pathway responsible for various cellular responses and in the general processing and maturation of the CHD.

Antibodies that specifically bind CHD polypeptides can be generated using known methods. Preferably the antibody is specific to a mammalian ortholog of CHD polypeptide, and more preferably, to human CHD. Antibodies may be polyclonal, monoclonal (mAbs), humanized or chimeric antibodies, single chain antibodies, Fab fragments, F(ab′).sub.2 fragments, fragments produced by a FAb expression library, anti-idiotypic (anti-Id) antibodies, and epitope-binding fragments of any of the above. Epitopes of CHD which are particularly antigenic can be selected, for example, by routine screening of CHD polypeptides for antigenicity or by applying a theoretical method for selecting antigenic regions of a protein (Hopp and Wood (1981), Proc. Nati. Acad. Sci. U.S.A. 78:3824-28; Hopp and Wood, (1983) Mol. Immunol. 20:483-89; Sutcliffe et al., (1983) Science 219:660-66) to the amino acid sequence shown in any of SEQ ID NOs:20 through 32. Monoclonal antibodies with affinities of 10⁸ M⁻¹ preferably 10⁹ M⁻¹ to 10¹⁰ M⁻¹, or stronger can be made by standard procedures as described (Harlow and Lane, supra; Goding (1986) Monoclonal Antibodies: Principles and Practice (2d ed) Academic Press, New York; and U.S. Pat. Nos. 4,381,292; 4,451,570; and 4,618,577). Antibodies may be generated against crude cell extracts of CHD or substantially purified fragments thereof. If CHD fragments are used, they preferably comprise at least 10, and more preferably, at least 20 contiguous amino acids of a CHD protein. In a particular embodiment, CHD-specific antigens and/or immunogens are coupled to carrier proteins that stimulate the immune response. For example, the subject polypeptides are covalently coupled to the keyhole limpet hemocyanin (KLH) carrier, and the conjugate is emulsified in Freund's complete adjuvant, which enhances the immune response. An appropriate immune system such as a laboratory rabbit or mouse is immunized according to conventional protocols.

The presence of CHD-specific antibodies is assayed by an appropriate assay such as a solid phase enzyme-linked immunosorbant assay (ELISA) using immobilized corresponding CHD polypeptides. Other assays, such as radioimmunoassays or fluorescent assays might also be used.

Chimeric antibodies specific to CHD polypeptides can be made that contain different portions from different animal species. For instance, a human immunoglobulin constant region may be linked to a variable region of a murine mAb, such that the antibody derives its biological activity from the human antibody, and its binding specificity from the murine fragment. Chimeric antibodies are produced by splicing together genes that encode the appropriate regions from each species (Morrison et al., Proc. Natl. Acad. Sci. (1984) 81:6851-6855; Neuberger et al., Nature (1984) 312:604-608; Takeda et al., Nature (1985) 31:452-454). Humanized antibodies, which are a form of chimeric antibodies, can be generated by grafting complementary-determining regions (CDRs) (Carlos, T. M., J. M. Harlan. 1994. Blood 84:2068-2101) of mouse antibodies into a background of human framework regions and constant regions by recombinant DNA technology (Riechmann L M, et al., 1988 Nature 323: 323-327). Humanized antibodies contain ˜10% murine sequences and ˜90% human sequences, and thus further reduce or eliminate immunogenicity, while retaining the antibody specificities (Co MS, and Queen C. 1991 Nature 351: 501-501; Morrison S L. 1992 Ann. Rev. Immun. 10:239-265). Humanized antibodies and methods of their production are well-known in the art (U.S. Pat. Nos. 5,530,101, 5,585,089, 5,693,762, and 6,180,370).

CHD-specific single chain antibodies which are recombinant, single chain polypeptides formed by linking the heavy and light chain fragments of the Fv regions via an amino acid bridge, can be produced by methods known in the art (U.S. Pat. No. 4,946,778; Bird, Science (1988) 242:423-426; Huston et al., Proc. Natl. Acad. Sci. USA (1988) 85:5879-5883; and Ward et al., Nature (1989) 334:544-546).

Other suitable techniques for antibody production involve in vitro exposure of lymphocytes to the antigenic polypeptides or alternatively to selection of libraries of antibodies in phage or similar vectors (Huse et al., Science (1989) 246:1275-1281). As used herein, T-cell antigen receptors are included within the scope of antibody modulators (Harlow and Lane, 1988, supra).

The polypeptides and antibodies of the present invention may be used with or without modification. Frequently, antibodies will be labeled by joining, either covalently or non-covalently, a substance that provides for a detectable signal, or that is toxic to cells that express the targeted protein (Menard S, et al., Int J. Biol Markers (1989) 4:131-134). A wide variety of labels and conjugation techniques are known and are reported extensively in both the scientific and patent literature. Suitable labels include radionuclides, enzymes, substrates, cofactors, inhibitors, fluorescent moieties, fluorescent emitting lanthanide metals, chemiluminescent moieties, bioluminescent moieties, magnetic particles, and the like (U.S. Pat. Nos. 3,817,837; 3,850,752; 3,939,350; 3,996,345; 4,277,437; 4,275,149; and 4,366,241). Also, recombinant immunoglobulins may be produced (U.S. Pat. No. 4,816,567). Antibodies to cytoplasmic polypeptides may be delivered and reach their targets by conjugation with membrane-penetrating toxin proteins (U.S. Pat. No. 6,086,900).

When used therapeutically in a patient, the antibodies of the subject invention are typically administered parenterally, when possible at the target site, or intravenously. The therapeutically effective dose and dosage regimen is determined by clinical studies. Typically, the amount of antibody administered is in the range of about 0.1 mg/kg-to about 10 mg/kg of patient weight. For parenteral administration, the antibodies are formulated in a unit dosage injectable form (e.g., solution, suspension, emulsion) in association with a pharmaceutically acceptable vehicle. Such vehicles are inherently nontoxic and non-therapeutic. Examples are water, saline, Ringer's solution, dextrose solution, and 5% human serum albumin. Nonaqueous vehicles such as fixed oils, ethyl oleate, or liposome carriers may also be used. The vehicle may contain minor amounts of additives, such as buffers and preservatives, which enhance isotonicity and chemical stability or otherwise enhance therapeutic potential. The antibodies' concentrations in such vehicles are typically in the range of about 1 mg/ml to about 10 mg/ml. Immunotherapeutic methods are further described in the literature (U.S. Pat. No. 5,859,206; WO0073469).

Nucleic Acid Modulators

Other preferred CHD-modulating agents comprise nucleic acid molecules, such as antisense oligomers or double stranded RNA (dsRNA), which generally inhibit CHD activity. Preferred nucleic acid modulators interfere with the function of the CHD nucleic acid such as DNA replication, transcription, translocation of the CHD RNA to the site of protein translation, translation of protein from the CHD RNA, splicing of the CHD RNA to yield one or more mRNA species, or catalytic activity which may be engaged in or facilitated by the CHD RNA.

In one embodiment, the antisense oligomer is an oligonucleotide that is sufficiently complementary to a CHD mRNA to bind to and prevent translation, preferably by binding to the 5′ untranslated region. CHD-specific antisense oligonucleotides, preferably range from at least 6 to about 200 nucleotides. In some embodiments the oligonucleotide is preferably at least 10, 15, or 20 nucleotides in length. In other embodiments, the oligonucleotide is preferably less than 50, 40, or 30 nucleotides in length. The oligonucleotide can be DNA or RNA or a chimeric mixture or derivatives or modified versions thereof, single-stranded or double-stranded. The oligonucleotide can be modified at the base moiety, sugar moiety, or phosphate backbone. The oligonucleotide may include other appending groups such as peptides, agents that facilitate transport across the cell membrane, hybridization-triggered cleavage agents, and intercalating agents.

In another embodiment, the antisense oligomer is a phosphothioate morpholino oligomer (PMO). PMOs are assembled from four different morpholino subunits, each of which contain one of four genetic bases (A, C, G, or T) linked to a six-membered morpholine ring. Polymers of these subunits are joined by non-ionic phosphodiamidate intersubunit linkages. Details of how to make and use PMOs and other antisense oligomers are well known in the art (e.g. see WO99/18193; Probst J C, Antisense Oligodeoxynucleotide and Ribozyme Design, Methods. (2000) 22(3):271-281; Summerton J, and Weller D. 1997 Antisense Nucleic Acid Drug Dev. :7:187-95; U.S. Pat. No. 5,235,033; and U.S. Pat. No. 5,378,841).

Alternative preferred CHD nucleic acid modulators are double-stranded RNA species mediating RNA interference (RNAi). RNAi is the process of sequence-specific, post-transcriptional gene silencing in animals and plants, initiated by double-stranded RNA (dsRNA) that is homologous in sequence to the silenced gene. Methods relating to the use of RNAi to silence genes in C. elegans, Drosophila, plants, and humans are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619; Elbashir S M, et al., 2001 Nature 411:494-498).

Nucleic acid modulators are commonly used as research reagents, diagnostics, and therapeutics. For example, antisense oligonucleotides, which are able to inhibit gene expression with exquisite specificity, are often used to elucidate the function of particular genes (see, for example, U.S. Pat. No. 6,165,790). Nucleic acid modulators are also used, for example, to distinguish between functions of various members of a biological pathway. For example, antisense oligomers have been employed as therapeutic moieties in the treatment of disease states in animals and man and have been demonstrated in numerous clinical trials to be safe and effective (Milligan J F, et al, Current Concepts in Antisense Drug Design, J Med Chem. (1993) 36:1923-1937; Tonkinson J L et al., Antisense Oligodeoxynucleotides as Clinical Therapeutic Agents, Cancer Invest. (1996) 14:54-65). Accordingly, in one aspect of the invention, a CHD-specific nucleic acid modulator is used in an assay to further elucidate the role of the CHD in the p53 pathway, and/or its relationship to other members of the pathway. In another aspect of the invention, a CHD-specific antisense oligomer is used as a therapeutic agent for treatment of p53-related disease states.

Assay Systems

The invention provides assay systems and screening methods for identifying specific modulators of CHD activity. As used herein, an “assay system” encompasses all the components required for performing and analyzing results of an assay that detects and/or measures a particular event. In general, primary assays are used to identify or confirm a modulator's specific biochemical or molecular effect with respect to the CHD nucleic acid or protein. In general, secondary assays further assess the activity of a CHD modulating agent identified by a primary assay and may confirm that the modulating agent affects CHD in a manner relevant to the p53 pathway. In some cases, CHD modulators will be directly tested in a secondary assay.

In a preferred embodiment, the screening method comprises contacting a suitable assay system comprising a CHD polypeptide with a candidate agent under conditions whereby, but for the presence of the agent, the system provides a reference activity (e.g. helicase activity), which is based on the particular molecular event the screening method detects. A statistically significant difference between the agent-biased activity and the reference activity indicates that the candidate agent modulates CHD activity, and hence the p53 pathway.

Primary Assays

The type of modulator tested generally determines the type of primary assay.

Primary Assays for Small Molecule Modulators

For small molecule modulators, screening assays are used to identify candidate modulators. Screening assays may be cell-based or may use a cell-free system that recreates or retains the relevant biochemical reaction of the target protein (reviewed in Sittampalam G S et al., Curr Opin Chem Biol (1997) 1:384-91 and accompanying references). As used herein the term “cell-based” refers to assays using live cells, dead cells, or a particular cellular fraction, such as a membrane, endoplasmic reticulum, or mitochondrial fraction. The term “cell free” encompasses assays using substantially purified protein (either endogenous or recombinantly produced), partially purified or crude cellular extracts. Screening assays may detect a variety of molecular events, including protein-DNA interactions, protein-protein interactions (e.g., receptor-ligand binding), transcriptional activity (e.g., using a reporter gene), enzymatic activity (e.g., via a property of the substrate), activity of second messengers, immunogenicty and changes in cellular morphology or other cellular characteristics. Appropriate screening assays may use a wide range of detection methods including fluorescent, radioactive, calorimetric, spectrophotometric, and amperometric methods, to provide a read-out for the particular molecular event detected.

Cell-based screening assays usually require systems for recombinant expression of CHD and any auxiliary proteins demanded by the particular assay. Appropriate methods for generating recombinant proteins produce sufficient quantities of proteins that retain their relevant biological activities and are of sufficient purity to optimize activity and assure assay reproducibility. Yeast two-hybrid and variant screens, and mass spectrometry provide preferred methods for determining protein-protein interactions and elucidation of protein complexes. In certain applications, when CHD-interacting proteins are used in screens to identify small molecule modulators, the binding specificity of the interacting protein to the CHD protein may be assayed by various known methods such as substrate processing (e.g. ability of the candidate CHD-specific binding agents to function as negative effectors in CHD-expressing cells), binding equilibrium constants (usually at least about 10⁷ M⁻¹, preferably at least about 10⁸ M⁻¹, more preferably at least about 10⁹ M⁻¹), and immunogenicity (e.g. ability to elicit CHD specific antibody in a heterologous host such as a mouse, rat, goat or rabbit). For enzymes and receptors, binding may be assayed by, respectively, substrate and ligand processing.

The screening assay may measure a candidate agent's ability to specifically bind to or modulate activity of a CHD polypeptide, a fusion protein thereof, or to cells or membranes bearing the polypeptide or fusion protein. The CHD polypeptide can be full length or a fragment thereof that retains functional CHD activity. The CHD polypeptide may be fused to another polypeptide, such as a peptide tag for detection or anchoring, or to another tag. The CHD polypeptide is preferably human CHD, or is an ortholog or derivative thereof as described above. In a preferred embodiment, the screening assay detects candidate agent-based modulation of CHD interaction with a binding target, such as an endogenous or exogenous protein or other substrate that has CHD-specific binding activity, and can be used to assess normal CHD gene function.

Suitable assay formats that may be adapted to screen for CHD modulators are known in the art. Preferred screening assays are high throughput or ultra high throughput and thus provide automated, cost-effective means of screening compound libraries for lead compounds (Fernandes P B, Curr Opin Chem Biol (1998) 2:597-603; Sundberg S A, Curr Opin Biotechnol 2000, 11:47-53). In one preferred embodiment, screening assays uses fluorescence technologies, including fluorescence polarization, time-resolved fluorescence, and fluorescence resonance energy transfer. These systems offer means to monitor protein-protein or DNA-protein interactions in which the intensity of the signal emitted from dye-labeled molecules depends upon their interactions with partner molecules (e.g., Selvin P R, Nat Struct Biol (2000) 7:730-4; Fernandes P B, supra; Hertzberg R P and Pope A J, Curr Opin Chem Biol (2000) 4:445-451).

A variety of suitable assay systems may be used to identify candidate CHD and p53 pathway modulators (e.g. U.S. Pat. Nos. 5,550,019 and 6,133,437 (apoptosis assays); U.S. Pat. No. 6,020,135 (p53 modulation), WO 01/25487 (Helicase assays), among others). Specific preferred assays are described in more detail below.

Helicase assays. Helicases are involved in unwinding double stranded DNA and RNA. In one embodiment, an assay for DNA helicase activity detects the displacement of a radio-labeled oligonucleotide from single stranded DNA upon initiation of unwinding (Sivaraja M et al., Anal Biochem (1998) 265:22-27). An assay for RNA helicase activity uses the scintillation proximity (SPA) assay to detect the displacement of a radio-labeled oligonucleotide from single stranded RNA (Kyono K et al., Anal Biochem (1998) 257:120-126).

Apoptosis assays. Assays for apoptosis may be performed by terminal deoxynucleotidyl transferase-mediated digoxigenin-11-dUTP nick end labeling (TUNEL) assay. The TUNEL assay is used to measure nuclear DNA fragmentation characteristic of apoptosis (Lazebnik et al., 1994, Nature 371, 346), by following the incorporation of fluorescein-dUTP (Yonehara et al., 1989, J. Exp. Med. 169, 1747). Apoptosis may further be assayed by acridine orange staining of tissue culture cells (Lucas, R., et al., 1998, Blood 15:4730-41). An apoptosis assay system may comprise a cell that expresses a CHD, and that optionally has defective p53 function (e.g. p53 is over-expressed or under-expressed relative to wild-type cells). A test agent can be added to the apoptosis assay system and changes in induction of apoptosis relative to controls where no test agent is added, identify candidate p53 modulating agents. In some embodiments of the invention, an apoptosis assay may be used as a secondary assay to test a candidate p53 modulating agents that is initially identified using a cell-free assay system. An apoptosis assay may also be used to test whether CHD function plays a direct role in apoptosis. For example, an apoptosis assay may be performed on cells that over- or under-express CHD relative to wild type cells. Differences in apoptotic response compared to wild type cells suggests that the CHD plays a direct role in the apoptotic response. Apoptosis assays are described further in U.S. Pat. No. 6,133,437.

Cell proliferation and cell cycle assays. Cell proliferation may be assayed via bromodeoxyuridine (BRDU) incorporation. This assay identifies a cell population undergoing DNA synthesis by incorporation of BRDU into newly-synthesized DNA. Newly-synthesized DNA may then be detected using an anti-BRDU antibody (Hoshino et al., 1986, Int. J. Cancer 38, 369; Campana et al., 1988, J. Immunol. Meth. 107, 79), or by other means.

Cell Proliferation may also be examined using [³H]-thymidine incorporation (Chen, J., 1996, Oncogene 13:1395-403; Jeoung, J., 1995, J. Biol. Chem. 270:18367-73). This assay allows for quantitative characterization of S-phase DNA syntheses. In this assay, cells synthesizing DNA will incorporate [³H]-thymidine into newly synthesized DNA. Incorporation can then be measured by standard techniques such as by counting of radioisotope in a scintillation counter (e.g., Beckman LS 3800 Liquid Scintillation Counter).

Cell proliferation may also be assayed by colony formation in soft agar (Sambrook et al., Molecular Cloning, Cold Spring Harbor (1989)). For example, cells transformed with CHD are seeded in soft agar plates, and colonies are measured and counted after two weeks incubation.

Involvement of a gene in the cell cycle may be assayed by flow cytometry (Gray J W et al. (1986) Int J Radiat Biol Relat Stud Phys Chem Med 49:237-55). Cells transfected with a CHD may be stained with propidium iodide and evaluated in a flow cytometer (available from Becton Dickinson).

Accordingly, a cell proliferation or cell cycle assay system may comprise a cell that expresses a CHD, and that optionally has defective p53 function (e.g. p53 is over-expressed or under-expressed relative to wild-type cells). A test agent can be added to the assay system and changes in cell proliferation or cell cycle relative to controls where no test agent is added, identify candidate p53 modulating agents. In some embodiments of the invention, the cell proliferation or cell cycle assay may be used as a secondary assay to test a candidate p53 modulating agents that is initially identified using another assay system such as a cell-free assay system. A cell proliferation assay may also be used to test whether CHD function plays a direct role in cell proliferation or cell cycle. For example, a cell proliferation or cell cycle assay may be performed on cells that over- or under-express CHD relative to wild type cells. Differences in proliferation or cell cycle compared to wild type cells suggests that the CHD plays a direct role in cell proliferation or cell cycle.

Angiogenesis. Angiogenesis may be assayed using various human endothelial cell systems, such as umbilical vein, coronary artery, or dermal cells. Suitable assays include Alamar Blue based assays (available from Biosource International) to measure proliferation; migration assays using fluorescent molecules, such as the use of Becton Dickinson Falcon HTS FluoroBlock cell culture inserts to measure migration of cells through membranes in presence or absence of angiogenesis enhancer or suppressors; and tubule formation assays based on the formation of tubular structures by endothelial cells on Matrigel® (Becton Dickinson). Accordingly, an angiogenesis assay system may comprise a cell that expresses a CHD, and that optionally has defective p53 function (e.g. p53 is over-expressed or under-expressed relative to wild-type cells). A test agent can be added to the angiogenesis assay system and changes in angiogenesis relative to controls where no test agent is added, identify candidate p53 modulating agents. In some embodiments of the invention, the angiogenesis assay may be used as a secondary assay to test a candidate p53 modulating agents that is initially identified using another assay system. An angiogenesis assay may also be used to test whether CHD function plays a direct role in cell proliferation. For example, an angiogenesis assay may be performed on cells that over- or under-express CHD relative to wild type cells. Differences in angiogenesis compared to wild type cells suggests that the CHD plays a direct role in angiogenesis.

Hypoxic induction. The alpha subunit of the transcription factor, hypoxia inducible factor-1 (HIF-1), is upregulated in tumor cells following exposure to hypoxia in vitro. Under hypoxic conditions, HIF-1 stimulates the expression of genes known to be important in tumour cell survival, such as those encoding glyolytic enzymes and VEGF. Induction of such genes by hypoxic conditions may be assayed by growing cells transfected with CHD in hypoxic conditions (such as with 0.1% O2, 5% CO2, and balance N2, generated in a Napco 7001 incubator (Precision Scientific)) and normoxic conditions, followed by assessment of gene activity or expression by Taqman®. For example, a hypoxic induction assay system may comprise a cell that expresses a CHD, and that optionally has a mutated p53 (e.g. p53 is over-expressed or under-expressed relative to wild-type cells). A test agent can be added to the hypoxic induction assay system and changes in hypoxic response relative to controls where no test agent is added, identify candidate p53 modulating agents. In some embodiments of the invention, the hypoxic induction assay may be used as a secondary assay to test a candidate p53 modulating agents that is initially identified using another assay system. A hypoxic induction assay may also be used to test whether CHD function plays a direct role in the hypoxic response. For example, a hypoxic induction assay may be performed on cells that over- or under-express CHD relative to wild type cells. Differences in hypoxic response compared to wild type cells suggests that the CHD plays a direct role in hypoxic induction.

Cell adhesion. Cell adhesion assays measure adhesion of cells to purified adhesion proteins, or adhesion of cells to each other, in presence or absence of candidate modulating agents. Cell-protein adhesion assays measure the ability of agents to modulate the adhesion of cells to purified proteins. For example, recombinant proteins are produced, diluted to 2.5g/mL in PBS, and used to coat the wells of a microtiter plate. The wells used for negative control are not coated. Coated wells are then washed, blocked with 1% BSA, and washed again. Compounds are diluted to 2× final test concentration and added to the blocked, coated wells. Cells are then added to the wells, and the unbound cells are washed off. Retained cells are labeled directly on the plate by adding a membrane-permeable fluorescent dye, such as calcein-AM, and the signal is quantified in a fluorescent microplate reader.

Cell-cell adhesion assays measure the ability of agents to modulate binding of cell adhesion proteins with their native ligands. These assays use cells that naturally or recombinantly express the adhesion protein of choice. In an exemplary assay, cells expressing the cell adhesion protein are plated in wells of a multiwell plate. Cells expressing the ligand are labeled with a membrane-permeable fluorescent dye, such as BCECF, and allowed to adhere to the monolayers in the presence of candidate agents. Unbound cells are washed off, and bound cells are detected using a fluorescence plate reader.

High-throughput cell adhesion assays have also been described. In one such assay, small molecule ligands and peptides are bound to the surface of microscope slides using a microarray spotter, intact cells are then contacted with the slides, and unbound cells are washed off. In this assay, not only the binding specificity of the peptides and modulators against cell lines are determined, but also the functional cell signaling of attached cells using immunofluorescence techniques in situ on the microchip is measured (Falsey JR et al., Bioconjug Chem. 2001 May-June; 12(3):346-53).

Primary Assays for Antibody Modulators

For antibody modulators, appropriate primary assays test is a binding assay that tests the antibody's affinity to and specificity for the CHD protein. Methods for testing antibody affinity and specificity are well known in the art (Harlow and Lane, 1988, 1999, supra). The enzyme-linked immunosorbant assay (ELISA) is a preferred method for detecting CHD-specific antibodies; others include FACS assays, radioimmunoassays, and fluorescent assays.

Primary Assays for Nucleic Acid Modulators

For nucleic acid modulators, primary assays may test the ability of the nucleic acid modulator to inhibit or enhance CHD gene expression, preferably mRNA expression. In general, expression analysis comprises comparing CHD expression in like populations of cells (e.g., two pools of cells that endogenously or recombinantly express CHD) in the presence and absence of the nucleic acid modulator. Methods for analyzing mRNA and protein expression are well known in the art. For instance, Northern blotting, slot blotting, ribonuclease protection, quantitative RT-PCR (e.g., using the TaqMan®, PE Applied Biosystems), or microarray analysis may be used to confirm that CHD mRNA expression is reduced in cells treated with the nucleic acid modulator (e.g., Current Protocols in Molecular Biology (1994) Ausubel F M et al., eds., John Wiley & Sons, Inc., chapter 4; Freeman W M et al., Biotechniques (1999) 26:112-125; Kallioniemi O P, Ann Med 2001, 33:142-147; Blohm D H and Guiseppi-Elie, A Curr Opin Biotechnol 2001, 12:41-47). Protein expression may also be monitored. Proteins are most commonly detected with specific antibodies or antisera directed against either the CHD protein or specific peptides. A variety of means including Western blotting, ELISA, or in situ detection, are available (Harlow E and Lane D, 1988 and 1999, supra).

Secondary Assays

Secondary assays may be used to further assess the activity of CHD-modulating agent identified by any of the above methods to confirm that the modulating agent affects CHD in a manner relevant to the p53 pathway. As used herein, CHD-modulating agents encompass candidate clinical compounds or other agents derived from previously identified modulating agent. Secondary assays can also be used to test the activity of a modulating agent on a particular genetic or biochemical pathway or to test the specificity of the modulating agent's interaction with CHD.

Secondary assays generally compare like populations of cells or animals (e.g., two pools of cells or animals that endogenously or recombinantly express CHD) in the presence and absence of the candidate modulator. In general, such assays test whether treatment of cells or animals with a candidate CHD-modulating agent results in changes in the p53 pathway in comparison to untreated (or mock- or placebo-treated) cells or animals. Certain assays use “sensitized genetic backgrounds”, which, as used herein, describe cells or animals engineered for altered expression of genes in the p53 or interacting pathways.

Cell-Based Assays

Cell based assays may use a variety of mammalian cell lines known to have defective p53 function (e.g. SAOS-2 osteoblasts, H1299 lung cancer cells, C33A and HT3 cervical cancer cells, HT-29 and DLD-1 colon cancer cells, among others, available from American Type Culture Collection (ATCC), Manassas, Va.). Cell based assays may detect endogenous p53 pathway activity or may rely on recombinant expression of p53 pathway components. Any of the aforementioned assays may be used in this cell-based format. Candidate modulators are typically added to the cell media but may also be injected into cells or delivered by any other efficacious means.

Animal Assays

A variety of non-human animal models of normal or defective p53 pathway may be used to test candidate CHD modulators. Models for defective p53 pathway typically use genetically modified animals that have been engineered to mis-express (e.g., over-express or lack expression in) genes involved in the p53 pathway. Assays generally require systemic delivery of the candidate modulators, such as by oral administration, injection, etc.

In a preferred embodiment, p53 pathway activity is assessed by monitoring neovascularization and angiogenesis. Animal models with defective and normal p53 are used to test the candidate modulator's affect on CHD in Matrigel® assays. Matrigel® is an extract of basement membrane proteins, and is composed primarily of laminin, collagen IV, and heparin sulfate proteoglycan. It is provided as a sterile liquid at 4° C., but rapidly forms a solid gel at 37° C. Liquid Matrigel® is mixed with various angiogenic agents, such as bFGF and VEGF, or with human tumor cells which over-express the CHD. The mixture is then injected subcutaneously(SC) into female athymic nude mice (Taconic, Germantown, N.Y.) to support an intense vascular response. Mice with Matrigel® pellets may be dosed via oral (PO), intraperitoneal (IP), or intravenous (IV) routes with the candidate modulator. Mice are euthanized 5-12 days post-injection, and the Matrigel® pellet is harvested for hemoglobin analysis (Sigma plasma hemoglobin kit). Hemoglobin content of the gel is found to correlate the degree of neovascularization in the gel.

In another preferred embodiment, the effect of the candidate modulator on CHD is assessed via tumorigenicity assays. In one example, xenograft human tumors are implanted SC into female athymic mice, 6-7 week old, as single cell suspensions either from a pre-existing tumor or from in vitro culture. The tumors which express the CHD endogenously are injected in the flank, 1×10⁵ to 1×10⁷ cells per mouse in a volume of 100 μL using a 27 gauge needle. Mice are then ear tagged and tumors are measured twice weekly. Candidate modulator treatment is initiated on the day the mean tumor weight reaches 100 mg. Candidate modulator is delivered IV, SC, IP, or PO by bolus administration. Depending upon the pharmacokinetics of each unique candidate modulator, dosing can be performed multiple times per day. The tumor weight is assessed by measuring perpendicular diameters with a caliper and calculated by multiplying the measurements of diameters in two dimensions. At the end of the experiment, the excised tumors maybe utilized for biomarker identification or further analyses. For immunohistochemistry staining, xenograft tumors are fixed in 4% paraformaldehyde, 0.1M phosphate, pH 7.2, for 6 hours at 4° C., immersed in 30% sucrose in PBS, and rapidly frozen in isopentane cooled with liquid nitrogen.

Diagnostic and Therapeutic Uses

Specific CHD-modulating agents are useful in a variety of diagnostic and therapeutic applications where disease or disease prognosis is related to defects in the p53 pathway, such as angiogenic, apoptotic, or cell proliferation disorders. Accordingly, the invention also provides methods for modulating the p53 pathway in a cell, preferably a cell pre-determined to have defective p53 function, comprising the step of administering an agent to the cell that specifically modulates CHD activity. Preferably, the modulating agent produces a detectable phenotypic change in the cell indicating that the p53 function is restored, i.e., for example, the cell undergoes normal proliferation or progression through the cell cycle.

The discovery that CHD is implicated in p53 pathway provides for a variety of methods that can be employed for the diagnostic and prognostic evaluation of diseases and disorders involving defects in the p53 pathway and for the identification of subjects having a predisposition to such diseases and disorders.

Various expression analysis methods can be used to diagnose whether CHD expression occurs in a particular sample, including Northern blotting, slot blotting, ribonuclease protection, quantitative RT-PCR, and microarray analysis. (e.g., Current Protocols in Molecular Biology (1994) Ausubel F M et al., eds., John Wiley & Sons, Inc., chapter 4; Freeman W M et al, Biotechniques (1999) 26:112-125; Kallioniemi O P, Ann Med 2001, 33:142-147; Blohm and Guiseppi-Elie, Curr Opin Biotechnol 2001, 12:41-47). Tissues having a disease or disorder implicating defective p53 signaling that express a CHD, are identified as amenable to treatment with a CHD modulating agent. In a preferred application, the p53 defective tissue overexpresses a CHD relative to normal tissue. For example, a Northern blot analysis of mRNA from tumor and normal cell lines, or from tumor and matching normal tissue samples from the same patient, using full or partial CHD cDNA sequences as probes, can determine whether particular tumors express or overexpress CHD. Alternatively, the TaqMan® is used for quantitative RT-PCR analysis of CHD expression in cell lines, normal tissues and tumor samples (PE Applied Biosystems).

Various other diagnostic methods may be performed, for example, utilizing reagents such as the CHD oligonucleotides, and antibodies directed against a CHD, as described above for: (1) the detection of the presence of CHD gene mutations, or the detection of either over- or under-expression of CHD mRNA relative to the non-disorder state; (2) the detection of either an over- or an under-abundance of CHD gene product relative to the non-disorder state; and (3) the detection of perturbations or abnormalities in the signal transduction pathway mediated by CHD.

Thus, in a specific embodiment, the invention is drawn to a method for diagnosing a disease in a patient, the method comprising: a) obtaining a biological sample from the patient; b) contacting the sample with a probe for CHD expression; c) comparing results from step (b) with a control; and d) determining whether step (c) indicates a likelihood of disease. Preferably, the disease is cancer, most preferably a cancer as shown in TABLE 1. The probe may be either DNA or protein, including an antibody.

### EXAMPLES

The following experimental section and examples are offered by way of illustration and not by way of limitation.

I. Drosophila p53 screen

The Drosophila p53 gene was overexpressed specifically in the wing using the vestigial margin quadrant enhancer. Increasing quantities of Drosophila p53 (titrated using different strength transgenic inserts in 1 or 2 copies) caused deterioration of normal wing morphology from mild to strong, with phenotypes including disruption of pattern and polarity of wing hairs, shortening and thickening of wing veins, progressive crumpling of the wing and appearance of dark “death” inclusions in wing blade. In a screen designed to identify enhancers and suppressors of Drosophila p53, homozygous females carrying two copies of p53 were crossed to 5663 males carrying random insertions of a piggyBac transposon (Fraser M et al., Virology (1985) 145:356-361). Progeny containing insertions were compared to non-insertion-bearing sibling progeny for enhancement or suppression of the p53 phenotypes. Sequence information surrounding the piggyBac insertion site was used to identify the modifier genes. Modifiers of the wing phenotype were identified as members of the p53 pathway. kismet was an enhancer of the wing phenotype. Human orthologs of the modifiers are referred to herein as CHD.

BLAST analysis (Altschul et al., supra) was employed to identify Targets from Drosophila modifiers. For example, representative sequences from CHD, GI#s 4557447 (SEQ ID NO:21), 4557449 (SEQ ID NO:23), 4557451 (SEQ ID NO:24), 17442585 (SEQ ID NO:25), 4557453 (SEQ ID NO:27), 2224557 (SEQ ID NO:28), 19421557 (SEQ ID NO:30), 7243213 (SEQ ID NO:31), and 18583434 (SEQ ID NO:32) share 32%, 32%, 45%, 45%, 41%, 27%, 39%, 44%, and 37% amino acid identity, respectively, with the Drosophila kismet.

Various domains, signals, and functional subunits in proteins were analyzed using the PSORT (Nakai K., and Horton P., Trends Biochem Sci, 1999, 24:34-6; Kenta Nakai, Protein sorting signals and prediction of subcellular localization, Adv. Protein Chem. 54, 277-344 (2000)), PFAM (Bateman A., et al., Nucleic Acids Res, 1999, 27:260-2; http://pfam.wustl.edu), SMART (Ponting C P, et al., SMART: identification and annotation of domains from signaling and extracellular protein sequences. Nucleic Acids Res. 1999 Jan. 1; 27(1):229-32), TM-HMM (Erik L. L. Sonnhammer, Gunnar von Heijne, and Anders Krogh: A hidden Markov model for predicting transmembrane helices in protein sequences. In Proc. of Sixth Int. Conf. on Intelligent Systems for Molecular Biology, p 175-182 Ed J. Glasgow, T. Littlejohn, F. Major, R. Lathrop, D. Sankoff, and C. Sensen Menlo Park, Calif.: AAAI Press, 1998), and dust (Remm M, and Sonnhammer E. Classification of transmembrane protein families in the Caenorhabditis elegans genome and identification of human orthologs. Genome Res. 2000 November; 10(11): 1679-89) programs. For example, the SNF2N domains of CHDs from GI#s 4557447 (SEQ ID NO:21), 4557449 (SEQ ID NO:23), 4557451 (SEQ ID NO:24), 17442585 (SEQ ID NO:25), 4557453 (SEQ ID NO:27), 19421557 (SEQ ID NO:30), and 7243213 (SEQ ID NO:31), are located respectively at approximately amino acid residues 484-765, 487-768, 739-1035, 126-422, 729-1025, 462-749, and 209-496 (PFAM 00176). Further, the helicase conserved C-terminal domains of CHDs from GI#s 4557447 (SEQ ID NO:21), 4557449 (SEQ ID NO:23), 4557451 (SEQ ID NO:24), 17442585 (SEQ ID NO:25), 4557453 (SEQ ID NO:27), 19421557 (SEQ ID NO:30), 7243213 (SEQ ID NO:31), and 18583434 (SEQ ID NO:32), are located respectively at approximately amino acid residues 828-902, 831-905, 1100-1174, 487-561, 1090-1164, 821-895, 568-642, and 930167. Still further, the chromatin organization modifier (CHROMO) domain (PFAM00385) of various CHDs are located approximately at amino acids 292-338 and 386429 for GI#4557447 (SEQ ID NO:21), 281-327 and 375-433 for GI#4557449 (SEQ ID NO:23), 628-670 for GI#4557451 (SEQ ID NO:24), 12-54 for GI#17442585 (SEQ ID NO:25), 619-661 for GI#4557453 (SEQ ID NO:27), 37-416 for GI#19421557 (SEQ ID NO:30), and 35-84 and 117-162 for GI#7243213 (SEQ ID NO:31).

II. High-Throughput In Vitro Fluorescence Polarization Assay

Fluorescently-labeled CHD peptide/substrate are added to each well of a 96-well microtiter plate, along with a test agent in a test buffer (10 mM HEPES, 10 mM NaCl, 6 mM magnesium chloride, pH 7.6). Changes in fluorescence polarization, determined by using a Fluorolite FPM-2 Fluorescence Polarization Microtiter System (Dynatech Laboratories, Inc), relative to control values indicates the test compound is a candidate modifier of CHD activity.

III. High-Throughput In Vitro Binding Assay.

³³P-labeled CHD peptide is added in an assay buffer (100 mM KCl, 20 mM HEPES pH 7.6, 1 mM MgCl₂, 1% glycerol, 0.5% NP-40, 50 mM beta-mercaptoethanol, 1 mg/ml BSA, cocktail of protease inhibitors) along with a test agent to the wells of a Neutralite-avidin coated assay plate and incubated at 25° C. for 1 hour. Biotinylated substrate is then added to each well and incubated for 1 hour. Reactions are stopped by washing with PBS, and counted in a scintillation counter. Test agents that cause a difference in activity relative to control without test agent are identified as candidate p53 modulating agents.

IV. Immunoprecipitations and Immunoblotting

For coprecipitation of transfected proteins, 3×10⁶ appropriate recombinant cells containing the CHD proteins are plated on 10-cm dishes and transfected on the following day with expression constructs. The total amount of DNA is kept constant in each transfection by adding empty vector. After 24 h, cells are collected, washed once with phosphate-buffered saline and lysed for 20 min on ice in 1 ml of lysis buffer containing 50 mM Hepes, pH 7.9, 250 mM NaCl, 20 mM -glycerophosphate, 1 mM sodium orthovanadate, 5 mM p-nitrophenyl phosphate, 2 mM dithiothreitol, protease inhibitors (complete, Roche Molecular Biochemicals), and 1% Nonidet P-40. Cellular debris is removed by centrifugation twice at 15,000×g for 15 min. The cell lysate is incubated with 25 μl of M2 beads (Sigma) for 2 h at 4° C. with gentle rocking.

After extensive washing with lysis buffer, proteins bound to the beads are solubilized by boiling in SDS sample buffer, fractionated by SDS-polyacrylamide gel electrophoresis, transferred to polyvinylidene difluoride membrane and blotted with the indicated antibodies. The reactive bands are visualized with horseradish peroxidase coupled to the appropriate secondary antibodies and the enhanced chemiluminescence (ECL) Western blotting detection system (Amersham Pharmacia Biotech).

V. Expression Analysis

All cell lines used in the following experiments are NCI (National Cancer Institute) lines, and are available from ATCC (American Type Culture Collection, Manassas, Va. 20110-2209). Normal and tumor tissues were obtained from Impath, UC Davis, Clontech, Stratagene, and Ambion.

TaqMan analysis was used to assess expression levels of the disclosed genes in various samples.

RNA was extracted from each tissue sample using Qiagen (Valencia, Calif.) RNeasy kits, following manufacturer's protocols, to a final concentration of 50 ng/μl. Single stranded cDNA was then synthesized by reverse transcribing the RNA samples using random hexamers and 500 ng of total RNA per reaction, following protocol 4304965 of Applied Biosystems (Foster City, Calif., http://www.appliedbiosystems.com/).

Primers for expression analysis using TaqMan assay (Applied Biosystems, Foster City, Calif.) were prepared according to the TaqMan protocols, and the following criteria: a) primer pairs were designed to span introns to eliminate genomic contamination, and b) each primer pair produced only one product.

Taqman reactions were carried out following manufacturer's protocols, in 25 μl total volume for 96-well plates and 10 μl total volume for 384-well plates, using 300 nM primer and 250 nM probe, and approximately 25 ng of cDNA. The standard curve for result analysis was prepared using a universal pool of human cDNA samples, which is a mixture of cDNAs from a wide variety of tissues so that the chance that a target will be present in appreciable amounts is good. The raw data were normalized using 18S rRNA (universally expressed in all tissues and cells).

For each expression analysis, tumor tissue samples were compared with matched normal tissues from the same patient. A gene was considered overexpressed in a tumor when the level of expression of the gene was 2 fold or higher in the tumor compared with its matched normal sample. In cases where normal tissue was not available, a universal pool of cDNA samples was used instead. In these cases, a gene was considered overexpressed in a tumor sample when the difference of expression levels between a tumor sample and the average of all normal samples from the same tissue type was greater than 2 times the standard deviation of all normal samples (i.e., Tumor−average(all normal samples)>2×STDEV(all normal samples)).

Results are shown in Table 1. Data presented in bold indicate that greater than 50% of tested tumor samples of the tissue type indicated in row 1 exhibited over expression of the gene listed in column 1, relative to normal samples. Underlined data indicates that between 25% to 49% of tested tumor samples exhibited over expression. A modulator identified by an assay described herein can be further validated for therapeutic effect by administration to a tumor in which the gene is overexpressed. A decrease in tumor growth confirms therapeutic utility of the modulator. Prior to treating a patient with the modulator, the likelihood that the patient will respond to treatment can be diagnosed by obtaining a tumor sample from the patient, and assaying for expression of the gene targeted by the modulator. The expression data for the gene(s) can also be used as a diagnostic marker for disease progression. The assay can be performed by expression analysis as described above, by antibody directed to the gene target, or by any other available detection method.

TABLE 1

| — | . | breast | . | . | colon | . | . | lung | . | . | ovary | |
|---|---|---|---|---|---|---|---|---|---|---|---|---|
| GI#2224556 (SEQ ID NO: 8) | TaqExp_100501 | 0 | 11 | . | 4 | 30 | . | 1 | 13 | . | 1 | 7 |
| GI#11345538 (SEQ ID NO: 8) | TaqExp_100501 | 3 | 11 | . | 4 | 30 | . | 3 | 13 | . | 2 | 7 |
| GI#7243212 (SEQ ID NO: 12) | TaqExp_102401 | 3 | 12 | . | 9 | 30 | . | 4 | 14 | . | 4 | 7 |

1. A method of identifying a candidate p53 pathway modulating agent, said method comprising the steps of:
(a) providing an assay system comprising a purified CHD polypeptide or nucleic acid or a functionally active fragment or derivative thereof;
(b) contacting the assay system with a test agent under conditions whereby, but for the presence of the test agent, the system provides a reference activity; and
(c) detecting a test agent-biased activity of the assay system, wherein a difference between the test agent-biased activity and the reference activity identifies the test agent as a candidate p53 pathway modulating agent.

2. The method of claim 1 wherein the assay system comprises cultured cells that express the CHD polypeptide.

3. The method of claim 2 wherein the cultured cells additionally have defective p53 function.

4. The method of claim 1 wherein the assay system includes a screening assay comprising a CHD polypeptide, and the candidate test agent is a small molecule modulator.

5. The method of claim 4 wherein the assay is a helicase assay.

6. The method of claim 1 wherein the assay system is selected from the group consisting of an apoptosis assay system, a cell proliferation assay system, an angiogenesis assay system, and a hypoxic induction assay system.

7. The method of claim 1 wherein the assay system includes a binding assay comprising a CHD polypeptide and the candidate test agent is an antibody.

8. The method of claim 1 wherein the assay system includes an expression assay comprising a CHD nucleic acid and the candidate test agent is a nucleic acid modulator.

9. The method of claim 8 wherein the nucleic acid modulator is an antisense oligomer.

10. The method of claim 8 wherein the nucleic acid modulator is a PMO.

11. The method of claim 1 additionally comprising:
(d) administering the candidate p53 pathway modulating agent identified in (c) to a model system comprising cells defective in p53 function and, detecting a phenotypic change in the model system that indicates that the p53 function is restored.

12. The method of claim 11 wherein the model system is a mouse model with defective p53 function.

13. A method for modulating a p53 pathway of a cell comprising contacting a cell defective in p53 function with a candidate modulator that specifically binds to a CHD polypeptide comprising an amino acid sequence selected from group consisting of SEQ ID NOs:20, 21, 22, 23, 24, 25, 26, 27, 28, 29, 30, 31, and 32, whereby p53 function is restored.

14. The method of claim 13 wherein the candidate modulator is administered to a vertebrate animal predetermined to have a disease or disorder resulting from a defect in p53 function.

15. The method of claim 13 wherein the candidate modulator is selected from the group consisting of an antibody and a small molecule.

16. The method of claim 1, comprising the additional steps of:
(d) providing a secondary assay system comprising cultured cells or a non-human animal expressing CHD,
(e) contacting the secondary assay system with the test agent of (b) or an agent derived therefrom under conditions whereby, but for the presence of the test agent or agent derived therefrom, the system provides a reference activity; and
(f) detecting an agent-biased activity of the second assay system,
wherein a difference between the agent-biased activity and the reference activity of the second assay system confirms the test agent or agent derived therefrom as a candidate p53 pathway modulating agent,
and wherein the second assay detects an agent-biased change in the p53 pathway.

17. The method of claim 16 wherein the secondary assay system comprises cultured cells.

18. The method of claim 16 wherein the secondary assay system comprises a non-human animal.

19. The method of claim 18 wherein the non-human animal mis-expresses a p53 pathway gene.

20. A method of modulating p53 pathway in a mammalian cell comprising contacting the cell with an agent that specifically binds a CHD polypeptide or nucleic acid.

21. The method of claim 20 wherein the agent is administered to a mammalian animal predetermined to have a pathology associated with the p53 pathway.

22. The method of claim 20 wherein the agent is a small molecule modulator, a nucleic acid modulator, or an antibody.

23. A method for diagnosing a disease in a patient comprising:
(a) obtaining a biological sample from the patient;
(b) contacting the sample with a probe for CHD expression;
(c) comparing results from step (b) with a control;
(d) determining whether step (c) indicates a likelihood of disease.

24. The method of claim 23 wherein said disease is cancer.

25. The method according to claim 24, wherein said cancer is a cancer as shown in Table 1 as having >25% expression level.


SEQUENCE LISTING (32 sequences)

SEQ ID NO: 1 - DNA, 5719 bp, Homo sapiens
tcaaattatagccagtcggatgatgattctgggtcagcttcaggctctggatctggttcgagttctggaagcagtagtgatggaagcagtagccagtcaggtagcagtgactctgactccggatctgaatcaggcagtcagtcagagtctgagtcagacacttcccgagaaaacaaagttcaagcaaaaccaccgaaagttgatggagctgagttttggaaatctagtcctagtattctggccgttcagagatctgcaatcctcaagaagcagcaacagcagcagcagcaacaacaacatcaagcctcatctaatagcggatcagaagaggattcctctagcagtgaagattccgatgactcatcagtgaggtcaaaggaaaaagcataaagatgaagattggcaaatgtctgggtcaggatctccatctcagtctggttcagattcagaatctgaagaagagagagagaaaagcagttgtgatgaaacagaatctgattatgagccaaaaaacaaagtcaaaagcagaaaacctcaaaatagatctaagtcaaaaaatggaaagaagattcttggacaaaaaaagagacagattgattcatctgaggaggatgatgatgaagaagattatgataatgataaaagaagttctcgtcgccaagcaactgttaatgttagctataaggaggatgaagaaatgaaaacagattctgatgacctactggaagtctgtggagaggatgttcctcaacctgaggaagaggaatttgaaaccatagaaagatttatggattgtcggattgggagaaaaggagctactggtgctactacaaccatctatgcagttgaagcagatggtgacccaaatgcaggctttgaaaaaaacaaagaaccaggagagattcagtatttaattaaatggaaaggatggtcccatatccacaacacttgggagacagaagaaaccctcaagcagcagaatgttagaggaatgaaaaaattggataattataagaaaaaagatcaggaaacaaaaagatggttgaaaaatgcctctccagaagatgtggaatattataattgccagcaagaacttacagatgatctacataaacagtatcaaatagtggaacgtataattgctcattccaatcaaaagtcagcagctggttatcctgattattactgcaaatggcagggccttccatactcagagtgcagctgggaagatggagctctcatttccaaaaagtttcaagcatgcattgatgagtattttagcaggaaccaatcaaaaaccactccttttaaagattgcaaagtattaaaacaaaggccaaggtttgtagccctgaagaagcagccatcctatattggaggacatgagggcttagaattaagagattatcaactgaatggtttaaattggcttgctcattcttggtgcaaaggaaatagttgcatactcgctgatgaaatgggccttggaaaaacaatacagacgatctcatttctgaattatttgtttcatgaacatcaattatatggaccttttttattggtagtaccgctctccactcttacttcctggcaaagggaaattcagacttgggcttctcaaatgaatgctgtggtttatttaggtgacattaacagcagaaacatgataagaactcatgaatggacgcatcatcagaccaaacggttaaaatttaatatattgttaacaacttatgaaattttattaaaagataaggcattccttggaggtctaaattgggcatttataggtgttgatgaagcacaccgattaaagaatgatgactcccttctgtataaaactttaatagattttaaatccaatcatcgtctccttatcactggaactcctctacagaattccctcaaagagctctggtctttgctacatttcattatgccagaaaagttttcttcctgggaagattttgaagaagaacatggcaaagggagagaatatggttatgcaagccttcacaaggagcttgagccatttctgttacgccgagttaagaaagatgtggaaaaatctcttcctgccaaggttgagcagattttaagaatggaaatgagtgctttacagaaacaatattacaaatggattttaactaggaattacaaagccctcagcaaaggttccaagggcagtacctcaggctttttgaacattatgatggagctaaagaaatgttgtaaccattgctacctcattaaaccaccagataataatgaattctataataaacaggaggccttacaacacttaattcgtagtagcggaaaattgattcttcttgacaagctattaattcgcctaagagaacgaggcaatcgagttcttattttttcacaaatggtgcggatgttagatatacttgcagaatatttgaaatatcgtcaattcccctttcaaagattagatggatcaataaaaggagaactgaggaaacaagctctagatcattttaatgctgagggatcagaggatttttgctttttgctgtccacaagagctggaggtctagggattaatttagcctctgctgacactgttgttatatttgattccgattggaatccacagaatgatcttcaggcacaggctagagcccatcgaattgggcaaaagaaacaggtgaatatttatcgtctagttacaaagggatcagttgaagaagatattcttgaaagggcgaaaaagaagatggttttagatcatcttgtaattcaaagaatggacacaactgggaagacagtactacatacaggttctgccccatcaagttctactcctttcaataaagaagagttatcagccattttaaagtttggtgctgaagaactttttaaggaacctgaaggagaagaacaagagccccaggaaatggatatagatgaaatcttgaagagagctgaaactcatgaaaatgaaccaggtcctttaactgtaggagatgaattgctttcccagttcaaggttgccaacttctcaaatatggatgaggatgacattgagttggaacctgaaagaaattcaaagaattgggaggaaattattccagaagatcaaagaagacgattagaagaagaagaaagacaaaaggaacttgaagaaatttatatgctcccaagaatgagaaattgtgcaaaacagattagtttcaatggaagtgaagggaggcgcagtagaagtaggagatactctggatctgatagtgattccatctcagaagggaaaaggccaaagaaacgtggaagaccacggactattcctcgggagaatattaaaggatttagtgatgcagaaattaggcggtttatcaagagctataagaaatttggtggtcctctggaaagattagatgcaattgctcgagatgctgagttagttgataagtcagaaacagaccttagacgactgggagaattggtacataatggttgcattaaagcattaaaggatagttcttcaggaacagaacgaacaggtggtagactcggaaaagtgaagggtccaacattccgaatatcaggagtacaggtgaatgccaaactagtcatctcccatgaagaagaattaatacctttgcacaaatccattccttctgatccagaagaaagaaagcagtatactatcccatgccacacaaaggcagctcattttgatatagactggggcaaagaagatgattccaatttgttaattggcatctatgaatatggatatggaagctgggaaatgattaaaatggatcctgacctcagtctaacacacaagattcttccagatgatcccgataaaaaaccacaagcaaaacagttgcagacccgtgcagactacctcatcaaattacttagtagagatcttgcaaaaaaagaagctctttctggtgcgggaagttcaaagaggagaaaagcaagagctaagaagaataaagcaatgaagtctataaaagtgaaagaggaaataaagagtgattcttctcctctgccttcagagaagtctgatgaagatgatgataaattgagtgaatccaagtctgatggtagggaaagatccaagaaatcttcagtgtcagatgctccagttcatatcacggcaagtggtgaaccagttcccatttctgaagaatctgaagagctggatcagaagacattcagcatttgtaaagaaagaatgaggcctgttaaagcagctttgaaacaacttgataggcctgagaaaggcctttcagaaagagaacaactagaacatactagacaatgtttaataaaaattggagaccatatcacagaatgtctaaaagagtatacaaatcctgaacaaattaagcaatggagaaaaaacctgtggatttttgtatctaagtttactgaatttgatgcaagaaaattacataaattatataagcatgctattaaaaaacggcaggagtctcagcaaaacagtgatcaaaacagcaacttgaatcctcacgtgattagaaatccagatgtggaaagattaaaagagaatacaaatcacgatgatagcagcagggacagttattcctctgatagacacttaactcagtaccatgatcatcataaagaccgacatcagggagattcttacaaaaaaagtgattccaggaaaagaccctattcttcttttagtaatggtaaagaccatcgtgattgggatcactacaagcaagacagcagatattacagtgacagagagaaacacagaaaactggatgatcacaggagtagagatcacaggtcaaatttggaaggaagtttaaaagatagatctcattctgatcatcgttctcactcagatcatcggttacattcagaccaccggtcaagttctgaatatacgcaccataaatcttccagggattataggtatcactcagactggcaaatggaccacagagcttccagcagtggccctaggtcaccactagatcagagatcttatggctccagatctccatttgaacattcagttgaacacaaaagtacaccggagcatacctggagtagtcggaaaacataacaaaaactgatacttcgtctttctggacttttcttttagccatatatcataaaccaacacagtaattgccttacatgacttgaaagatataaacagatcttctatcagtagcagtattgttacttctttccaggatgcaaggtctattatcccaacagaagagaaaatattttatatttaaggattatgctgcactgtactacaaaattgtagtacttttttttgttttcttttttaaagaaatggaaaatgtttactattacagggacctcaacactgccctcccatacaggctggataaaactgtttttaagtcagtgattttagactgacctccatttaaattatgtttatatatgaactttactctgacctgtgatcatgtttcaggaaggaatgaaagagagttctttcttaataaagaaaaacactcaaggactttgttcatttccaaagctacttgtttacattgtacactgcgaccaccttgccgcttttcatcacaagcttgaatatttaaattctgtacttatatctgtaaaatagccaggaatttcctgtttgtgatctattatgcctttttacaaaaaaaatggctgtaaattattgtaaatattaaaggaactttcctta

SEQ ID NO: 2 - DNA, 5947 bp, Homo sapiens
tctttagagcaaaaggtgaatcaaaggagagtcccagaaaacctgtgactgttgaagaaaattcatctgtgaatttttatattcaaggagtcagtatttatattcatcttttaaactgggaagatttatattttactttaaaacttcttgataataatttacaatgaatggacacagtgatgaagaaagtgttagaaacagtagtggagaatcaagccagtcggatgatgattctgggtcagcttcaggctctggatctggttcgagttctggaagcagtagtgatggaagcagtagccagtcaggtagcagtgactctgactccggatctgaatcaggcagtcagtcagagtctgagtcagacacttcccgagaaaacaaagttcaagcaaaaccaccgaaagttgatggagctgagttttggaaatctagtcctagtattctggccgttcagagatctgcaatcctcaagaagcagcaacagcagcagcagcaacaacaacatcaagcctcatctaatagcggatcagaagaggattcctctagcagtgaagattccgatgactcatcaagtgaggtcaaaaggaaaaagcataaagatgaagattggcaaatgtctgggtcaggatctccatctcagtctggttcagattcagaatctgaagaagagagagagaaaagcagttgtgatgaaacagaatctgattatgagccaaaaaacaaagtcaaaagcagaaaacctcaaaatagatctaagtcaaaaaatggaaagaagattcttggacaaaaaaagagacagattgattcatctgaggaggatgatgatgaagaagattatgataatgataaaagaagttctcgtcgccaagcaactgttaatgttagctataaggaggatgaagaaatgaaaacagattctgatgacctactggaagtctgtggagaggatgttcctcaacctgaggaagaggaatttgaaaccatagaaagatttatggattgtcggattgggagaaaaggagctactggtgctactacaaccatctatgcagttgaagcagatggtgacccaaatgcaggctttgaaaaaaacaaagaaccaggagagattcagtatttaattaaatggaaaggatggtcccatatccacaacacttgggagacagaagaaaccctcaagcagcagaatgttagaggaatgaaaaaattggataattataagaaaaaagatcaggaaacaaaaagatggttgaaaaatgcctctccagaagatgtggaatattataattgccagcaagaacttacagatgatctacataaacagtatcaaatagtgggacgtataattgctcattccaatcaaaagtcagcagctggttatcctgattattactgcaaatggcagggccttccatactcagagtgcagctgggaagatggagctctcatttccaaaaagtttcaagcatgcattgatgagtattttagcaggaaccaatcaaaaaccactccttttaaagattgcaaagtattaaaacaaaggccaaggtttgtagccctgaagaagcagccatcctatattggaggacatgagggcttagaattaagagattatcaactgaatggtttaaattggcttgctcattcttggtgcaaaggaaatagttgcatactcgctgatgaaatgggccttggaaaaacaatacagacgatctcatttctgaattatttgtttcatgaacatcaattatatggaccttttttattggtagtaccgctctccactcttacttcctggcaaagggaaattcagacttgggcttctcaaatgaatgctgtggtttatttaggtgacattaacagcagaaacatgataagaactcatgaatggacgcatcatcagaccaaacggttaaaatttaatatattgttaacaacttatgaaattttattaaaagataaggcattccttggaggtctaaattgggcatttataggtgttgatgaagcacaccgattaaagaatgatgactcccttctgtataaaactttaatagattttaaatccaatcatcgtctccttatcactggaactcctctacagaattccctcaaagagctctggtctttgctacatttcattatgccagaaaagttttcttcctgggaagattttgaagaagaacatggcaaagggagagaatatggttatgcaagccttcacaaggagcttgagccatttctgttacgccgagttaagaaagatgtggaaaaatctcttcctgccaaggttgagcagattttaagaatggaaatgagtgctttacagaaacaatattacaaatggattttaactaggaattacaaagccctcagcaaaggttccaagggcagtacctcaggctttttgaacattatgatggagctaaagaaatgttgtaaccattgctacctcattaaaccaccagataataatgaattctataataaacaggaggccttacaacacttaattcgtagtagcggaaaattgattcttcttgacaagctattaattcgcctaagagaacgaggcaatcgagttcttattttttcacaaatggtgcggatgttagatatacttgcagaatatttgaaatatcgtcaattcccctttcaaagattagatggatcaataaaaggagaactgaggaaacaagctctagatcattttaatgctgagggatcagaggatttttgctttttgctgtccacaagagctggaggtctagggattaatttagcctctgctgacactgttgttatatttgattccgattggaatccacagaatgatcttcaggcacaggctagagcccatcgaattggccaaaagaaacaggtgaatatttatcgtctagttacaaagggatcagttgaagaagatattcttgaaagggcgaaaaagaagatggttttagatcatcttgtaattcaaagaatggacacaactgggaagacagtactacatacaggttctgccccatcaagttctactcctttcaataaagaagagttatcagccattttaaagtttggtgctgaagaactttttaaggaacctgaaggagaagaacaagagccccaggaaatggatatagatgaaatcttgaagagagctgaaactcatgaaaatgaaccaggtcctttaactgtaggagatgaattgctttcccagttcaaggttgccaacttctcaaatatggatgaggatgacattgagttggaacctgaaagaaattcaaagaattgggaggaaattattccagaagatcaaagaagacgattagaagaagaagaaagacaaaaggaacttgaagaaatttatatgctcccaagaatgagaaattgtgcaaaacagattagtttcaatggaagtgaagggaggcgcagtagaagtaggagatactctggatctgatagtgattccatctcagaagggaaaaggccaaagaaacgtggaagaccacggactattcctcgggagaatattaaaggatttagtgatgcagaaattaggcggtttatcaagagctataagaaatttggtggtcctctggaaagattagatgcaattgctcgagatgctgagttagttgataagtcagaaacagaccttagacgactgggagaattggtacataatggttgcattaaagcattaaaggatagttcttcaggaacagaacgaacaggtggtagactcggaaaagtgaagggtccaacattccgaatatcaggagtacaggtgaatgccaaactagtcatctcccatgaagaagaattaatacctttgcacaaatccattccttctgatccagaagaaagaaagcagtatactatcccatgccacacaaaggcagctcattttgatatagactggggcaaagaagatgattccaatttgttaattggcatctatgaatatggatatggaagctgggaaatgattaaaatggatcctgacctcagtctaacacacaagattcttccagatgatcccgataaaaaaccacaagcaaaacagttgcagacccgtgcagactacctcatcaaattacttagtagagatcttgcaaaaaaagaagctctttctggtgcgggaagttcaaagaggagaaaagcaagagctaagaagaataaagcaatgaagtctataaaagtgaaagaggaaataaagagtgattcttctcctctgccttcagagaagtctgatgaagatgatgataaattgagtgaatccaagtctgatggtagggaaagatccaagaaatcttcagtgtcagatgctccagttcatatcacggcaagtggtgaaccagttcccatttctgaagaatctgaagagctggatcagaagacattcagcatttgtaaagaaagaatgaggcctgttaaagcagctttgaaacaacttgataggcctgagaaaggcctttcagaaagagaacaactagaacatactagacaatgtttaataaaaattggagaccatatcacagaatgtctaaaagagtatacaaatcctgaacaaattaagcaatggagaaaaaacctgtggatttttgtatctaagtttactgaatttgatgcaagaaaattacataaattatataagcatgctattaaaaaacggcaggagtctcagcaaaacagtgatcaaaacagcaacttgaatcctcacgtgattagaaatccagatgtggaaagattaaaagagaatacaaatcacgatgatagcagcagggacagttattcctctgatagacacttaactcagtaccatgatcatcataaagaccgacatcagggagattcttacaaaaaaagtgattccaggaaaagaccctattcttcttttagtaatggtaaagaccatcgtgattgggatcactacaagcaagacagcagatattacagtgacagagagaaacacagaaaactggatgatcacaggagtagagatcacaggtcaaatttggaaggaagtttaaaagatagatctcattctgatcatcgttctcactcagatcatcggttacattcagaccaccggtcaagttctgaatatacgcaccataaatcttccagggattataggtatcactcagactggcaaatggaccacagagcttccagcagtggccctaggtcaccactagatcagagatcttatggctccagatctccatttgaacattcagttgaacacaaaagtacaccggagcatacctggagtagtcggaaaacataacaaaaactgatacttcgtctttctggacttttcttttagccatatatcataaaccaacacagtaattgccttacatgacttgaaagatataaacagatcttctatcagtagcagtattgttacttctttccaggatgcaaggtctattatcccaacagaagagaaaatatttttatatttaaggattatgctgcactgtactacaaaattgtagtacttttttttgttttcttttttaaagaaatggaaaatgtttactattacagggacctcaacactgccctcccatacaggctggataaaactgtttttaagtcagtgattttagactgacctccatttaaattatgtttatatatgaactttactctgacctgtgatcatgtttcaggaaggaatgaaagagagttctttcttaataaagaaaaacactcaaggactttgttcatttccaaagctacttgtttacattgtacactgcgaccaccttgccgcttttcatcacaagcttgaatatttaaattctgtacttatatctgtaaaatagccaggaatttcctgtttgtgatctattatgcctttttacaaaaaaaatggctgtaaattattgtaaatattaaaggaactttccttacaaaaaaaaaaaaaaaaaaa

SEQ ID NO: 3 - DNA, 7764 bp, Homo sapiens
ccctgggatggaggatctgtctctctctctctctctcctttttttttttttggtggagatgaaggggtgggtctatggtacatcacctgagttgtggggtaaatgtagagagtgtcaatcaaaggcagagctctcagagctgggaaggaggctctagatggcggctgtgccttagagagagcgcgctctgctccctgcctttgcctcactttacgcaactttccctaactttcgggcagcctcagggggcccccgtagccccctgcctttcctagggacttactggggtcgattcgaacctttttttgggagaaaagcagcttttaggagctttcttttcgtgccttgttggaaagaagcagccgtactgagagcccaggtcgttgttttttccagcttagaagccatggcgcacctccatttttgtgcgctctcctaatgaggttttttttctttcggacctgttttagtattaattattgctttatttttttgaccagttaacatatttgagggttattttatttatttttcgttttttaacggaggattttgcctttatttttaattatttgggatctgatatttttctactagtagataggactcttggtttggacatactacatggatcagtaaatacctgggcacaggacttcaaagcaaacacagattccccctcccccttaatatttaagaattaaaagatgatgagaaataaggacaaaagccaagaggaggacagttcgctacacagcaatgcatcgagtcactcagcctctgaagaagcttcgggttcagactcaggcagtcagtcggaaagtgagcagggaagtgatccaggaagtggacatggcagcgagtcgaacagcagctctgaatcttctgagagtcagtcggaatctgagagcgaatcagcaggttccaaatcccagccagtcctcccagaagccaaagagaagccagcctctaagaaggaacggatagctgatgtgaagaagatgtgggaagaatatcctgatgtttatggggtcaggcggtcaaaccgaagcagacaagaaccatcgcgatttaatattaaggaagaggcaagtagcgggtctgagagtgggagcccaaaaagaagaggccagaggcagctgaaaaaacaagaaaaatggaaacaggaaccctcagaagatgaacaggaacaaggcaccagtgcagagagtgagccagaacaaaaaaaagtaaaagccagaagacctgtccccagaagaacagtgcccaaacctcgtgttaaaaagcagccgaagactcagcgtggaaagagaaaaaagcaagattcttctgatgaggatgatgatgatgacgaagctcccaaaaggcagactcgtcgaagagcggctaaaaacgttagttacaaagaagatgatgactttgagactgactcagatgatctcattgaaatgactggagaaggagttgatgaacagcaagataatagtgaaactattgaaaaggtcttagattcaagactgggaaagaaaggagccactggagcatctactactgtatatgcgattgaagctaatggcgaccctagtggtgactttgacactgaaaaggatgaaggtgaaatccagtacctcatcaagtggaagggttggtcttacatccacagcacatgggagagtgaagaatccttacagcaacagaaagtgaagggcctaaaaaaactagagaacttcaagaaaaaagaggacgaaatcaaacaatggttagggaaagtttctcctgaagatgtagaatatttcaattgccaacaggagctggcttcagagttgaataaacagtatcagatagtagaaagagtaatagctgtgaagacaagtaaatctacattgggtcaaacagattttccagctcatagtcggaagccggcaccctcaaatgagcccgaatatctatgtaaatggatgggactcccctattcagagtgtagctgggaagatgaagccctcattggaaagaaattccagaattgcattgacagcttccacagtaggaacaactcaaaaaccatcccaacaagagaatgcaaggccctgaagcagagaccacgatttgtagctttaaagaaacaacctgcatatttaggaggggagaatctggaacttcgagattatcagctagaaggtctaaactggctagctcattcctggtgcaaaaataatagtgtaatccttgctgatgaaatgggcctaggaaagaccatccagaccatatcattcctctcctacctgttccaccaacaccagctgtatggcccctttcttatagtcgtccctttatccaccctcacctcatggcagagagagtttgaaatctgggcaccagagattaacgtagtggtttacataggtgacctgatgagcagaaatacgatacgggaatatgaatggattcattcccaaaccaaaagattgaagttcaacgcacttataacaacatatgagatcctcttgaaagataagactgtgctgggcagtattaactgggcctttctgggagtggatgaagcccatcggttgaagaatgatgactctttattgtataaaactctgattgatttcaagtccaaccataggctcctgattacggggacccctcttcagaattccctcaaagagctctggtccttgctgcactttattatgccggagaagtttgaattttgggaagattttgaagaagaccatgggaaggggagagagaatggctaccagagtcttcataaggtgctagagcctttccttctccggagagtcaaaaaagatgtggagaaatcccttcctgctaaagtggaacagattctcagggtggagatgtcagcccttcagaaacagtattacaagtggattctgaccaggaattacaaggctcttgccaaaggaacaagaggcagcacatctggttttcttaatattgtgatggaactgaaaaaatgttgcaaccactgctatctgattaaaccccctgaagaaaatgaaagggaaaatggacaggagattcttctgtccctcataaggagcagtgggaagttgattttattagacaaactgttgacaagacttcgagaaagggggaatcgagtgcttatcttctctcagatggtgagaatgttggatatcctggctgaatacctaactattaaacactatcctttccagcgtctggatggttccatcaagggagaaatccgaaaacaggcactggaccacttcaatgcagatgggtctgaggacttctgtttcctgctctcgacaagggctggtggcctgggaatcaatttggcttcagcggacacagtcgtcatctttgactctgactggaacccccagaatgacttgcaggcacaagcccgagcgcatagaattggtcagaagaagcaggtaaatatttaccgcttagttacaaaggggactgtggaggaggagatcatagaacgggccaaaaagaagatggtattagatcatctggtgattcagcgcatggacaccactggccggacgatcctggaaaacaactcaggaaggtccaactcaaatccttttaataaagaagagctgacagctattttgaaatttggagcagaggatctcttcaaagaactggaaggggaggaatcagaacctcaggaaatggatatagatgaaattttgcggttggctgaaacgagagagaatgaagtgtcaacaagtgcaacagatgaacttctatcacagtttaaggttgccaactttgcaacaatggaagatgaagaagagctagaagagcgtcctcacaaggactgggatgagatcattccagaggaacaaaggaaaaaagtagaggaggaagagcggcagaaggagctagaagaaatttatatgctgcctcgaattcggagttccactaaaaaggctcagacaaatgacagtgactctgacactgagtctaagaggcaggcccagagatcctctgcttctgagagtgaaacggaagactctgatgatgacaagaagccaaagcgcagagggcgtccgaggagtgtgcggaaggacctcgtggagggatttactgatgcagagatccgaaggttcatcaaggcttataagaagtttggtctccctcttgaacggctggagtgcttagcacgtgatgctgagctggtagataagtcggtggcagatctgaagcgcctgggtgaactgatccacaacagctgtgtgtcagcaatgcaggaatatgaagagcagctgaaagaaaatgccagcgagggaaaaggaccagggaaaaggagaggtccaacaatcaagatatccggagttcaggttaatgtgaaatccattatccaacatgaagaggagtttgagatgctgcataaatctatccctgtggaccctgaagaaaaaaaaaaatactgcttaacctgtcgtgtcaaagctgcacattttgatgtagagtggggggtggaagatgattctcgcctgttgctggggatttatgaacatggctatggaaactgggagttaattaaaacagacccagagcttaaattaactgacaaaattctgccggtggagacagataaaaagcctcaggggaagcagctacagacccgagcggattacttgttgaagctgctcagaaagggtctggagaagaagggggctgtgacaggtggggaggaggccaaattaaagaagcggaagcctcgggtaaagaaggaaaacaaagtgcccaggctgaaagaggagcatggaattgagctttcatctcctaggcattcagataatccatcagaagagggagaagtgaaagatgatggcttggaaaaaagtccaatgaaaaaaaaacagaagaagaaagagaacaaggagaacaaggagaaacaaatgagttctaggaaagacaaagaaggggacaaggaaagaaagaagtcaaaagataagaaagagaagcctaaaagtggtgatgccaaatcttcgagtaaatcaaagcgatctcagggtcctgtccatattacagcaggaagtgaacctgtccccattggagaggatgaggatgatgatctggaccaggagacattcagcatatgtaaggagaggatgaggcccgtgaaaaaggcactgaaacagctcgacaaacctgacaaggggctcaacgtgcaagaacagctggaacacacccggaactgcctgctgaaaatcggagaccggatagccgagtgccttaaagcctactcagatcaggagcacatcaaactctggaggaggaacctatggatttttgtttccaagtttacagaatttgatgctcgaaaactgcataagttatacaagatggctcataagaaaaggtctcaagaagaagaggagcaaaagaagaaagacgacgtgactgggggtaagaaaccatttcgtccagaggcctcaggctccagccgggactctctgatatctcagtcccatacctcacacaaccttcaccctcagaagcctcatttgcctgcctcccatggcccacagatgcatggacacccaagagataactacaatcaccccaacaagagacacttcagtaatgcagatcgaggagactggcagagggaaagaaagttcaactatggtggtggcaacaacaatccaccatggggaagcgacaggcaccatcagtatgagcagcactggtacaaggaccaccattatggggaccggcgacatatggatgcccaccgttccggaagctatcgacccaacaacatgtccagaaagaggccttatgaccagtacagcagtgaccgagaccaccggggacacagagattattatgacaggtatgcaaaaggctgtgagacaccaggtgccaacctttgccaggagctgtttctagggagaaagtgacgtatacatgaatgtatttatctatcaaattactgaagatctcatcatgcatgtgtcagccacagcgaatcccatgtcttggttataggttttatgttttgttttctgggtcatagggagcacatttcacctgtgcaggaaaagagttttctgccgtcttttgaggaaatctagtgaagaggtcgccataaaatattagagtcaacaaccaaaattattaagctctgtgcgaggctgtcagccacactaggtatcagggatcccgagatgggtaccagcccacagtccttacctgccacgagcccataattgaagagtcaaagtcttctgaagctgcaccctctttacttcagtacaatgccaccagtagtacgatgagccaaagctttacattgtgagagtagcaagtccagggagagctaaagaggttttatctgtatttcctaatttcaaatcttggataatttaacctcatagcagctttggttttccctgggctgatgatgtgcgtcatttgcactgtaccttgaatttacagtgggaaaatttcatataaacgtgtcaaagtcgtgctttgtttttggaagatctggtaacagcagcccgcattagcagagagctgtagctgagtagctgccacctcgttgggagactgcccctcgctcccacccttctctattgtctggacccagtgggcatcttgccctgcgttcttctagtaggtctgtatttctatttgatgtcactttccttttgcctgaaggactttttctgctggtgataaactctttcagtgtttgtatatatgcctgaaaaagtattttgccttcatttttgaaagtagtttttgctgagtgtatacatttttggctttacagtttctttcagtgctttaaagatgtacctctgctatttacttgcattgttttgtgatgaaaaatctgtcatccttatctttgttcctctttacataatgttccttttaaaaaaaatcactgattatgatgtgccttggtgtatttttccttggtttcttgtgcttggaaatttttgaacttcttggatctgtgggtttattgtttccataaaatttggaaatttttacaatcttcttcaaatattttttctgatcccccactctctcttcttctttggagattctcattacacctatattagcttgcttgaagttgtctcacagctcacttgtattctgttgacttttaaaaaattatgctttctgtttcactgtggatagtttctattgctacctcttcaagttcactaatactttccttttcaatgtcaagactgctgtgaggcccatccagtgtactttgcattttatacattgtagttctaaaagttcggaaagttgtttttgggtctttttatatatgttctgtgtctaaccttttaaaacctggaacacagatataacaatggttttgatgtccttgtctgcgaatcttatcacttgggtcagtttcagttgatacctcctcactgtgggtcttgctcccctggtgctttctgtgcctagtaatttttgtcagatgccagatgtaacatttaccttgttgggtgctggatatttctgtattcctgtaagtattctggagctttgttatgagttgcaggttatttggaagcagtttcctttttcaggtcttgctgttaagattcgttaggtagaaccagagcagtgctcagtcaagggctaatgattgcccacccccaaggtaaagagcctcattgcactctacccaattgcgttagtctgttttgcaggaatacctgaggctgggtaatttatagagaaaagagttttatttgg

SEQ ID NO: 4 - DNA, 6331 bp, Homo sapiens
atctttgtttgggtctcccatactgcgtatagatgaatgggtcaggatatctggaacaaaatatggaggtgaagggtgagatcgggaaacaaagggtatggccccctagttcccaaagggagcagggagatgggaatagaattgaaggtaggttttaggctacttgggaggaggaatatttaggtaattgtggagactttctcctgtgtgatgaaggcggcagacactgtgatcctgtgggcaagaagtaaaaatgaccagctgaggatttcttttcctccaggactgtgttggggtgacaggatgcctgataaggatgacattcggctgctgccgtcagcattgggtgtgaagaagagaaaacgaggacccaagaagcagaaggagaacaagccaggaaaaccccgaaaacgcaagaagcgtgacagtgaggaggaatttggttctgagcgagatgagtaccgggagaagtcagagagtgggggcagtgaatatggaaccggaccgggtcggaaacgaagaaggaagcaccgagaaaaaaaggagaagaagacaaagcggcggaaaaagggggagggagatggggggcaaaagcaagtggaacagaagtcatcagcaactctgcttctgacctggggcctggaggatgtggagcatgtgttctctgaggaggattaccacacgctcaccaactacaaagccttcagccagttcatgaggcccctaattgctaagaagaatcctaagatcccaatgtctaagatgatgaccatccttggggccaaatggagagagttcagtgccaacaaccccttcaaggggtcagcagctgctgtggcggcggcagcggcagcagcagcagcagctgtagctgagcaggtgtcagctgctgtctcgtcggccacccccatagcaccctccggaccccccgcccttccaccaccccctgctgctgatatccagcccccacccatccgaagagccaaaaccaaagagggcaaaggtccaggccataagaggcggagtaagagcccccgagtgcctgatggacgcaagaagcttcggggaaagaaaatggcaccactcaaaataaaactagggcttctgggtggcaagaggaagaaaggaggctcgtatgtttttcagagcgacgaaggtcctgaaccagaggctgaggaatcagacctggacagtggcagtgtccacagtgcctcaggccggcctgatggccctgtccgcaccaagaaactaaagagaggccggccaggaaggaagaagaagaaggtcctgggctgtcctgcagtggccggggaggaggaggttgatggctacgagacggatcaccaggattactgtgaggtgtgccagcagggtggggaaattattctgtgtgacacctgccctcgtgcctaccacctcgtctgccttgatcctgagcttgaccgggctccagagggcaaatggagctgccctcactgtgagaaggagggggtccagtgggaggccaaggaggaagaagaagaatacgaagaggagggagaggaagaaggggagaaggaggaggaggatgatcacatggagtactgccgcgtatgcaaggacggcggggagctcctgtgctgtgacgcgtgcatctcctcctaccacattcattgtctaaaccctcccctgcctgacattcccaatggtgaatggctgtgtccccgatgcacatgccccgtgctgaagggtcgagtgcagaagatcctacattggcggtggggggagccacctgtagcagtgccagcccctcaacaggcagatggaaatccagatgtcccacccccccgtcctcttcaaggcagatcagagcgagagttctttgtcaagtgggtaggactatcctactggcactgctcctgggccaaggagcttcagctggaaatcttccatttggttatgtatcgaaactaccagcggaagaatgacatggatgagcccccacccctggactatggctccggcgaggatgatgggaagagcgacaagcgtaaagtgaaagacccgcactatgctgagatggaggagaagtactatcgttttggcatcaagccagagtggatgaccgtccaccgcatcatcaaccacagtgtggataaaaaggggaattaccactatctagtaaaatggagggacttaccatatgaccagtccacgtgggaggaagatgaaatgaatatccctgaatacgaagaacataagcaaagctactggagacaccgagaactaattatgggggaagaccctgcccagccccgcaagtataagaagaagaagaaggagctacagggtgatgggcctcccagttctcccactaatgatcctaccgtgaaatatgagactcagccacggtttatcacagccactggaggcaccctgcacatgtatcagttggaagggctgaactggctacgcttctcctgggcccagggcactgacaccattctagctgatgagatggggctaggcaagaccatacaaaccatcgtcttcctctactcactctacaaggagggccacacaaaaggtcccttcctggtgagtgccccactctctaccatcattaactgggagcgggagttccagatgtgggcacccaaattctatgtggtgacatacacgggtgacaaggacagccgggccatcattcgtgagaatgaattctcctttgaggacaatgccatcaaagggggcaagaaagcttttaagatgaaaagggaggcacaggtgaagttccatgttctcctgacatcgtatgagctgatcaccattgatcaggcagcacttggttccatccgctgggcctgtcttgtggtagatgaggcccatcgactcaagaacaaccagtccaagtttttcagggttctcaatggttacaagatagatcataagttgctgctgacaggaaccccattgcagaataatctggaggagctcttccatctcctgaacttcctcaccccagagagatttaacaacttggagggcttcctggaggagtttgctgacatatccaaagaggaccagatcaagaaactgcatgatttgctggggccacacatgctgcggagactcaaggcagatgtctttaagaacatgccagccaagacagagctcatcgttcgggtggagctaagccccatgcagaagaaatactacaaatacatcctgactcgaaattttgaggccttgaattcacgaggtggtgggaaccaggtgtcgctgcttaatatcatgatggatcttaagaagtgctgcaaccatccatacctttttcccgtggctgctatggagtcccccaaactccccagtggggcttatgagggtggggcacttattaagtcgtctgggaagctcatgctgctccagaagatgctgcgaaagctgaaggagcaaggacaccgagtgctcatcttctcgcagatgaccaaaatgttagacttgcttgaggacttcttagactatgaaggctacaagtatgagcgcatcgatggtggtatcacgggtgccctgaggcaggaggccatcgatcggtttaatgctcctggggcccaacaattctgcttcctcctgtccacccgagctgggggcctgggcatcaatctggccactgctgacactgtcatcatctttgattctgactggaacccccataatgacatccaggcctttagccgggctcatcggattggccaggccaacaaagtgatgatttaccggtttgtgactcgcgcgtcagtggaagagcgaatcacacaagtggccaagagaaagatgatgctgacacacctggttgtgcggcctgggctgggctccaaggcaggctccatgtccaagcaggagcttgacgacattctcaaatttggcactgaagagctattcaaggatgaaaacgagggggagaacaaggaggaggacagcagtgtgattcattatgacaatgaggccatcgctcggctgttggaccggaaccaggatgcaactgaggacactgacgtgcagaacatgaatgagtatctcagctccttcaaggtggcacagtacgtcgtgcgggaagaagacaagattgaggaaattgagcgagagatcatcaagcaggaggagaatgtggaccctgactactgggagaagctgctgaggcatcactatgagcaacagcaggaagacctagcccggaatctaggcaagggcaagcgggttcgcaagcaagttaactacaatgatgctgctcaggaagaccaagacaaccagtcagagtactcggtgggttcagaggaggaggatgaagacttcgatgaacgtcctgaagggcgtagacagtcaaagaggcagctccggaatgagaaagataagccactgcctccactgctggcccgagtcgggggcaacattgaggtgctgggcttcaacacccgtcagcggaaggctttcctcaatgctgtgatgcgctgggggatgccaccacaggatgccttcaccacacagtggctggtgcgggacctgaggggcaagactgagaaggagtttaaggcctatgtgtctttgttcatgcgccatctgtgtgagcctggggcagacggctctgaaacctttgccgatggggtccctcgggagggactgagtcgccagcaggtgttgacccgcattggagtcatgtctctcgtcaaaaagaaggtgcaggagtttgagcacatcaatgggcgttggtcaatgccggaactgatgcctgaccccagcgccgattctaagcgctcctccagagcctcctctcctaccaaaacgtctcccaccactcctgaggcttctgctaccaacagtccctgcacctctaaacctgctactccagctccaagtgagaaaggagaaggcataaggacacctcttgagaaggaggaagctgaaaaccaggaggaaaagccagagaagaacagcagaattggggagaagatggagacagaggctgatgcccccagcccagccccatcacttggggagcggctggagccaaggaagattcctctagaggatgaggtgccaggggtgcctggagagatggagcctgaacctgggtaccgtggggacagagagaagtcagaagatgtaaaaggtgaccgggagcttcgaccagggcctcgagatgagccacggtccaatgggcgacgagaggaaaagacagagaagccccggttcatgttcaatatcgccgatggtggcttcacagagcttcacacactgtggcagaatgaggaacgggcagctatttcctcggggaaactcaatgagatctggcacagaagacatgactattggcttctggctgggattgtcctccatggctatgcacggtggcaggacatccagaatgatgctcaatttgccattatcaacgagccatttaaaactgaagccaataaggggaactttctggagatgaaaaataagttcctggcccggaggttcaagctcctggagcaggcgctggtgattgaggagcagctgcggcgggcggcctacctgaacctgtcgcaggagccggcgcaccccgccatggccctccacgcccgcttcgccgaggccgagtgcctggccgagagccaccagcacctctccaaggagtcgctggcggggaacaagccggccaacgccgtcctgcacaaggttctgaaccagctggaggagttgctgagcgacatgaaggcggacgtgacccgcctgccagccacgctgtcccgaataccccccatcgcagcccgccttcagatgtccgagcgcagcatcctcagccggctggccagcaagggcacggagcctcaccccacaccggcctacccgccgggtccctacgctacacctccggggtacggggcggccttcagcgccgcacccgtaggggccctggccgccgcaggcgccaattacagccagatgcctgcagggtccttcatcacagccgccaccaacggcccaattcaccgattttttaaaaaagttccagaaatccagtgacgaatgtggtatacaaaaaaatatataaattctttcaacttagaataattaagtcataaaatacatagggtacaaataccacattccgttctaaaatgatatcttaggatcatcaaaagaaaaagaggatttggattatgcaaaaaatgattcctatatatataatcaattatctaactgacatttttgcaaatctaccacaacttcgccttttattgcatatgctaaacaagcagatgctaagtctgtaaactgtgaattaacctcctttttaattaattgttcg

SEQ ID NO: 5 - DNA, 7849 bp, Homo sapiens
gaagaggaagaacaaggaccccctctatgccaagatggaggagcgcttctaccgctatggcatcaagccagagtggatgatgattcaccgaatcctgaaccatagctttgacaagaagggggatgtgcactacctgatcaagtggaaagacctgccctacgaccagtgcacctgggagatcgatgacatcgacatcccctactacgacaacctcaagcaggcctactggggccacagggagctgatgctgggagaagacaccaggctgcccaagaggctgctcaagaagggcaagaagctgagggacgacaagcaggagaagccgccggacacgcccattgtggaccccacggtcaagttcgacaagcagccatggtacatcgactccacaggcggcacactgcacccgtaccagctggagggcctcaactggctgcgcttctcttgggcccagggcactgacaccatcctggccgatgagatgggtctgggcaagacggtgcagaccatcgtgttcctttactccctctacaaggagggccactccaaagggccctacctggttagcgcgcccctctccaccatcatcaactgggaacgcgagtttgagatgtgggcgcccgacttctacgtggtcacctacacgggggacaaggagagccgctcggtgattcgggagaacgagttttcctttgaggacaacgccattcggagtgggaagaaggtattccgtatgaagaaagaagtgcagatcaaattccacgtgctgctcacctcctatgagctcatcaccattgaccaggccatcctgggctccatcgagtgggcctgcctggtggtagatgaggcccaccgcctcaagaacaaccagtccaagttttttagggtcttaaacagctacaagattgattacaagctgctgctgacagggaccccccttcagaacaacctggaggagctgttccatctcctcaacttcctgactccagagaggttcaacaacctggagggcttcctggaggagtttgctgacatctccaaggaagaccagatcaagaagctgcatgacctgctggggccgcacatgctcaggcggctcaaggctgacgtgttcaagaacatgccggccaagaccgagctcattgtccgggtggagctgagccagatgcagaagaagtactacaagttcatcctcacacggaactttgaggcactgaactccaaggggggcgggaaccaagtatcgctgctcaacatcatgatggacctgaaaaagtgctgcaaccacccctacctcttccctgtggctgccgtggaggcccctgtcttgcccaatggctcctacgatggaagctccctggtcaagtcttcagggaagctcatgctgctacagaagatgctgaagaaactgcgggatgaggggcaccgtgtgctcatcttctcccagatgaccaagatgctggacctcctggaggacttcctggagtacgaaggctacaagtatgagcggattgatggtggcatcaccgggggcctccggcaggaggcaatcgacagattcaatgcccccggggcccagcagttctgcttcctcctctcaacccgggcaggtggtctgggcatcaacctggccacggcggacactgtcatcatctacgactcggactggaacccgcacaatgacatccaggccttcagccgcgcccaccgcatcggccagaacaagaaggtgatgatctaccgcttcgtgactcgggcctcggtggaggagcgcatcacgcaggtggccaagcgcaagatgatgctcacccacctggtggtgcggcccggcctcggctccaagtcggggtccatgaccaagcaggagctggacgacatcctcaagttcggcacggaggaactcttcaaggacgacgtggagggcatgatgtctcagggccagaggccggtcacacccatccctgatgtccagtcctccaaaggggggaacttggccgccagtgcaaagaagaagcacggtagcaccccgccaggtgacaacaaggacgtggaggacagcagtgtgatccactatgacgatgcggccatctccaagctgctggaccggaaccaggacgctacagatgacacggagctacagaacatgaacgagtacctgagctccttcaaggtggcgcagtacgtggtgcgcgaggaggacggcgtggaggaggtggagcgggaaatcatcaagcaggaggagaacgtggaccccgactactgggagaagctgctgcggcaccactatgagcagcagcaggaggacctggcccgcaacctgggcaagggcaagcgcatccgcaagcaggtcaactacaacgatgcctcccaggaggaccaggagtggcaggatgagctctctgataaccagtcagaatattccattggctctgaggatgaggatgaggactttgaagagaggccggaagggcagagtggacgacgacaatcccggaggcagctgaagagtgacagggacaagcccctgcccccgcttctcgcccgagttggtggcaacatcgaggtgctgggcttcaatgcccgacagcggaaggcctttctgaacgccatcatgcgctggggcatgcccccgcaggacgccttcaactcccactggctggtgcgggaccttcgagggaagagcgagaaggagtttagagcctatgtgtccctcttcatgcggcacctgtgtgagccgggggcggatggtgcagagaccttcgcagacggcgtgccccgggagggcctctccaggcagcacgtgctgacccgcatcggggtcatgtcactagttaggaagaaggttcaggagtttgagcatgtcaacgggaagtacagcaccccagacttgatccctgaggggcccgaggggaagaagtcgggcgaggtgatctcctcggaccccaacacaccagtgcccgccagccctgcccacctcctgccagccccgctgggcctgccagacaaaatggaagcccagctgggctacatggatgagaaagaccccggggcacagaagccaaggcagcccctggaagtccaggcccttccagccgccttggatagagtggagagtgaggacaagcacgagagcccagccagcaaggagagagcccgagaggagcggccagaggagacggagaaggccccgccctccccggagcagctgccgagagaggaggtgcttcctgagaaggagaagatcctggacaagctggagctgagcttgatccacagcagaggggacagttccgaactcaggccagatgacaccaaggctgaggagaaggagcccattgaaacacagcaaaatggtgacaaagaggaagatgacgaggggaagaaggaggacaagaaggggaaattcaagttcatgttcaacatcgcggacgggggcttcacggagttgcacacgctgtggcagaacgaggagcgggctgctgtatcctctgggaaaatctacgacatctggcaccggcgccatgactactggctgctggcgggcatcgtgacgcacggctacgcccgctggcaggacatccagaatgacccacggtacatgatcctcaacgagcccttcaagtctgaggtccacaagggcaactacctggagatgaagaacaagttcctggcccgcaggtttaagctgctggagcaggcgttggtcattgaggagcagctccggagggccgcgtacctgaacatgacgcaggaccccaaccaccccgccatggccctcaacgcccgcctggctgaagtggagtgcctcgccgagagccaccagcacctgtccaaggagtcccttgctgggaacaagcctgccaatgccgtcctgcacaaggtcctgaaccagctggaggagctgctgagcgacatgaaggccgacgtgacccggctgccatccatgctgtcccgcatccccccggtggccgcccggctgcagatgtcggagcgcagcatcctgagccgcctgaccaaccgcgccggggaccccaccatccagcagggcgctttcggctcctcccagatgtacagcaacaactttgggcccaacttccggggccctggaccgggagggattgtcaactacaaccagatgcccctggggccctatgtgaccgatatctagccgtcctcgagacttccctgtgttgcagcgctcatttccagctgagccacgcctgccgggccacctgcccgacccacatgggagagaaaagctgccacctttttaggagccagcgccaccttgggacaaaaagggaaacctagtaatgccatcacatggaggacgaggcccagctcagctgggccagagcccagaagtgccacctcatcataattcaagtgttcttccacacagcgttgcccccacaaccacgccggacgtgccccctcgccaccttttccagacgacttcttagaagagatttcatttatttgtacatcttttgcactttcctattgaagacttgaacacgtttgtcttgataaaagttggatgacgtatggaagattcgaacctgcagcactgatgtctctttaccgatgggttccagacccaaggtagtcctggcactgccctgtggactcagcccagctggggaggacatggcgcccggtgccctaggagccctcagtgtcccctacctgacctgtctgcacctgtgtgacagccccttctgatttggccccctgcccgcttggagcctcccagcaccagacagggcaggtttggggagccgctgtccagccctatggtgagaccctggctgacatttcccctccttcctgccaaggctggaggccgccaggtgtttgctctctccttgtggggaggtggatcctctgccagcaggtgtctgtccccggcccagcgcatcttgaaggccctggactctgttaccagtggggggcctgtggccccggctcttcacagcaggcagaagcgtatgatcccagggagggtgggctggggggggggcagcacttgcctcggagccactgctgcgttgggtttgccttctgccagaggtgtggttaagggcagtgatagctgcgccccagcaaggagggtcatgggccctgagctggcccaggagaccctggccctgccctgtgccctgggatgcctaccctgcccacctccggaggtggaaccgggctgcccctgggggcctggacttgtcccattcccctgtccctggaaaggccttcccgggggctttttgcctgaggctgcactcttggaaggcgtggggagagttctgcctggagggggactggacccagtgccctctgcagacccctcccagccgcagcaccaagggcttgctgccttgtttcctgccaggagcgccctgacatcccaaaaccatcttcccacatgtgggttgttggaatcccacccactcccccggaccctgcccctccaagttggggacggagatggggagcaggtcaggccctccctaggcctgtttgtgtgtgatccttctcctggccccaggtcgacctggaaggcgtttctgagtcttgtactgggaccttcccagggaaggtgccttggggtgcagaatcctgggaatgttaggaaagggctgcgtgaaaaacccagctgggcctggggtggaggtgcacacgggctggggctcagctccttggagagggcctgcctgggactgagggggccagggctgggtcaggattcaccagcttgtgttgcagacactggagcgattgcctggccctccctaagccctatatgcctcactcataatgaggctgtctctgaaccccggagggcgggacctacaagtccttcctcttggcgcattcccacactccagtctggatccaggtctgcaaggccagcccgaccctgacactgcacgtgggcgtagtggggagcaccctggagtggagatgattgtcagcgggctcacatgttatggttcatccacatgcgtgttgtgtgctctgcggtgcctcctggcaaagggtcctggctcgggtcagaagctcactcccgtgccctcgtccccaaacaagcagtggcaagcactggggttggccctcgttgggagcagtgcccaccttccttggcccacagcagataggtcccgagcagcaggactggaggcctgtggcggtcagggcaggggtctgtgtctccaccccaagggctgatggtccctcccctgtgcgccttcctctcaggcaggcttgctctgtcccttcctctctgcttccctcccaggcctcgccagggctcccttctcctctgcctggttagactcgggcacccaggaaagcctggccagggctcctttgggcctgggccccctgcactgcctggtccagaaggggtggtgctgtccgtggccagcaccccggggcccgggaggggtgggctactagagtcagagcgggtttggggctgaccagtttgggagaggagaaaagatctgagaatgtccttcttggtttgtcagtcatctctgccaaaagtggtgatggtggtgtccgtatgtttggcgtctttgggctgggtttggtttttgctgctggtagaatcagggtcctcgggcatgaacgcgagcccaaagtgccagtctgcgattggaaatttccagccactttaagccagtgctgagtagggcttctgcagagccatgtttgagccaaggtcttggaaggcattgccccatgggctcaggtgactcggggtggagtgagcacgtctgcagggccctctcatacacgcctgaggcagaagcagcgtcccccgtgaaagccaccttccgaagctcctgcgttttttgcaaacttggcttcccccaggggcaggctggactttccctgccccctatgattgaagtcctcctgcttttgggggctgccttcccagagtcccccgggtgctcccctgccgaggtcaggagctgaccaagccttggcccggtgacacctgcagccctcactcctgtcatcccaggacacttgaggcccaaggaggtggagtggagagtgggctcgggtacatgggagccagaagccagatggacttggtcaagtgtcggtcacttggagcctccagtgtgcgtcagggtctgtgggcaggggacagggcgtgggtgggggccgaggctggcacgcccctctgccctcaccgtcttggtgacctggcctcggcccctcccccaagtctcttctgtgcaaggcccgcctcggcctcggcagctggttcctgtcctgttttctgtgtctgaaagtttacaggttgtggtgcatcagcccaaactcactggcgttgtgttttttttttctttaattttcagattttttttttaaacaaagtatttttttaggtgcgataacccagaaagggcccgttgggtgtgtgtgtgtgtcctgaactcctcaagcagcgattggagcccaagcacccctggagaggaagggagggtccccactggcccgtggggtctgagttcaggggtgtggagggagcagactccaccggcccaggcccagctaagagggggccgacccctttccccaggcacagccccaggctggcaaagggagggccctgggctgggtgcaaggcgcgccaggagtcccagccagggtggccccggcgggggcgggtccagctttggaagccaggctcccctgtgagccgtggcttgtctggtcttcgcccacgggaggctggacagaggctgtagccaacacaatcacctttactttgtactctgtgtgtatgttttggttttctgtgttttaataaatcctttgggaaagg

SEQ ID NO: 6 - DNA, 6417 bp, Homo sapiens
gcggctccgggtgactcgggccagtgtagaggtcctcaggccgccggcaggagcagctgggccaattccctggccgggagcggaaggggatggcgtcgggcctgggctccccgtccccctgctcggcgggcagtgaggaggaggatatggatgcacttttgaacaacagcctgcccccaccccacccagaaaatgaagaggacccagaagaggatttgtcagaaacagagactccaaagctcaagaagaagaaaaagcctaagaaacctcgggaccctaaaatccctaagagcaagcgccaaaaaaaggagcgtatgctcttatgccggcagctgggggacagctctggggaggggccagagtttgtggaggaggaggaagaggtggctctgcgctcagacagtgagggcagcgactatactcctggcaagaagaagaagaagaagcttggacctaagaaagagaagaagagcaaatccaagcggaaggaggaggaggaggaggatgatgatgatgatgattcaaaggagcctaaatcatctgctcagctcctggaagactggggcatggaagacattgaccacgtgttctcagaggaggattatcgaaccctcaccaactacaaggccttcagccagtttgtcagacccctcattgctgccaaaaatcccaagattgctgtctccaagatgatgatggttttgggtgcaaaatggcgggagttcagtaccaataaccccttcaaaggcagttctggggcatcagtggcagctgcggcagcagcagcggtagctgtggtggagagcatggtgacagccactgaggttgcaccaccacctccccctgtggaggtgcctatccgcaaggccaagaccaaggagggcaaaggtcccaatgctcggaggaagcccaagggcagccctcgtgtacctgatgccaagaagcctaaacccaagaaagtagctcccctgaaaatcaagctgggaggttttggttccaagcgtaagagatcctcgagtgaggatgatgacttagatgtggaatctgacttcgatgatgccagtatcaatagctattctgtttctgatggttccaccagccgtagtagccgcagccgcaagaaactccgaaccactaaaaagaaaaagaaaggcgaggaggaggtgactgctgtggatggttatgagacagaccaccaggactattgcgaggtgtgccagcaaggcggtgagatcatcctgtgtgatacctgtccccgtgcttaccacatggtctgcctggatcccgacatggagaaggctcccgagggcaagtggagctgcccacactgcgagaaggaaggcatccagtgggaagctaaagaggacaattcggagggtgaggagatcctggaagaggttgggggagacctcgaagaggaggatgaccaccatatggaattctgtcgggtctgcaaggatggtggggaactgctctgctgtgatacctgtccttcttcctaccacatccactgcctgaatcccccacttccagagatccccaacggtgaatggctctgtccccgttgtacgtgtccagctctgaagggcaaagtgcagaagatcctaatctggaagtggggtcagccaccatctcccacaccagtgcctcggcctccagatgctgatcccaacacgccctccccaaagcccttggaggggcggccagagcggcagttctttgtgaaatggcaaggcatgtcttactggcactgctcctgggtttctgaactgcagctggagctgcactgtcaggtgatgttccgaaactatcagcggaagaatgatatggatgagccaccttctggggactttggtggtgatgaagagaaaagccgaaagcgaaagaacaaggaccctaaatttgcagagatggaggaacgcttctatcgctatgggataaaacccgagtggatgatgatccaccgaatcctcaaccacagtgtggacaagaagggccacgtccactacttgatcaagtggcgggacttaccttacgatcaggcttcttgggagagtgaggatgtggagatccaggattacgacctgttcaagcagagctattggaatcacagggagttaatgaggggtgaggaaggccgaccaggcaagaagctcaagaaggtgaagcttcggaagttggagaggcctccagaaacgccaacagttgatccaacagtgaagtatgagcgacagccagagtacctggatgctacaggtggaaccctgcacccctatcaaatggagggcctgaattggttgcgcttctcctgggctcagggcactgacaccatcttggctgatgagatgggccttgggaaaactgtacagacagcagtcttcctgtattccctttacaaggagggtcattccaaaggccccttcctagtgagcgcccctctttctaccatcatcaactgggagcgggagtttgaaatgtgggctccagacatgtatgtcgtaacctatgtgggtgacaaggacagccgtgccatcatccgagagaatgagttctcctttgaagacaatgccattcgtggtggcaagaaggcctcccgcatgaagaaagaggcatctgtgaaattccatgtgctgctgacatcctatgaattgatcaccattgacatggctattttgggctctattgattgggcctgcctcatcgtggatgaagcccatcggctgaagaacaatcagtctaagttcttccgggtattgaatggttactcactccagcacaagctgttgctgactgggacaccattacaaaacaatctggaagagttgtttcatctgctcaactttctcacccccgagaggttccacaatttggaaggttttttggaggagtttgctgacattgccaaggaggaccagataaaaaaactgcatgacatgctggggccgcacatgttgcggcggctcaaagccgatgtgttcaagaacatgccctccaagacagaactaattgtgcgtgtggagctgagccctatgcagaagaaatactacaagtacatcctcactcgaaattttgaagcactcaatgcccgaggtggtggcaaccaggtgtctctgctgaatgtggtgatggatcttaagaagtgctgcaaccatccatacctcttccctgtggctgcaatggaagctcctaagatgcctaatggcatgtatgatggcagtgccctaatcagagcatctgggaaattattgctgctgcagaaaatgctcaagaaccttaaggagggtgggcatcgtgtactcatcttttcccagatgaccaagatgctagacctgctagaggatttcttggaacatgaaggttataaatacgaacgcatcgatggtggaatcactgggaacatgcggcaagaggccattgaccgcttcaatgcaccgggtgctcagcagttctgcttcttgctttccactcgagctgggggccttggaatcaatctggccactgctgacacagttattatctatgactctgactggaacccccataatgacattcaggcctttagcagagctcaccggattgggcaaaataaaaaggtaatgatctaccggtttgtgacccgtgcgtcagtggaggagcgcatcacgcaggtggcaaagaagaaaatgatgctgacgcatctagtggtgcggcctgggctgggctccaagactggatctatgtccaaacaggagcttgatgatatcctcaaatttggcactgaggaactattcaaggatgaagccactgatggaggaggagacaacaaagagggagaagatagcagtgttatccactacgatgataaggccattgaacggctgctagaccgtaaccaggatgagactgaagacacagaattgcagggcatgaatgaatatttgagctcattcaaagtggcccagtatgtggtacgggaagaagaaatgggggaggaagaggaggtagaacgggaaatcattaaacaggaagaaagtgtggatcctgactactgggagaaattgctgcggcaccattatgagcagcagcaagaagatctagcccgaaatctgggcaaaggaaaaagaatccgtaaacaggtcaactacaatgatggctcccaggaggaccgagattggcaggacgaccagtccgacaaccagtccgattactcagtggcttcagaggaaggtgatgaagactttgatgaacgttcagaagctccccgtaggcccagtcgtaagggcctgcggaatgataaagataagccattgcctcctctgttggcccgtgttggtgggaatattgaagtacttggttttaatgctcgtcagcgaaaagcctttcttaatgcaattatgcgatatggtatgccacctcaggatgcttttactacccagtggcttgtaagagacctgcgaggcaaatcagagaaagagttcaaggcatatgtctctcttttcatgcggcatttatgtgagccgggggcagatggggctgagacctttgctgatggtgtcccccgagaaggcctgtctcgccagcatgtccttactagaattggtgttatgtctttgattcgcaagaaggttcaggagtttgaacatgttaatgggcgctggagcatgcctgaactggctgaggtggaggaaaacaagaagatgtcccagccagggtcaccctccccaaaaactcctacaccctccactccaggggacacgcagcccaacactcctgcacctgtcccacctgctgaagatgggataaaaatagaggaaaatagcctcaaagaagaagagagcatagaaggagaaaaggaggttaaatctacagcccctgagactgccattgagtgtacacaggcccctgcccctgcctcagaggatgaaaaggtcgttgttgaaccccctgagggagaggagaaagtggaaaaggcagaggtgaaggagagaacagaggaacctatggagacagagcccaaaggtgctgctgatgtagagaaggtggaggaaaagtcagcaatagatctgacccctattgtggtagaagacaaagaagagaagaaagaagaagaagagaaaaaagaggtgatgcttcagaatggagagacccccaaggacctgaatgatgagaaacagaagaaaaatattaaacaacgtttcatgtttaacattgcagatggtggttttactgagttgcactccctttggcagaatgaagagcgggcagccacagttaccaagaagacttatgagatctggcatcgacggcatgactactggctgctagccggcattataaaccatggctatgcccggtggcaagacatccagaatgacccacgctatgccatcctcaatgagcctttcaagggtgaaatgaaccgtggcaatttcttagagatcaagaataaatttctagctcgaaggtttaagctcttagaacaagctctggtgattgaggaacagctgcgccgggctgcttacttgaacatgtcagaagacccttctcacccttccatggccctcaacacccgctttgctgaggtggagtgtttggcggaaagtcatcagcacctgtccaaggagtcaatggcaggaaacaagccagccaatgcagtcctgcacaaagttctgaaacagctggaagaactgctgagtgacatgaaagctgatgtgactcgactcccagctaccattgcccgaattcccccagttgctgtgaggttacagatgtcagagcgtaacattctcagccgcctggcaaaccgggcacccgaacctaccccacagcaggtagcccagcagcagtgaagatgcagactgataccacctccaccgctgagcagtgaccttcctcactttctcttgtcccagcttctcccctgggggcctgagagaccctcaccttccttctgcccatcttccatgttgtaaaggaacagccccagtgcactgggggaggggagggagtgaggggcagtggtgcccttcctgcagaagagacatgcagcagtagcgctggcgccatctgcaggagctggcgggctggccttctggaccctggcttctccccactgtaacgcctgttacacacaaactgttgtgggttcctgccaggcttgaagaaaatgatctgaattttttcctccttttggttttattttgttggtttattttgtgttttcttttctcctttttggggggtattcagagtgggctgggcccctgggcgagacacagctacctctgttggcatctttttaataccaggaacccagcggctctagccactgagcggctaaatgaaataaagtggaaaaaaaaaaaaaaggaaaaaaccaaaagcataaaaaaccacagcaaatttcttgatgaaaattgaaaataaaagtttccttgt

SEQ ID NO: 7 - DNA, 6417 bp, Homo sapiens; identical to SEQ ID NO: 6

SEQ ID NO: 8 - DNA, 6452 bp, Homo sapiens
ggaagaaagtaaaaactcaaacaagctcatttgatatacaaaaagcagaatggcttcgaaaatataatcccgagcagctccttcaagatgaaggctacaaaaaacatataaaacaccactgtaataaggttttgcttcgtgtgagaatgctgtattatctaaagcaagaagttattggaaatgagtgtcagaaagtatttgatggagttgatgcaagtgacattgatgtttgggtaccagaaccagaccactcagaagttcctgctgagtggtgggattttgatgctgataagtcactccttattggagtttttaaacatggatatgaaaaatataacactattcgagcagacccagcattatgcttcttggaaagagtgggaaaacctgatgagaaagcagttgctgctgaacagagagcgaatgattatatggatggggatgtggaagatccagaatacaaacctgccccagccatctttaaagatgatatagaggatgatgtttcctcaccaggagatcttgttatagcagatggagatggtcaactgatggagggtgataaagtatattggcctactcaatcagctttaaccacacgtttgaggcgtctcatcactgcataccagcgtactaataaaaacagacaaattcaacagatacaaccgactttctcggtgcctaccagtgtaatgcagcctatttatgaggaagccactcttaatcctaaaatggcagccaagatagaaagacagcaaagatggacaagaagagaagaagctgacttttatagggttgtatctacatttggagtggtttttgaccctgacagaggccaatttgattggacaaaatttagagctatggctaggctacataagaaaactgatgatagtttggaaaaatatttgtacgcattcatgtccatgtgtcggagggtttgtcgtcttccttccaaagaagaattggtggatccaaatatttttatccagcccatcacagaagaacgtgcttctaggactttgtatcgcattgaacttctaaggaaagtacgggaacaggcccttcgacatccacagttgtttgaacgcttgaagctttgccatccaaatccagatttaccagtctggtgggaatgtggccctcatgatagggatttgcttattggtgctgccaaacacggggtgagccgaacagactatcacattcttcgtgatcctgaactctcatttatggcagctcagaggaactacagtcaaagtaagatggctcattcaaggacttctaccccacttctacagcaatatcaagtagcactttctgcttctcctcttacctctctacctaggctcctagatgctaaaggtattattctagaggagatgaaagttaaaagtgaaaaccttaaagaggagcctcagtcttctgaagaagaatctatgtcttctgtggaaaccaggacactgataaaatctgagcctgtaagtccaaagaatggtgttttaccacaggctactggagaccagaaatctggtggaaaatgtgaaacagacagacgcatggttgcagccagaacagaacccctaactccaaacccagcttctaagaaaccaagagtccacaaaaggggatcagaatctagttctgattctgactcagattctgagagatcatcttgttcttccagatcatcttcttcctcatcatcctcttcttgctcccactctcgatcaggctctagttcttcttcatcttcatcttgttcttcagcatcttcttcatcctcttcctccacctcttcctcctcctcctcctcttcatcttcatcagaagaaagtgacagtgatgaagaagaagcccaaaaacgagaaagtactactcacatgaaagcctatgatgaagaaagcgtcgcgtcactgagcactacccaggatgagactcaggatagttttcagatgaacaatgggacaccagagtctgcttatatcttacaaggtggatatatgctggcagcctcgtattggccaaaggatcgtgtgatgatcaataggttggacagtatttgtcaaacagttctgaaaggaaagtggccttcagctagaagaagttatgatgctaacacagtggcttctttctataccacaaaactgctggacagccctggagcagctacagaatacagcgagcccagtgtacccactcccccaggtgccggtgttaaagaagaacatgatcagtcaacacagatgtcaaaggaaggtggtttgaagttgacatttcagaagcaagggcttgctcagaaaagaccatttgatggtgaagacggtgctctggggcagcagcagtacctcactcggcttcgagagcttcaaagtgcatcagagaccagcctcgtcaatttcccaaaatccataccagtatcaggtacttccattcaaccaacccttggtgccaatggtgtgatattagacaaccagcctatagtcaaaaaaaggcgaggaaggaggaagaatgtagaaggtgttgacatcttcttttttaacagaaataaaccacctaatcatgtttctttaggcttaacctcctcacagatttccacagggataaatccagcactatcctatactcaacctcaaggaattcctgatacagaaagtccagttccagttattaatcttaaagatggaacgagacttgcaggagatgatgcaccaaagagaaaggatttggaaaaatggcttaaggagcacccgggttatgtggaagatttgggagcttttattcctagaatgcagcttcatgagggaagacccaaacaaaaaagacaccgttgcagaaaccccaataaactagatgtgaatagtctcactggagaagaacgtgttcaactgattaacagaagaaatgctagaaaggttggaggtgcatttgctccccctttgaaagatttatgtagattcctaaaagaaaattcagaatatggagtagctcctgaatggggagatgttgttaagcaatctggatttcttccagaaagcatgtatgaacgtattctcactggtcccgttgtgagagaggaagtaagcaggcgggggagacggcctaaaagtggaattgcaaaggccacagcagcagcagctgctgcatctgccaccagtgtttcaggcaatcctttgttagccaatggactacttccaggtgtggatctcacaactcttcaggccttacaacaaaacctacaaaacttgcagtcactgcaagtaactgctgggttgatgggaatgcctaccggccttccttctggaggagaagctaaaaacatggctgctatgttccccatgctgctgtcaggaatggctggattaccaaatctgttgggcatgggaggactcctgacaaagcctacggaatctgggacagaagacaaaaagggaagtgactctaaggagtcagaaggaaaaacagaaaggacagagagccaaagttcagagaatggtggagaaaactctgtgtcaagttctccttccgcatcctctactgctgcattaaatacagctgcagctgccaacccattagctcttaacccactattactatctaatatactttatccagggatgcttctcactccaggccttaatcttcatattccaactttgtcccagtccaatacttttgatgtacaaaacaaaaacagtgacttaggctcgtctaagtctgtagaagtaaaagaagaagattccagaattaaagatcaggaagacaaaggaggaactgaaccaagtcctctcaatgaaaacagcacagatgagggttcagagaaagctgatgcttcatctggatctgatagtacatcgtcgtcatctgaggattcagattctagtaatgaagactgattcccagactctgcacttaaaatatgaactgattttggattttttctttaataattaattgtaaataccccagtgttgagtgcatcaataacttactgaccgaacatttcagttatttgtttagaagtgcaaactgctttcagagactttttgcatgtaatatttcttaagattcataagtttctgaactcgtatgtactatcaaatacataaaggtgtaaaattacaacaaaaggcattataattttgttgggggttaattttatgaaaattatgctcaataagagttgtatatttaatatatttgcagtgaacacagaatactttatgcatattactgatttaatttgaatatagttttacagcctccttgacacctataatttacagatcaaaactcagcaataatttgggcagctaatgaatgtcatgaaagctgtagaatctacatcaccatccattgctttaattacatgaaaatgctctagtgttgtgatgcactgctgatgtttccaattcaggtacaagtatgttttaaagaagaaataagtttcccaatcagccaatttaactggctacctgttacctcagctgagttagtttaggaagtttacattcgtttctaattctatacttgttttcaggggttttttaaacacatcctatatatcatgtcaatctggcaagaaatatgacttgctttttgctgagcttaattcagatatcagtaaaattaagtcataaaataatcatgtgtcatgtgactttggcaccctatagacatacttagttttaacttttcaaagtttggcctcctattagaaataatcatgtctcagatgagtaatgtctgtttccagggttcagaaaaggcaaactcatgaaatgccactgaaaagaactttcaacacagcatacttcatgtaaaagaaattgtttgtttgctttctttgtgtagatttctatttgtgttttatgtcatggaaatattccagaattaacagataatagtggtaaagtaatatgcagatagtctaaattcattttgagtttctaggtgtaagcagactaaatgttgcccagaatcagtgttgggttatcagtttatattaaatatactgagttgcccgttttgaaaatgcactttgaataatctcaaaaagatgtacaagttatacctgtaaaccacaaaagtgaagcctgaggcttctgttcaatttcatagactcctttaccatgtaaaatttgtctgatatttgatttgtgatacaatttctcctgctaaagctgctattattctgacaaggtagaggtccaggttcacctttatatatatttaaaacaattagtactgaattggacataaaaatattgacattctaaggagagatatatgttagcatttttctggtactcaaataagttagtagtaaagtctgcaagggcataaatttagggggaaaaagtgtcccagttctctcctacagaaaaaatactttcagtatgttttgataaaactgttgctttgtcatgagttagtcaattgtatcaggttttccaagacctttaccagtaaattatgtttctgtatgtaaaataaccccttattagagagacagtgttatatgtatttacaaaattatataagttccattgggattgtattgattttgtattttcccaaaatagtactttgaattgatagtcctttatgcaatgtcttagcaatagtctctataatgcccatccaggagaagtgggtagtaattcttcatcatgaaaatgatatattacatatttagtatcttccctttgcagtattgcacttttgtttaactagaatacacctatgagatagccaaagtttcaaacacagttatcttagtttaccggtggagtatttcaacaccaaccacatttcccttcctccctctaattctacccacatgatctttattccttcctttcgccaattaaaaaaaaaaaaaaggaaaaaaaatctgtagatcttgtcactaaaatctaatttatatcaaatttatgagagaaagtattttcctaattatggtcaaataaatttggttaacatcctagtgattctctttctatataataaggcaattacagttttcaaagcattaagtctaacataactttaaacattctcttaggtttcaagacacttctatttaatattcattggggaaaagttgtccagctatcagctaagaaaacacatgcaaatatggttgtgtaaagttaagggttataaggaaaaaaaaatcagtagaattacataatactaaagttgcagttgaaagaatatccaagtatgtgttggtagttactaaaagaattatagctgttattgccttgtatttatagcccttgtttcaggttttatgattcaagtcttagtccaatctttcttttggacatttgcaatatttaccagttgtgttttgtgtagtctgaatttgctttctgtagttgagcaaacgtcttaaaaagtcatttgtaatttattaaattactttctatgatgttctatagagc

SEQ ID NO: 9 - DNA, 142519 bp, Homo sapiens
gatcagggcccacctactggcctgcagccggaggggaggccctctccatcctggtccacccaggcctcaggactctgtgcaccgtccagctgagtccactgcctggccgttctcttcaagtcccctccctgcaaaggttggaacatcagagtcccacagttgggctgggcaggctaaagccagagaaacagatgggggctggctggtctctgatggctctatgccaccctgtcccccactctcagaaatgggtacaagttcctgtactgctcggcgcgggccattggcatggcggacctcaccaaggggtacctgcagtgggtgagcgaggggggctgtagcctccccaagggccccatccttctgtctcccagcagcctcttctctgccctccacaggtaaccacccctacacatacaagcccgtggccccctggtggggaaaggcaggcccactgccaggccagccttagcaggctggcattaagctctcaggtggcaaggagggtggggtcagaccccccatcacctgagcctgccatcccctctggcttcttccctactttggcccccttccctgctgtggttctggccaccccagagaggtgatcgagaagaaaccagaggtgttcaaggtcgcctgcctgagtgacatccagcagctgtttctgccccacggacagcccttctatgctgcctttgggaataggcccaatgtgagtgtgtcccctccactctgctgagccacctctccccagctgcctgcaggcctccattccatgggcccctcctgtctcccacaggatgtctttgcctaccggcaggtgggcctgcctgagtcacgcatcttcacagtcaacccccggggagagctcatccaggagctcataaagaaccacaaatccacgtgaggctaaaccctgccatgttccccatgccctaccacgtccccctgccctggcttctccctgggccccaattttacctcttaccggggagtctgtcccttactctgggacagacccatggccaaggccatcgtttggtggcagctcaggctcagtgctggcccccttctgcctgtaggtatgagcggcttggtgaagtggtcgagctcctcttcccacctgtggcccgtggccccagcacagacctggccaaccctgaatacagtaacttctgctactggcgggagccactgcctgctgtggaccttgataccctggactgaacctgccctggctggctcctcctccctggcccggcccaggactggctaggtgtcctggggtataggagggtgggaattggagtgtcatggggcaaacccactgaaggggaaggaggaggctgcaggttggttggcagctagagagactcccccatcttccccgtcatatttttgccagctaagctgcagctgctccaggcgtcagtgtggcactgtcctggggcaattagcttgtcatctgggcccttgcagggttctttttttttttttttttttttttttcctgagacagggtcttgttctgttgcccaggctggagtgcagtggcatgatctctcggctcactgcaacctccgcctcccgggttcaagcgattcacctgcctcagcctcccaagtagctgggattacaggcgtgtaccaccacgcctggctaattttcgtgtttttagtagagatgggttttcaccacattggccaggctggtctcgaactcctgacctcaagtgatttgcccacctcggcctcccaaagtgctgggattacaggcatgagctaccatgccaggcctccttgcagggttttctatgcccttgatatctgtctccctgtcaacctgggaccttgctgtaagtcttgataggacagggagaagagggaggccctaccgaggctcgaggcttcagtgaagggtgacagcagtgggagtgtggtacagcctctggaaggacacagtgttctccccgccccttgtctgggagccaggactgtaccctccgaagccagacatcactgccaacatatcccccttgctggtgccctggcatctcagcacatgacacacacccacacctgcaggctgtggttccggcttggcctgctccccgtccggctgctgccgctgcctctctccagacctcgcttaaggacagtcccaaactcagctggggcaggtgttggcctgaaagtcctcccccagcctctgctggccagcttggtgctcacagctgctgggtaagctcttgcctaaggagctgtgggaagcagggctgatgccccagcaacctctcctcccactgtctttgaagaaagtagctttagaccggctaaaagctttaatccagagcctgccctactctgatagtaccagagtggagggcagaataccaaatgtccaggaaccaaaggcagggctgtggggacctgaagagcagcacagtggggcccgtgctgctgtgggggaaactgaggctgggagcctcagcagagaccggtgtcaagagtctctgggaactgcataggcctgaggaacatgcattttcaagttgtccattgatggtttcgtacctgaatttctcaccttttgtgaacatcttgggagggtgggggttttgcaggggtgttaaaagcaaggcttggagcccctttcctccagctggtggctccttctcagggcctggcctcattcaggccactttgtagagaaatgccctgacctcgcaggaaggatttccccacccccaagtggaaggaagaggacagtgtgggcaccagagggccctggaaacatcttaggggaaggaaaggaaaaggataaatttggagtggggggtctctaaacagattgcctggattccgttctttcctggggttctacagctgcctaagccctcaccttgggggaggatcaaagggaataaagagaactcttggctgagtctttgtgcttctgttctgtcctgaaggacacgtgaggagccagaaacatctggcagcctctggtccttagttcagatacaacatccatgaagctgccaggtgtgaggagagttccagtgggcatcaccttcctgcacaaacctagtcctgatggctgcgaagagaagccgaggggacaaggaacagaagccaccaagggctgtggactgttgcttgtgggcacagggcaatgcggacaggccactggctgtgggcctgtaggtctctgcacagttgctgaggctggtgctgactgcctgcagggtcctctcaaggccttacatacggacactgatcaaggccagtttgccacagcagctgcccctgccagccatgctcaggccctgcagctggctgtccagacactgatttgctgacagtggaatgtgaactctctcaagtctaccctggccttcaagcatgacatccttgccttgaccgctgtccggaactgtccagtttctgcagcactgtgcatgggttttggtggctgggacagccacgtggaggattccctcagtggcctccttagtgccatttgggctagccagggaaggctggaaggcgctgctggataaggctgggctctggggtgatgttccccgagttggtgggatctaggggttgggtcctggccagcctgtctagttggcttgccctggccgcacctgctcagccagctgggcctggcagtccagtaggtcatcccggaggcgggcaatgtcctgcgtgtgctgggccagcgtacgattcagctggtccacatggctccacaggccctctctgggcctcagtggctcagtgggcaagctgtccaggcccgcggccagcaggcccagcctcctgcacgcaccctccacttgtgccacccgctggtcaaagtgtcccactgtgtgctgaagtttctgggctgtgtcctggcagcggctcagcccactggccaccttggccacaccagccttgagttgctccagctccccacgcaggttcaggacctgcctgtggccagcctgaagcctggagccttggctgctgacctgctcctggatggcctggacttcggcttccaccttgcgttcccgctcatccagggacgtgttggcagccaagaaggcatcagagtactggctgacagagtcactgaggcctgtgagcgacttgctcactgagttcagattgaccttgagcagagtgatctcgccttgaagtgagctgcccgtcccttctgccagctgtccctggacctcggccacagtgccattgagctgctggagcagtgctgcgtggctggccacctggcgtaggagggcctcgctccggctctgccaggccttcacctctgccacgaggctgtccaggatggctgaggtggtgctcggggtgcacgaagtctccagtgagaccaaccgttgctctagcacagccagctctgtctgtacaaggggccgagctgacctgcccggagaggcgctgtcatggcttagctccccagccaatgttgctaggcgctcctcgaggctctgcacgcgctcttccagcatggtcccaaagccgcccactccccacccccccatgtccaacctcagacagcatccccttgctccaccctctgtcccattgagcgtctccagaccctcaagcagcccgtccacacctccctcgagcatggcagcagagagcctcgtaagctcatccccggccggggcaccggggcccctttgggtctcggtgactgcctgcagggccctctcaaggccatccatacgggcattgatcaaggctagttgcccacagcagctgcccctgccagacacgctcaggccctgcagctggctgcccagctgtgacagctcctggcgcagggccagctcccggccatcaaggctctggtgcagcctccggctggcggcctgaccctcctcacattgccgccgtacctcctgcacccgcaggtcacactcactctggacgccttgcagcttctgctcaaagccatccagcaggctcccccagagtcggtgcagccgtcggtccacgtactcctccagcagggccagggaggtgagcggggatggtggggcttcccgcagccgctgcaggtgggcctcatggccaagtgccagcccatgcaccttgtctagaagctgcaccttggtctgaagagtgttgctcacctctgtcaccttgcttaggatctcgtctaagggaggtgttagtggccctctggctctgtctcctgggcccacaagcccctcagggatgaccccaaagcccacagggacagcaggagccctggggccaccagtcatcctgttgggatcctcgtggctagccaccaggccactgagggtaccatatgtttgagccaggcgctggacatcaccctccaggcgttccagccgctcaccaaacagccctgggcctttccttcctgggaaagagaagagagccccaggggcatcactggcctcaccctctcagcttgtcacaatgccaggccacccaagagactttctgttctttttttttttttttgagacagagtcttgctcagtcgcccaggctggagtgcagtggtgtgctcttggctcactgcaagctccgccttccgggttcatgccattctcctgcctcagcctcccaagcagctgggactacaggcacccaccaccacgcccggctattttttttctatctttagtagagatggggtttcacgatgttagccaggatggtctcgatctcctaacctcgtgatccacccgcctcggcctcccaaagtgctgggattacaggcatgagccaccgcgcccagcccagactttctgttctaagcctaccttctcactgggcataactccctcagctttatggtggggtgaaagtggggcttggggcttgcccctccccatccatgccggagctcagccagcctgggatcagtcccctccctgcccctgccactgagcccaagaaacccaatcaccttggagggtctccttgggcagactcaccatgagggctgggggctgctctgctgtaggaagggggcctggggcctgggtccagctgccctgaaggaatctgaggctcaggctccagctggggtgaggcagccccatggtccgtgaggtgctcagggcagcgtttcccagtgaagccgggacagcaacgccaggcgaggtctgtcactgtcttgtagccaaccttgtatttgggtctgagtactgtgcggtacctgggccagggagggcaagagagtgggatcctgcacctgagggctgtgcttctccccctagctggcactcagggccgccctcagacacttgcataaggctgggtatctccccacctccatagcacccccagggctgtactttttctgtgcttaaggtgacaggggtgggtaagggcaggccttcctgaagctcacagtcatgttagggaaggatgcaggtagaagtcccacccatcctggtagcttggagagaagcaggaattttgccccatccctcccaagaccacttggaccccctctagctcaggctgccatgagaaattccactcaggctgcagtagcacctgggagaatcagacacagccccagccctccctgtgggcagggaggggcactgccctggagctggcgctggcagccccctcactgccctgggggccactcttggctcctacctgctccccttagttctttccactcctctctagcctgctccgggacctcggctctggtcagagaggcagatggacgggtacctctgattgtgagcagggaagggaaggtccagggtacaggcaggctcgcgagggtccatgccctcactgttcagccacgtacctgttcacccactgacacaccggagacaacaggtttgtttccacacacacacgtgaacctctccaggtacagagctgtgcatcccatgtgcgtgcagccctgctgctccggcttcctctccctgccccatggcctgcctccttgtggtcaccacagtcctccctgggcctgccaggcccccctgtggcatcacccgttgcctaaggggccctctgctccctcctctcccctcaccttctgcctctgtcacttcctcctagccttgtgctgcctctgcccccagaaccttccccttgtccctctgtggttcctgggctccaacccaagcctgcccctgtctgtgcgggtgatgctctgctgcaagcccaaatccctggggtcctccttggcaccctcttttctggggccgccttctactctggcctgtatgggggctcaggtgggctctctggagtccaagaagccccttgtggagttggcagctgctctcagctgaaggcaggactggcgcttgtgccaggggattccaagtgtgtaggtgaggagtgcacttacgtgactgtcccggggcacttgggcccccatctacactgccggtattcagcctttacgtagctctccgctccctcctgtaggatgcaggtcacattcctgtgcaccacataggcacagagggccctacaggagaaaatggcacccctggaatatctggctgagcgaggcccacagaggcgcctaccctcccacctccatctgtgggcctcagcaggactccaaactcccatgtctcttctgtctgttctctgtgtccctgggagccaacgcccttggtctggggtccagccccgctgctggtctctgattccagcctccgtgatgggtggcccctgggctaagcactatttggaaccaggagcctgatgtggctgcccctccctgcgtggggaagaggccagcccggattctccctcaagtgccctgccacacccccccatccttcgctgattccaaactccttatcttggcatttaaggcttccgagtccccactcagtgactcctttgagagcaccatgtcttgtccttcctccctgcctggctcccggggaaatccacctctcctcgtagttttgcagcccctgcctttggtcctgttcccttactctcagagcagttcttgtaccacctcctccaggaggctcgctgtgatgtacccaggcccactgagtgtcggtactcttcctcctacaacagctctcaaattcagctagaattcaagttcagccagcaccccaggcagagcctccagcccacttaactgaggccagttcgcaagccactgcccacgaaacaccagaagctgaggcggggaaagcaggctccttgatgagccacaagggctgaggaggtggctgcctggcataagccgaccgctgtggacggctgtgccacagcacaggcccggagccaggagcccgctctggctctgcagcccggccacgctggggctcccatggggttgctggggccaggctgactgcgagagggcagcggccgcccggatgcgcagctctatctcctacctggccgggccaggggagagaagggaggcccggggcgggggagccccgcgtgctcagcgcgcggaccgcctttcctcctctccctccacctggtccgctcgcccggagtctgcacacagcgcagctggaaaatgttacttcgctggaaaggtttccgctggcactgccaggatcccaagccgccgcgccaggccccctcggttcctttttcctcgccagccccgcaacctcccggagcgtagggcgctcgcctgggcaggggctcggccccgggcgccgccccctctgtgcagcggcctcggggtgcccggggcctcgacgccccccgcgggtgcgggcaggtgggagcggcgacgcgcgcgggcccatccctccctcttcccgcccgccgcccgcccgcgcttacttgtgcggccccgggcgcagccgcgggcgccatcccgtcgtgtagagactgtagcgggaggcaccgggcggcgcaggccgcgccaggagcggggtgcccctggcctgcgcccccgagagcagcgccgcgacggcgcacagccagacgagcaggcggcggcggcccatagcggcccccgcgcctctgcccggccccgcgcggtgccccccgccgggtgtccgcctgcagcgccgcgccgcccccgccgctggtcggcccgggtcccgccccgagggtcccggggtccccttcggcccccgagctcgctggcccggccgcctggcgcttcgcggcggctgcgtcccgcggagtggccggcgctgctagcggctggggccgcggaggggaggcgggaggcgggctctttgcggattagcataaacttggggccgcgccgctcgccgccgccagcccctcgccccagctccgagcggacggccgaggagccgcctgcgaacaggaggttccaaagggagcagccccaggtggcaggggcgcaggccgccgcgccctgggaggccccccgagcacaccagccactctgtcgcctgcgggcacggggcgagacgcagagaagcggacccctgaggccagcctgggcgtggactttccaggagtcgtccaccctcaggatacgcttgtcttcagagtccgtgtgaacccgggaactgctctccagggctctggagccgagatggccgtgactgggggcacagcagcaccacctcccccgccacctctttggaatagaccccagatcatccccgtttgctcgctctgcctcctggccccatcattgcccgggaaccttagtttgataactccgctgacctctccccaggcatcccacctcccccttaccccgcaacctctgccacgtagcctggtggcccggttgcacctctggttctctggaggcgcctgtgttgcctctctgtcttaggtatgggttgcaccgagcccgaaaggcctatcaccgccccagcccagccaaaagtgctgtgcttttcgtgcaactgaagctctaagccggctggtcagagaaaatcctcaggtctttctcacaaggcactgccgcggtgaagccaggcgtcgtccacattttgcttgtgaagttgactcttggaacccaagagttgggttttatcgtcgtcctcattcagtatcaccttgctaactctctaacctgcacagagtccttctgattctgtcatcagagcggtactttcctggccttgtctcatccacaccggtggctacagccgtctgtgtctttgccccaggagctgataggtgtacagaactctggggcttgcttggagagacctccctccaaattgaccttgagatatgaattaatgccttttggatagggtgatggttatcaaggatactggaaggggcagctccgaaacccacatctatttcctgcccttagctttagtggcttttgggttctgtggggcggacatctgaccccagctaggcccatcctgctttcccccaggctttggaatcagaacacagaagctgaaggaatccagcttccaggtgtaggttgtgtgagtttctcagggctgtggtaacagccacagactcagtggcttaacacacagaaatgtattcttgcacatttctggaggctaactccaaaatccatgggccagcagggcaatgccccctccagtggctctggggagaagccttccttgcctcttccagccactggtggttcctggcaatctctggcgttctttggtcaaggattctttgtagcagcatcactccagcctctactttccctccgcatatccctcttcaggtgacctttttttctctgtgactgtgatctgtgtcctttcctcttctttctgtttttgttttttgagccagagtctcgctctgtcgcccaggctggagtgcagtggcgcgatctcggctcactgcaagctctgcctcccgggttcacgccattctcctgcctcagcctcccgagtggctgggactacaggcgcccgccataatgcccagctaatttttttgtattttttttttttttttagtagagacgtggtttcaccgtgttagccaggatggtctcgatctcctgatctcgtgatccgtccgcctcggcctcccaaagtgctaggattacaggcgtgagccaccgcgccctgcctttctggttgtgtttttaaatttgagacagggctgggcgcgatggctcatgcctgtaatcccagcactttgggaggccgaggtggggggatcacctgaggtcaggagtttgagaccagcctggccaatatggtgaaaccccatctctactaaaaataaaaaaattagccgggcgtggtggcgggcgcctgtaatcccagctacctgggaggctgaggcaagagaatcacttgaaccctggaggtggaggttgcaatgagccgagatcatgccattgcactccagcctgggtgacaagagcgaaactccatctccataaataaataaataaatagagacaaggtctagctccatcacccaggctggaatgcagcgtcgacctcctgagctcaagtgatcctcctgcccccacctcctctgtagctgggaccacagttgtgcaccaccatgcacagctcatttttaaaatttctgtagagatgggctctcactttgttgcccaagctggtcacaaactcctgagctcaagtgatgctctcacctcagtctcccaaattgctaggattctaggcgtgagccactgcacccagcccagcctcctcttcttataaggacaccagtcattggatgtacagcccaacataacctcatctcaaatgattacgtctacaaagaccctatttccaaataaggtcacattctgaggttccaaatgttcatgaattttgggaggggcacaattcaatccactacaggggcacttggctggtaatgataggagagaccacgagagctctgggggaatcatgggaaagggagaaagagtgtggtcaggggagagaggggcagagtcaggggctatgtggtgagtaaccatctggggacaagctgtgacttccctatctgcttccagtagtccaccattggagctagcttgaatgggtttctgatcccagcaaccaaatgtgctctacagagagaaacttctctagtcatgaactgtagaaatgatccctgaaagtatagtcttacagcaaccacatggtccctaagacagcctttcactctccctataaaagcaccgaatgtgctattatatgctccaacctctaagagagatgttatgaaccattttgctgagggcaagatacaccatatccccaacaagctcttgatcttgtaaccactgactttgcgctttttaggaagtgagaaacatctgagccagctcctcatcctaggctgggtgctgactgcctggcagcattgcccagcctggctctgggaggaactcccccaccccagaggctgtgatctttttccaagtggtcccaccccttcctctcactcccttccccagccccagatgtggggcccaaaactcacaaggaagaggctggaccctgtttgagcttggaaatatccacgtcataggggctaaaaggatagctggaccccacttccccctttcccaggtggctgacatcaaacaccatgctccctctttcactgcaacagttttgggatccaggatctgggccccattctagtgggggtggaggacctcagtacagggagatgctcagggtcttggctggatcaggccagagcgggggtccccccatagacgatgaggccactttgcatctgccagacaggaaacctagggaggccagaagcagaggaggaaaggaggcagtcatgttcctctgactcagactggagtttctacacctggatggaaacgggccatacacgggagtgggagtgcagtttcagttcacagggacatgcacaaccacacccaacccatcccccataccactatcatgcatgtgcatgagtttttcctttcgtacagatggggtctcactatgctgcctaggctagtctcagactcctggcctcaagtgatcctcctgctttggcctcccaaagtattgggactgcaggggtgagccctatgcatcctgcctttcttttctgttcttttcttttttttcttttctttttttttttttacggagtctcactctgttgcccaggctggagtgcagtggcgtgatctcggctcactgcaacctctgcctcccaggttcaagcaattctcctgactcagcctcccaagcagctgggattacaggcacatgccaccacgcctggctaattttttgtatttcttttttttttttttagtagagacagggtttcgccatgttagccaggctggtctcaaatgatccgcccaccttggcctcctaaagtgctgggattacaggcatgagccaccacatctggcctttccttttcttattgagggcccagcacataagagtattaaaaaccacacaaagttgtatgggatgcagttcccctaaaagagacagaaatagaaagcactgaagctgctggctttctggtacaagctcttcctgctgcagatggacccctcctgcacagggaccacagcccttgaaggcaccagccctaaggtgcagccctttgtccctgaccatctcccttaccctctttttttttcttttttgagagtgcacagctaggcctccctgcccacagagccacatttggaagttctgtgcagcaaggctctttgcccaggctcacttttttctccacatcatcaccatcagcattttgattatatctgcaccatcataaaaggttataccaaatgcacatgagtttctgaggcaggggtttgcactgaccccatccagctaggcagaagcagggctcgtccaggatcctaggggggatagatgtgcagcctggaggcaaggtcctggaagagtccagaaatcgtggaaactgggctctgtaaagcctggacggacttccattgagtgagagtagcttaggaacagcttagaaattgtgactgctgatggactgtcaacatggacatatgcaaaccagatgcaacattacgcaaattatatacaaaatacatgcacaggtctcccaactattgcaaatgccagatatggtccttgaggaccctaagtttagctgagacctgttgtggggcagagagatcaccctggacttgccagtttctttttttttttttgagactgagtctcactctgttgcccaggctggagtgcagtggcgtgatcttggctcactgcaacctccgactcccaggttcaagtcattctcgtgcctcagcctctggagtagctggcactacaagcgtgagccaccacgcccagctaatttttgtattttcagtagagatggggtttcaccatgttggccaggccggtcttgaactcttgacctcaagtgatccacctgccttggcctcccaaagtgctgggattacaggcatgagccactgcacctagctaggacttgccagttttcttatggtgggcaggaagggctttgctccaaggtggttcacacactgtggggaagaaatcaggcaaggagtcaattagactcaggtttgaatcccagctcttccactcacaagttgcatgacctcccttgagtcacttcacccctctgagcctctgctttcctatacgaaaatgattattatgaggaatagatgagaactttggccaaggctcccatgtgcacagtcgtgcacataacagacgcccagtgagtgtggcagggctctgcacagagtcctttcttgggggttggatatctgggcctcctttctagtcactgcagtagcagctgtgccccctcaggctgtgggcaggaccttcttgcaaagcagcacatgcagctctgggtttggaaaatgtcagtgcttggatggaccaggaatccatgtacctctcactcgtcaagaaatctggcagaggtcacactcaacaggggtgctgtgggtaccccagccagactccctttccctggtcagtgtacccatgaatcccctggctgcaggtgtgattactgggaatgctcccagctgccccttctgtgaattgcccttggctgaggagtgactggctgatagggtgaaatggtttggctgtgtccccacccaaatctcatcttgaattgtaactctcacaattcccatgtgtcgtgggaggaacctggtgggaggtgattggattatgggggcaggtctttcctgcactgttctcatgatagtgattcagtctcacaagatctgatggttttaaaaaggggagtttccctgcacaagcgctcttttcttgtctgccgccatgtgagatgtgcctttcaccttccaccatgattgtgaggcctccccagccacgtagctgtaagtccaataaacctctttcttttgtaaactgcccagtctagaatatgtctttatcagcagtgtgaaaatggactaatatacagggggacaaaaaaggcagcctcctcgcctcatggtggacagcccagtgttagtttatgttgcagagcccccttggtcaggcccctctagactctacctgggatccattcttgctctatttccatcctctcctgttttcctacctcccttatgtatctctcccaggagcccccttgataaatgacttataactagatccctgtctcaggatctgcttttgtggaacccaacccaagagagggggacagacctgcatgcagagggggacatgaacttgtatagtcaggaaccatagggtagcgtgggctcacagaggaagaagtgcctgggtgcctgagcttgggggaagggatcagagggtgtttcacaaaacaggcaacactagagttcaccgcacggggagaaagcaccgtgggtggtgggtgcggcatgtgcatggcctggaggcaggagagagcctggtatgtatggggaaccccgcagagctctgggtagcccaggctccaagagaggttggcaggggccagacaagaggggccttgaataccccagtgaaaagtctggactttacagctggaaacgatgcatgcagaactgtcgggaaaaacatcctgtggctgcagctgtgtggagcagacacacgaggtggggtgccggtggggtggccctggcagctgtcagccaccaggctaacagggaccgagggatgaggaacagggctagattctagaggctttcaggacaaaggactaaaaggccttggtggctaatctggggctctgggtgtgggaaggagtctagaaggtaccacaaagagaggagacaaaacatcagggctgaggcagggttgaatgagctgtacagggcaggggtctgcattccctgtgcccaccacagctagagccaggctgagcctccagttggaaggtccggacactgaccagagtatgtggcacagctggcctgaccccagtaaggacctggggctgccccttgctggaggggctggcctccaggcctgattaggacagggatggggagaggctgggggcgggggagtagagtgcagagctgctcaggcattgatgcgggggtgtggttgcaaggccctgggaagggctcaccatcagctctcctctaactcttctattcatggacaccagagtaaaaactacacaagattacccactggtgatgggggaggcaggcttgcaatgcagggtgtgtgcagagaggccaaaacaagcaggggagggggctaccctccatggggggaaggaaggggagggtgttgccatggaagactcatgcttggacaggcagaacctgaccttcacccagacactgtccttatctcagacctgctctgccttaattccttaatttgctgggacatttgctgggcacccggtgctggaagcacagacaggtcggctccaggctctgtgctcaaggcatccagaggggcagaggtggatgagagttagagaagccccaggaggggccagaaaggggcctggggaggtgagaggggaaagagcatccatggtgggagacacatgctccaatcagagggaggggacttaggaggggacatggcaggaggggagagggctgcgtgcaggctgaggacttgaggaggctgggggttgctgcattttgatgcttggccttcagccccttgaatccactccagaccacttcctagtgccactgccattgtctatttctgagtcgagctccaatccagactggcagtcctggatgcggggggccatctcttgcccaagtttgccagatgaatggaccgtgggtgttagatgggtcattctgtagccatgaggagatggcactaggcaggctggcactggcatccaggcaagagatgctggagatgagagagggtgggcattttggagagagagtcaccagccttcaatgagctggctggagaaggctcagggctaaggtcagggtcatgggccccaccctggtggggccccaggattctgtcttatgcttagggctagatgatatatctggcgtctattcaaaaggggtccctgccagggcctgaccttctgacacccctttcccctccagagcagcccagccttctccttttgcccgggtcccacccacctgcccttgccatgtgacctttgaggccttccccaggttgagtctttgcagccagggctcccacattcctggctatgctggtgttggccagcagccctagttcctttgggaaagagcggacaggaggctcccatggcggggactgtatcctttccttccctcccttgccccaggggctgcactgggattagggtatcttcctccctggccatatcaccacagtcccacctccagtcacagtctcccctcccctccctctccctccctctctccatcctcctcctgtttctctggcctcatgaagagccccatcatcccccagctgcccaccatggagtgggtggttggtagtgccagttaggtggcagtgcaggtgcctgagccagggtgaaggctggaaccatgttggtgaggatggaagggaccagctgaaagtatccaggcgatgaggccttttgacttttgaggggctccaggatgactacaggtttctggcatggggggtctcttttgctgagacaaggacagaggggtacaccagtctggagggaagccacactgaattggaggtgccttcaggtaccccgggggagaaggctgtctggagggcagcaggatatgcacactgttaaggtgtcccatttgttggctgcccctttgaagctgggggcagggcagggggccagagcatactgtagtgcaaggtcagaggcccaccctaccccactgctgtccagggctcctgttagagccacagggaagtcgctgactggctgtccttgtgacccctcctcccagatagctgatgaaggcacttggtcagaaggcacggctggctgaggccacagcccctctgctgccccgaggccatccttccctgttccacgggcctggcacttcccaggcggctgccgcaccactgaccacccttccagctttgggtccctcgtgggtttttcgtcctttcccccatccctagcctctcttctcaccatctccttcccagggaccctccctttctcctcccgcaaacctcattcttctccttttcccataatcaggaagagctttgattgcccattatacctggagcccaaattgtcagcaggagagaaacgggtttcccccaccttgagggcctgaccttagacactcaagtgtgccagccagggcagcattcctgggaagagaggggagacgtctaggttccaaccagacgagagtcaccctccccagaagaatgggccaaatgactcctaatacaagggaaaatagagacaaccccccaaaaaaagttttacaacaaaaccagctgaatgttcaaaacctgccccatctcatttctttgaaattggagagaatttaactcatcagcaaaaataaaggcaatgggaatgaagagatctccaaaggagtttgtaggccgggcacagtggctcacgcctgtaatcccagcactatgggaggccgaggcagttgaatcatttgaggtcaggagtttgagaccagcctggccaacatggtgaaagcctatctctactaaaaatacaaaaattagccagggatggtggtgcacacctgtaatcccagctactcaggaggctgaggcaggagaatcgcttgaacctgggaggcggaggttgcggtgagctgagatctttgcattgcaccctagcctgggcaacagagtgagactctgtctcaaaaaacaaacaaacaagcaaaggagtttgtgtggtgtaagaagaaaactggggacatttaaggcgaaaactaaggaaaatgattcagtgaaggagatcagaaagatgtggtcagaggagctggaggaggggggtggaagaggaggggagagagagagggagaaagatggcgggatggcggggggagacagagacacagagagagagagagagacaccagccaagataagagagcaagagagggaggagtttccaagagactcagagaagctataggtgaagagttggagttcacaacccagagggaaggtggtggtgccctcttcttggaccaagttgggtggaagcagggtggaggtgagggagtcatggagtggaggcagtgactgtggggagggggcaggaagaagtcgaaggcagctgaaggagaatgagtgagtccagggaagggtagggtgctgagctggaagagctgagatgtttctaggcaaaggggagggagccagagagaggaaagcaaggcccctagcaggcaggacccaggctggagggaagaggggcaggaggaggaggaggcggcagacaggctgggaggcagagtacctgcaggtgtggcgggggctgacggggtggcaggaagcagaaatgacctctgcttattggtctgaacgggaagtgaggttttctcctgagatctggagagaggatggaggaatatttagatgaggctgaggagaagtgaaggtttgaggacaggctagaccatagcctctgcggtggctgcaaggctctggtgactgccccttggccacagccccccagcagtttgagggtggggccaaaacgttgcctacaggggcctgtccagggctgtaagtgtggatctagggagggagagggaacctggtgggagtaaggattgctgctccccaaccagtttcctagggaaaaccaaatttagaatcaacagaaacatgactgggcttggtggctcatgcctgtaatcccagcactttgggaggctgaggcaagtggatcacttgaggccaggagttcaagaccagcctggccaacatggcaaaacctcgtctctaccaataaaatacaaaaaattagccaggcgtggtggcacgtgtctgtaatcccagctactcaggagttagaggcatgataattgcttgaacctgggaggcagaggttgcggtgagccgagatcgcaccactgcactccagcctgggtgacagagcaagagacagtcttgaaaaaacaaacaaacaaaaaaaatcaacagaaacctggcaggggcttcggaattgtaaaatgtgactatttgctttgtggcaatgtaaaaaaaaatcacattttcactggaaagaagaacaaaggaagaaaactgaatgtctgggaacaaattgatgctgacatttctctaccttgctggtgtccacctgcccacgtgcctggcttgtttatcgctgtgcttacaaatatttcctactctttacttcttgacatcttgaccaaggtaactgtgatgaggtaagacacagtctgttgtcattttccagcaagaactcacccaaggagttaagtgatggcccagagttttccagaatcactatcacctggcactacaatagaggcagagagcgaatgatactgtccaagacaggtgtctcttaccgtgtgaagataaatgctggttgatatcgcccattaggccgggtgtggtggctcacgcctgtaatcctagcactttgggaggccgaggcaggtggatcacctgagatcaggagtttgagacctgccttgccaacatggtgaaaccccgcctctactaaaaatacaaaaattagttgggcgtggtggtgcacacctgtagtcccagctactcaggaggctgaggcaggagaattgcttaacccgggaggcggaggttgcagtgagccgagattgcaccactgcacttcagcccaggagacagagcaagactccatctcaaaaaaaaattataggaaaagaaaccttttgatcaatgaagatgagtggtgatctttgcaaaagtgtacagattctaaatggaatgctggggaaaataaaaactgatacattacagggttttatatctgtcaatgtatccccagcttttaaaaaaggagtcattggaccctagtgttaggtccacagggattcaatatgttatggctccccttttgtgtggtgtgtgttcagaagtttccataattgaccagcctgtaatcccagcactttgggagactgaggcagaccgctcacttgaggtcaggagttcaagaccagcctggccaacatggtgaaaacctctctctatatatacatatacatacatatatatatgtaaaatatatattatatattatatatataatggttccgtaataaagtcttaaattttttcttaaagatggaacaattgacaatagacacctgtcattcttctttttcccttcttattttggaagctctgctagatgctgctgtccgcaagtactgaccttgccttttctgattttcattaaaaagaaactcatagcaacaaggtaccagttaattcctctcaccagagagctcttctccctgtttctcctccctcaggcaataggagatatgtaaaatgccttttcaggctccctttcccctcctcaatgcctaactttgccatcattcagcactttccctttagggaaattcttattgcatctctcttctgtttaaaaaaatatacttatttagcccttacattacaaattccatcctatcattatttattcggaggtaattgtacatgatttggtcagctcacggctcaccacaggttcctcttttcttcatctccttgtctccaatctgattcatctatttcctgattagattcctttctatggtatttcccccatatgggaaaattatgtggacctttgcatttcttctatcctaggaaggtgaatggcaacctggttggtttaggaggcttttctctgagtggtctgtggaggtgattcatattggtttctagcttgcaaataagatgtacgatttcagtctgattctcttctttctatgcaacttattttctcattagggaagcttgtctgaattttctctttaatcgtggaattcaggaaccttaccaggctatgcctagatgtgtgtcttttttccatcattcctgcctggaactcaatgagccttttaatctgcaaacccaagacgttcttcaaaccacggaaaattttctttctcatttaaaaatgattgcttctcttctatttcttcttttttctgagatttttattcttattttcatcattcacctattacaactcctggatctctctcctctgaggcgcttttagatttttactttgggttttgcattatctcttccacatggtttcctctcctccaagtcattttaaattgaaaacgatgtttcttcgttcaggaaagtatttgatttcacctttgactattttattttatttttgagatggagtttcgctcttccacccaggttggagtgcagtggcgtgatctcggctcactgcaacctctgccttccggtttcaagcgattctcctgcctcagcctcctgagtagctgggattacaggcgcccgccaccaggcccggctactttttttatttttagtagagacggggtttcactatgttggccaggttggtctcgaactccttgtgatccgcccgccttggctgcccaatgcctttgattttttaaaatgtgagcttatggttttgtttgcttttcctgttatgcaagttgtggttggtctcctcttgcagtgctgagtgaattgcaggctcttccttcctggccgctggattcccttaggtgccaactgtctttctgccttcttgcagccctaatcatctccaaggctcagatccagctgacaaattggctgcacctctctgtatcccaggagagagcgctctgctcattctgctgctctgcatccccgtctcccagcctcggggcagggagggattctgcagggcctcccaggaccacccaaagctgctggctctgctctcctctgtgcatccctgcgccaagttctctctttggagccttggagatcccattctcacccagggctctctctgctgcgggtccctactgtctgccccagagggagactaagtcctaagccagagctatcaagacaaaggccctgtccaaccgaccaggcctggctgctggctgacccaggagctctgctgtgggagcccattggatattggaaaggagggcagcagggcggtcaggctgccttcaccctggaggtttaattaaagagaagttcttgggtttcatgatattgaagagatgactgggatcaatcaaaagtttacttatacttgcaaaagtatatcagaaaaaaataaaaagcaaaatggaatggatttgaataaaatcagttttttttaaaatttactaggtctatgatgatgtatctagcatgcatagaaaattgagtgacattgaaggagttctgaggaattccgcacatttttttgttgtggtaattatagcattttgtgaggaaaagaataaaccttggctgggtgcggtggctcacgcctgtaatcccagcactttgggaggccgaggcgggcggatcatctgaggtcaggagttcaagaccagcctcaacatggagaaaccccgtctctactaaaaatacaaaattagccgggtgtgatggtgcatgcctgtaattccagctacttgggaggctgaggcaggagaattgcttgaacctgggaggtggaggttgcggtgagccgagatcgcgccattgcattccagtctgggcaacaagagtgaaactccgtctcaaaaaacaaacaaacaaacaaacaaacaaaaaacaaaaaccttaaatgctctcagttctctccttggcacaacaccatgtacccaccaaaagagttttccaaacctttggacgtgggtgaagatgaagtgtgcagggaacagggtgatttctttttttttttttcctttgagacggagtcttgctctgtcacccaggctggagtgcagtggagcgatctcggctcactgcaacctccgcctcccaggttcacgccattcttccgcctcagcctccccagtagctgggactacaagcgcccgccaccatgcccagctaattttttatatttttagtagagacgggctttcaccgtgttagccagaatggtctcgatctcctgacctcgtgatccacccgcctcagccgcccacagtgctgggattagaggcgtgagccaccgcgcccggccaatttcttgaaaacacattggactgggcgtcacggtccatcttcagctgccgatgaagaactttcagataccattagaccacaggaagttgtaacaaatgactcttcagctcttgcaggtgagcgaagtatacaattgctatcactgattccaaatttgattttatcttttgttttactttcttttaaccacatgataagtgtcccaggatttttttctaaagagcttcaaagtgaaactgtagacattttctttgcctttaaaagccaaaaaagatcaccatctgagagaaattatgagcctttcaaggccacctgggacagagtggagaaatgatgccaaaaaccaattcctaaggacttcaaagttgaaaaatgtttccttcagaagaaaagaactgctgagataaaacaaatgatggcaattccaattatgcatttttcctactttctcataagaaaagtattaaattaatgtctcttatgaaggatgggttcctagattacatttaaagttacagtttcatagaaataatcatattgaagtgaagcaaatttctgagcagtcccattcagttgaaataaaataatacacagaaatagtcaaacacgtgcaaaatgtgtgcagcctggaagcactctgaatcccacaattcagagtgtgttttatgaaactccaaaaggatcacttctgctacgattgtatcaacttaattgatcttggcagcctgatgatttgacactgtcttcactaccctgctccttagtctcagcgattacaatagcagtgcagaaagctcattttctactcttagtgacttagaaacccgcttctgctgctctgtccagtgacaggctctggccccaacagctgcagagtgagacaaatcacatcgaatgtttcctgagtctttccttttccaccctgatacggtttcttcacccaaatctcatcttgaattgtagctcccgggagggatgggattgggagggactgggtaggagacaactgaaacatgggggtgggttcccccatactgttcttgtggtagtgaataagtctcacgagacctgatagttttttgtttttttgagacagagtacactctattgcccaggctggagtgcaatggcgcaatcttggctcactacaatctctgcctcccaggttcaagcgattcttgggcctcagcttcccgagtagctggaaccataggcacgcaccaccatgcctggctaatttttgtgtttttaatagagatggggtgtcaccatgttggccaggctagtcttgaactcctggcctcgtgatctgcccgcctcggcctcccaaagtgctgagattacaggcatgagccactgcgcccagccaagatctgatggttttacaaggggaaacccctttctcttggttctcattctctctcttgcttgccaccatataagacatgccttttgccttctgccatgattatgaggcctccccagccacgtggaactgtgagtccattaaatctctttttcttattacctagtctcaggtatccctttgccagcagtgtgaaaagggactaatacacaccctgaattcattagttcattcagtcttcaagcaaccctgcaggaagaaatcactagtcccatttgctgtaggaggaaagtgggggtcagagatgttaagtgacctgcccaaggttgcaaagcaagtaagagtcaaacctagcctgattcccaagccggtgtaatcactgtgttcagtttgcagccaaagggatgaagatctgaggctcctgtgatatcatggatcatctatatttgctgagataaaaatgatgtgctactaatttaatctcatctgtaagaacttttctttcccagagttatgatgtcttttatcaggatggttagacatccacgtatggttccgaagctcctgctgaatagggcgggccatggaatgtctggtttgttgctgggatggcataaagggcatacactgaagtgaaatgctgacaaaccattcctctgttcttaaaaaaataaaaaatgtgcctggctaaacaaactgtggaatattaatataatggaatactactcagcgataaaaaggaacaaggtactgattcttgtagcaacatggatgaatcccaacagtcttacactgggcaaaataaaaagcctgacacaaaagggcacagagcaaatgactttatatgaaattctagaaaagacaaacttagtgtattattgtgctagggctaccatgacaaagtaccaaagacggggtggcttaaacagcagaaatttgtttcctcacagttctggaggccagaagcctctatcaaggtgttggcaggggtggtttcttcgtaggcccctctccttggcttgtaaatggtagttttctcccagtgtttccacgtggttgtccctctgtgtgtatctgtgtcctaatttcctcttcttataaagacaccaggcatattggattaggttccaccctaaggatctcattttaaggtaatcacctctttaaagacccaacctccaaatacagtcacattttgcagtactagggattaggactttaacatatggattttggaagaacacaattagcccacaacacctagactatggtgaaaataagcagaatagtggttgcctctgggaggagtgtgggttagggatatactaggaagtggtttgggagactttctggggttgatggaaaattggatttggtgatagggtttgggttataaaagttatctacatttgttgcccgggcgcgatggctcatgcctgtaatcccagccctctgggaggccgaggtgggtggatcacgaggtcaagagactgagaccatcctggccaacatggtgaaaccctgtctctactaaaaatacaaaaattagctgggcatggtggcacacacctgtagtcccagctactcggcaggctgaggcaggagaattgcttgaacccaggaaacagaggttgcagtgacctgagattgcaccactgcactccagcctggcgacagagcaagactccgtctcataaaaaaaaaaaagaagtatctacatttgtcaaactcactggaataaaacatgtagtccacatggtacatggaatagtacaatttatgcatttaattgtctgttaattttgcctcaaagaaaaccagaaacaaatattgaactctagttaataatatgcatgcctactgttaagggataaagtgtactgctgtctgcaaatagcttttgaattgatggctgggtctacagatggatagatatttgaaaatgcacatatagcaatctaccaacaattgcagaatttagatggtggattccctgtacaattattattattattattattattattatcattattattattattattattgagatggagtcttgctctgtcgcccaggctggagtgcagtggcgtgatctaggctcactacagcctctgcctcccaggttcaagggattcttctgcctcaggctcctgagtagctagaattacaggttcccaccaccatgcccggctaatttttgaatttttttagtagagacggggttttgccatgctggccaggctggtctcaaactcctggcctcaagtgatccacctgcctcggcctcccaaagtgttgggattacaggcttgagccactgcgcccagccctttcttttcttttctttctttttctttttcttttctctttttttttttttaagacagggtcttgctgtgttgcccagactggggggtagtgttgtgatctcagctcactgcaacctccatctcctgggctcaagcgatcctcccacctcagcctcaagaataactgggactataggcgtgtgccaccacacccagctcatttttgtattgttttataagagttgggtctcaccatgttgcagaggctggtcttgaactcctggactcaagcgatcggcctgcctcagcctcccaaagtgctgggattacaggcatgagccactgtgcctggcctcattgtataattctttcaacttttctgtatgtttgaaatttttcatcataaagtgttggggggggggataaaatttccatttgaaaatattagctagacatttctcataaaatctttcttcattgctagatcaaggttgccacgagactatgccaagtcaattgtccttgtgtcccccaccaatatgcccaggaagaaagagtagggagacaattccgaaagaggccttgagggtgctagggagttctagggactagctaaggcgtttgggctctgtgtttgaggaaactgaggctcaggctgttttgcgttgctgtttagaatccagggaagtccaggatgttttttcttaagccatgttggggtgcttaggctcagtggagaagcggggcatggccatatgaatgatatgcagttggcagcacatagtaggcacacaagtgtttgctgaatgaatatttacatgaaattggaaaggagccttccaaagatggagccagcaggagacagtgaagttgcctgtgcgggccgggggagtctgtctctgtctctgccccagcccacctctggcctggcctgagtaccttgagaagtgggcccaggagaggcccctggatccctgggttcctggacagccagggtgagggcgctttgccacattcttaggagagagcacatccaaaacaatttctttttctaaaaactcagtgctaacggattccagatgtccgtttgttatataatggggtttgttttaatagcgtcaacttccagatgtgcagcctggaatcaagaagcccattggccccctgaggacactgtgagccaagctgagagctgcccgggggagagacatggacgtgcagaaccagggatgggagaggagccagggtgccttcttgctgcccactgggctgctcacctgccctgcagctgtccccagactgaaagacaggaagggtgttcccgcttccttttttttcagtcctgactctgagcaggggctcccaggggacaatcatagacaggcagcttggggagggtctgctttggaatctggttcaagcaggcctagctggatgagcctggagaagtccttggcctccctgtctttgaatggaaaatagaataacgccattctacccagagaggtcattgtgcaaaatgaaatgaagtaagcagcacagcacggggctcagaaagccttgactttccctgagtggagagaacaggggcccaagtcaccgagcatcctcccctactcctctcagcaacacggatgcccctgggcagcacgggctgcattttccctggaacactggaaaagagccaaggtggggagctcaccacctcccccagtggcttgttccatggccctgcagctccaacggatagaacattcttccgttcactgagccaaaggcggcctctgtgtgacccccaactggtctgagttctgctctctggagctctgctgagcaggccacaccctctttcacctgataccctcgagtatgggaagacagagaccgagtctcccaacccttttcctagtaggcagctgcagttatgacatcagatggacgaagattaagatgctctaaggagagatgttcaaaatcccaaacaagaaaagtctcaacgtctgggggaatgattctgtggttctccctgtgtagatgttaataaaatctgtatgccttcactccaattcaaagagaaaagggccaggctcacgcctgtaatccccacaatgggaggccgaggaggatcacttgagcccaggagtttgagatcagcctgagcaacatagagagacctgtctctactaaaaataaaaaaattaaccaggggtggtggcatgtgtctgtggtcccagctaattgggaggctgaggtggcaggatcacttcagctcgggaggtcgaggctgcagtaagccatgattgtgccactgcactccaccctggatgaaagagcaagaccttgtctcaaaaggaaaaaaagaaaagtcacaaaatctgaatgcagtcacaggggtgaggtgtccacaaagagataaagtcatcatcttctccaaatgaatctatacacacaatgcccttccaacaaatttccaatcaaattggtgggggtagggagagggtgcaatataaagttgaacgttaagtgtagaatactcaccatgtctccgcagatgaaaaacattaaagcgggacttaccctctcagctgttaaaacacaggtaacactaaaatacattgaacattatggtacttgtgccggcgcagcagagagggaaatggcacagaaaatggaaacacgagcagtagacccatctccatatgagaatgtggaatgtggcaaagaaatgcatgtcacactaaagcaaataggaaatttcaacagtggttacctttggggagagggacttggcgtgggacagtagtgagtgagagggaagtgttcacttttcattttgtatcttttagagttgttttattttccaaccttatatatttttgtttgtttgtttgtttttgtttttgagacagagtcttgctttgttgcccaggctggagtgcagtggtgcaatcttggctcactgcaacctctgcctcctgggttcaagcgattctcctgcctcagcctccttagtagctgggactacaggcgcacaccaccatgcctggctaattttttgtatttttagtagagacggggtttcaccgtgttagccaggttggtctcgatctcatgaccttgtgatccgcctgcctcggcctcccaaagtgctgggattacaggtgtgagccacgtgccctgccatatgtattcttttttttaaaaaaagccaacatatatatatacctatatatacacacacgtatacatatatacacacatatacatatatatgcatatatatatatatatatattttttttttttttttttttttttttgagacagggtctcactctgttgcccaggctggagtgcagtggcgtgatctcagttcactgcaacctccttcacctgagaagccaacatgtttttatgccaaaatatgttccagaggttttgatcccttttgggttttctcttttgaatatttctgcacttaagagaaacctagtaagtgttataacacgaataataataggatctataacaaatgatgaacagttattgcctctacctcagggaggtaaaaggggagggcttttacttccttagccatacatttccacacttctcatccccctcctgctcgttcctcttcttttttctggatagcatgtataactttaaaaatcgaggagaggggggaattgaaaatttgaaaaaaaaaaaaaaaacagggcagaaatgatggattacttaagatgtgggactggacaactggctaacaatttggaaaaaagttagatttctaccttaaatctaaatatactgcagatggcttaaagatttaaatgctagaaggtaaaccataaaagaatccgaatacttacacaattttaggataggggacaacttcctaagaagacatcatatgaacagactgatagatttcatgacataataatgggaggcttctttctaacaaaacataataaaataaggtcaaactgtgaaaaaaaagacaaacaatatttaacagaggatcaagcattttaatatagaaaaagctcttacaagaattcctgcctcataattgttttccctaagccaagaatgggggggaatttgaccacagtgaaatcagggtttctaagggcaggaggaaggtaggaggggaggggctgaacggagttttcaggatgggggtggacagctcagggggacagctccagggccagggttgggtgacctccatgtggcaggggttccccactctccctggcattcacagtcttatctggtgccagtcacttccccagccagcctgacctccctgcctgggacatttaacattttgccatcttcctcacacatcaggcatttccctgtttccatgacttttgcaaatgctggtccttctgcttggctcactctctttcatctctacctgaaaaaaaatccaactgctccttcaaggctcagctcaaatatcccctgttctgcaaaactgtctttaatccatctgcccccactgcccccaaggaactccccttgggctcctatggcaactgcccatcagtctattcagcacttactatacagcattgtcattttctgctcactggctcactctagacagggagctccagggacccctttccccttctccaggtccggcaaagagcaagtgctcaattaatatttcatgaacaaatgcatcgtactgtgagtgacagagcagggcaactgaactgacactagttgagcttcaggttcagtccatttcttttatattttttgagacagggtcttgctcagtcactcagactggagtgcagtggcatgatcatggctcactacagccttgaactcccgggctcaagcaatcttatcacctcagccccctaagtagctgagactacaggtatgtgccattgtgcccggctaattttttattttttgtagagatggggccttgctatgttattaaggctggtctcaagctcctggcctcaagaggtcctcctgcctgagcctcccaaagtgctgggattataggcatgagccattgtgcatgactgatttatttcctaaaacagcatgcttttatttaaaataagggcaagttatttaactatgatgcaattaagagaaataattatgtataattcaattatgatgcaataatttatcataactgcatcatagttaaaagatgccaagttactgtaagatcagaagacctacgctaaggcacagtggtacgtgaatgcacttgaactattgaaagccgctgtcagactccacaatggctttcatttttatttatttatttatttatttatttattttatttattttttatttttatttttgagacagagtctcgctctgtcgctcaggctggatggagtgcagtggcgcgatctcgactcactgcaagctccgcctcccgggttcacgccattctcctacctcagcctcccgagtagctgggactacaggcgcccgccaccacgcccggctaattttttgtatttttagtagaaacggggttttaccgtgttagccaggatggcctcaatctcctgacctcgtgatccgcccgcctcagcctcccaaagtgctgggattacaggcgtgagccaccgcgcccggcccatttttaaaacatttataaacaaaagaggtttaattgactcatagttctgcatgactgagcaggccttagaatcatggtggaaggcaaggagaagcaaggcacatcttacatgagggcaggaggtggcggggcagagagagagagcgcgcctcaatggctttcaatagttcaagtgcattcacaccaggcgcattcctgcgcttgagattcctagcatcagccaggagcggcttagtgggagaaagagctgcgacggtgatgatagtgacattccaaagttttgtggcaacatgaaggaggaggtgtgtctgcgcttgcacagtgactgcagcccttgggcagatgtggagaatgtgtgaggctaaaggctcccacgcagcatggggcatggggagcctgaatgtaagccccagggcaggaagttttgcctatttgctcaccgtgtaatctccagcatctagaatagcatagtaggcctttggtaaatatctgttgaatggaaggactaaaattagttctggaagtgtctggggatggggcttaagttacctctgcagggtctccaaggctaaatccttttcctgagttagaaaaacagggctgttctcactgaagttttggccttcagagtgtgatgaggagtgcaaaggggcacggggcagcttcaaggacagggctgggaccacagagggtgagaaaagggcacaagggaggaggggtgctcccaagatggcagcccctagagatctgggctgggtcttgctttcctccctggggccagtgcagcaggagagggtgctgggtaggggtgctgggggctgagaggaccaacccagcttcaccaggcccagaagagaagatgcacacagggagaaggcagctgtatgcagtgggctcaataaagaggatttgggctgggcgtggtgagtcatgcctataatctcagtgctttgggaggccaagtggggaggatggcttgaggccaggaattccagaccaacctgggcaacacagcaagaccccatctctatgatacatttaaaaaaaaattagccaggtgagtggtgcacacctgtagttctagttacttgggaggctcaggtgaggggatcagttgagcccaggagttcaaggttggttacggcaagcactccagcctgggtgatggaatgagaaatgaaaccctgtctctaaataaataaataaataaaaagatttgcctctgtataacaggagtttgaatttagggctcaaaaagtcaagtacattctcaaaatcactcagagagtgacaaaatcaggattcaatttcacatctttctaattccagagcctgactttttttcactccgctaaaaaaagtgacgtcacaggcacatggcaggcatggagccccaggcgagccaggtctcagtctgggtcagtgtagccccagggctgggatcagaaccacagtcgttctggagacagggccaaactagggttagaggtcagggaggccaggcctgagaacaacaggctttccgggctttgtgcccccagccagacacagtgtcctgggattggtggcagggcctgctggagcccccagctccctctttccaccccccatctagggcctggcctgaccttcctccccagggcaaggaactggcctggctcttatcctggaggaggctgcagctgctcctgcagccctggtcccagcagacaaaggcgctttctttcctagtacaaggtcttgattgcatgttgccctggatccaaaagttcctatggaggaagcagtttcctcccagggtgtgtgtgtgtgtgtgtgtgtgtgtgtgcacgcacgtgtgtgctgggtttcagggagttgggctgtcagggagtgcaagccctctcccagcctcctgggtttagcctggcattgctcaatctgcatcctttgtgtacatctctaagtgtctatgcatgtgtgtgtacgtggtggtgtgtgattgtatgtatgagcatgtttgtgcatgtccctgcagctgcaggtgcgactgcctagtatgtacctgtgcatatttctgtacacatcagcacatgcttatacacagtcgcaagcctacgtatctgtgtaacttgtgtctgtgcccacatgcgtttgggggtgtatgcatatgtttcatgtgtgtgtctacatgagacacctggtaccaggtccctaactctgctgcctctggctgtgcctccccacaccctctgctgctgtgtgggggctctgcagtatgcaagtcctcctaaaatagccccctttcttcttcccaacccccggccccgcctctgcatttgtctgggaacaacctggcctctttcagctctgggaggaagacaggtttgtacagcttggcttggcccatacactttccaaaactctctggctcccagccaaactctttataaaaaggctcatggggaatagcctaacaggaaggtgggaactctgcctggggggctcaggaaccctgctccaagccccaagattaaatgggagcatcagtcagctgaacttcagccagagacggccctgcagcctgaaggtgaacttggtcaatggacccttcacccccaaagatggggacacagcatctcaaggtctcatgtccagggtggcagagctcggaggggtcccagggctcaggcagctcaactccccatgggcacactggagggagaggcccagagtgatgccccagaccaggcaggagctgagccacttcctagcatcctgcagccccaggccctggtgaccctgccatggataaccctgggtctgcctcttctgcagggaggtgggtgccactgccagacttcagagtagcaggctctgagcctggctggcccttttgctcaaggcctcctgagtccatatcacaaagtcagtgtttgagcacagggttggtttacccagcacttgccttacttggggtgcaggggacctcaagctgtttctcctagtcccactgaggcacaagagaggggctttctgtactgctatctcccctactggggctcaacccagtcttttttttttctttgagactgagtctcactctattgcccaggctggagtgcagtggtgcaatcttggctcactggaacctccgcctcccgcgttcaagtgattctcctgcctcagcctcctgagtagctgggattacaggcacgcaccaccatgcctggctaatttttgtatttttagtggagacggggtttcaccatgttggccaggctggtcccgaactcctaatctcaagcgatccacccaactcggcctcccaaagtgttggaatcacaggtgtgtgccaccgtacctggcctccactgggtcttaagggtgccctagggtgaagatgggagggcagtccaatgggtaagaccctatctcaagtggatgctcagggcacaggtggcacccccctcactggtctgtcacaggagacacaggggctgctgctggagatgccacagccgtgtgggaggggcaggctggtggggcaactgaggaagaggatcccagaagcctgggttcagacatggctccagggggctcctgggtgctggaaacagacgagacaccctcaaagaggctggttccttcagtccatccccactgaccgctgccaaccacccctttcctcttcccttcagaaacctttttccaggcgggagggatggtagctgggaagctttgctcttatctggcatgtgcccaggccaggcaccgaaactcaaaagccaagcagagaatctttttttgtctgcttccgattgtcgtaccttgtccctgagagacactcatggaccagcccagcttgaaaaggagccagagaacacaagaaacaaccagtaactccaaagaaaggtcagggtttcaagaacattgtgcccccccggttgactgtgcagagcaagtacctctttggatggaggttttatttctttgaactcttgttccttacagaggctgggagtggttgggcccacagggtgggggtggtggactccttgtccttgggcctgttttggggatgctgggttgcgcactgggagtggagttggtgggggctctgaattctcccacgctcagtctagggatgggagctgcctctttcttccctcatgggatgctaattatcctcaacttgtctgggagaagaaaggaagtagctgcaggggcttagccaggaggaacacctgctcccctgccccaccgccctccagctctccctccctccctccttccctctctctcagatgcacaccaacactaggagtatccttatatacgtatgtgctgcttcttgacggggaaatttgacaagggactggggtgagggtaggggtgtcttctacctggggaggcccaaggggctgcggctggggctttcctcagcctcatacaatccaccgccccgcccccaacccctgcccaaggcactcacggtgccttgtgcctccgggctctttgccggtctggaaagtctgggatctgtgaatggaaaagcctgggtcagagctggagtggggagtgggtgggggttgggagctgtgtctacagagtttagagttttcttggtctgagccaggaaagagtctaaacacccagcagtgctgagtccttgcctgccctgtgcagcgtggaccttcctcggcccagctctcctgccctatgcaggctttgggagtccctgaggattttcaaagagccccaaggcgctggggacagagaaaggtgaggccaggttgtcatcctgagggggaacaggcacacacagccagcaggggcctgatggtagatgagaccagggtgacgtggcaaagctgcttcagtctggccacagcactgcatcgacctggactcttgagcctctcctagtgtcctgcttctgagagccagctggacatctcagtggggaacaggacagtcactctcgaggcaggaggatcgcttgagcccaggagttcgaggctacagtgagctgtgatcacaccactgcactccagcctgggtgacagagggagattttgtctcaaaaaaacaaaaacaaaaacagacaaacaaaaaagactcagtgtgaagaggggagggggacagggcaactccagtgtttcctccaaagtgaaagtatgccctcgggtttggaggctggccctcaagaagcatgcccaaagccaagggcttcttggaagcattttatctcaaaagtagacatgaatcttatattccactatgtgattcatttttatacaaaaccatgttggagttaatatgaaatatatatatatatatatatatatgtatgtgtgtgtgtgtattttttgagatggagtctcactctgttgcccaggctggagtgcaatggttccatctcggctcactgcaacctctgcctcctgggttcaagcaattctcctgcttcagccttcccagtagctgggatgacaggctcctgccaccacacctggctaatttttgtatttttagtagagatgggcttttgccatgttggccaggctagtctcgaactcctcacctcagatgatccgcccgccttggcctcccaaagtgctgggattacaggcatgagccactgtgcctggccatatgaaatattttaaatgagtttttgcattgaatctatgaataaaattgatgctgcaggatggggatcgacaaactttttctgcaaaggatcagacagtataataaatattttcgactttgcaggctgtgtggtctctgtgaccactgctgtcgtagcccaaaagcagctttgtatagacaatacacaaaggaatgagtatagctgtgtttaataaaactttatttatggccactagaatttggatttgggatcttttttttttttttttttttggagacagagtctcgctctgtcacccaggctggagtagagtggcgcgatctcagctcactgcaagctctgcctcctgggttcacaacattctcctgcttcagcctcccgagtagctgggactacaggcgcccgccaccacacccggctaattttttgtatttttagtagagatggggtttcaccgtgttagccaggatggtctcgatctcctgacctcgtgatctgcctgtcttggcctcccaaagtgctgggattacaggtgtgaggggattatttttatgttcatgaaatatttccctttgattgtccaattatttataaatgtgagtcctggttcttggcttgtgggccgtagctctcactccaccctgggaagaaagcgtgttgggcctgtggctccacatgcaaagccaaggcctgagcaagaacactagattctggaatcagacccatctctaaattcttgttcttcctcattagttatgccactgaaggcaggtgacaacctacctgagcctggtcctctcctggagaacgtgttttggaatgagctcacagaaggcctgcacagtaggtattgactaagcagtgctgtggccacctttagtggtgcatgtagctgagctgcctgggtaggaggaaggtcatggccagcagatgagtgggtgagatgtgcctcttccccctccctagtcagctcccccatccttttccccaggtcattctagctgaacagttgtcctactccctgccatccttctttccaccttccaagattggacactgcttctcatggggctccttggccctgggctggtgagtccaagcacttagacacttactccctaggataagccccagctcagaggcctcccaacaccagcactgcgggatgggagggaaaaactgaggctaactgcctccccactggcctcagggatggtagctgcagtgcaagggccagagcagcctctctccccttgctcatcatttgtgcttagtgctattttatgaagctgcactctagagggatggcctgctccaagcctcagactaaagatgcccagctcccctcctggtcagtttacagttctgggtttactccttctgagttcccgaagcccagccctgacattttccagctggatgattgggcaagttacttagcctctttgggcctgggcctgtttccttgtatgtatgaggataacctcatagggttattgcggggattaagaatttggccataacatatgtagaacaaatcctactcacaggaagtgcctagtaaatagcacactattgctactaagtaaggttaccagataaaatacaggctgtctggttaaatttgagttccagataaacaacagattttttaggctgggggtggtggctcacgtctgtaatcccagcgctgtgggaggctgaggtgagaggattgcttgaggccaggagtttgagaccagctagggcaacatatcaagatcccatctctacaaaagataaaaaattagctgggcatggtggctcatgcctgtggtcccagctacttcagaggctgaggagggggcgtcgcttaagcctgggagttttgaggttgcattttttttttagagacagggtctggctctgctggccaggctggagtgtagtgttgtgatcacagcttcctgcagcctcaacctcctgggctcaagtgatcctcccacctcagcctcctgagtagctgggaccacatgcacacaccatcatgcctggctaatttttcaaaatattttttgtacagatgaggcctctgtatgttgcccaggctcaaactcctggattcaagtgatcctcccccttcagccttccaaagtgctggaattacatggtgagccaccacacccagccaaaaatatttaaataaatttaaaaagggaaaatttttaaaagtgcatataccaaatactgtatgagatatacttactctaaaaaatgtctacatgaaattaaaatttaactgggcgttaactttccctcctaaatctggcactcctacactgagggaggctccacaaacccaggaactttcctgttccccagccccaaaacagcccccacatccctctagtccacccagcacaatgcgctcatgcagaaaaaccttctttcctgcgggcaggacagaaagctggtttcccacagtgctctgagaccaatcccagatccactctgtacttgccttgggacttggaacaagctcttccttgccccttccttatcctgtttttccagtgagactggtaaatcctcttgctggctatccagaaggagtaaaagacctatgattcaacagaatcagcagtattaagaatctaattgattgggccagtggctcacacctgtaatcccagcactttgggaggccgaggcgggcggatcacctgaggttgggggtttgagaccagcctgaccaacatggagaaaccccatctctactaaaaatacaaaattagccaggcgtggtggtgggcgcgtgtaatcccagctactcaggaggctgaggcaggaaaatcgcttgaacccaggaggtggaggttgcagtgagctgagattgcaccattgcactccagcctgggcaacaagagcgaaactccatctcaaaaaaataaaaataaacaaagactctgattaatcatttcaatgaaccccatcaacagccctattagtgcaaactatcattatccccattttacagatgaggaaacagacttggagaagggtgttccaagtggctgttcctcccctgcaactggccctcattctaggtcttcccattgtcctccaaggcttgggctgagccagaactcaaaccccaaggccatctaggaggcaccaacttgtcacagccatgctcagactccatgtccaaattaacctcatcagcttcccagaaggagccagcgttgaactcacccttttccctattccctattttgtcccatagataccttgctgagtttggattatgaaagtggccacaatgcccaccactaggcagccaagatctaaattactgggctgggtgaaccctacttctgacctaataaataaatcctgtggtttccagaagaggtaaatgaacaatacttcttacaagaggccatttaaggaatctggtgtaaatgaatctagcatcaagttttccagagggtgtgacacttatttgagtgttctgtttttcctggtgccaatttttccgagtgtaatttcttcacttttggtttctgagatagaggccccagaatgttatcacgaaaggcaaaattggggtgctgtctccccagctggtgggactgcggggtctagcccagctctgctgttcatgattgacaagatagctttggcagcctctctgctggctttcaggaggcaaattcgatgttggcatccctctacccagtgcccaagatgctgtgaggacaaatgaggtcactaatggcaaacacacaccaacagtagtccagtgtgctgaaccaactattcctattctgcccttgtacaaggtagtatttcctgggacagtgttttaggtaaaaaatgaaaccaagaccctttactacttattgtttaagttattatgagggctccctatatcttgattagaggcctataaaacctaaggccttcctgtaacaatgcccatccatgcttcaggccattcatgcacccatctgatggtgcttagttgggacccaacaagatgaggctgcgtcagtctcttgctcagccttcaaagaggctttggcactttcctgccagcctcattggtaacttctgacccaccccttttggggttgagacaccatgctcaggagcacagagttctgaaaaaccttgagaatttcagctaccctggtgatcctgtgttttatgagcctactctaacctatgcaaatctagttcccccatacattccaggtgtgtgattgaggtgggactcttggtacggagcttaagaagtggcactggggatgtaccagctgagagcgttcaggcctccagtctgtggagcaggatgctgtgctgccaattcctttttttttttttttttgagatggagtctcgctctgtcgcccaggctggagtgcagtggcatgatctcggctcactgcaatctctgcctcccaggtttaagcgtttctcctgcctcagcttcctgagtagctgggattacaggcgtctgccaccacgcccagctaatttttgtatttttagtagagacggggtttcaccatgtcggccaggcaggtctcgaactcctgacctcaagtgatctgcctgcctcagcctcccaaagtgctgggattataggcatgagtcacctcgcccagccccaactcccttctagggcatgttttaaccacgtgttctggtcagttggtcaagagacaggccaagtcaaccctcatgatgaatattcgacaagccctcagtagggtgcccagcacggatcagacacctcggaaaggcgatgggcataccatgtactcatcaatgccccaggcagcaggagacacccaagtgccactgaattcctcaaatacaaatgtgacttttaaactttttcttttgtttttaaatccaggatcaagaccacaatttattatgtaagacatggggtgaagaatggtcaaggaaagttatggccgtgagtgacatggaattagatgaaaaggctcaagtttgctgaagagagtttaaatttggcttttgctcttggaaacgtcaaaataatcataagaagcacttgtgccttacagagcaaataatccacagagtgtcatattcattttgcaaacagggtcacaacagcagtcaaatagaagcctgaacacccagagagttaacatacagattccataaggataacaagggattgagcatgctggtgggtttttaagtcagatccacattgaaccctgtgacctaccggaggttataagtggaacccggggaaagcagcttttccatacaaaacaacaacaacacaacgacaacaaagaaaaccagactctgctggatgtctataatactcatttgcagtaaggctttcaagatacaggaatttttatagcatttgtattttaaggatttagggcaaatacatttttttttctacttgataaaaagaaaattagtacttaaaaggttcaaaaatatattgattgagttatttttcttacataaataaattatattgatttttaggatttaacagctgaaaaaaccctttctgcttccactggaggcaaaactgaacaaaatgttagttaaatagagagagcagcatttctaagaaatctgtggtcagcattatagaccatctatgctacaaggatgtcattaaataggatttgttcaattactggattcttcttctatgatcagttatagaatttctggtttatatctctgattcataaaactgggactccactttttgaagatacatctgattgatttttttcagtcatgatttaacagacttctttgagatgctcattttaacatttacataatttataatcccaaatgtataaaagacaatgaaaaaagcatcataaataaataatgcaaaatgaaatagttatgtcagacttttggaccttctgataaattagcaaaactgtaacagaaaaagtaaaaaatacagtaaattgtgacaacaaaaagtgaaactggtactagtaacacttgcaacatttccaagggtcctgcgcagccctgcgcccccagagtactgaaccatgagcttacttcaagtctcagagtgtgaactacctgtgaagagtgagaccatcagaagggacgttaacatgaaggtgaaaggacatggggaagtgctgcttaggcagtttctttctcagttcctaaacatggagaagctgaggaagaagagaaaataatgttgacttgcaatgtagtttcgattaactgataatttggaatttgggtccaactgtaagatataaacagaatggagaaattaatggagaagtaacttttcatagctgtattataaagggtggcacacatttgacagcctcagacactcttgatcaaaggacctactagcaagtgtcaaagtgttgggcaactgtcttcttgcaggctccagaaagaaccttattcttggtgaaggaaagcctgaagtgaaaatccattcggtcctggtgctctttaaacacagagaggcaaattaatggctagagaaatctgtaagcgaaccaggtgagagcagagcgctagccgtgtgcttgtgaagcagcgtgtagctctacggagcgcgggtccttgccccacccccgtcgacagcaataactcatggtgggtaaagctttctcgcagcaagaggaatcttttcactggtgagagggatgtatagaaaataatgcctagtcagtcagtatttcttcttgctgcaggtgtctgaaaaaccaccaagggggaaattatattactaccggtaaggtttttgttttttataaagaaatgaatatatgtattttcaaccattagttatatacttctgtctatactactcacttagtaatcatgataaaatagggaaatattttaactcaaaaatatgcaccagcacttcctttttctgtgctttttggttccctgtgacattcttcctgtgcaacccagctcacagaaaaagagctcctctttgtctctgttcttccacccttcaatggtaaaaccctagacagctttcttttgccatttttcctcctcaagtgagtgggaaacttggaagagaagggggtagggcgtgtcaccaagtactgtattaactatgattgctggaatgaactggataacagaatgagaattctgtgcctcctagactaggtagacaacacttatctaatgaagtggtgagaccctgcaactattaacatctgttaccatagttctcagacaggaaatcaggtacgtaatcttacttatggaaacacaggttcttatggaggtgaagtgagggaagtaacaaacctttatgggataagaaacttacaagtcacaataatttcttaaatgaaaaaagttctaattggtgtcgttgttggagtctttgagtgccccctccccagcctgtgccccatgttctctctctgcgggcaaaggggcactgggttcggcacagttctcatcaccgctgggctccctttcacagctgggagcaggctctgggtgggagttggggttgtccccctttgtcttcttcttcttcctcttctggctctccagacctactatttccgagtgtctggcctgctgcatggctggcagagccatgcccataccaggggagaggaacatggatgggtaaatgagtccaggagatactcctgggatgagaaatgggttaaaagccacaggactactagtagttattgcaggttcagactgatcagaaaatggacctggaccaggcttgtcctcagctaaagtgtctgttttcacatcatggctactcggcttgtcttccgcagtcttttcagtcactgccgtaccacttttcgttgtgcttgatagagacgccggagcagtggaagtgcaggtggttgccatgggtggactgaggagtcccccaacaccaaacatctggggcacagcagccatgcctggcagcatcatgggcagcatgctcagggtacttttgacctcttcacctgttggcatcgtggcaaagccagctggaaaccccaccagcccggtgagggggatgcctggcatatttctcatgttctgaagtcctaccaggtccatcccagcaatcagtccattcatgaacagtggccccattccagagggagagtctgccacaatggaaggagccttcaggagttcgctccgaggccgcctccccctcctgcgggggcccgtatctcgaagaataggctcagccagagtgtgattgaacttgttttctggaagaaacccctggaaaaacaaagaaacacaaaatgctgaaggcaacaggatggcaggtggtcagctgagggtgatgaccagggctctgcactggcctggcctgtctccagctgcacgaagggttcccagtacaaggagcacctcccgcctacttgtgcagctctctggcctgactgtacctgacaggcttctccctcagcagctgagaacattttgtggcatgggcacaactagggcacctgtcactgcccagtggtaattccaacatgctgactggcaaaagtggcatccagtctgtgagaagctggcagaagagagtggggagctggagaaagcactaaaagtgctctggttctcggtgagactgttatggctgagaccccttcaccagatcccaacacttgccttcctcttgacacaggtttttattgaagcaacaaagcagaatatgtaagacagggttttaatcccaagtttaaatcctgactgcaacctcctagctgtgggactcaggacatgtcagttagcctctgagtttaagtaaaaaaggacaatggtacctatctttagagtcacggaaagtgacgtgtttaatacattacctgatccataggaagcagtgcttgctaatggcacattacttatttattgtgtgtatgccccactcctctctgaatacaatgggatgtgtacatgtttgctgaagaaatgaaggacttcttgaacactgttggggaaactgccaccaaaataagtggtggttctccctaagggagaccatctattttttctctatttggaggttcactctccaattttcttaattagcagagagttctgcagtaaagcacttctgctccacgtggaccacactgtgacaggacctgggccctcaacacgggtgatctggcatctgcactggacattcccataacccatcaggatggctgagctgcacagaatcctgggggagggcagattgctccagcagcatcagcgagcggaaggcagcacgtggaaaggaaggcgcaggctgcagccccacaccctccttcctgagagtggagctcaggagctctgcagggcagagaggatgcagccaggaacctggtccctggagcgtaaacaaaccactgcaattacgatggatctttctctgaatttataatccctagagattgagagaatttcttcccacagatgggaagcgatttctttcaggacttagtgccgcagtccctgcgcccctggagaactgcatgcctttctctctctcgactgccctccacacctcactgctatcagctagtttatcaactagtacatgcccatggtattaaaaaaggtgaactgtacttgtgcagttcagcagtctcagcgtttcctcacccaggggaggtgtacagtttgaggacaaagccctcccctgctggcaccctccctgactgcctgggcatcttcttacggttcttgctacaaagtcagtgctgttttatccccttccctttgattacagcaatggtgaagcaaaatgttcagagtggcttcaatggatcttgtcacactggattccactgaggtcaatggtcaccatcaaggccggaatgcgcagcctacggagcagcatgagacaggagagctcaacagtgaggggtgaggcctctgggctgctgaggcaactcccagggctgcacctgcggccttttcaggattctcctttggaataccttcccttccactagagtctgcagagtggggggtggggatgtgacaagcagcagctgaggctccaccagcccacagcctccttgtctgtgggaatcagaggcagggatctggctgttgctctcactggggtacactcttggggtgcgaaaggagatgtgggtccaccaagagacaccagaatgaccagaaaaaccccacagaacgcagagaaagaagccagggaagctgaggatatctctgggctgagactggccaccaggcctgagggcagttaagaaatctatttacaatctgcctaagggcacctacttttaactttaatttggcagcccaggcatttcccatttttcccactgcctctcttctccataaactatgaaataagcaaatccttagaagacccagcagatttttatttaaggggcagctgtaaccttagctggtggtactggtttcaggttctcatgggctgtaagaacacacgtgtgtggtgggccccaaatgagcagcagctggactgtgttggttcccacactagtgggcactcagcagcgaggggaacggctgtcagaggcagcagggaaggcggtgctgataagaggatgagcagcagaagagtgcgagacgccacgaaggaagcactgatcacgtgtcaggcactgtacgaagggcttcagcagcatctcaacaaatcagtgaatcagccctgtggagtcagtactatcatcatgcctgctttacagagaatttacagattcatagatgtgtgaaaacgtgcatatctgaatgtttctgagtccccagaaatattttttgaactcctacacctgtccttcagtttcccaagaatattaaaaaaataaaaaataaaaaaaaaacccagttcatcatagacaaaaaagcttcatgatggcaactttaagaaaattcttctcaaaaagccttggtttgcatcacagagctggaagaaacccctgaaactgttatatttagtccccttcgttttcacaagctgggggtgggggtggggagggtacagttggtcagggacagagcaggtgagaacacaggccctgtggttcccagcccaggcctctttccacagcacaccacaccctctcttcttcccagcatgacctgccacttgcaatttgctaggctggcaactgcacactctggctctagggaggatcactggggacgcaattcctggctggttcctgttttgaaagagagacagtaggacttgatgggtcggggccaggctaaaaggataatttctcaccagtctgtcttgagctcatcaatgcaaatcgtctcattctgggccagccactctgtctacacagcacagtctcagtcccttgctggcatggcaccacagcccagggtgcttcagccagggtaaccacaacttgaccacatatctttctgagccctcttcctgcagcctacccacaaccgagaggcagagaaagatactggggagtgtctttcctttacatccacaaatgtgccctcataactccagagagagggggcatattccacctcagaggtctttctgttaggaagaaaacaagcccagcacttgatgaatggtgcctccttcttcttttcaagcaatctgctataaatcagatcttctttaacatgctttgagaccctcctcaggggatctaggaccagactatgccccagaggacctttggccaccttggccctggcccagtaagtgcaagtagcactctcaggatctgggatgtcaaaagcatcctttccctgctcttgctgagaaccatgagtgcagacacaatggtgggtcctttcagaaatgagaacttgcttagaaggcacaagctaatctttcttcccctctagatgaggagaatagcagcagaaatgccccgaggataaagagaacgctcttcatttgcagcagagcaagagcgctggcaaaggaagtcagctttaaccatgtcctgtggaggagtcaccccagccaccagctgctgcccactccagtcaggtgcccctcccagcagcgcggcgaccacacacagcggccaggcgggctttggcagctctccctcctggtaattataccctgggagcaaacctgcttccccttccaggaaaggcctgcggagggggcaaagggaacaaggaaaaggagcttccttatgttcaagcacactggaggcaaatctgctttctatctaattgcacaggcattccagcctaagaagaacatcctcatcaatgcatggtggcattattccaactctaaaacttaactctgggtggacttaagagaaggtggtgaccagaacttcctttctcctccgaaggttagtacattcatccaacaaacgcacgctgggtgaaaagagacctaacgacaaaatgcgacgtgtggctcttcatttaaaaatcctaaaaccaaaccaaaacacaacaaccagctctaaaagacatttttgagataactggtggaatctgaatatagactagataacaagtattattaatatgcatgtgtaataagggcaataattatgcaggaaaatgttctttatgagatgcatgctgaagtatttaggggtgatatgtcatgtctgcaatttactgtcaaatggcccagcaaaacctcaaaactatacacatacaggtaaaaccaatatatacatatgtaacttacatatataaactatattaagtatatgtgtgtgtatacatatatttaatgagtaaccactaggtagtgggcattaaaaattgaatcccacaaccttccatgcagcccgcgcaatgataattctgtggacaccacccgtccaaaccacagcacgtgtggcagaagggccatgtgagctcgaaggaagggaggaggagttccttgtgagatttaaatctatttctaaaaagaggggtagggtttccgctataaatgtttcaaattactatgctgatatcctgtgccccaaggtgatggtaataggatgcggggggtttaggacggtgattaggtcataggggtcgagcccttatgaatggaattagtgaccttatgagagctggctgtcccttcctccatgcgagggcacagtgagaagacaccagtctggatgatgtggtatggggcctcaccagatacccaatctgtcagtgccttgaccttggacatttgggtctccagaactgtgagaaataaatttctgttgcttataagcctcccagtattctgttatggtagtcggaatggactaaggttctatggcaaagaaaaagggtacagtgcaccctagaaggccctgtcccaagaggagctgcaggagatacacgggtgcccctgttcggaaggctgagctgcccgaggtcttcttcttgccttactgcaggaaaagggcctccgaggcagggagtgtgtttatttaataaaagaggctaacacatctctgggaagaggctggcttgcaagaacagcttagaaatgggttttctggcaaagggtcagcatctgtagggagggatgaacactaatttccaaggaacatgagggaaactgttagacacggggaaagaaacaagcaagccaagggggaaccacatggctcttcctgagacatctctaaagcaagagctcagagactacaacaaagcaagaaccggagatcttcccttgatggcgtatctgagacaccgtgtgccgagacaaaaggagaaggtgggaggcagcgtgaaggggtgaggaaagacagaggggacacaaacaaaggtgaactagctcgtactgctcacgcaagtggaagaaagtgagggaagagtcctaagaaaagggtagaagggacattttctgagactaaagcaagaggggaaaaaagggcagcttctccaacgtgacaaactgtctagggcccaagtctcccccaaaggggctgtggtagcagatggagatgagcaacggccactggtggagctactagaccagcggtcatccaggtgacactcttgaacaagttccctgagaacattaaatgccaaactctttctgcgagaggaggaaatgactagggtgggcggtcttttggagctataaaaagaaatcacattcaagcaatcccaccgagaagggaattcctactccaggaaagatgtagctggcagagtacttaagagaaggcattcaggaaagcttgagtttatggaacaacctgggaaaggcaaaagtccccatgagcatcttcaggagctccctatttgggcagaaagtgcttttaggaggctaaaagcacatttatgtataatctgcccaaactcaacacattttcaagttgaaatgttgttactgccctctagaggttatgacttttgtagtccaaatttgtactaatctgtaaactataattaactcggattcgttatactagaagtattctgcagaagtctttgaagtcccttccagtgtcatgatgctataataggaaaagggtagtgcttagaggcagctgtttttcattgccagtgaggctggaccaggaagaaatgtcttaaatggtagccaaaaaaacctaaattaggaaatgaaaacttccaataatgagttttgttcactgacaaaggttaaaatttctttcccctcataactctacaaataggagaaacaaaacacaaacccaagttttggcaatggtttaacaaacaattctggaggtaagggttgaaaacaaatgaccactgggagtcacagagcgctcaaaaatgccacagcaccatttcttgctgctaatcaacttcctgtgctggcttctagtcctcttgaaagatgtctcccagcgaaccccttcccagctggggctttctctcaagtatgtaaggcacggctcaaccaggatgctcctcctggccccacactcacgtggccttcttccttaccctcagtcctggctccttggggatggcaggaactctctcttccccgtcagggagctgacatctaattttccaggttctttacagcgtggcctcctctgctttggtttgtctgaaaaattctaggatgaaaacggagaccaatattacaaaacatgctatcccagattcttctgatgaggctgatgcttttcaactggttttgctattgttctcgtcaatggttgtctgcacaggagctggccaggttaaactaaaggaaatagccacctggcttcacctaggctttctctcctgaagcaccagtgttcctacagtaatctggaaaacaggctgtgtgggcacccccgccctccagactagaacacctgagccgattcaccaggctgccagggccctgttaatccctggccttttcctcttgatcatttccttgcattctcttgtcactgctacttgcctggctggtttcgcctgtctctggagggtgcacatctgtgaatgtgttctcctcttcctgtgcgctctctacctacatatcttattagttaggcgcacccaggacaaagacagtggtggggtagatgagcaagtgagaagagaaaaaccaaaaaagacatctgtgctcgagtcctctcctaaaaatgtctagtctgagggacttaggacaaagacagtacgatgacttgaagggaatgtcctaaagcagaataaccataaaactaaggacagaccgtcccccaaccaccgcaaatggaatgtacataaaactaaggacagatggtcccccaaccaccacaaatggaacgtattaacttgaaaagaattcaaaggaacattttcataggtggcagagtttatgcaacctaaagatttctctatgagcttatgatactgtgtggcaatttttgtggggacagcggataagttctgtcctcttaggattttgcatgtctactttttggcatagtattttgagatgggctatcatttctattactgaagaatttaatcaaaatttaaacttaaaaacaaaagtcctttaacatgatgttgtcagctgacagatttctcccaaacagctcccaacttcctggttgccctcagcatgctttcatcaaaaatagcaacaagtcagcaggaagtaaacagtgatctcaggcagtaccaacaaacacttactgccccaaagccaggaacttctaaggagtagtcagcctgctgccttagccagtcaagcagactcttgctgggaatggatttctcggcttggctgctggctgccgtagctggctcaggagccgaggtggcaggccctggcccctcagggtgtgtggtgctcaaggtggcctgccctgggtcttcatggacttcctggatgaaggaaaaacgagtatgtataatcacgacacaatgaaaattagtttctaaagaaagaaaacatgtaaggtgttatttgaatcactccacctatttccaccctgatattactcagctggggtgcctgaattagaaccctacacacaccactgacccacgcccacataagctcttgcaagtgatttggtgttcccttcacctgagcactcctctcccgctcttgtccacttggtacactttaactccatcctgactgccggattcagttttcctcctctgtcaccactgcccactgtcccaggcagcactcactgctccagctcctgtccccacagcactcggttcctagctctctcacataaagaaaacgccataccacaatttactcaccctctatagtttgaatgcctgtcttacctctgttttactaatgcctataatggggtctataacaatgaccatgctaaataagtgtttgcatcaataaattaaaaaaatactctgaggacaagggggaaaatttttatcggtcattccattggaattctatgatcattatagagaaactatggtgttgcacatctaaaaaaggtaaaagacttcttaaaagcttgcaggattgagcagggaaagaacgtgtcacttcagcttcccagattactttgaaagtctaagaggaagacataacccaagcaatatggcttagcagtaaagttctcagcccctagagaaaaaggtccttggtttggtttcctgctttgtcacttactagctggataatcttggggaaattatttaatgctttaaagccttattttcctcacctataaaatgggaataacaaagcccatcttcagagtggttgtgaggaacaaacaagataatcatgtaaagccttgaacatggtaagcagtaggcactcaataagtactagttattattattgcttttaaaataagaaaacataaaatgtgattcctgttttggagctcagaatccagtaaatgggaaaacccaaaatacaacatgaaacattaaacaaagcgtaaaagggatctctcttggccaggtatacggtttctgaccttttctgttgtttgcaaatgatttcctttgagtttggaacaaactgtcttagcatgatctccgtaaatcttcaaccctccgtggtagatgctgttgccccactttacacaggagcaaagtgagtctcagttgtagcccaatatcacccagtaagtcaagcagaagagccaggatcacattcaggtagccctgactcctgttctttctcccataccccagtcttggagagcctcactaccccggagaaaagccgctgcacactcaaccctgccatcttgctctgccgtgccgtcaacagtggctcttctgaaaccctggaggagcggggttgttcctatttgaaatggcaagacctcctggctcatttctccaggctgtaagcagattaagcgccattaatgagttagtaactatctagttctacagaagatggagggcatcttataatgagagaaaataaagcaggacaaccgaaatagagataaaagatccacaaattattttgcgtctgaggaagaagggtttgtttctcagtgtagaaaggtaaatgtttttaatctaatgacctcaatacccaccaagattcccgcctgaagtgtctgctccttcaaaaagatgaggtccatccctccttcaacatgtttccgcctccctctccgcctcctcgtggctgcatcatctcttctgaggcttccattgacaatctgtccagtcacaggatggatcaggccagcttgcagaatcccagacaagtccatgccaagggctccctgaagtgaactgatagccccaatcttaggggtgctggcgctcactgggaaaggggatgtggctcctggggacccctctgatgagcaggagaggtccatagctgactccccatgccagccattgaggatgataggggtgtgcccgtgggtggcagagaactgctccaggccccgagcctttgcatccctctccacctcaaactcatagggacgcctgtgcttttgttcatcctttgtgaatactggagctaagaagctctcctgtgaacacacaaacagcaagagaggtctgaatgtcacacaagtggctgaatgctagctctttctccaggcctgatttccctaggcctcatattgtttccaatcatctttttaggagttcttctaactcaggcctcctcaaacctgaacagcttgttaaaatgcagattctacttcagtgggtctgaggtagagcctgagaatctgcttttttaacaggtttccaggtgctgctaaagctgctatgctagtccgtgaactacatttggtgttgcaagatgataactcagaaatgctcaacctcacctgtgcatcagaatcacctgggaagtttaaaacaactcagtacccaggacacactccagactgaataagtcagcatcgttagcttgtcacggtccccagctgattccaatgtgcagttgagcatgagaaccagctagttaataatgtatctcaaaactcatcacccccaccaaaacgctcactcctgccttgatttccctaatttctatcagcttccattgttagagctcagttctctaagggtcaaccacactgttccattcatgcttccctttccttatatatttattagtcatggggtcatctttgccagttcccccctatctttgtatccactctgaaacaaccggggtctagaacctctctttatcagtttcctgcttggcccccctagccaagctgtattctactccaagtgagctgcaaacctctgctttcatcatgccttctcctgctccaatacttttactagctcttcactacatacaggataaagtctaaattccccaatcaacactaatggcttggcaaaaaaaaacccctatccactcagtaaatacttgctgaatgaatgaactcaacagagacgtggaacacgcccaccattctttgttttgtggtcactccattcttgccggctcaccttgtggatctgggccgccaatgctgcgccgttgctgaagctgtgttctgctgcttccggctctgagaggctggttcgagaaccagcactgctggttaataccggggtgggcagtgtgcctgagggctcatactgctggctagagggccacttcccctttaacaccacgtggcagatattatctaggcggttaattatcacgcggtcctagaaaaaaggataaagctctgatgaataaggatcgagttacctaaattcacaaaatcctttgagatcaagggttaagcttttcaccagaaggagtcgataagaaaagagcacactcaaacatactcactttataaggcttaaaataaaaatcatagtctggacaatacaaattaaagccatcgtactataaataagaggacaatttgacaccttcatcttacagtcgaattagttaagcgcatgtatctttaatgttcaaatgctacataactaatatcttttaaaggtattaaacatttctgagtttcttgttcagaactatttctaaaagggttaaaaaaagcaattcaaagctgggaatggtttgggggtagggtgggaaacgcaagctctggggtacctttggccactcagagaaggaatagagagttttctcctgtagcagctgagcaatagtgggagctcgagcctcctgtagctcatccccaatgtctcctgtgatgcccgatgttgagctcttgctctcctcttcagagtacttccctggataatctgggaagaggttaaaaaattaatcaggcacttaactatagtagtgagatactagtgatctgagagattaggatatcctcttttccttttgctttttaaattagctcattctgtgctattttaaatggaatattaattatgatatcttctatttccaggagaaccgtatcagttaagccataaaagcaaatcctgctctggggctcccaaggcaagtcttctagtgagcatttgcatccatatcccaacacaagaaaagagaaaggaaatttttcttgtgtatccaagagtgaaagagatgactaaccacaaagaggcttctcacttagtgtagtggtcaagggaaagctgaacattctaacactttgagatctttgaagggacagtctatgaaatgtaacacggcaagacggtgattaatatgagaaaggatgaaagtcatgaagcttacaacaaaggtaagacagacctgattcatgtaaaaaaagatgccaagaaactcagaacatttcaattacttcactcatggaaagaaatggacatgtaacttattctttcaagcttcagtaatggtgaaagtttatcatcaatgtgtcaacgtgaatggatcttcaaagatccacttacaggaaatgaacagacaatggaaaaacacctaaggcaggaaaagttgttgacctctttcttggttaaatgacaaggagcatccatcctttgaaatgtggctatttcacttctcaatgtgcaacaagtctcctagggagctgcagcgacctgaccatgaaaaacagggccttgctaggcctggtagactcaggataagccagacagataccgcccaggtcctcaagattcttacagccctatgagggaagacagccaggaaagaggtgacaataaaggagcaactaaaattatcagctgtgataagtactatcaagggaaagaaacaagacgccatgatagggaacaagagggaagaacctactcaagtaggttgttagagagtctctcggaggagggagctcccatgaagatgaggaggcaacagggaagggtattgtggacatgctgaggaacgaagtgagggccaggaggaaagcacaaagatgaggctagaggggtggaaatcatctagaacatgctgagtcctctaggccagaatagaaaatttagattttattctagattcaatgggggaccaccactgaaaggttttaagcagaggagttgaatttaagttttttttttttaaaagcaaaatcatgtgagacagaaaacctggaaaaagccaggagacctgggggctggccctgacaggtgtaccaattaattactgctccttcaggactgtagatttctagtctcgaaagcagggctaatcctctctggcctacctgtcccacacctcatatgtgtggaggatcccttccagggtctgcaaaagcacttttaaaagcatcagtgaaagggactcccacccacgcgtgcacatgatgctcaccaaagcttattaaggtcactgctccctctactctgacttagcaaggctgacgctgccatcaaaggacaccagttcaggaacataatttctcctctaaaccatacttatctttaccttgtactcacttgattgcctgggtttccaaggactgataatttggttatttttacccgcaacagggttcaaaaagcaatgtgggttttataattcaacacatggcagcaatgcttcgtggcctgccatgaggaggatcaggcatagttgtagactggctccttctgggttggctggggttacaactctatcaagctactttcaatttggcttctcggtttttatcttagctagtatgtaaattttaatttgggttcagactcttcccaagagttctgaataactaaaaccaagacgaggagtttaaaaataaaaagctagataacctgaggctgggtgcagtggctcacgtctgtaatcccagcactttgggaggctgaggcaagtggattgcttgaagccagaagttcaagaccagcctggccaacatggtgaaactctgtactaaaaatacaaaaatcagtcagacgtggtggcacacacctgtaatcccagcttgggatgttgaacccaggaggcacaggttgcagtgagctgagattgcgccactgcactccagcctgggtgacagagcaagactctgtctcaaaaaaaaaaaaaaaaaaaaaaaaaaaaaaaaaaaaaggctagataacttgagggacttagcttagctcctctctttccctctttaacataaaatcagtgctccagaaagggacaatgtgtctgtctacacccctcagcaccacagtggaccaggcatgaaaggagttgtttttggttttttgttcaccctaataggccttcttggatctgaaaaaaattaaaactacagctaacaatcattttgttgctgttgttcctcaggatccctgatcactgtatgtctattcttgcaaagcccatagaatctcagggtcacctagattcttaacattttgaaattccttctagcttaaacccttatataagatttccctataggatcagatctattttcttcattgttgtaaactttttatttctgttgagttactgaaaggaatctaaatcaaatgcactgaccttaagccacacagttttgacaatgacatgaataaatggcttttttgctcaccttaacaaagatcaaatttatttgtatttttgttcagttcctgaaaaattaactgcccctatattaaagtgcaagttcctaacatcccaaggctgttctactttagccagtttgaagcaccttatctaggggaagaacaaactatttccccttaacaccatacatagcatctattgaatgaaaacacacaagcccctgcccaaattatatgagtgaggcagccaaactgctatgattgtctatgatcttcaattttctacaacatgaccacagttactaagctactcgaattttcagtccaaggcctatgacctttggtgagggtaggcaggtctgtttcagaagctgtaaaaatatgaagcactctctcccctaaaacccaaccccccgtcgtgtgtgaactagtttatccaaaatgccacaagatcctacatactgtaccttgactatggcagtttccgtctctatcataatcatctttattctcaaaatccatgtcttctgatttgagctcacttccttcaagaggatatgtggggaaaacgttttgcccctctgcactttctttccaaggttcttttaaaagctcatgcttcactttaaacggctgtgatggaatagcagtgggttcaccctccatggcgatagtattggttttacttttgaacagatctgggatataagccttgggtttctcgatttcaaattcatcattctcgaggccatgcatccacctctccatgtgtttgcagtggcactgacaggctgctggagcggggaaagccctctgtagcccaggagtctggtttgccacctctaagcttcctttcttggtttcatcttggaagccttggttcttttcccttgagatgttagtagtgggctccgtgagatgcaatacctctggcctttcccccatgcctactgccatggctaagttttcctcctcgttttcctcctcttcttcctcctcatcactgtggttctgactcaaaatcaatttactttctaagcttttgttttccaataaatcaattaattgctgatctgatgacaggtttcttttggagtcacagacactaagactgcacatatctacaaacccactttcatttcctggattcaaggaaggggaagctaaacacttggcttccagctggccaatgttttcaggtctctgtcctgcctcactgcagcagagctccccttcctttgaaatagacatcaacaaatgttttccatttttgatgtttgcttgagctactcctcctgcttctaagctacccataaaagtagggccagaagctatttctgcctcagggccaccagactggcagttcccatctttgcttattgagatggtaataacatcttttctagattcagtattggtacttgggctctcctgaaaagagcttggttcttggctgagcaccttcttaccatacatcatgctctctaaggactcaggcagcagactttcatcatggttgatggaaatgacatcctgaactttagaaataaagttttcacatgtcacatcaggcaggctgagatgatcatctctgctttctactctcactagattttcaggttcattttcaagggactctgaagtcctactcatttgagaatatgtaagagattcatataacttggagttggtctggtaaaggcaacagagatttcctggtgcctgggtgccagatcttttatgctgggcatagtttctataggcatccaggaaggacagctgggggtcgttcatgatgtaacagtcagtgcggttcagcccatgtttggcagtgccgatgagcaggtctcgatcatgcttcccacactcccaccagactgggaggtagaggctgggcctgcacagctggaggcgttcatgcagctgagggcacttgagcacttgctctcggactttccgtaacagttcaatgcggtacagagttcttgcagcacgttcctcagtgatgggttcaacgtagatggtggtatctgggggacctggagagaaagggaaataaagcctatcactgaaggtgacctcagacactccccgcccacctgagcccctgcatctttggtcttggagtttagctccctggctcctcagcatggcagtttcaggtgtgtgagcccttgccaaaataggaaactgaaaaagaatggaaggcttttatgaaacaaggacagacccaaagggtgtgaaagaagagcataaaaattccagttctgaaaacagtattagtttcctttaaataagcatgtactaggccaggcatagtatagtagtagttcacacctgtaatcccagcactttgagaggcagaggctaggaggatcacttgaggccaggagttcaagaccagcccgggcaacaaagtgagaccccaccccccatgtctacaaaaaataaaaaaaattatctgggcatggtagcatgtacctatagtctggctgcttggaaggctgagacaagaggatctgggaacccaggagtttgaggttacagtgagctgtgatcatgtcactgtactctacactaggtactttatacacatctcttagttctcatgaaaatcctaccataccatcatcatgcctgtcctatttcaaggataaggaaattggctcagataaactaccttgctccacgtgacagaaccaggatttaaatccaggttttgtcagtgtgactccaaggtccaccatctttccaatatattcagctccctggcttggatgacaacactttagccctgccatataggaggcaaaggctggtatgtcttgtgcgtgctgccatcagccctgaacccacactgagtaggctcaatggttgctttggctttcctaaaatattgtcccatgatccagaggaactccatctacaaatacgaggctacagggaggcctgaaggaagctgttgcaagtgccaaccatgaagacatttttcaaaagggttctctattgcatcaggggatatttaaatgacagccacttactttctatagttacaagtcgacatgtgaggaaatacttaaagttattaagacttaatctaaggaaggtgcaaagttcagagtaggataaatctgaacatgacttaaaaaaaaccatgactttaaaaaaacccttatactttggagaaagtggttggcagtcttccaatatttctgtgatgttttaagctgaatttcaactttcactgaagttacatcattttgggagagagattttattttatgaggtggacccaattagaagttcatgaatttctaaaagccactcatctttacctaaattcctcatgtagactctagtttttataggtatacctaaagtaattctgcaatttgaaaatcagaatgttctttccttgtatcatctgacaatatactgatagttttctcaccgccatctttccatgtgggtagacgacagacattccggcacatggccacaaaactataaaaatactgttccaggctctcatccgacttcttgtccaaacgggaaatgatgcggaactgtgtccagtcaaaggttttcttttcttgatcgtaaacaacaccaaaggaagacactgttctatagaagtctgcttgttctctcctagtccaccttgagatttaatcagaaaggaagagtgagctctttcttctttttttaaagccaataactgcttattgagagcctactatgagagcccataaagtcaagaaaggaggcagagctggtttgtctcttttattatttttatttaagttaaactgggctgaagggcttataatgaaaacagcctattatccaactcccaaagaggtaatcacttccaaatctttcagctctttcttctggtatttacctccctatttctaaaccaggtatacacttgctatctcttgattcatcaattttaggaatcacctagttaacttctgcctgtagatgatgaagatttttaaaatactatatatgcttaaatatctagccttatttttttttacccgtatggcaatatagaagcagagaatcctgaagggatgttccagaccattaaattcaacctccttatcttactggtgtcccgaagggaagtgactacttcagtgacacaacccctgcctccccatctcatattgcttttctttgtggttttatgtaggtaggttgtccatgacaattatcttctctgggacacaaaggtcctctagtctttatatcctatacgagtaagtcaatgacttaagggaggtggataattatataccaccatagccattatgaggggctcctgggcagggtgctctccaatccagagggagtctagctaggcatgggggtaacagggtgaggggttagagaaagagtgagaagcagggaagaagtggctctggttcttctctttagagatgtgcaagaaggaagacatatattccttgaactatcgaatttcataagtcaggagtacagagaagtaattaaatttgttatgcagtcttcttttaattcatggggtgtttcatcagaaagagagatatgactcaaggaattgcagctttgttgataccttctggtggcatcatttctagcctatgctggccttctgtaagccttttagggcttaattctcagatgggcaagtcactgctggtaatttattctttccttctgggcttgtattttacggtatttattgccctggaaggcttgataaggatttagctataataagacagcttcagtgcatactgttccctttgttactaaaataattagggggtctgaaatggtctgacacattccattgtggtgtttctaaaccacatccccagagactggggtttaacttaggaagagtgtcaagttagaaaatcataaaaatctggaaactgtaaagacagggtgttcctagacctcatcagactcccatcaccatgataccatcacaactgaaggctgaatgtaatgaattctggagacaacctaacttcacatgagaacagaaatatactcgccctccaagctaccggctttaccttttgaaaactttacccagtgggtggctgagcatgcccctttggccactggcttttacctcttttgggcttccttgttgatgaggtccatttctgaggttctcctgaacatctcttcctgaacccaatatccttggttacctggtcccagaatttcaggccggcacagttccttgcggttgcagcgctggtaaacagtgaccagacgtctgagacgagctgtgagggcggaggaaactggccagggcgatttgtctgggtcggagccatcctgggcttcaaacatcagtgaggatattagtatttacaaacagaactaaaacaggagtgactgtcttttggttttgtttaaataaaaacaaagctgactcaataatttactcaaagtagttactgttttaataattggcccaggagcaaggcatgactggaagggctgcaattttgccttggatacagcttatgttgtggtcaattaattctgttatctctcaaacacagaagtgtctttttgtggtggcagaagttatgtgaggactttgctctaaagcacaggaatgagcagatcctttgttgcctgcaccttccaattaccaaagctcctctcacgtctgaattcagaaaattcaaaaaccaaaccaaactaaaccaacccccacaacctttctttaaaaaaaaaaacaaaaaaaacaaaaaaaacccgatgtcatcacaattcctttttacctctcagaccttattctaactgaagatattaacatttacttcactttcaaattttaatgccaggtagaggaagccagagccatgctgaagctccatgtgtgactctaggggagtttgatgatggtcaacttacttggctttttaaaagagcctccttaaaaaactacttattaattacatataaactccacgataactaacaaactaagaccttactttcctatgccttcagaaggacatagtctcacctgcccggctgtcatccttcttttcgctaaaaacgccatcacctccatcactggaactctcctagggataaataaagccatcccatcagcaaaactgcagcttagaagaaaccagctcagaaagtcttgaaacggaaagagtaagcatcacgcataagagctgtcccctgcccatgaacaactcagaccaagataggacctgataaagcttacttctgtacaatcttaaaagagccagaaagcagtttatttaaaataagttttttttttcttttcctggaaccatgtggaatgcttctgcctgtagagaatcggcttgagcaatgatgaactggcacgctctctctggcaattacattttacaagtactattttagttgtgaatttaaatatttgccttaccatcgtcacttgcaatttaatactgtcatttcatttcacaaaggttgtgcctgcatttctgttcttttgctgtgaaaaagtgtcatgctcatgttcattatggttagcttacttcacagcaacagacactattttcataaaacactactgctccagacatgaaacctgatggcgcaaaaccccaggaatttaaaattaaaatcatgacttgagctctaagtgaatctggagtaatatcacaagcactgagcttgcccaagtcggtgtatgtgtcaaaccagctttaatgaattccttcgcaaatccttcttggtctcaggaatggaactttaaaaactcctcatatgtaattcaggtgggaatctgaggggtaacagaagtgactgtgctatttttcaactggactctaatttcaacactaaaacttactatattttgactagcatcttttgacctgaatgacagtacagaacaacctcaggattcaagactttgctattttagagcacagggtcagaaagaagggtcagcaaactgcggcctgcagactttctgtgtggccctcaagctaagaatggttcttacgtttttaaagggtgaaaaaaagggaggaagaatatgcaacagaagccatatatggccttctagcccagggtatttactatctggtcctttacttgctacttcctggaatacacaacatggtagaccagtaatctagtatggtaggaaaggagacacccatgactgccaacagtgaaaattcaaaatgctcttttgggcccctgatacgacgcaaagaacaggagcccattttctgaatgaatttctctcttacgcagaacagcgttaacagctaatattacggagaactacgtgttaagcattgcacaaaaccttttacatggctcatctcacttagttatcagcactgtctcatgagatagaaaccattactatgctcattttacagatgcagaaaacaagccctagaaatgttcagtaacttgcccaaggtcacacagctggcaagtggcagagctgggattcaaatacaggtaatctgatttgagagccaacactttcaactaacaagctgtctaaaaaaggacaagagagtcttagatgaaaatttgtcttggagagtgaaaacatatttataacagaagtggggaacactagctaacctcctttgcctgggccagaattgtctcctgttatctaatgttaatgaagagtgaaaacccagggctgatctactgtaccattttgaagtggcagttaaatataccattttatcttatactactatttaggcagttaaaaaaacaacctactatgccttatataatttcctgccagagaggactttttaaatatccccacagtatctgtgaagatcctttgaatgctgaaatgtataagtgtagtacttccagctgagaaatgctctacagtctacacaacacaggtggcaggccccagagctaccatgttccttcgtgccatgtagtggtggctgacatgggcatcatcataccccaagtccatgacaacaggctcccagcttggtgtgaggcaggcatacagcaaatgctagcttcccttccagtccacctagtagtttctgttggtacagttagtaaacatgaatagttttaggcaaaactttcaagtaaggaaagctttgagcattcatagtgtttttctgttcctgtcctctcttgaaggtacttcctctctctagaaatgacagaatgaagaagacctagtttgttagtagctcaagtgaaaaagatgtcagacttttagttaataatccgtttaatgtgagtgtgtgtgaggtgctgtagcagtgacagtcctgagcatgcagaggccctgcatctgaactatgtagtctaatgtgtgctacctcaatttctgcttacatgattaacttgaaagaaatgttcatcttcaaagaggtatgaatcatctcacagagaagggccccagagtgctgacaagaatgtcaacaacacagcaaactgattggttacaatgtgcaatttctccatcttgaggacacacgtggtttggagtcccatgatccactgctctctggtctcgggcaagttgttttgatctctctcaagctcatttcttcttttgcaaaatggagaatggcataaacttcaggtggaaagcagatatacctcaaatgccattaacacaatattcaaggattttattatggaaagactgcagaatgtagaaaaagaagagggcaggggagatagcactcttgtgtcccctggctgtatggcttttttgcagctgattaaaactgagctccctcgtctctacaatgggcataagaataatgcctaacagatggggaaatatgctttgtaagtgtgaaagtatacacaaatactagtatttgagttcacagaaccctcaaactcctaactcacaagaagaaggtgcttgttcagttcctaattaaacttttggaacaatggaatttggccattttaggcattaaattttcctgcaagttcttggccatgacttcatccttttatgtactgacctcatggtttagagagcacctgagatgtcacagccacctacggtaggaactgtggcacagcacggacatgcatgcttttgacagcataactacaatgatgaaaattgtgctgttacttattgctatttctgttaaaagatgtgttactctccccacatactggctgtgatagagaaagattttgtttacaatttcaggctccagaccattccaacccaaatatactttcagactaactgctttagaaggtttctgtaggttcactgaatctggctgcacgtgacatctgtaatgatttgccaatcaaaagtgctactggtattaaaacagagggtgggggaagctgtaacttagatcatttcaaatctcccttcaaattctaacattctatgactctctgaacgccacattttaagaggacaacaaccatagtgtgctctggaaggcgctggaatagcaaagggtctggatatgatatgatatggaaaaatatagaggaactagaggtatttcacctgaaaagaaatttaaagaaattaaggtcatgagctggtctcagcaatttaaaattctatcagagaggggaaaaacaacagttttgttctttgtagtaccataaagaaagctagatggaacccatggaaccagctgggaggcagatttctggtgtggtaaaaagaagtatcatccaacaataaaatagacttctttggaaggtggcatgttcctcttgttttccccatgaactcccccctgacagccccgctaaagagggaacgccacactgagaccactcaggcttgtgccagggctccttgcaatgctaagactctaccaatctactaaacggttgcctttctatgcctaggtctgttgaattcaggggaactgctctaatgaacaggctctttttatacaatgctttgggcttctttaactctgcctgaggacagagctgggagaaagaaacacccaaacagtgtcttcagtgcaggaggtagggagcaagcgtgaagcagggtgggcagcaggcctggctcagatgccggccatccctctatgtgtgtgtggttttagcatgcagactagaagtaaaggtgaatccgtttataatctgtccacctcagaatctggggaaatccactccccatttcaactgcttttaatgatattctataccccaggttttaaggatatgaagagcaaatcataaatgtgttaattttgtaaatgaatacaaaagtaattccagagtttaactctaatatataagagcgcttaagttatttttattgattctgccatggccctccagtgtcctcctaattgattagctaagcatgtccagtttaatgtcagtggctttcagaggctggcctcttcatatgagctactgctgggaggaaggggcataaacccacacctgaattactctctattgtctggactacgaaaagaatacttgactacaacaaagaataccggaatatctagagaattacaagtcttagctctgtgatctagtggttgtgagaatttggtcaagtcctttaactttcagggagggcctaaagtttcctcagccctcaaatgaagacagggtgaggtgaggatcaacttaaataaatggagtaactggtttgaaaagagtcaagttccatatgagaataaggtattatgttcatttctttggtacttgagttgtttctacagggaaaacgcatcccaaattccagacaaaagcaataatataaactaaggactggaacagcagaacatgaatttttttttttttttttttttttgagatgaagtctagctctgtcgcccaggctggagtgtggtggctcgatcttggctcactgcaacctctgcctcagcctcctgagtagctgggagtacaggcgcatgccaccacacctggctaattttttatatttttagtagagacagggtttcaccatgttgaccaggctggtctcgaacttctgacctcgtgatccacccgcctcagcctcccaaaggctgggattacaggcatgagccactgcgcctggcccagaacatggatttgaattgtcaagatgagaacctaacttccttcatgttaccggggtctgaggcttcatgctgatttgcttcaatccactgcttctcaaaccttgaggacacagggaaatgcatctttgaaaaattattattatctattttaagaaagggatgaagccattgttactgttacaggggtcagagctgatggaggccaaagtaaataaataccatacattctgcaagctagaatcaacagaagagaatggaaaataatggataatttttgggtagtgatcaggagaagaagagggaggaccctcgcaggggagtcacttggggacaatgctacagtaacttctgggatccagaatccataacattctctctattactcaatgctagagaaaatttggtcatatggtgctggggcccaggtagggtttcagtctacttatgcaaaatctcagaaactcattttaaaaatgcaatacagactgccgcttctgggatgcatttccctattctttaagtataattaaaccctctggatagtaaatatgaaacaagcataagaaaactctgaaaggtgaaaaaaagaaaggacaaactagagacctcaggactcaaggaatgacatgatgaaaaaacatgctttttttttttttttgcttcatatatcctagatttggaggtaaagaagcgggctacccggaaatggcagaggcagacaaaaaaggccctcaaaaaaaaccttccgggcgcggtggctcacgcctgtaatcccagcacttttggaggccaaggcgggtggatcacctgaggtcaggagttcgagaccagcctggccaacatggtgaaactctgtctctactaaaaatacaaaatattagctgggcatggtggcatatgcctgtagtcccagctacttgggaggctgaggcaggagaatcacttgaagctgggaggtggagattgcagggagccaagatcatgccattgcactccaacctgggcagcaagagtaaaactccgtctcaaaaaaaaaaaaaaaaaaagaagcgaggtggcttcctgctctctctagttaaggactaggaagaggtaggttagcaggttagggacacagaaaatattaagacaataactgctataccccagcccaacaatgcagaagaaactgtctcaactctacccatggcaacaacggctgagtgggagctcagacttctactttttccaggtggtaatagggtaccccattcccttgctgggctggtatcagaaaaagctaagcaggaagtcaggactttcaaccctgacaagtagtaatgaggttccccacccctacccatggtggagctaaacttccattcctgctcaaagtaatgagaaacatcccctttaggggttaatgggagcagactgggaacatgaatttttgcttccacctcacagaaacaaggtgccccccccgacctctgttcccctgccaagaaaggtgctgaaagagaacgtttaaataagatccagagtctcacaatacaatgtccaaaatgtccaggttccaataaaagatcacataccaagaaccaagaagacctcaaactgaatgaaaaaaagacaaaagtggccaatgctgagaggaaagatcttagaatgatctgacataagaatttaacagtcatcagtgagccattctgaacatggcccactgttcagaatgaaacagtgaaatgaaatgaaacagaaagtttcagcaaagaaacaaagatccaaataatatttggattcaaagggaaattttaggtttgaaaagtacaataaactggaacaaaaaccaaaaccaaaaatgaaaacccaaaaccctcaggagaagactcaacagcagaatggtggaggaaaataatcagtgacctcaaagacagaaaaatggaaagtacccaatgtgaataacagaaaaaaaaaaaaaatggacagagccttagagacccacaggtctataacacaagatctaattttcattttattggtttcccagaaggcgaggagaaagaggacagggcagaagaaatacttgaagaaatcatggctgagaatgcttcacatttagcaaaagacagaaatcagatttaagaagacgagtgaattccaaatagggtaaactcacagaaatccgtaagcacataaaatcataaacttctgaaaactaaagacaaagaaaaaatcttgaaagcatcaagagagaaaagacacattaccaataggggaaaaactgcttgaatgacagatttctcatcagaaaccatggaggccagaagcaagcggcagtaccttaagagctgaaagaaaaaaactatcaacccagaatcctccatttggtgaaaatatcctttgggaacaaaggggatattaagacattctcagatgaagaaaaactaagagaaccgtctgacagttctctagactaccttaaaagaagagctaaagagatttctctaaaaagaaagaaaacaataaaagaaggaaccttggaacaccaggaagaaaaagaacacagtaaaaaaaatatgggtaaatatagcatattttccttctcctcttgagttttctaagtgatgtttgacagttgaggcaaaaattttaacattacctgataagaaatggttaagacaactatattataaatggaagagggtaaaggaacaaaaatggaagtaaggtttccacacttcacttaagctgataaaatgatagcattagtagacggtgatacattacatataaatatatgaaacacctagaagcattaaaaagctacaaaaataaagataaatcagaataaaatcatacaaaatactcatgtaaaccagaggaaggcaggaaaaataaaaaatcagggaaaacaaagagaagacaataaaatgacaggcttaagctttaatgtatcaataattacattaagtgtaaatggtctgaataatatcaactagaagacagagaccggcagagcgtattaaagattatgatctacctgtatgctatctataggaaactcacttcaaatacaataaaggcaggttaaaagtaaaataataaaaaagttatatttatgcaaacattaatcaagggaaagtagccatattaatttatcagataaagtacccttcagagcaaagaaaattatcagagtcaagagaaagacattatatagtgataacagggtcaatctgccaagaacatgtaattctctctatacatttttttgagacagaatctcgctctgctgcccaggctggagtgcagtggcgtgatcatggctcgctgcaacctctgcctcccgggttcaagtggttctcatgcctcagcctcctgagtagctgggattacaggcatgcaccaccacgcctggctaattttgtatttttcgtagagatggggtttcaccatgttggtcaggctggtctcaaacccctggcctccagtgatctgccttctttggcctcccaaagtgctgggactacaggcatgagtcaccgcgcctggcaagaacatataattctaaatgtgtatgcaccaaccagagctgcaaaatatatgaagcaaaaactgatagagctgaaaggagacataaataaatccacaattatagttggaggcttcaatattcttctctcaacaactgatgaaacgactagacagataatcagcaaggatacagaaaaactcaacaacatcattgaccaacaggacctagtcaacatttatgaaatactgcatccaataaaaccaaaagcatgttctttccaagtgcccatggaacatataccaagatataccatacactgggccataaaacaaacttaacaaatttaaaacaactgaaatcatatagaatgtgttctctgattataacagaatcaaactagagatcaataacagcaaggtaatggggaaatctccaaacacttggacactaaataataaacacctaaataattcatggaaagtctcaagggaaattaaaaaaaaatacattaaactgaatgaaagtgaaaatacaacacaaaattaatgaaacataactaaagcaatgccgagggaaatttacagcactaaatgcatatattagacaaacagaaaagtttcaaatcaataacaaagaaactcccacctcaaaaacctagaaaagggctgggcacaatggctcatgcctgtaattccagctctttgggaggccggggagggaggattgcttgaactcaggagttcaatactagcctgggcaacagcaagaccctgtctctacaaaaaaacagaaaaatagaaaaaattagctaggtgtggtggtatgcacctgtggtccctactacttgggaggctgagctgggagcatcacttgagcctgggaggttgaggctttagtgagctatgattgcatcaccacactccagtctaggtgaaagagtgagaccctgagtcaaaaaaaaaaaaaaaaaaagtaaatgcagaggtatatacagtaagattcagtactgtaaaaactcaatgaagatgtcaattctccccaaagagatataaaggtttaaagcaatgcctatcaaaatcccagcaacagtttctgtggataagattattctaacatttatatggaaaggcaaaggaacaagaaaagcaaaaacaattttgaaaaagaagaaaaaagtgggaggtatctatctatcaaattttaaaacttacggtattgctacggcactcaggattatgtgctactgacagaggaaaagacaaatagatatatggaaaagagcagagaactcagaaatagaacccacaaatatgccaaactgatttttaattttttttaattttttaattttttgtagagacaaggtctcaccatgttgcccaggctagtcttgaactcctgggctcaagggatcctccctaaaaagagacacttacagggtgggagaaaatacttgcaaactacgtatctgacaaaagactagtatctagaatatatacagaactctagaaaatcaacactaaaacaatgaaattagaatataggcaagagacataaacatttcactgaaaaggatatacagatggcaattaaacacataaaagatcttcaacatcatttagccatcagggaaatgcaaattaaaatcacaacaaggtattgctacacacctaccagaatggctcaagaaaataatagtgaccacaccaaatgttggtgtggatggagaatacagagaaagcggatctctcatgcattgctaatgggaatgtaaaatggtacagccactctggaaaacaatttggcaatttcttaaaaaacacaacatgtaactatcatatgacccagcaattacactcctgggcatttattccagagaaatgaaaatgtatgttcacacaaaaatctatatgcaaatatttacagaggacttaatagtcaaaaactggaaacaacccatatgcccttcagtggatgaatggttgaacaaactctggttcttccataccactgaatactactcagcagtgaaaaggaatgaactgttgatacagacaaccacctagatgaatcccagggaacttttctgcatgaaaaagccaatccccaaagtttacatactgtatgattccatttacagaacattcttaaaatgacgaaatcataaaaaatagagaacagatgagtggttgccaggaatttaaagaggggtgaaggcagaaggaagaggatgtggttataagacagcaacaggatgaatctttgtggtcatgagaatgtcctgtatcttgactctatccatatcagtatcctggttgtgatattgtactacaatactgcaagacgccactacgaggggaaactggataaaaggtatatgcgatttctgtattatattttgtgactgcttctgaaatatacagttatctcaaaataaaaagtttaattagaataaaaatgcaattgcagtctgtgtattttaagtttttataataaaatgttgggcaaaatgcaatgtaactgtagtaatgcaatcaagttcccccagatcactaccaagatagggatttctttcttaatatgaaagacggtgtaaatgaccaatactgcacatttgactcaccgtttgtttctggaggccatctactttgtcctcagcattgtcttctttatctgtgttgcctctaaataaaagtaaaaaaaggcatcacatactacctaggtcccccttatccacagtttcagttaccctggtcaaccacagtccaaaaatactaagatattttgggggagagacagggcgagagagagagatcacattcacattacttttattacagtatattgttataattgttctattattattgttgttaatcccttactgtccctaatttataaattaaactttatcataggtatgtttatataggagaaaatacagtttatacagggtttggtactatctgaaatttcaggtatcctccatgaataaagtggaacttccaagtcagccgtctgtacccatggaggattggttctcagatcctcagggataccaatatcggcagatgatcaagtcccttggttggccctctggatctgagagttctgcatccacagacaaggagggctgacaatagatcccttcttctgaaggcattccaagccttagtttttccctcccttgtgggagaattcaagagacactgagaatgagagaaactatcaccaaacagttatcacttctacaggcaaggacctttattcaactgagactgcagcatgaggctccagcctcacgtctactcaggcaagttcagaaaaaggtaattcctgcccatcaagagtcatctgaacatgtgactctgataaaagaacacactctagaaactgaaggctgtgcacatcaaatgaggcctaatgttgcaactccctggtcacagtaggatgaatcttcccacagaatcacatttggcgaaagtcactccttggcttggaagggagctctgctgccactggttaacaaggacattttccccttgttaccatcagacccttgatgctagctcagctgcctaatgtcttctcttttgtgagcttaactactgctgatgcctacctgtttttcatgattcttttaaaaactgactttgagggtgggtgcagtggctcacacctgtaatcccagcccttagggaggctgaggtgggcggatcacctgaggtcaggagttcgagaccagcctggccaacatggtgaaaccccgtctctactaaaaatacaaaaattagccaggcgtggtggtgggtgcctgtaatcccagctacttgggaggctgaggcaggagaattgcttgaaactgggaggtggaggctgcaatgagccgagatcattccactgcactccagcccgggtgaaagagtgagactccatcacaaacaaacaaaaaacccccaaaaaactgcctttgaaatatttgcgtcccaaatattcttgtccttgatgctcaagtcctagaggcaagataagctccttgctttaccacttaaccgtagaaagaaaagccaccccacaaaaaatagcctttatgccacattcaggcgcccattcagagagtgttttccctgaatcagtgaatgaacatgagaatcaccagtcatatgggcctctgttaacatctcccagaatgcatctcggttatctgtcctcaagaaaaatgctggggcagctgaagtgttcatcaggggttaaggacagatggaaaatgcactgcacacttttcttggcctgagcaaatagttaggggctcctggcagtgccacattctcaggtctggccacagacctgggcaatgcaaagcagtatctgctacttttggggagaaactaagagataaatgctaagagataaatgctatgttttggccgggtgtggtggctcacgcctgtaatcccagtactttgggaggctgaggtgggtggatcacaaggtcaggggttcaaaaccagcctggccaacatggtgaaaccccatctctactaaaaacacaaaaatcagctgggcatggtggcacgtgcctgtagttccagctactcaggaggctgaggcagaagaattgcttgaacctgggaggcggaggttgcagtgagccaagattgcgccactgcactcctgcctgggtgacaaagcaagactctatctcgggaaaaaaaaaaaagctatgttttcaaaaacatatatataatttgaagaactacaatttttagctgtttagttttcctatgtatttatgtcaagagtaaataaaaatcagtcaaaggagaggccttccatctacttttttgcacacagtccatggacaagctgaaatgtcatggccgtcaaggagcggcccctaaatccatcttgctcatggactatgcaatccttagacaatactggatctgaggaactgcagttcaaagtcaggaaggctgtacttgtttctgttcttagctattctgccaggcatgggaggtctgaattctcccacaggaatcctctgaccagacaagggatggacatggttagaaagctgggcaggcttttagaaaggtgggcagcctgggaaactcatgaggaacaggaaccggtatttggttcaaagtgcttggaagtcaaagagcaggtctgaggtagagtagggtgtaaccaaagtagcaattatggaaaacagcggcagtgtggcaaggtgtagggagatgccagatgctgaggaagagataaagagtgctgggtttatgaggaaggggtcacagatcagtggcatggcatgtcacatgagggctggcctacgtggcttctcacctttcaggaatgtctgaggtcccatctgtaacaccctgttctgcagaaagggacttctcatcgggcatcccaactttctccaggaagcaaagtgctgggtctgctcgcatggcattgtacctttcataacctgatcaagagtggtgagaaatgccagaagtcatgttagaactcacaatatttgctttgggcactagtctttttgttttccttatttagtaaagaacaaaacaaacacaaaaaaataattatttaagattaatgagatcctataagactatcagggaaaaatcctggtgctaaagcggacacctttgagtagtgtttgaccaacaaacaatggcttacggaaatactcagtgtccgtgaaaagacaattgtatagatacacaattctgtttgctgcttcccgtcagctggttttgcaacttcttcccccatcctcaactctggataccttgttatatcctttatgggccttgttttcttacagctcatttttatcccatgtgttagaaagggaaggtgaacatgatggatggcaggactgaaatataaagagaactacgatgattgtttattgcattaagacagacctgtggcctctgtatcacaaactcagtacaaatcttaatccacactggccagggctttggaattactcctcatcacaccccaaagttaacaacagacagtgttttgacccagaagaggggcctcaaaaaggaagcaaaacaattttaaaaaccctgattttgtttgataaagtccatgtgtgaggatttattttattgaacctaatgtactcttagaatgagacagcatttggccacataaaaggcagagtccccatcagaggagatggaagagctgagtgtaaatgagaaccagcctggctcttaggaacaaacctgatttaactgtgtttggtatttcatttctattttggtttttgttctcgtatcactgttgatgcagttgtatccttcaagctgtaggtagcacctggttcttttccctgaggagctgtgggtagctgatcttttaatccacagtggctctcctgccatctccatgacaggtgctgcccacatgaggtacctggcacgagaagctctgcagtgccttatgttctttactcacttactccttatggcaatgacttgattgctctaagtctctcatgaggtcgtgagcttcctaggagcagaacctgtatctgtttttgctcatcattatatagtcagtacctagcaaagggcctgacactgtacagactcaacaaacaactgaccagatgagtcggttttgcctagattcttttctcttccaatcactgttgtaggaagaagaaccactctcaccctttccccacgttcctgccccacctcacatcaaacagaaaattccccaatactcctgcttttaaggaggttccagtcagtttctccacctgcttctcatgaagagaggggtaatttaaatgacaggtgagatggtgaaatccttcccttactgtccccttactactgagggaggccaactgtccaggagtataccaccttcacactctgtgctcaggcatggagcaagctgcgaccctccctgacctgggcaagcttctattctgtagactttctcttgtcatgtgtggatctgaagcctagagacagattcagtatttttatttctcaattagctgtgaagctggaggtgaggaagaatgactctttcagtcccctagaaaaccctaacaactgtcctgaagagaagccagtaggagctgcaacagcaatacaaagacaaagagcaacgtgacagacccatgagggggtgtcggctgcctgcctcttttcacttttagtctataagctgctctctaagaatatcaacagaaatactaaagtctgtatgtcaaggaaaatttcacacatccaggctaccttggaggggcagcagtcactcaactcaactgagctgtcaagagcataatttagaataggtttgccacagaaatctttttgctgcaggctaaagctccaaccaaaaactaagccaatgctaatggggtcagcttggagatcgatctatggctatattcaactcatttaacagggttacagcaggctatgtctggagtggactaaaagatccacaatcccaggcatggtggctcatgcctgtaataccagcactttaggaggctgaggcaagaggattgttttgagtccaggagttcgagcccagcctggtctgggcaacatatgagatcccctctctacaaaaaattaaaaaaaaaaaaattagccaggcatggtggtgtgcacctgtagccccagctacttgggaggctgaggtggacagatcacctgaacctgggagttcaaggctacagtgagctgtgatcacaccactgtactccagcctgagtgacagagtgagaccctgtctcttaaaaaaaattcacaaaacctcagtcaatgaacagtcatgtgtttcttaacaaagaaaatggttgcagctctacagctcatacttaaccaaggcatgcaggtgaagtggagcgggcttcctgacacaatatccctgaaaagtgaacactagacagactggctttctgaaaatggctaagtatcacatctccagagctgctgagtatctgagaagctgtcacctggtaagacatgttgtgtgggcttggttatactgtgataagggcgaaggttagaaaggggtggtcagatgtgttacctagatgaggacatgtctaagcctaagctcatggaataggcagcacccaggcaggggcaggatctgatatggactctggcgggtgccaaagaggatgggcccatgaggcatctgtccaacctcatctaagttgactccaatgtaaccatccaactaaactgatctggccctatgcagagtctgcatttatggcttaggctcagtctcctagcactgaaaacagaggctttttcttctcttgtctttatagcattatctgctaaactcatcctgtacaatgagacacttcagacactgcacaaatcaccttcaaggcctctgcccctcagctgtaactgtaaattccaagttcacctcaactgacaatctgtgagaatctgaatggaattgagtgatgaagtgtcatcttaacatctcacaggtgtgatcagcaaacacaaattaatttatactccccaaaactgcttcaacgagcagaagaatttgggagcgaccttatgtatcatctgggctgactctatctccaatatgagactaagagatcaactgattaatcccaataccctgaacagtgattagggattaggaaactagaaggtagggcaccaataataacctgtcttttccaggtgagaaggcatcttggacctccttagattcctgtccatttttacacattctaaggctatgtccttcacgtgagaattattaattatatcttcctggtgagttgtatctatcatcattgtgtaatgaccccctttcttccaaattatgctttttgcctcacagactgtgtgtgatattgatttgccttaccgggttgttagtattttctggggatgtcttgtttcacctcttggatgtcaacctcactctgtcctctaagtctcatcttctctttcatgtcttcatcttctttatcactcagggatctgtttctttagctctgctttcttggaccacagagctttttcttgcttccctttcctggaggggcagccctttcaaagtcatgttttccatttctgtgggtattaaaaacccagtccttctggttcactgtcatattggcccctaagctttcagttctggctttaaatgacttctgcatttggggacttcccttatttcttccaagtcctgatccaccttggttttctgtatgtctatggtgggtgtgttggggtgggggaggaagaagtagcagcatgtaaattctacctgccatacttctgggacttcagatgtgttctcttttctcagcatgttgttgccatgcagggacttggcaatgtgccctaaaaccaccactgaaatgagactcagggtcactaggccttcccccacaaaccaaattcagatgctctggaatctggctgcctttagcagcctactgtcttctggggtccttccaaaactaggttaaaccaagtctgggttgcactgcggtaccttgtgactaacagggagatcacgcacttccttctactttggagctgagttctatccctgcactgtccaacagaggagccactagctacatggggctatttaaatttaaattaattaaaagtacataaaaatacacttgctcagttacattagtcacattttaagtgctcaatagccacacagggctagtggcttccacagcagacagtgcagaattagggcatgtctatgattgcatgtagttctaccggacagcaccgctttagcctaaccccggcggtttgacaaagggttctcagcagcactgcaccacctgaagcctgcattctcaataccagtatccaagttttgagtggattccaaattaaaatatgtataggcatggttgttacttaaatatagctggttagcctgtaaaagttgaagaattccctttaaagaatacgtattagaaagttaacaaccttccaaaggcagagtaatatgggagagtggaatagcacccagcacctattatgtaccaagcactgttctaggtggtctacatttaatgtaatatttaatctttaaacaacccctaggtaggctgttttgatactatacctcctccattgtataagtgagaaaataagagaagcttatgacttgcccaatgtcacacattttagtaagtagtggagttgatagctaaagccaggtatacctaactcttttcactttaaaccttgctcctcattttaaaatttctgttgattcacctggcagggaaagtgtgtatcaggcatactatgggtgactatcacttgacactttcatcattcattttaatggctggcagttggctgaaagcaagggcagttgtttcaaacactgccagaggtgaagaaagggagcacttacttcagtttttccaagtgtttaaaaaacctctcagtcccctttagtggctcaattaccacatttttaaaaagacagtctatgctatcttctatgcttagtttttaaaatgccagtggggtaaaagatacacactaggaagatgtagattatcgagaatgaccttataattctgcatatataagaaatgctgaaacttagcatttcccgccatttatttactccttcattcaaggtgtctactatgtgccatgcactgggactattagaggcagtcccatatccgacagcatggacaggcgagggaggaagatatacaccaaacaggttctttgtggcctgaatgttcctcaataatataaaattcgcagtattattgtgagccatacattcatctggtggaaattccaagagggcagctgtgaagctctactttttaagatgaatgtgtaacagaatgatagagttttgcaggaacatgccgagcttagttggaggggctgaactcatccttcctgggggaaactgatacagaagggaacgccttcagagaaatacacaccatgcttgaacacgccaatgagaagtgacttatcggcttcagcatcccaccagtccactgggatctccatgtagtcgatgtcaggcagaggtacatccagctccctagggaaggaagggtgtagactcaaaacaacacaaaagctactggtccaaccctggtattaggccacagtcctactggcatgctgagatgctaaagtaacaataaacaaaagggttaggaaggctgtgggtcattgtttctcaaccagggctttgcatgagaatcacttaggcagtgatttaagcataagtacttgatttccaggctctacctttgacagactgacacagtcttgaagcacctcttggagaacgcaatgtcagttggtccagggtggcatcttgggatccatatgtatagagttctaatgtgcaccttatgtgaaccacagcaaaggccattgatatattggacatctgaaggtagagcttccccaagagttgaattcatacatgattgtatgtttaaaatagtgcccaaatcatcgtggctcagtatgttgcctcaacaagtagagatcttacgcaccatttaagaccaagagtccacactggaacaagtcggtatatcaggagggcctttcagagggtaagtgccataacaggtggcacctcctggcagtggtctattagatcaaaattgatcaaaaactgcattgttcctggccaaagggatgcacataactcaccccagggcacagacatgcaggtgttagcccctctctgagtttctgtaacgtgtttggagagctcacataaaccctaagttcactgtcttcattcatctgagtacagtcctcgctctttaaggctttgaggtttagggaatagcatgcatctggatggcttctccctccccctccagctgcctctgaagtttcttcttccgtcagactcttactgtgacacaggaccgaatgctccctactagtcccagctccctctgtggggatagtgtttcctgacccccacctttccagggatccatggctctgcctgcccagtgccatggctgaatgacttttatgggatgaactgctgttcatgcctcatggcaacttctccacaagaaggtattcaacctgaagagcagcagtaagagacagaggaacgcagaggaaggaagagggtgggcagcagacttactctctctgtgttctacctagctaccacctatgacttcctacacatgacagcggtaagttcagagtacttctgccaggtaacacttgatggatattatcaggtggaattctgctgcatttatacgactcaaattgaacctgtctaccaaacacacaacctccccatccctgctggaaattacaatagtgactctgaaaggcatagaaagtggtaacagttgctgaggagcctggctctgattacaagttgccccatgggtcgggttgtctatgtccctctctctcttactttagtgctcctatgacctctacagacactagcattgctcaatggaaagcaagagaaagaagaaaccacggccccaggtaatctcaacacaaagtccaaatatcagcactcttggtattctgtaggaatgacaggtgtgcacagggctagcaaactgcacctggtgatcccatttataaactggggctttctgacagctttattaagtggaagctgagaacctggtagcctcaatcttggaaatcttgggatactcccacaggattctatagctttccaaacactaatttgcaaaactaacatgcaacaaaatgaataggccgcacatattattaatatgattagcaaataaagaaaatatgtatatgagtactaaggcaaagaacaggatggtgggtaaaacttccagtctatacatgtcagatcagtaaccgacactcgttctacagtaaatgactgttttaaaagaaaatttataacacttttgagggaaataaaatgaactaactcaccaaaagctaggtttctaagaaatggttttctcctttattttatttgtagatgacctgatgaattgtccttattctacttgcctgttctcccatctgggacaccggtcctgggcagggggtaggaggaacatgggcctctttaagcacaggcaagtttggccagctccatgaataggcattttatgtcaattctttaacgttgatatgttaagtttcatttgctttacctggcaggagatccttcaaatgctttctcagctgcttctcccagtatttcagcttttaggtagtacagcatccggactcgcaaaagtactctatgaaaggtgaacacattaatgcaaacaaattagaacccataactgtctgtgaaccacatccccaagttaacactgggaaattctgggcattcccatgtagttaatattatgaagtaatagaagagaaggcactagaaaaacaaaacattatggcagacattattatgatcattcctattttacaaaaaaaaatgaccacatcagttatctgttagatcatcctgtgagccacaggaggcagcaggaacagacagctcttctcatctttaagagtactcaccccttccatgtggccacagtgacctcttaggcagaggtggcttccgagaggccagctactgaagagtgagcagggtgaacagtggtgttttcaaacagtttcacttgtacacagttaaatgaataaatgaataaaggcaagtgtctatttttcgtggcaatatgtcttctcttctgaggtgctcaattcatttttattctgtatggaccacactcaggctccaagggaatgcatcaaacatgtctcagacaaagcaattaagcaagtatttgctgggcaactactatatgctctgcatcatgccgggtgctataggtatataacaaaagtaatgcctttcgccatcggatgacccttgcccacaagccgttatcgtgctcttgggcatcccggcctgcagagtcatgagccaatacccagtctgtggtattctgttatagcagcagaaaatggactatgacaaaggggtaaggcctctgagaaagcatcatttctacctattagatctgggtagaaagtgccactgtgggctcaagttgtaggtggaaacacacaaagttagaacttatagattggaaagaacccttattatctctggtataacatttctcatctcagctgtcaatcaactcaaataattctgagagtatttttttttttttttgagatggagttttgctcttgtcgcccaggctggagtgcaatggcgcgatcttggctcactgcaacctccccctcctgggttcaagcggttctcctgcctcagcctcccgagtagctgggattacaggtgcccaccaccacattcggctaattttttatatttttagtagagatggggtttcaccatgttggccaggctggtcttgaactcctgatctcaggtgattcacccgccccggcctcccaaagtgctgggattataggcgtgaaccactgcacccaacctgtttgtttatttatttatttttaaagcccagagaataagaactcttaaaaatttacaaccactgccaatccttaatttgtagatggtcgtagctgtcagctttagtaaaaagtggaacagaatgtcagagtcctgcctgctgagacttagctcaattagagtagggctttcctgaaaatgtctgtctccctaagacagggagagctgtttccatttctataaagagaaacaaatgtgttgacgtctaacaatatctgcagcatgagagaacagaattgaaagccaatagtattcaattctcctccaaatggaagggaaatcaattcagaggatggatggacaattccttatagccagccccctcctccaagctggtgtctgaactcaaagggacacacaactgcagcaggtttgtaacacccttggtttagccaaagaaaacaatccattaatattttgggtggcgacatatccttttttctttttaaaagtattttactgatgtgaaatatacttccataatgtgcacactgcaaacatgtatagcttaatgaattcctacaaacaaaacatacatgtgtattcagcatctagatcaggaagcagaatattctccctgaagtccccttggtattcccttacagttacatcccatccccagtggataaccattatcctgtctgcttttagcttagatttgaagcagtgctttttaactttttaaaggttcttttgatgacaatttcatgcctaaaataatatagtactaataataacaacactttgagtgcttactgctgtctctgaagagcttgtacttttgtgtacattagtttttattacagtattgggcatgagacttatcttcaaatatttgaggtgatcaagggaaaggattatactttatctgtaaaagaaaaaaaacaaaacaaaacaccccacaacccaacaactacaaaatcattaagaagatgttatagggagacgggaaaacccaagcaaaagctcttaacctataaagtaataaactcttgattaccagaggtgtgcgaccggaggccctcacactgcctaccaggaatgctgggaagataactcttgcatgaccttgacatgaattagatcatttccaagggtcagatacattgcacagtggtgcaaatatccacatgcaaacttgccattgttttgtcgctgaatgccccaagtccccaagactaaactaaatatgaacaaagttttgtttttcaatcagttttttttttttttgctgtctccatcccacccgaccccagctgttagcctacagatacaaagcatactatctcaaatgcatgcctatcaaataactgaaggccatgaatggcaacagcaaactggaagaggtggctgagagtggcagcgaccaaggccacaatgttctgcccttgttaatgtgacctcaggggcagaggatgacctgactggtttttgagagtcaaaatagaaaaatcctcaggggtatatacaatgcagtggcagaggggccaggttctgcgggagagcagaggtactgttaaaggagttataggagctgtaacatgaattctgagccagcttagaccacagataacacatcctgtctttctagaccttgcacagaagtagcacaagagcatgaagtgctctccctgtggggttcccaggaatcgaagcagtctgagccgggctgccaccagggtatggggaggagggacactcacttgttgcagtgctgtttgaggtgtttcttatagccatcatcatgcaagaccacctcggggttgcaggtggctagccaatctgcatcctttagctctgggattagcaactggttcttcgtcttcttccccttcctccctctggggactggggcagataagcctgaaacagaaagacagcatagggcaagtggatagccaaggggggtgttacacacgggttttagaagagctgggctggggtttgttagaacagagttgggctgtgctctggatggcagaccctactccttagcccacaggtggtggtggtaggaaggaaggccatgcacttggtctcctccttagtaaacaaataaactccaggaccatttagtttgtcagtaagatttcccaagtaccctgtgaggattaagaaaggtgtaacataacctgtgccctttgggaacttacaacctggctgggagaaaaggaacatttgacctaaaacttcaaggggagccttagccaaggatagggaaggaatgtagcacaagaaaaggcacagacagaggtgggacagggacaggatggtgaaatgcaaaaggcaagtcactgtgcatccctaattcagagggggagtctgtgtgagactcaggttagtgggcaggaccagatgtgggaaactttaagagggtccagttgtacttcaacaaaatatcacaggtaacactgggagattctgaggcagaaacgaaagcaacatcttttcccactggtaacattctgaaaaacacccgagaaagagaagtcccccaaagcacagcaatacggccccgcagaggaagaggggatgaagtgcatgggaatcgtgcttcttaggcatggccctgccacctctcacctgagtggttctggagggtctgggcttgcccatctttggtaggtgtgatcagttcccaaatgaaactcttgatcttctcgtcccccttataatgcttgacacagtacaccaggagggcacggcaaatcatctccatgtccttctcgttcagatgccacttgaatcggccatgagtcaggatgtccttccaccggccccagctgacagtggacatacagacaggtgttggagggagaatggaccaggccatgcaggcagcctccccacaggaggagaaacaagagccatacatgctttttgttctctgtaggtctgttaatcatcccaagggctttgtcccgaaaggcctttgaggaccacctgactgtagcacaccgaatatcactaaagtgacctggacgttggacactgagagcttcatatggccactacaatgttaaaaagggagggaccaaactactgggagcagggaataaatttggaatcaaaaagagtccaacagcactattcaaaagcaatgatgagctcatggcagctgaacctttgatttctaccagagccaaagccctgcacgaatcatgccttggcgataacatgtgttgtgtggactccttgtatatccttgcggggtgggaggaagcaggaaatcagtaccagacagcaaattaggaccagagatttgttgtgacaggcatatgagaaaatcatggcatgtctgcattgtggttgcggagcatacggtgactgagacggattctgggcagaaggcacagttctctcttgctccaacagatccccctttgccctataattccaaaggtgactggagagacatcctagacaaatctcagggactgaaaaacagaggggaacaaacaacaataacaacaaaactaagcagagccaatacccacccaaagatgagcaggttcttctctacccggaagcactccgctcggaggtagcgcctggctttgtcattgaggcgcctggatctcgtgggcctttcgtctgagtcgctgtctaactctgaaaactccatgagctcgtcttcctcaaacgagttgtagtgtttggtctgctttctcacgcgaggtcggtcgatcactaagctttcctagaaatggagaggactactgggaagaaagtcctcttttctctactcctttcacaaagcataagtgtatcctcaggacacatttgggatagaaccatgccccgtctcatcctccagcacgaagggctattcccagcacacctgctgtctccatgaccacaggacaagcagagaagattctctgggacttgctcaagttgatgtgctccctggcccccagtgctgcttgtcaccatctgttgtttctgcagtcattctacccggggtgtgggcaggcaaggcccccccccagtgaccaaagtggtgtcggctctagccaagaaagactggagtagtgaagacccagctgttctgcccagcatgcagagagcagtctgcactctggagacaagacaagctagagaattccatctgctcagcaactttctgaagacaaacgcatattcctgctttatttatctgccttcgactggttctatccactaaccttttgcctttcactgacaataccatctccagctcaggagcctcggtcattctgatggttcccctggctatgtgtcttctctagtctccattgcttcctgctcctcactccttcccccatacctcttccccaagccaggccagcctcctgactactcctccacacagcccagataacaaatgtcgtgttatccctgctctgatcaccgtgcttcccaacacggaaggctccaactgcttctgcctggctacagggccacagtgctttctccccttgcttggacccagtgtcctgcacacacagtagcttcttgattgcgacactgtgaaacacttagaacaggttactaaaggggactgcaggatcttcttaaaagaggaaaatcagcatttctatttataggagaaaagagggtggattaagcaacctgtggtcgttctggctgggtgctggttactgaattagctagctacaagcagggactgtctcctggctttgtggaaccatgagagttcaatactcacctgagaagacactcatcctgaagtgaggtgacagggtcagagatgaaggacgggaaatcaacatttaaggggtgcctcacagtgggctgggcactggtgccagttatacccacaaatgttaccttgtaaccccacagtgtgggcctgaccacctcctcttctactaatctcagcgggggtcaaaagcttcatccgaaggcatactctggtttcactctcaagtccattctctttctcatgttcagaattaagaaaacatgagacactgaaagaatggaggtaaagccaacaaacttttgtcatgctttttccctctattatgtgtgaagtttctctcacagaaagtgttgaaatacaaataaaatagtcacatgccagggatagaatgttggtttgaggcagaggcctgaacttgacctgtgtatccagttccagttctaattgccttcatctaatttcccaggtgcccaacagactgaatggccatttaaaaatgacaacagttaagatttattgagtgatgtctatgtgccaggcatattttaagcatggtacaaataccatttaagaaccaagactcaagagtaaataatgacttgagctgtgtaggaatttattgtcatgtatgacataagtgaatgttccatggaataaaatattattttgctgtaccttttcattctttgcttcagtgtctagttcagctattttagcccatttctgccaaaagttaggatcatctaaggaaatatctgttctgtttcctgaagccacaaagctagcctgtgaagaagacaagaattaataaactgtgcttgaacacaatacaaactaataaaacaccagtgttacttcaagagaaacctaagtatattatctatttgcattcaaaatcaaacccttaacagagcttccaaaatgaattgaaaatctcaaggctattctttttttaccttttattttaaaaattgggtgaggaaatcataatttcaactgaagggatcatttttttttggagcttaagcttggaagcaggaaggcaaaccccggaagacaatggcaatgtgacacttggtgtctagtttcctatgaaagagttaacacttaaaacttttctagatttttctttcagtgcaacagttaccaaatgtctgctatggaaaaggtattaggctgggcagtatgaggtgtagagacaagggatggttcacccatatcctccttataaaacactgaacgtcccactaaaaaactttcaggtgtcaccaaacaggagatttaatcaaacaggtcacaaagctcacacaggaatcagcagtggttgtggctagggaaacctgagagtgatctgctaagttttaaatccagtggcctaaacagcagagaaaagaggcacccagactagaagctgggaattttacgtaaaatctctttcccattaaacaaacaaacaaattgcttgagtttggggaatggagtgttcactggagcttccactctctatcagtgatcaagaagtgctctacaagggaatcaagatgtccactggaaggtttcagagcagtccttgcaaacagagccaactgagaataggagtgtttattctgtctgttaaagatcagctctcaaaggtgaggagtggtattttcttggtatagtccctgactccctattcagacttccttcctctaaggatctatatctttagatagtcacgcagactaaatgtgcagccaatttctaggatttgcattcagagaaacagaagccctgctaatgggtttcagagaataaacatttttttttctctcccacccccaacagctctggagagaaggccctgtaccttggcaaaagtggaccctttcccctcagactggatggtgatggtgtgcgttcgcctctgcagaatctggtctatgtcttcttcacagaacttggagccttcatcttcttcatccattaaggctccataagcacctttccggagtaggtcctccacctccatttttgagagctgctgtacctggacaggtcaccaaaggctactcacaaagtggaaaatgtataggagaaaaacagcctcaaatctggtttttgtgattcagtgcatagttcctccatgaactacatgttagaacaaaggacgtgggcaacaaacttcagctagtgaaatcccagatgcgatactctggggagggcaggcagacagtcctctataggcagctctgaaataaagctcagcacatgatccaaggtccctaacattcttcaggatgttggttattatcagatgtcacaagccactataactatgcatcattgaagatggtacttttgacatctttatattttagttagagcttgtatctttaaaaacctccagatgtaccaaacaagcagaagaacaatgatttattcattattaaataaatatgtatattagtattgtaaatgaggtcatttctcaactaaaaaaaaaatgatcccatgataaatgtgatcttctcaaaaaaaacaggaaatggggtcccaaacacccttttctggttctaattaagaatctggttctctttatatcaatcacaagagggaattccatgactctctgcatgactaacttgaagtattatacttagcaacatatgcatagctaagaggatatggaacatctggactaaagacaactcatttagaaggtttaagcatccaatctttgatgccctgagcccagagattaaggaaatattctgctttgtttgcctatactttaagatactttaggacaatttaagaatcttagctgtaattcttatgcccccaaagggggaaaaatattttaactgccttattttaagctgctaatttattcattcatttactgaccatccagcactttatgtttttaatatgccgagcactgttaggggccgaggggacatagataaaattggtcccagtacactctcaaagctcaaagtctgaggtatgagataaggcacagtaacaaatacaatgttaagatctactggcttctaacgggcacagttaaaaactctgtttactgaaatgttacaccaagctgggcaatcctacctcataactgcttctacggatttcctttaaccacccagaaaacacagacacctgactcttaacgatgccctgttgtagggaattacccaataatgatgtgagggctggctcaatgcagcgaaagtgaacccttaaacagctgtaagaagagaagcctgtctgggctgtgcgaccaatgttccttaagactccagagcaggcccatcacactcaccccattggtgccgccctttcggttgatgtcctgaagaacagccttgtccagccccagctttaggctggccttgtcaaacatctcgcgctcgtaggaatttcgagtgatgaggcgatacaccttcacagctttgctctggcctatgcggtgacatcgggcctgagcctgggaagaagaagagtcatatgcatcacgtcaccgtatgtataaacggcagcaaccctgggaataggggagtgcttaaatgtcatgtggtgtgagtggccaactctcggttacaagtaacaggactgatttcagaacagaatttatacccaaaggttggctgccaccctccgtggccactcatttcttctcccattgtgtactgactactgcgtgccagggataaagcaatgagaaggacatggtgtcccatcctgtgcccccgccctgctgtggtctgtgcaaggcaacttgtgagtggcacacattcccaggatgctgtcctgtcctcttggaagatatgcttcagagctggtagcactgaaaatttatttttaaaattttgctactgaaattttcaaacatagatcaaaatatagggaagtataatgacctcttacggacaatctcgtttatttttaccacactccaccctcccagattgttaggaagtaaataaatcctaagcatcatactgtttcattcttacatatttcaatacacatttctaaagatacaaactcttgtaaaatatatattattatcattgtcacacttaaaaacaactgcatttctgctgtgtcactgaacaggtacctttatttcctgtattttctctaaactaggagttctaccttggaacctgatcagcttaagatttgagcatctgaatgtacaccaagaggcagatggtatctggtcatctttcttttgaattgatgatcattgcctagatctgttaattcatcaggggctaccctaatgtacagttcatataggagggacaagctaaatatttgattctctttatttaatcagtttccaaaataacatcccaaaaagtgaccaatgaaattttttttattacttctttatgggatacaatgaaggcatgcacttaaatatatctgccatgttttactgcactgcagttattattcttattgctgttcacatttggctcatgggagcctctctaagttggctcctgagttccttcaacatgacctagtcaaatgtggaaaaaaggaagacgagaatcaactctgcagtatttgaatggactgaaggtatcagtatgaactcatggttaggtgtatatgtatatatacacacatatacatacacatactcacacagatatagaaataggtatggatatgtgtacatttatgtttgtatgttatatcaaagactggtagtctttcaatagttcccttgctttcaacatattccacactcctcttgtacatttcctgtccagacctggcttcagccattttgtttccagaagtcctagtctcttttagcgggaatgttttttctccctgagtaagcaagcagaggggtgattactgacactgagaggaagaggtggccatgccatgtgagtgagccctgcaggtatcactggacaccccaggtccttccttatcttggcccagagcagtaagacatgtggcccttgctcctctgtgagttagtggcagagcaagtctggagccttggacttctgctcctgccccgtgatcttgatcctggagtccagaattgcaagcaatgggtcccctgtgtcagaggccaggctgtttgagttggatttgaagttactgggcccagagaaaggggtaagtctaagcaggaatcctatgtttgcccataagggactcacccagtgccagaatccctggatatggaagcccccacccatgccctgcccacattcttcccctaacctcatggctgaaaatgacaccagaattcctatacttagtactaaagcggcccaaacactaagaagcccagtaagtactgggacagaagagaggccactggggcgggggtacaggaaatggccatcaaagcagggctaaaggagggtggatcccaggcacctcctgccacttggcccactgtgttacagaggtcctccttgcaataagaaagcttgaagttcatcttggtgcccaaaatttgcatatttctgtgatacgatttggctgtgtcaccatccaaatctcaacttgaatttatctcccagaattcccatgtgttgtgggagggagccagcggggaggtaactgaatcacgggggccagtctttccagtgctattctagtgatagttaatgagatctgatgtgtttatcagggatttctacttttgcttcttcctcatttttctcttgccgccgccatgtaagaacagcctttcgcctcttgccatgattctgaggcctccccagccatgtggaactgtaagtccaattaaacctttttttgttcccagttttggatatgcctttattagcagtatgaaaatgaactaatacagtaaattggtaccagtaaagcagggcgttgctgaaaagatacccgaaaacgcggaagcgactttggaactgggtaataggcagaggttggaacagtttggaaggttcagaagaagataaaatgtgggaaagcttggaacctcctacagacttgttcaatggctttgcccaaaatgctggcagagatatggacaataaggtccaggctgaagtggtctcagaaggagatgaggaacttgttgggaactagagcaaacgtgacttttgttacatttaagcaatgagaccagtggcatttgcccctgccctcgagatttgcggaactttgaacttgagaaggatgatttagggtatctggtgggagaaatttctaagcagcaaagcattcaaggggtgacttaggttctgttaaaaacattcagttttataagggaaggaaagaataaaaatttggaaaatttgcagcctgactatgcgatagaaaagaaaaatcaattttctggggagaaattcaagctggctacagaaatttgcataagtaacaagaagcctaatgttaatccccaagagcatgtggaaagtctttaggccatgtcagagaccttcatggcagcccctcccatcacaggctcggaggcctaggaggaaaaagtggttttgtgggctgggcccagggtccctgtgctgtgtgcagcctagggacttgttgccctgtcccaactgttccagccatggctgaaaggggccaatgtacagctcaggctgtggcttcagagggtgaaagcaccaaaccttggcagcttccacgtggtgttgagcctgagggtgcacagaagtcaagaattgagatttgggaacctccacttaaattttagatgtatgcaaatgcctggatgtccaagcaaaagtttgctgcagaggtggggtcctcatggagaacctctgctagagcagtgcggaagggaaatgtggggtcggagcccccacacagagtccctacttggtcactggctagtggagctgtgagaagaggggcaccatcctccagacccagaactgtagatccactgacagttcgcactgtgtgcctggagaagccacagacactcaatgccagcccaagaaagctgctgggggagggaggctgtaccctgcaaaggcacaggtgtggagctgcccaagactacctcttgcatcagcatgacctgaatgagagacctgaagtcaaaggagatcattttggagccttaaaatttcaatgctccggtggattttggacttccatgggccccctttgttttggccaatttctcccatttggaatggctgtatttacccaatacctgtatccaaatcatatctagaaagtaattaacttgcttttgattttacaggctcataggcagaagggacttgccttgtctcagatgagactttggactgtggacttttgggttaatgctgaatgagttaagactttcggggactgttgggaaggcatgaaatgtgaagacatgagatttggaggggccaggggcagagtgatatgatttggctgtgtccccatccaaatatcaacttgaattgtatctcccagaattcccacactgtgggagggactcagcagggaggtaactgaatcatgggggctggtcttttccatgcaattctcacgatagttaatgagtctcatgagatctgatgggctcatcaggggtttctgcttttgcttcttcctcattttctcttgccgcttccatgtaagaagagcctttcgcctcccgccatgattctgagtcctccccatccatgtggaactgtacgtccaattaaacttctttttcgtcccagtccccggtatgtctttatcagcagtgtgaaaatgaaccaatacatcctggtttaccaagttgttcttgggggcaggagggaaggcagaacttgttctccactgtcactttcttagactcaacaatcctggtcacttccggggagacccagaccccactgaggaaggagcacatagctggcaggcaagagcttatgtgtgaggccaccaagcatgggcaacagaacagaccctggggtctctgtgcacagcagggccaccaggaagaccagagcaaaggtcttcatagcatgggaaaaactaacttttcctctctcttgccaggctgcagggcaagtggctaagaaacagtccagccatagacaggggtagaaaacttacagacaacaatctggataccctagaggtggtccctgctactagggatgatcatggtttctagtgcctactcagtgggcaaatataggaagtgtttgtgtgtgtagagtgtatataaacatatgcatatataagacaaaatacatcatgaggtcatactgatatttttagttcaaattatgattgaagaattttagtttcatcaacctcacatctgtatttctttaatcatgttgaaaaactcaattttcaaccacatcaatatcagtactcatttgatttgtcccacaatataacattttcagaaaaccaataccattacaactcactcaaaacataattaccaaaaaatttcaagatttttctctccccagccacttagggatatatcctgctaagtatgtatagtgttttaaatacacacttgaaagtaggtttattttctgtagttgcaaggttagcaatcctgaaactactttctgtttaatctaggtttcagcaaatgagtaaagataccgggggtagcaggagtaagggctttcactgttggagaaggaaattataaatatggaaaggaggaaaactaaaataaactctgtggtgctagaatgaattggaggtatcagtattaactcatggttaggtgtacatgtgtatttatatacacacatacatatacataaaggtacagaaatagatacggatatgcatgtttacgtatgcatttgtgtctatacacacatatacaaatgaatgcatatatttacacatatgcatatttatagtctctctttctgtgtgtattccagctatgtccactgagagggcctagaagaaacatcattgcagtagcaatgaacacacccaactcctgaatcttgattgctaaaaaccattacctatacaacagagccacagtgtgctagagaaatggctaactgtagttagggctgtggtagagaaagtatagactaagcctgaaacatattactgtgtcagatgtaagaaattactcaaagaacgagggagacatatgagaataacacttaaagctcgaagagactcccactggctaagtctagaactatatgaacagaacatcgaaataaatatagtaacagattataactcactaaataaaaaaagaacccatgtattcatattgacataaaataaataaatgggagaaaaagggaacactcttccttatagtagaatgctaactaaccagtgcagagcgacagttagaaaatcatcactttgtaaccatcatagtgatgactgattcagccaaaaataatcaatggatgctaaaaccaatgggtgatactttgatgaggaacaggtacagttccaatttatttctccaaaattacttattattttcaaagggaaaaacagaaactttataagtggagaaatcggacagatatcactgtactaagtgatcaaagttaccatcatcagacaaatggacaccatgttattcctgatgtgatatattgagatggctgtgtcagttctgtagtagtactaaacctagatccaatcctgaggaaatacagacaaatccaaacggagggacaacctacaaaaaaactggccagtactcttcaaaaacttgaaggtcataaaataaaaagaaaaactgaggaactgatccagattaactaagactaaagggacatgacagctaaaggtaatgggtgaccctggattgaatcctggactggggaaatgacagctataaaatatgttgaaacaaatgataagacttgaatatgaactgtggattggagaatagtatttttatatcttgattctgagaactgaactgtgaatatacaagagaaagtccttgtacttagtgaaatacacactgaaagtatttaggggacaagggggtacaatgcctgcaacttactctcaaatagctcagacaaaaagtgatgtgtgtctatgcatgtactcagagaaagaaagggacagagacacaaaggagagagtgagaaagggcaaaaatgatagagtcgaggcaaaatgtaaacacagctggtgaatctggataaagggtacactggagttgcaaaatgaaattatatagaaataaaaatgtataaacatatgaaatacagacccttgaaataatccttttctgtgagttataagctattcacttgatacacagctagattcaattgtttcattttgcttttgagttttaggaattgatgtttccccctattaatttaaccacactgaagcaggtcgtgaagaaaataactaaactaagcaactttggcaaatagtattctgattatatactgtaatactaaagaccaaaaaacccactatagaccaacattatattctggtaaatgtatttttcacaaaatatgggttagcaattctaaaactacttgatgtgtatactaggatggaataaatgaataaatatactgtgttatgataacaaggtccaggcttcatcctgccagagaaagaagttacaaataaggaagaggaggagactataatgaaccctgtgatgctgcactgtaattagaaaattcactatggatgaatatatacagaataaatatagctgtgtggtatgagtgcttgcttaagtatgcatacatctgtttcctagctccatgtgcacagagggtctagaagcaataacaatctagtagcagtgggcatacctagtagccagagtttggtttctaaatgctgttctcaagtaaaagaaagcaaggatcgttggagaatggtcaattccaacgtgggggcacgaaaaatgtaacatggacttgaaccgtaaggaagcactcaaaaaacaatgggagcatgtcaaaaaaacacagaagccaatgtaaaggagctaccaacggccaaatctggacaacctgaaaacaaaataaatagagtaatggattataaaataaaacaaaatccacaagtcaatactgatataaataaatgggagacaaaggacaacttcgttataatagaattcttactaataaatgtagaagggatgatgagaatagaaaactaccattagctaaacaccatggtaataatttactgaaagcaagaaccattgatggaagctaaaattaatggacagaagtaggatgaaaaacaggatagagtctcaaaatattcactcagaagatactaatttttagagataaatacagttattttatggtggagaaacttggcagatggcacgttaaccaagtaatcaaagttacatcacgagtaataagatgtattcatatcacgcatcccctgatacaatgcactgaggaggacataatatgatttctatggtactcttgcctaaaatgcataacctcgtatctcaccataagaaaacatcaggcaaatacagactgagaacagtccacaaaacaactggtcagtacccttcaaaagtgtcaaggtcatgaaagataaggaaagacaaaggaattgtcacagactgcagaaggttagggagtcacaacttctaaatggaatgtggatactgggccagaaaaaaaatatttctaggaaactgaaggaatgaatatgaataaggtctgtagattagttaaagagtatcatatcaatgttgctttcttggtttcaatcattatactacggttatataaaatgtcaacattaagggaagctgggtgaactgtacattggaattctacataatttttgcaacttttaaaaaaggttaaaaaaattaattttgttttaataatgaagtaaaatgcttaaaatggttccaaagtcaaatacacaaaacagcatatttggaaaaaccagcttctttccctgtcccttccatcctgttctctaaaggtaaccatttaaaaacaaagtttgtaggccgggcgtggtgggtcacgactgtaatcccagcactttgggaggctgaggtgggtggatcacaaggtcaggagttcaagaccagcctggccaacatggtgaaaccctgtctctactaaaaatacaaaattagctgggcgtggtggcgcgtgcctgtaattccaactacttcggaggctgaggcaggagaattgcttgaacccaggaggtggaggttgcagtgagccgaggttgcgtcactgcactccagcctgggtaacacagcaagactccatcccaaaaaaacaaaaacaaaaacacacacagtttgttttagttttacccttggtagggaagggacagacaaagacacacagccttttaagataaatgctagcatacagtacaaattttttccaccttattttttttccccgcttaacaatatatccagggtacaatccagggtatagcaggatatagtgatagtcttcattcttttttcaaagctgaatatattacagtttattcaattagtcatctattgctgaacaattcagttgtgtagtctttagctattataattcgttgctggaccttctttctgtatctttagcggtagcaaatctctgctactgggaggtttgatgaatgattctgcaaattaaggaagtgatcatggtgaacaatatggtttatgagaccttcaagatagtaatcttatacggcccttaagtactgaccctgatttctttttctggctcatatggtgttgagggtaaattacaagagggagctgaaaagttctgagcaaaagatctgatggaggagccctgcagacactgctaatggccttcgtagcatccatctctttcttgctcactgccagaaccctgactcttgtacagggcagcaacattttcaaccgaaactactcccagcctcccagggatggccaggtaacacagtcctagacaatgaggtgtaacagaacctcctgaagatatgtgccaaagttctgctttcctgtaaaaaccaacatgcctgctttccttatttttcctttctgagatggaaccaagaggctgtaaaccacacatactgaggccagtggagcagaaagacaggaggaacccgggactccgatgacactatgatgtcacctcactagtcctcaactgcctacctctggacttgttaactgagaaaaataaacccctgtttgggagagccgaggccacaagcagctaaattacatagaaataaacatgttctcccccaagatgacttacctgaaggacaacaggataagtcccatcacatttaagagactcatcattttacagtcaaatctcaaaccattctttattttcctaagagaactcaataaaaaaaaacatgagatagagaaagaaacattcagaactagggccaggaactcagatgaagtccaagggaaatgcatcagcaaaggtatacatataaaaacctttggagtaaaacacaactagggggcagtggtgctataatcccccagccctaatccctggagaaaattatagtgctctaattatagtgctcacaatttcttctttattttttaatacatttggggtggacaaaagccaaagagaaatgaataacgacgttctgacaatgaaaaaaaaaaaaaaaaaaaaaaaaaggctgggtgttgtggctctcgcccatcttcccagcaccttgggagtctgaggcaggactgcttgaggccaggggtttgagaccagcctgggctgtcaacataacaagaccctctctggggttaaatctgtcagataagtcagtcaagaactagctgagaacaggaaggacagatgaattcacagaatgggaaaggggtgcttttgggcaacagaaaggagttaaaaaaaaaaaaaaaagaggacattgacaaggagaggcagcggcgtactcaaaaggaacgtgactcattctatttaggactggtgctgtgtaataagtgcctgtcccatcctgtgcttcagacgtgccgcctattcacatcccactctacatggtcatactctttgttttgtctggaatattcttccttcttatctcataattttagatccttttcattatttctgctcaaatgtcatcttcttaaagaagtcttcctagaccactctttctaaatcagggcaaccaactcctatcattcttttttttttttttttgggagatggagttttgctcttgttgcccaggctggagtgcaatggcatgatcttggctcagtgcaacctccacctcccaggttcaagcgattctcctgcctcagcctcccaagtagctgggattacaggtgcccatcccgacgcctggctaatttttgtatttttagtagagacagcatttcacgatattggccaggctggtcttgaactcctgacctcaggtgatctgcccgcctcggcctcccaaagtgctgggattacaggcatgagccaccgcgcccagccccaacttccatcattcttatccccttattttattttctttgcagtaaatactgcttatctgtaattatcttatttttcctcatttattgtctgcctatcctactgaaataaaactgtcagtctagctcaccagtgtctctccaatactttaagtaacacctggcatgtcccagtgatcaagagatagtcactgaataaatgactacatagtatagtatttgtgtatacatttacaaattattactttctcatagcaaagaagactcaacaggtgcaatcctccaaactccagacacacgtactgtcaagaaacaggggatgtaggtggggcgtggtggctcatgcctgtaatctcagcacttcgggaggctgaggtgggtggatcacttgagctcaggagttcaagaccagcctaggtaacatgttgaaacctcttctctacaaaaattacaaaaattagccaggcatggggctcatgactataactccagctattcaggaggctgaggtgggtggaggctgcggtgaactgacttcgagccactgcactctagcctggataacatagcgagatcctgtcttctgaaaacataaaattaggtaagaaatagggggtgcaagcaatgtggtggcagcagaggaagaggcacaggggtccaatacctcttcccctcggagcagtgtgggagtttcctgagtcctgcttctggcgattgtggaaagggcctgtctcctcactctggactcccaactgacttccaaaatcctgcagacatgcagcagctgcattatcttgtggctgtgctcttggccaggacatctgctcccacactttctgaattctgccttcagggaccttagcagcaggaaggacgcagaactgtgcagcccaccgttgttatcttgaaccctcttcaaccctaaggatctcttgcctccagatgcaaaaagtcccccagagaataagaagcagaagtatcacagaacaatggaagaggaaggtttgtggtctctgaggggagcatgatagcatgttattcccaagaggtctgcctttgccttcaggggtagcagcaacagtggcctactcctaagaagaaagaagacaaaaacccagtttttcctttgtctttccttctcaggctactgttatcgtcctgtgccacttcttaaccttaggtgttatgggaaacctagacaacacttgctgctgctgatgtgccaggactaaattagagcctctgtcttctaaatgatggtgagaaaaggcaaactgataaccttcaattttgtacacacattgatgcgaatacagacatattcaaggtttgagagagggtgtggcacttacctgactctagataagatctccccattaaggggacgtaaaaattggttttccttttaatagtccagataaaacagtaatattgctaagatctctcagtgctaactttaatctcttttgttattcctaagtagtacccagtttctatcctggatcctaatgaacctgtgccttcattttctggaaagcaagaggaggacaggaggatttgggaagaggaacacaatgatgtcagcgtcccagcctccacactctctctggagtaaatattctatatttatagcacgtgggtgcaagggcaaatgccaaggggcgtgggaatgtgtgtgtttgtgtggggagagtgcatcattctgtggacagtctccagccctcttgcttttggaaagccctcttgctctcttcctcatcacaccaagcaagcaagaaccttgctccatctctgtgggagacacagaatttggaccttcctctctctatcacatctttttatagcccaccagtcccactaactaatagccactcttctagtcccctgtcctaaaactgctctacatttctcaattccttcatctctacctagactggctagactttaaaaaaaaaaaaaggatgattccttccgagttgtacacatgttctctctcacacagtaaccttttgctgcattctccttacgaactcccaccctgcatcttccaccttggatcttcctcagtgctggtcttcttcgtttcttcattcaggctgagcattagaggaagttcagaaactgactcaacacactagtaaagttcacactatccaatttcagtttggacttcactgctattccttaattcatctcttgccagctccccagcagtggacattgagaacagctactccaaagcttctcccttcatccaagacccttattctggacagtcccacccaacttccctggttgctacacatataaatcagtcacctcagatgactttgccttctccactaggtttgactattcacgttcttgcttttcccatggcattgcacctattcaactggtaatgtgtagacttcacctaggtgaagcatggtcattccaaaatgtttgcctctggccatgagctgttgccagccccatgcttccactgatcctcctggatatcgcttcaggcatgtccagccgaggtgtcctcaaactggccattcaactttggtttcctgtgccactattcttccagctgccatgcctcacagtcaggtgagcatcacaaagaagccgagaactttgtaactgtcaaggacttaagaaatcatgttttggggttggcaaaaatctgaaaagggcctgatagtaaatattttaggttttgttggctatgaggtctctgttgcaactactcaattctgccactgtaacatgaaagcagccacagattatatacttacaaacgggtgtgattatattccaataaaactatttttaaaaacaggcagcaggccaaaaggcaatcattttccaccccttaatctgtataattgtcgtaagtaggttcttgatagtgagctgatcaaatattagtttttgcttattattattttagtgttgagtgtgttactcctttaaaaatgggttattcattacatccagcaactcatattcagccatcttttcaccagtagaaagatggtaagctagttataatcactggttttctttgagatcactggttttctttgaggttgcatgttgaacatagtttactttatgatgtataccatgaaatattttcagtttttttttttttgagaccgagtctcgctctgttgcccaggctggagtgcagtggcacaatctcggctcactgcaagctccgcctcgtgggttcatgccattctcctgccttagtctcccgagtagctgggattacaggcacccaccacgcctggctaatttttttttgtatttttagtagagacggggtttcactgtgttagtcaggatggtctcaatctcctgaccttgtgatccgcctgccttggcttcccaaagtgctgggattacaggcataagccaccgtgcccggccattattttcagagttttaaccatcactaacttaatttaaagcactagttttcaactagtaggctgctttcttcaaacacaagtttatgtggaaactcattaaatgaaaaagacaaagggccgggtgcagtggctgacacctctaatcccaccactttgggaggccgaggcgggcggatcacctgaggtcaggagttcaagaccggcatgcccaacatagcaaaatcccatctttactaaaaatacaaaaattagccaggcgtggtggcaggcccctgtaatcccagctacgcaggaggctgaggcaagaggcaagagaatcgcttgaacctggcaggcagaggttgcagtgagctgagattgccgcactgcattccaccctgggggaatggagtgagactctgtctcaaaaaaaaaaaaaaaagaaaggaatagacaaagcagcaactctgtaggagtgggtgggtagagaagaagcccagacacccattggcttagtgccctcttccctgcatcccttctcttgcagtatcccctagagtacctcttgaatcctgtcaaacagcagcctccaaacttcttttgatcaatcacctgtattatgaaaaaggttttgagccacccacccaccttcttcaccacatacccataatcctagtgttagtaaatatctcaagatatatgcgatatacacttaagatgaagttcttcattaatgataatggctgtaaatctatattccatattgtcttattgacaagttagaatctttttggctgacttcgcgtcccatatagttagaatgccattgtttttactttgaaacagggttgatgaagaactccaaactggaccctgccattttcttgcttgtttatgttagctttcaaaaaatatatcaactccaatctttgcagtaatcttttaaagccaccggttatttagtttggctaaaaccagaaaatagaatccataaagccacttaactggacacatataaactctaatgtgttgcactgtgtggagaacaaaggaactagtgaggctgccactgttgcccctgccacaccatggaactttcccacccctctgggttaactcgtttactgtacatgacacatgaccatcttaaccaacaaactgagtgaaaatgcccgaatcgtctacatatcaagaattagagcattagtcaagattagtatctttatttctgataataataataattagtagttctagtctatatctggaggctaccactgtgaaacatcatttgatttaacggaaagaaccacactctctaattagttggaagggtcgacatttacttttcattcagtttcaccccctgaccaaggccctaagcctgtcatccagctgacaccatagcatagagcatcacggatgctctgtagccaagccaggccctatcatgatgttctgggatccagggcagctaaggtaaacagcaatcatcctagtggttacctgcaagtcattttgtgggttccagtcagaatcaaatatgatgcaggtatcagcagctgtgagattgatccccaggcctcccgctctggtgcacagaagaaagacaaagcggtctgaatctggcttacagaaccggtcgatggctgcctggcgcaggtttccccgtactcgcccatcaattcgctcataggtgtatctgaggggacccaaatgaacgaaagcccaggcgttaatcatgacttcaatggagaacagcacaaaatgcaggaagctgtcgactgatggccttaaatccctcacttctaaatttggaccaaatgacagcagtctagaaggaatcctgccccctacatatgcagcagggcagacacttaaaactgtattgcaaaatgcccccctctcaaaacagaacaaaacaaaaaaacaaaacaaagcaaaaaaaaaccagctgtaaactaagaaggactagaagaattaaatcataaaataccgatccccatagcccaagcaacttattctctcaagtattcccagtttatacttaaatttaagcataagaccaggaaaaacaaacaaaagcctttcccatctactttgagtagtgctcacaaaaaggactcagagttttcatcagtttccagctactttagggtaaaccagaaatggtaagaagattaaactgtgaggcagtccttttatgtcacttctagcctaccaagttcctcaaagctttggaggtacacagcaaaacctccaagtttatagctgaaaccaaggtcttctggacagtagtacattgccaagcataaatcacaggaaaggtttttgaggtgtgtgtgtggggtgaaggaactgatgaatagttagaggaaaataaagtaggaaagtaaagctgagtatcattgagaaggcatacaactgtgtggaaaatcaagaaacaggggtggagtgcatggtagaggtaagggtaaaaggaaaaagaataagaaatgaaactgttaagggaacatattacccatgcccatctagagggaaaacagaagagacctttacgttggagatcatgggattaacacagaagatatagcagcaatctggacagctgctgggcgtcttattcttttcctatttacccctgacaacatatgaattaacaagggggatgttactctgggcccaattttgacccacaaacatgaactggttagtgaagcagaagtaataggaaacgtgtgagaaaagaaccaaatcaccccaaaccgacaacagccaagaccatgaatcctggcaaaatcctatcagcagcccagaatttgggaaatcagatctctaaaacttacataaataagcaggaagaaaataaggatagataacctaggggtagataattctgtgtgaggatggctttggaaaactaaatctggcaatatgatcttgacttccttgaagaaaagaaaaagccatgtaaagaaaccacccaaggaaatgtacatgaagaaacctgaccaactcagacttataagggacatgtataaagcaaagaaacaagtctgaaaatgctgtctcccttttataaagccacaatacaaccatacctaaaattaagggtacaattttttgtttcctctttatttaaaattataatacactcaagaataataaatgagaactacagcagcaaatgaaacaggcggtggtgataggattgtcatgaaggcagactaaaaagattttgtgtcatagacaaaggctaaggattagatgtctgaattctatagatttaattatcaccattattcctaacaaccacaataataatattagcagcaaattcttaagtgtcagttctgtgccaaaccctgtggtacatgcttggtacacatcattacatttaattctcacaatagtcctatgaggtagtttctattatcatcctcattttacacttaggaaacctaaaaacagaacaaaacaaaaattcatacaagtaacatatcttgctcagtcatctagctggttaagtgagttaaacccacatccatttgacttaaagctttcacactgtaccttacatttcaatgcagtaggacagaaaggttttcctaaacacttaagaagagaaatggagaatacataagggacactagtaaactttggaagcttgtaactctaacaggagatggaaacagaaactataactaggtatcaaaacatttttgataaatcagtgctcaggatctgtaacagttacagaagtttagccatgtatgtgatgcaatagaggaagcacaatacttggagtcaaaaacagggttgagattcaattaaagaacagccatttccccccctctaccctccaccaccaacacacatatgaatgattaatgctgcaggaaaagactccatgctctttgaaaaaacacccctagtcatcagagttagataccacatagatcagagtgaggctgggaattagcacagccttaagggggaggggaaatcaatacatagtgatgatacagctcagccatcacctagggaaaccgttcccataaaattagataagtgtttttgtgtattataatgtaacaacacttttagaacttttagaacaagatgaatggaataattctctatcacttgtttagttttgagggagtgctctcaaagaacacaaaacttaaaaaaaaattgaattatttgagctcctccctcacttttgagccagatcctctggccatgccctcattcctgtgtgagagtctggcatgaaattaaatgggaacaaaaccaaactgacagctggagaggcaaagaggctcaagaacaaagaaaaccttgagggctaataagtctgaatgaagaaatcagacatcataaaattttccacactgaacttcagcacagcaattaaccctctgtgacaactgtatttttaacaagatccctaggagttgatatggttcaaactgtaattctgagtcactatcaaatactatcaagtatcaacaagcagcaggtggcaccctggaaaaaacctctatgtgactggccaggagtagactgacatgtttttggcacaagaaatgttgactttagaaaggcctgaagggaaatgaacaactttctggtctcccagcaataatcaactggaaaacatgaagagttaatttaaaaaaagaaaaccaaaaaagcaaaacaaaacaaaaccaacaccaaaaaccattcacaacagttacaagcatataaaatacttaggatttaattcagtaagaaatatgaaggcttggtaacataacactaaggggccatgctttgttcaccattatgactgtaatacctacagaggttctagcacaatgcaaacactttacaaatatttactgagacaatagtgtatatgaaggacccaagttaagtgtagtgaaaagcatgttctttgattttgcaaagatgtcagtctaaaaaaataaagaaagaaagaaaaagaaaaaaaaaagctaaaaaaatcccaacacacgaatatatgtgtatatataaacatgtaaatgtgtatgtatgtatacatacatctatacatacacacacatttttctggagttaggggctggagcagacagagacagataaataagccaggtatggtggctcactcttgtaaccccagcaatttgggaggccaaggcgggaggatgacttgaggccaggagttcaagaagagcctgggcaacatagtgggacctcacctctacaaaaaagaaagaaaaatcagacaggcatggtggccaatgtgcctatcgtcctagctactcaggaggagtaggtgggaggattgcttgggcccatgagttcaaagttgcagtgagctatgatcacgccactgccctctagcttgggagacagagcaataccctgtctctaaatataaataaataaataaataaataattctgaagttcatctggaatattaaatatgcaaaggaataaaactactgatcaaaagactagaaaggctagacatttatccagtatacataatatctccctatatttcatagaattttatgagtcagggaaatagactattttactaaacagaaaattagaaaattcaatatatttggaaataaatgaaaataagacttcgattatatgccatttaccaaaattacagaagggctaaagacttaaatgaaaaaaaaatcaaataatccagttaaagattatgaatttaacatacacatttaattctttgatctctccaaacctctagtaaaatgacagtaaaggatgttttaaaaaggtataatcccttccccaattaaatccaccaaaagaacagaagtgataaaattctggcatctggaaagtcgtatttgatttgaagaaggcagaatcccaggcaagacacaacctaatttaatagtgcagaacacgtgacgggatcaaacattggtggcactaggtatccccgaaagcagggacgcagagtctaaaaacagggaaactggtggaatatttgctgaagtatttacttagcctctagatcacctaccccagccttgaagagggtaaaataaaagatcttgatcctctctactctagaagatactaaccacagttaaaagtgaatgtaccatgctgaaaaggggaaaagagacagagatagtgggagcccttactctttctcacccctcccccacaattagctctaaatacagtcaagtttccacctataggcaggggagagtaagaggaacaggacctcaaaatgctgatatagaagggtccccaatgaaaatggtccagccagatgcacagtgctcccatatggaaagtgaagtgcacagtcaacaagccttgctcacatactcagagcttctggttggctccacagtgcccccccatctgatggcctaagatatcagacatgaggctgatatgaaacacacagcctctgcttctggtaaggaggagcaaggtcctaacacaccaaccctctagcaaatgacaaattctggacaaactataaaaaccaaaatgaatgaacagaaaaacctacctgagggcactggcgagttaacaaaagcagacagatcttggagaagaatagaaatttgtggtttcagccagaggggaggccacaattatggtggcaaggtgttgatcagctgtaattgataaaagctgttgtctttctgacacacatcaggggaaggtacctagggcaactaagactgccagagagtgagaggggacccccagaaagcagagaaatagagatgggaaccccaaactctgggtataaactctgcccaagtttctggctgacttctgactcacacttgctcgggcctgattgtggtagtctgaaatgggaactactgtctaccagacacattgacagagtttgcactttgagcacaacctagttaactgcttccaaataaaacccatcaacatcttcagagcaatataacagaattctaaatctctagaacataatattcactagggctagggaaaaattcagttattctcattctcaagggaacaggcaattaacagatgtcaacgtcaaacctacaatgttataattcagacaagaactttaaagctgctattatgctgacgcttaatgaagtcaaggaatgttaagtaatgaataaaaagaaaagaaaactcagcagagaaacaaaacgttaataaacatagaaccaaatggaaattttagaactaataaataaagcatagagtgtgcttaataagcatgatggagatgataaagaaaagtcagtgaacttgaagacagataaaagagagagaaaaagggtttttaaaaaaacagagcccatggacttgtggggaattatctcaaggtcaaataaacaggtaattagtctcacaaataaccaagagaatggggcaaaaacaaaaaagaaattgaagaaataatgatcaaaagcttctcaaatttgatgaaagacatacatttacaaattcaagaagctcagtaagcaggatgaatcccaatgatcacagttaaactgctgaaaaccaaaaattaaaaagaaaatcttagaagtagccagaataaattaatgtattgcacacagggagcagcaatgtgaatgactgcagaacttttcataagaaaccacggggaccagggcacagtggactaacactgtaaagagctggagggggaagaaaaggaaaaaaaagtgaacctaaaattctaatatgaaagacagatacaaacaaatgggaaaaaatatgacttggagaaaacagatgaagataaaaaaattctatcctgataagccttagaaagacaggagaagacactgtacttgtgaaataaccagtgctattaaaaatggaaaaattacaaaaaatgttctgtgaaattaaaaaatataatgaaagataaagtcaagaaaattccccagcatctgaacttagatttcagatgggaaagatctatttagtatccagcaccaatgaagcagttcactgtaaaatttcataggactggggacaaagagaatacataaaaacttctaaaaagaacaaacataacacatacacagactaagaatcctacaggcttcgaacttctcaatggcaaccctggaagctagaagacagtggattaacaccttcaaaattctgtagggaaatgatttacaacctagaatctcaaatcctgcgaatagattccttaagaatgagagcagagtaaaacattttcagacaatgtctcaaaatttacttatcattcaccctttctcaggagctcctggaagaggtgctccaacatggaatccaccaacaagaagatctgatttaagaggaaaaaaaagaaagctgtcagcatgagaaacaaggcagatcacagctgacaactgtgtactctgtggggaacagcaacaggccagggaggaggaagtcaggaggccccagaaggtatggcctagagaagaggaaactgatgtgttggaacataaccagagatttagacaaccagggtaaagtttgctgctgaattataataggtgtatagaaaaataagcaatcaaaaagataatttttagtttcagagaaaataaaaactgtacaaaaatgaaaagtaaacagcacatgacataactcagctgtaatttcacattttcctaagtcataataaacaacaaatattaacctaactcaaagtattatgtggcatactgggaagatgagaggacaggaagttgtatacatgacggtatgaggagcagtgaaagaaggctaaacattcatcttccacagtgagaacggataatacctacaactgaaaaatatcaagaaattacaactaatatcattatttagatacagctataaataccaaaagaaatagcataaagagttgaaagtaaatgcttctgggatatgagaaactgggggaaggtagagagtgaattagcaaataatttttcattaagtgtaatagaatgatatggctctttaaattatgtgcacatacaactttaatgaaaataaaaactaaactaaaaagagaaaggcaatgataatattaaggccagaagaaccctgttggatgcagcttgggtgaagaagccataaacatgaaaagaagagctgggtatggtgatctatgcctataatccagctactcaggaggctgagatgggaggaccacttcagcctaggagtttgaggccacagtgaccgagccactgtacttcatcttaggtgacagagcgagacattgtctcagaaaaagaaaaaaaaatatatatatatatacacttcagaatatttatatgggtctcacggttaagatgagagagaagccttttaaagcacattaatttaagcttaagttcaggcctaaaaaaggatttaaagttgttttaagacaaagatttgactattataaaatttaggacatcaaaaaataaaataaatgaaaacccatactcataaataaaattaaagaacaaaaacaacactggggaaagtttttgcaacaaattcttataaataaacaaaaagtcaaagaccaagagaaaatgcatgaaaaacatgaatacacaatccacaaaaatgaaatataaataacaaatatgaaaaaatactcactctcattagtaatcacaattacaaattaaagagagaataattttgaattactggattagcaaagattaagaagtaaaaacaatattgatcagaatatggaaaaaagcattctcatatgatattttggaaaggaatataaataaaacactcttaactagagggcattttggaaatactgagtgtatcaaatgtttttaaaaatgtgcactgacatttatcctaaagaaataatgaaaacgttaagtactttaaagggatattcatttatagagtcccttaaaatgctgaaaaattggaaatgtttaaaatagagtatcacatagatcattaaaattttgttgtagaaaaataaagaaaaaaatttcataatatattaaataaaaaataagagttacaaagtgcaatgtagaacagcactgcaattcaaacaaacaataaagctaaacatatttatgcagaaaaaaacagactgagaagataacccccaagatgaatggggagttgtgcaggtggtagtgttacattatttccgcattttcaagagaaactacaatcttaagtaatttgaacacaaaatgagcaataaataataaagagaaaatagcataaaagagagctaaataaagacttctgtacttaccaaaagtctgttatatacacctttactcattttggggggttcttttttaaaaatctgcaaactaacagttgtacatattttaaagagtaagataatataagaagcaaatgctctggcaataggctcctaatctatagtgaattgaaagctacaaaactgctttgcaagttttgatttattcatgtaatacaagtctcaagaatcctttacatagaaatctctcctatagagctctgaggaattattttattgtctaatcagactttgaagaatcatttatgttacattaagtgtcagcatacccatagtttaatttgtcatttcttttaatcctcttgctataaagtacatgtaatgtacaagggcaaacagaatgttaaagaataagaacatctgttaagacacccgagatttgctatgaaaacaagattttctttagacttagcaatgcctaggctaccagtttctatgatcttgaaagtcttttatcattaagaatgaatttttaaaaactcatttgtcatcttcagatcattgatgtgtcacaatgacggaaaatctaaagtaatacacatgaggtaacaaatgtagtttccatacagaatccaaataacccacacagcaccacagactttttacttgcatgaggaatgtaacttagattcaaggaattctaagagtatgaacaagaaaataaattgggatgccatagccagtgcatcataaaaccttaagagggcttgctgagaaaggacataaaatatccccacattctttctctgtgagttgccagtttatgctctttcaggaatttgatatagtgatgatttccaggatatatttctttcaccattattaagcaacaaaaatgtaaatacattattttcttagccctttctaatgctatttgaattatttcctccaactaaagtgaatataaataaatatgtggaattttttttttaagcaaagcaaggactgtacatcataggtcatgaaatttactcttgacagttaaacttgggtccatttggtcataaaatttaactctgaatttcagtctatctttaaaaggagattcctttcctccgtttttgaatgttttgtgtttaaacaaaatttttgaatggatcaaaaaaatatcaaaggaaaggaactgtcccattgttgaatgcagagctttgacacaagtctcaccttctctggatgaggtaatcttctaggatgtcgaggcagcgcaccatctgggagaagatgagtactttgtggccacctgcaatcagcttagggagtagtttatcaatcaacacaagctttcctgctgcctgaatcatggcctgcagctgaaagtcaggggcatcagggctgtgggtttttcggaaatcttctagaattttctcctctgctcctgaaaaggaatggaacaaaactattcctcggacagaatataaggataaaggttgtactctgctttacagatcctgggaaaaatgaagtgaattcttaggtcctagaccagcagtccagtgaggagatgctgggtgcactctttggtctgtgccttcagcagagggctctgcagtggcagcaggagagaatgcgggtgctccactctcctgggaagtctcaccaagtagagagaggatctgagataccaagtcctaagctgactcacccaagtaaagaaaccttcccagacaagaaggactttcatacctcttgacatcacaaagactgtaagataatccaagccacattttgattttaaaatttctccaaaagtttcatagtaaaaaaaaatcctcccaacattttctctaactagtttggttaactgttaatttgtctacagagagttttaatctcattgcagcactttcactgattcaactaagtaacacatttccctgtccccaagtagaaagtgtgggccagactttgaacaaataaagggcagcaaactggtgagtgatatccatcttctgcccggcagagtacatgctgtgaggcagcttgttggcagaagttaagttcaaaccttggctcttggtccttagtagctataccacttaggcaaattcatcaagctctctgaatctcagttttatactctgtaaaaaggtgagaatgcgtacatcctagcatataatgaaaattcaatacatgagttatttgattatcattagcttctcgcagaggtcatggcagtcctgagttacttcctagtgcccctgactcaccattgatcaggtagggatggttacagcacttcctcagctccatcatggtgttgatgagattgggcatgttgtgctgatttgcccccttggtcaggaaggaaaagttcttctcgaggatggcacggtagtactttttctggatattggtcagttccacctcaatgatcgtctcttgtttgggagcaaggttcttttccacatcatctttcagccgccgaagcatcattggttttaggatagactgcagtttctttacctgtccagggaaatgagacctagttacctgcctcaatcccaagttaacgattactggggtattccaaccttcactctaatttcttgaacatttacccatagtactgtggtagtagactaaaagaaagattatgatcgacccctgaaaatggagagtttataatagtactgaaagggaataagggatatgattttaattcacaagcaattactgagaatctagtggcaatctcagaagataaaaagggagaaaggtataaaagagtacctattctctaggatggagaaaacatgtctctctgtcattctttctctaatacacacaccacatgcacagtaattacctgaagagtattttaaattatgttcggaggtatgagtgatatattacaaggaaacttagacgctgagagaatggagtaagaagtagagatgatctggaaaacaattcctcctataccttttaagtaacaatagcagcatacatgcttgggtgtagcagtatgtaaagaactaatgttataggtgacatctactctatcatggaatctgtcacatcaggggcactttaagtctgatggtgttctttgctacaatttgtctgttcttcccctaaaagcacaggcctggacagcagatctttatgcaactgactgcaccagcaaggtgacttgttccccctcccagtgagacaatgacagactgagggattcaaagtcaaagtacctgagttaacctggagctaaaacatgataccggtaagattaactctgatggtaaaacaaaataagggaagatgaaagtgcaaaagaaatcacgtttcaagtcagaaacggctgttcaaaaatttaaaaggggtagactctgcataaacaagttgtgtgctgcgagattgcttcctgaagaaatataaagtgatttacagagagtgatttagaagagttacttacaaaagcacattttagacaatgatgaacatgctttattttgcaaacagatgctttccagaacgtttatttactgtgttttaaaatacagacatctcatagagcagagattagagtagtggttaccagggactggggggtggggcggggtagggaggtattgttcaaaggatacaaaattttagttagacaagtggaataagttcaagagatctactgaacaacatggtgactacagttaataacaatgtacagtatttctgaagattgctgagagtggattttgaatgttctcacaaaaagcatgtgctaattagctctatttagccattccaccatgtatacatatttcaaaacatattgtacgtgataaatacatattttgtcgattaaaaaaataaagccaaaaaatttaaaattaaattgttatatataaatattaaaaagaagttttgatgtcatttttatggttttctcctacctaactccaatcttaatctttttatgagactttcatttttaccaaccaagggtacttagaaatcgaatcccccatatgggtaaggaattactatataagaacctaaagagaaaagacacagctagctgattgttaaagatagggacaaatgtcaatctgccacatgctgggttcagcttttgcccttctcttggttcataccttgaatcatttagagtttactatatgccagacattgtgctggtcaatacaacaaacacaacttgggagaagatgtgggccttgtcctcaaggagatcttttgtctaggaagaagaaaaaatgcataccacaaattaagtgcaatgggagaagaggatgcatttctaatggtccaaaaacggagagaaggttagagctggcttttgaaaatcaatagatagggcaaatgtaggaattaagtttgattattcactgctctgatgcaagcctgacttgcatcagaagtaggaagaaagagattggaaccccagggtgtacgcttgacatgtgggcaaaattcccaaaccttttcattcccctcattctttgccccaacccacttacacaccattgctggattaatttggcagagctaattatgtcattcctgtaataaggtcttgcatggcttctctgttacctactgaattcttcaaactacctttcaacatcttcccacaatataaccctaaacacttctgcttatattttccaattcatccactctgttttcagtttaatcagatgtttccccactagtgaataactaccatgactctgattatgctgggtttttttttttctatgcagagtggttaccacctcttctctccacacctgttaaaagctgcccatccctcaagatgcattttcaaattgtactctctccctagagtcttttctaattctcaaatagatgtaacctgtctctcctttgaaggaccacctctactatttgttcctcttatggcacgtacatttgtctgggttatttctataatccctagtggagattctataatccctagtagagtatttattactatcagttgttttttattagctttgagtccaacaaggtagacttggaactggaccttgaaaactgcagaaatgcagttgatctggacaaaagtggacttatcaactactccaggtatgtgactgccttcttttcctcactacctgtgtattgtgctgtatgcaagaagatgggcctgggaacaagttgcagattttgaagggaaaaggatgaaggatgagggaagcaaaagaaagcaccatgctcctgagaccagagctgtaagcaaaatcagcgaacgcacaatatgtgaaattagtgaagcaaggaggcccgatacaccaacataaaatattttgggcgcataagatattttgggtatggacccatctatcctctggggcaataagaactgctaataatcagcgtacccgccccaacatacacaaacacataaaataaaggaggtaacgctcattttaccaggaacgtgacaacttgctaaatcctggccccatagcatcctgctcttttcttgatgaagataacgatcacactgtctcctcaattctttgggtccctaattacctgctcctctgttttcagatctccaaattcctccaagaaagcggtctctgaaggaaactgtgatggctccagaaaatttaacaaactgaagagctcctccacagagttctgcaagggtgttccagtgagaagcactttatgttcctgcgcaaattaaacatacatgatggacagtgcttgagccatcaaaatagtgcattttcgtggggaaaattaagccagggcattgagtgctcaatttggggcaattctgatctgctttaatgaaaagataatatgccatcataggggtacagtcaataaacgaatcaaccttcctttcacatcactgattccagcagcacgttcatgttaattaaagcaactattcactcattcactaaatattattgagtgcctccccaccagatactgggcactgtgccaaggcagtaatgttaaaagggcatcatctttggtaccagttcatttaggggcctggaaaccagccaaaacaccttacatttccaagtcatatatttcatggtatttaagagtcctcctaagtctatggaaatcaatctgtggcaaaggtcaaattgttgacccagtttgtaaccaaaaaatcttaattcctatactattgcagttatagagaccaaaaaagaaaatctaatgtaaaacgactaggcagaaataagttacatgcagaatggctaaagagtgtttcctccaagctgaacaaaaggaacctgtgtttatgtaaagagatgctaactctcaccagggccataagctttagaccctccagaagtttgcagttcctattcttcagtctgtgggcttcatcaattatcacacagctccagtgaatcttcttcaactctgggcagtctgctaggatcatttcaaatgttgtgatgacgacgtggaacttgaagactcctgaaagggggtttccctgaggatgacacaaccaaagtcaaagctttacaccatggctatagaattctgctaatatctgctggactacaggaggcagcactacagatgctggggcctaggtgagaagacaagcactgcttttcctcatgtgagaaaagagaatataagaattaacgagctagttagccagaaggcctcacaccatcaatgaaacagaagcgagacaacatctgccctcacaaaggtgagaaggatgggcaatatggaaggcacagcaagggagcatttttatttattagctgtcatgataaggaaatcagataggtgttgttcacaagtccccagccaggagtccatctaaaccatgcatagtttgaatgcccaaaggaaactctctgctatttttcagacttaatttcaccttataatttgagttctagcacagattgaaggtaatgattctgatgcacctaagacaagcgctactaggcaggacacagctttgtttttcagactgtcttggtgttgccatcacatttcaagagcagagctgcatcatgttagagcacttcttaaatgtcttccaagtgtctctgctgtttttgaaacctctggagagaccactggctttcctctgattgaaatcgtttaatttttaccttaattataaaaaccaatgtgcaggacaaacacacaactatattcagctaagtgacagcacttaaaaacttaaagagaaaatgttgctgttgggtttctaatgatttgaaacctattttctctttatccttctctgtcaaatctgttaacctcctctccaaactccctggctgggaatccattctctgtatagtaattcttttgctcactttcttcactgaacacataacaattcagataaaatcttctgcatataaaagtaacttcataaaccaataagtaagtcaaactttcagtgtcctaaaaaaggtatatctcagagtttagacagcatgccctacaatcctgtgttgtatcataaattaaaacgagctaccagggagagcaggagtcagccttgacaaacatattacaaagtcattccacattaatacaagatgcagggcactggaacttctgaagataagtgacagaaaggctgttctgagcttaggcagtccctgggtctctgatctcacgtgaggctcacctgggcgtctctgtacaccatttcatactgctggatcatctgcctgctgatctggctgccgtggtacacaatggcattcatctctgtccatgtccggaactcccgctcccagttagtgatggtggagagaggggcgataatgagaaaagggccgtggattcctctcagaaatatttctgaaaggaatgtgatggactggatggttttccctaggcccatctcatcagccaaaatacagttttttctgcagagagtgagaaatataggtaattcattatcaatataggaaacttatagaggctggttataacttgaaaagatgcttcacatacctgtaagattatcattgaaggctttatcatcatttcacatattaaactgaggttagttattattattattcctactttgcctacttccaaaacagaaccatgctaatttgggagaaaatacagtcatgtgtcacttaaggatggggtcacactccgagaaacgcatcactgcgtgactgtttcattgtgggaacatcatagagtgtacctactaaacctagatggtatagcctactacatacctaggctatgtggtatatagcctatggcttctaggctataagcctatacagcatgtcattgcagtgaataccgtaggcaattgtaacaccatagtaagtatttgtgtatctaaacatgtctaaacacagaaaaggcaaagtaaaaatagagcaaaatatagattaaaagtggtatacctggccaggcgcggtggctcacacctgtaatcccaacattttgggaggccaacgcgggcagatcacctgaggtcaggagttcgagacgaacctggccaacatggtgaaaccccatctctattaaaaatacaaaaactagcagggcgaggtggcaagtgcctgtaatcccagctactagggaggcagaggcagaagaatcgtttgaacccaggaggcggaggttgcagagagctgagatcacaccattgcactccagcatgggtgacagagcgaggctctgtctcaacaaaaataaaaaataaaaaaataaaatggtatacctaggtgataggttgacaggtgcagcaaaccaccatggcacacatttacctatgtaacaaacctgcatatctggcacatgtaccctagaactaaaaaataaacattaaaaaacaaaaacaaaaaaggtatacctatacaggggacttaccatgaatggagcttataggactggaagttgctctgggtgagtgagtgagtgagtggtgggtgatgtgaaggccgaggacattaccgcacaccactgtagacattataaatactgtacactaaggctacactcaatttatttaaaaatattttttcttcaataataaattaaccctagcttactgtaacttttttactgatgaacttccaattttttaaaaaactttttgactcttaagtgttattacaaagagtcaagaagttttaaaatatttaaaagtttttgtatagctgtatattcccatatatatatatatatattcctataaatatatatatatattttttggttttgttttgttttgttttttgtttttttggttaaggatttagaaatagaaaacagctttaaaggatattctggattttgtttcatctagattgagcccacccttttggtgtggctggccaaaaaaaaaaggcacaaagtcagtgatgtctaaagatcattccttttggttggagaaacagctgacaaaaataaagtaagtcttggatgattaatgtgcaaagactaccaccctaaattgatggccatgctgctccctctcaccagtctgctactgcgactctaggtgttccaggctaaggcaataatatacctctgggtacaaacatctgggctggttttggttccttacctgttataccagttaaaaagaagccagttcatcccttccagctggtactcccggagctggttactgttcttatactcgcgagacttctcaagtttctgccaggagtctgaagcaggccgctcctagggaggaaagcaaacagcataatagatagagaatgatgttttaggagcataacatggatactaacgaggacagtggtaaatctcactgtcccaaggctggtttcaaactatagttattaccttctgagtaacgtataggttgggggaactaagtaggagtcaggctgagagaatggttaggaatcctgattgcttctagataataacttacaattagcttcagaatgaaactctccctgctttctatctggtccgtcactgcattacaccacacaccacaattagcctggtgctgtatttatttatccatgcacacacatgcttcttccattagactgtgagctcctgagggcagggcctgtaccttgtttcttaccatatctcacacaagaatcaacctacagcaaatgcttaatagatgactgacaagttgaattaaaagcagaaaacagtatttatgggagagggttgaaagaataataatcaaccatgcaaacaaaacactattaactatgtaagtttctaatcacagttctgcatctcgagacacctctatctacctttcatatgccaaagaaaacaaaaacaaaacactgctcttgaatggactcctacagggacctaagtttaatgttccgagtatcaaccttccagtgatgtaaaagcaagaaataatattaataaggaactggataacagtttttcatataaaaatggcaatgtttttttaaacagaaactgatcattccagcctatagccatattctttaacaactgattctcttttcaagttagttacaccttaataaagctctgggctgggtgtggtggctcacgccaaagtaatcccagcactttgggaggccaagacaggcggatcacctgaagtcaggagttcaagacgagcttaacatggtgaaacaccatctctgctaaaaacatacaaaaattagccaggcgtggtggcacacgcttgtattcctagctactcaggagtccaaggcaggagaattgcttgaactcgggagatggaggttgcagtgagctgagattgtgccactgcactccagcaacagaccaagactccatctcaaaataaaaaaataaataaaagctctgatcccaactcccctccttttaattgttggataatctatatacttctcaggcgccctgggagatcttggcagttgagtggtaataatccagaatcagaaaaacaaataaattcaataatgtttatcaaaaacctatgttctctctgtgggagtcacagggatctgcagataaaggacagttcttccacttaataatattccagttgagtgaagaagggaaaatcaaagaaaagaggtagctgatgaagagataagaaacaggaaagatactactgagcttctcttgcacaccagccattacttacaaattttattttactgactccatacaacagccgcacaccaagaaatctaagccaatatcaaagcttcttaaatgtcacatacatcaaaatagacctgagacctaaaggtgacaagggcgctgggaaagttcgatctggggtggaaaacactgaattgagggttcaatgagaagtaacaaaggtaaaataccacagaaaccacctaggaacaagccaggtggacttccatgattgcaaagttcataaaattacatgttccgagaagtgccagagagagacaaaactactttaccacatgcttaatttcagggagaacttgaagagattcaaattctttaacttttgcaggatctacatcttcctctagctcccacgtgctttcttcatatggtagtgagcaccacttcaccaggtaatgtgttacctcctggtgggaaaacaggaaagaaacttaatgttctattaggttaaaggagtcagtagtatctgccaacctctgaaaaaccaccctacgtgactagacaggaataccactaagacaagggtcacatgttaaaacaagtgaaatacgtggaaagaagggaagatgcttcaggagaggcaaatacccacctcccctgtttctgcatccttggtgtgggccacctccaagatgcgatcaacttctacatagtctggattgaacaagtcttcatcaggctaacacaaacagaggagaacagttaggaagtatgtccccttgcctcaaatccaacacctagggtgttatctctaagtgatccctactctaggccctatcaacaaacacaaaaagaaagggtttgcttaagaataaaagtcacggcatttattcactgtctccaaatcctgctctccatgactaaatttaaaggatctaaccatcttagtggctacaccctagccaggtaataattccctgttggtcattatagcaatgtacaaaaatcagcagaagggttcccttttccttactggtccttatttcaaacacagcccttggcaactaggaattacagtttctttttcttaataacaacaatagttaacactggcagggggcatgagggaagcttcctgggtgctgacaatgttctatttgctgatctaggtagtagttacacaaatgtgtttacattgtaaaaattcattgagatgtatacttttatctttatgcacactatgcttcaaaaaaagtttactacaaaacaacaagaagcaacatatagaaatacttaaggtatgtccagagtaatgctaagtactttcatttaagctccaaaactaccttacggagtaggtactataattaccttacagattaggaaactgactcagagatattaagtaatgtgaccaagttgacggagctatgttaactggaccgttcacccaggtctttcttacccagaagctgaagccctcctccaggagtgctgaagagctcttactgccacttaggctgcaaggcacttttggggctaatttcatcgtctaatcaatgcaggatactttttacaacatccctggcaagcctctcagattcaattattcccaactaagaaatttactacccttcaagacctgattcactgtcgaaacagctttatctctacctctttaggagtagcatcatgatctttatcattagtaaattataacttaacttccttccttataactgttgctcattggcatgaactgcttcctttacagcagcaataagaggattctttcctctctctttaggctcttcagcaatcccctccctatctgggttaaaaaaaaaaaacccagtttgttcaacagttcttcatataccgtttccatcttaatgatacgaaaagctaatacttatggagtagtgcctatgagctaaacactgctataaacattgtgtcctttattattcacaatagtcaagattacggaaacaagctaagtgtccatcaagggatgaatggataaagaaagtggggtgtatatacctagacaatagaatatcattcagctttaaaaaagaaagaaatcccatcatttgcaacaacatggatgaacctggaggatgttatacaaagtaaaataagcctgacacacaaagacaaatattgtatgatctcacttatatgtggaatataaaataatcaaactcatagaagcagagagtaaatggtggttgccagggatgaaggggaggggaaatggggaagacattggtcaaagggtataaaatttcagttacgcaggatgaataagttctggagacctaatgcatagcatggtgatgacagttaaactactgtattatatatttgaaatttgctaagaggataaatcttaagtgttctcacccttaggggggaaaaaagaaagaaaaaaaaccccctacttatgtgaggtgatggatatgctcactagcttgactatagtggttatttcacaatgtaaacatacgttaaaacatcaagttgtacaccttaaatataattttttgacaattatactccaataaagcttaaaacggcaagaaaactatcatctaatccttgcaggtattctgtgaggctgattctatcaggagtcctgctttttcctatgatactgagtttgaaagagcttatgccatggcctgaggccacccaggtgtgtgtgaggctgggctgggaggggcagtttgactgcagagcccaggctcttaccattgtgcagtccgctctccacctatgccccattcccaccatgacagtcctcttgcagctgccctcaggttttgcaaagtccctcactgaatcctggtacccaaatgaacatagcatttaggaatgaaaagggcatggtacagcaggactgttctattggtgcagtctaggattagtaggattcgtttactttttaaacagccatgtcctactcttgacacgcagtaaatgtgtagttaattgaactggcaatatgttttctccctagaactgctacccagtcattttcctcatttcagaaccagaaagattccaagaacaatgtaggatattacatttattcctacatttattcatcctaccctgtccaggcaaacccaggctgatcctgacatttattctctcagtattggatctggttagaacgtctcctgggtgttcaggagttaggatttcatcaggagtctatctctgcgcatgtgggaagttgcaggctcttggagcctgagtttcctccacgtgtgctttagctacctcataaagcagctggaaataatggatacaaagctctattcaaaactaagttactatttgtatcacaaaaatatttacccaagttactaacaaatatgttgaataggtcaagaccaaaggcagtgttgaactactcgtcagtattattcctccttaatttcatgtgttccaatcttctcaagagcctttccttcccacaagaatatgaactcttgttgcattatttctatatatcccagcacagtttcctctattctcggccaaggaaaaaccactactttgggaatagatcagaaaacagacacaaagggctgtgtaggctctattacagggtttagtctttattccaagaataacacattaaggttaaggtttgtggcattttctgatatatagcattatagatttgtttaatattaaaatccatcagtccttcctcttgctataggtacaatacttagaaattcccaagtttacataaataaaattatttatttgtttactttctctcacttttagatttgttttgttttttacattcaaactttaggaccttctgaaatgtcctttttgggttgcagtgggttgagggagcagggacaaaagaattgtgtgcgtattcctctcactctagctatttattagtattcagaccaagatgcacgccgttatgagttgtcagtttataaggtttgatccttaactttggtggatagttttctttgacttgaatctagactagttttgttgcaaatcagcacacatttatcaatttgacttgagcaacagatctgtataggaatttaacttggacttagaaacgtaaacacagtatttactgctgcaagaaaagcagcagtcaaatgagtcagtaaatattgcttcacctccgtaaaaatgtgcttcatctgggcttgtttattcctaaatcgcttgatcttctgtgcgatgcgaggatccttttcgagctcttccattgtggcccatttacaatgtaagtaggaactatccaaagacatacaaattgtcaggcaagcacataactagcaaatgatcgagctttaaggtgttcaataaacacggtgaccacctaacacagaaatagtaaaatggtttattgtgaaactgctggagattgggcttagaaagaccttccccttaagggcaacaacagaaaacaaaacaaaacggagtaattccaagcaactgatctctctgcagcttctagagaaactggctgagaacagaggcagggcagctgtgggcaggttatcattaacacacattagtgaaagtgggtctgtagcagaaaggaaaatatacaagaggggaaaagagcatatgctacaggagagtggttctctaccctggttgcatatgagaattgcccaaggagtttttaaaaacacaacaatccttgggccccactgagtctgagccaatggtcttgggtggagcaaaggctttagaggtttaaaaaagctctccaggtaattctgtgtgcagctataattgagcgttactgcctaaggtaaacttgagtcagccaagacctgtgggtcatgccttagaggcaagactctgaagatgtagaagataataaagtcacaagagaaaaaaagttttattcatataaaacccaaatacagagaatactttaaatagacgtacaaatttctatacttaacgtagaacagctccaagtcgaacggaggttctcctgggtgaacctgtaacaaacagcaagcagttatcagcccagatcccaggcagatcacccttactgagccaaaagaaaacaggtaatcaatatctctgcctgatttccataaagatttcttaaggcatattttcattttcccaggccttttcctgacacctattggtcaggttttccaaaggtggaatacacagctgcaaggtaaaaaactgatcttcctgatgtccttccaaggtctaggctgggacggcactcagctgataaacccaaactggcccaggacaatgagaaatgtcaggaccagagcctggtgtggaagtgactacattcatctctggcaccccactaacgcttcacttctgaaacaccaatggtagtcactctccaagtaaggaagtttactttcataaattcttgaagtaaagatgaagccgtgctctcaccagcaccaacatactctagcactccaagaatttcctataggagattcctacaatggccctgagatgtctgaagaaaaaaacccccacgtttccattaggactcatatgggtgtgtatgtgtgtatgtatgtatgtatgtatgtatgtatgtatgtgtgtgtgtgtgtgtgtgtgtgtgtgtgtgtgtgtattttaaatagagaaagggtctcactatattgcccaggttggtcttgaactactgggctcaagtgaacctccgatgttggcctctcaaagtgatgggattataggtatgagccaatgaacctggcctctattaggactttttgtccatctcttcttcttggccctctgctaaattgtggtgggctacagcaactaagggatatatggcttttgctaggatcacaatggaggaaatcatcagcttgagccattctagatattttaagagttttatgtcctaaaatgctctacaggggtaaaaataagtgttggctagccagacaatattcttgcctagctctcactccagccaataatggtcattctaggttctctagccaaaaatctcttcacagaagatgtaatctgtgctaatgatataaaagctagaaacatgagccaccaacctcctggacagtcttagatgccaggatcttctcaatgatgtttgcatcatcttctggaggctcctggggacaaagataaaaaaaaaagaaaattgctggaaccacagaatccaagaaaacaattctgtaagaaatactacaatgggtactacttcattcaaaagttcacctttgagtaccaaggcctcccatcaggccacagagtggtcaaaaggtcacaagcattttgcaaagctagaatttaaagtggtggcttctaagctctggacacaactctacaagagcagagaagtttttcagatgcaatttcagatctgcatttaacatacctaaaattggtcctcagatttaccttctttgcaaaactacctacatgttgctgcatacaattttcagaaagctctaagagtatggggtcttaaaattagttctaatatctaccagctattgactttaggcaagtcaattaatacctcctagtctccaaatatattaaaataggagttaaaaatacatatctcactgaattagtgggaagataaaatagtattactattggaaaaatgttttcacatactattcaaaatcaaggtataattattctctattaaattgtaccatctttcccctttgtagctggaaaaggctgtttcactgatcatcctttgaacgtaaatcccaactgaaaacctaaattcctttttaccctagataaccatcaacacttctatcctccaatctccatgaaatctctttctacagagtatttatagttaaaaacacaaaatgtttaacattctttctagaacctcaagcagcattatttctcagtggttcatatgcacaatgaccttttcagatc

SEQ ID NO: 10 - DNA, 5121 bp, Homo sapiens
atttccatcaaccatgatgaaagtctgctgcctgagtccttagagagcatgatgtatggtaagaaggtgctcagccaagaaccaagctcttttcaggagagcccaagtaccaatactgaatctagaaaagatgttattaccatctcaataagcaaagatgggaactgccagtctggtggccctgaggcagaaatagcttctggccctacttttatgggtagcttagaagcaggaggagtagctcaagcaaacatcaaaaatggaaaacatttgttgatgtctatttcaaaggaaggggagctctgctgcagtgaggcaggacagagacctgaaaacattggccagctggaagccaagtgtttagcttccccttccttgaatccaggaaatgaaagtgggtttgtagatatgtgcagtcttagtgtctgtgactccaaaagaaacctgtcatcagatcagcaattaattgatttattggaaaacaaaagcttagaaagtaaattgattttgagtcagaaccacagtgatgaggaggaagaagaggaggaaaacgaggaggaaaacttagccatggcagtaggcatgggggaaaggccagaggtattgcatctcacggagcccactactaacatctcaagggaaaagaaccaaggcttccaagatgaaaccaagaaaggaagcttagaggtggcaaaccagactcctgggctacagagggctttccccgctccagcagcctgtcagtgccactgcaaacacatggagaggtggatgcatggcctcgagaatgatgaatttgaaatcgagaaacccaaggcttatatcccagatctgttcaaaagtaaaaccaatactatcgccatggagggtgaacccactgctattccatcacagccgtttaaagtgaagcatgagcttttaaaagaaccttggaaagaaagtgcagaggggcaaaacgttttccccacatatcctcttgaaggaagtgagctcaaatcagaagacatggattttgagaataaagatgattatgatagagacggaaactgccatagtcaagattatccagggaagtactctgaagaggagagcaagagctcaacatcgggcatcacaggagacattggggatgagctacaggaggctcgagctcccactattgctcagctgctacaggagaaaactctctattccttctctgagtggccaaaggaccgcgtgataattaaccgcctagataatatctgccacgtggtgttaaaggggaagtggccctctagccagcagtatgagccctcaggcacactgcccaccccggtattaaccagcagtgctggttctcgaaccagcctctcagagccggaagcagcagaacacagcttcagcaacggcgcagcattggcggcccagatccacaaggagagcttcttagctccagtattcacaaaggatgaacaaaagcacaggcgtccctatgagtttgaggtggagagggatgcaaaggctcggggcctggagcagttctctgccacccacgggcacacccctatcatcctcaatggctggcatggggagtcagctatggacctctcctgctcatcagaggggtccccaggagccacatcccctttcccagtgagcgccagcacccctaagattggggctatcagttcacttcagggagcccttggcatggacttgtctgggattctgcaagctggcctgatccatcctgtgactggacagattgtcaatggaagcctcagaagagatgatgcagccacgaggaggcggagagggaggcggaaacatgttgaaggagggatggacctcatctttttgaaggagcagacacttcaggcgggaatcttggaagtccatgaagacccagggcaggccaccttgagcaccacacaccctgaggggccagggcctgccacctcggctcctgagccagctacggcagccagcagccaagccgagaaatccattcccagcaagagtctgcttgactggctaaggcagcaggctgactactccttagaagttcctggctttggggcaaatttttcagacaaaccaaagcagaggaggccacgctgtaaagaacctggaaaattagatgtcagctccctgacggggaagagagagttcctgccatccccaaggagccaggactgagggggtttcttccagaaaacaagttcaatcacactctggctgagcctattcttcgagatacgggcccccgcaggagggggaggcggcctcggagcgaactcctgaaggctccttccattgtggcagactctccctctggaatggggccactgttcatgaatggactgattgctgggatggacctggtaggacttcagaacatgagaaatatgccaggcatccccctcaccgggctggtggggtttccagctggctttgccacgatgccaacaggtgaagaggtcaaaagtaccctgagcatgctgcccatgatgctgccaggcatggctgctgtgccccagatgtttggtgttgggggactcctcagtccacccatggcaaccacctgcacttccactgctccggcgtctctatcaagcacaacgaaaagtggtacggcagtgactgaaaagactgcggaagacaagccgagtagccatgatgtgaaaacagacactttagctgaggacaagcctggtccaggtccattttctgatcagtctgaacctgcaataactactagtagtcctgtggcttttaacccatttctcatcccaggagtatctcctggactcatttacccatccatgttcctctcccctggtatgggcatggctctgccagccatgcagcaggccagacactcggaaatagtaggtctggagagccagaagaggaagaagaagaagacaaagggggacaaccccaactcccacccagagcctgctcccagctgtgaaagggagcccagcggtgatgagaactgtgccgaacccagtgcccctttgcccgcagagagagaacatggggcacaggctggggagggggcactcaaagactccaacaacgacaccaattagaacttttttcatttaagaaattattgtgacttgtaagtttcttatcccataaaggtttgttacttccctcacttcacctccataagaacctgtgtttccataagtaagattacgtacctgatttcctgtctgagaactatggtaacagatgttaatagttgcagggtctcaccacttcattagataagtgttgtctacctagtctaggaggcacagaattctcattctgttatccagttcattccagcaatcatagttaatacagtacttggtgacacgccctacccccttctcttccaagtttcccactcacttgaggaggaaaaatggcaaaagaaagctgtctagggttttaccattgaagggtggaagaacagagacaaagaggagctctttttctgtgagctgggttgcacaggaagaatgtcacagggaaccaaaaagcacagaaaaaggaagtgctggtgcatatttttgagttaaaatatttccctattttatcatgattactaagtgagtagtatagacagaagtatataactaatggttgaaaatacatatattcatttctttataaaaaacaaaaaccttaccggtagtaatataatttcccccttggtggtttttcagacacctgcagcaagaagaaatactgactgactaggcattattttctatacatccctctcaccagtgaaaagattcctcttgctgcgagaaagctttacccaccatgagttattgctgtcgacgggggtggggcaaggacccgcgctccgtagagctacacgctgcttcacaagcacacggctagcgctctgctctcacctggttcgcttacagatttctctagccattaatttgcctctctgtgtttaaagagcaccaggaccgaatggattttcacttcaggctttccttcaccaagaataaggttctttctggagcctgcaagaagacagttgcccaacactttgacacttgctagtaggtcctttgatcaagagtgtctgaggctgtcaaatgtgtgccaccctttataatacagctatgaaaagttacttctccattaatttctccattctgtttatatcttacagttggacccaaattccaaattatcagttaatcgaaactacattgcaagtcaacattattttctcttcttcctcagcttctccatgtttaggaactgagaaagaaactgcctaagcagcacttccccatgtcctttcaccttcatgttaacgtcccttctgatggtctcactcttcacaggtagttcacactctgagacttgaagtaagctcatggttcagtactctgggggcgcagggctgcgcaggacccttggaaatgttgcaagtgttactagtaccagtttcactttttgttgtcacaatttactgtattttttactttttctgttacagttttgctaatttatcagaaggtccaaaagtctgacataactatttcattttgcattatttatttatgatgcttttttcattgtcttttatacatttgggattataaattatgtaaatgttaaaatgagcatctcaaagaagtctgttaaatcatgactgaaaaaaatcaatcagatgtatcttcaaaaagtggagtcccagttttatgaatcagagatataaaccagaaattctataactgatcatagaagaagaatccagtaattgaacaaatcctatttaatgacatccttgtagcatagatggtctataatgctgaccacagatttcttagaaatgctgctctctctatttaactaacattttgttcagttttgcctccagtggaagcagaaagggttttttcagctgttaaatcctaaaaatcaatataatttatttatgtaagaaaaataactcaatcaatatatttttgaaccttttaagtactaattttctttttatcaagtagaaaaaaaaatgtatttgccctaaatccttaaaatacaaatgctataaaaattcctgtatcttgaaagccttactgcaaatgagtattatagacatcc

SEQ ID NO: 11 - DNA, 5123 bp, Homo sapiens
atttccatcaaccatgatgaaagtctgctgcctgagtccttagagagcatgatgtatggtaagaaggtgctcagccaagaaccaagctcttttcaggagagcccaagtaccaatactgaatctagaaaagatgttattaccatctcaataagcaaagatgggaactgccagtctggtggccctgaggcagaaatagcttctggccctacttttatgggtagcttagaagcaggaggagtagctcaagcaaacatcaaaaatggaaaacatttgttgatgtctatttcaaaggaaggggagctctgctgcagtgaggcaggacagagacctgaaaacattggccagctggaagccaagtgtttagcttccccttccttgaatccaggaaatgaaagtgggtttgtagatatgtgcagtcttagtgtctgtgactccaaaagaaacctgtcatcagatcagcaattaattgatttattggaaaacaaaagcttagaaagtaaattgattttgagtcagaaccacagtgatgaggaggaagaagaggaggaaaacgaggaggaaaacttagccatggcagtaggcatgggggaaaggccagaggtattgcatctcacggagcccactactaacatctcaagggaaaagaaccaaggcttccaagatgaaaccaagaaaggaagcttagaggtggcaaaccagactcctgggctacagagggctttccccgctccagcagcctgtcagtgccactgcaaacacatggagaggtggatgcatggcctcgagaatgatgaatttgaaatcgagaaacccaaggcttatatcccagatctgttcaaaagtaaaaccaatactatcgccatggagggtgaacccactgctattccatcacagccgtttaaagtgaagcatgagcttttaaaagaaccttggaaagaaagtgcagaggggcaaaacgttttccccacatatcctcttgaaggaagtgagctcaaatcagaagacatggattttgagaataaagatgattatgatagagacggaaactgccatagtcaagattatccagggaagtactctgaagaggagagcaagagctcaacatcgggcatcacaggagacattggggatgagctacaggaggctcgagctcccactattgctcagctgctacaggagaaaactctctattccttctctgagtggccaaaggaccgcgtgataattaaccgcctagataatatctgccacgtggtgttaaaggggaagtggccctctagccagcagtatgagccctcaggcacactgcccaccccggtattaaccagcagtgctggttctcgaaccagcctctcagagccggaagcagcagaacacagcttcagcaacggcgcagcattggcggcccagatccacaaggagagcttcttagctccagtattcacaaaggatgaacaaaagcacaggcgtccctatgagtttgaggtggagagggatgcaaaggctcggggcctggagcagttctctgccacccacgggcacacccctatcatcctcaatggctggcatggggagtcagctatggacctctcctgctcatcagaggggtccccaggagccacatcccctttcccagtgagcgccagcacccctaagattggggctatcagttcacttcagggagcccttggcatggacttgtctgggattctgcaagctggcctgatccatcctgtgactggacagattgtcaatggaagcctcagaagagatgatgcagccacgaggaggcggagagggaggcggaaacatgttgaaggagggatggacctcatctttttgaaggagcagacacttcaggcgggaatcttggaagtccatgaagacccagggcaggccaccttgagcaccacacaccctgaggggccagggcctgccacctcggctcctgagccagctacggcagccagcagccaagccgagaaatccattcccagcaagagtctgcttgactggctaaggcagcaggctgactactccttagaagttcctggctttggggcaaatttttcagacaaaccaaagcagaggaggccacgctgtaaagaacctggaaaattagatgtcagctccctgagcggggaagagagagttcctgccatccccaaggagccaggactgagggggtttcttccagaaaacaagttcaatcacactctggctgagcctattcttcgagatacgggcccccgcaggagggggaggcggcctcggagcgaactcctgaaggctccttccattgtggcagactctccctctggaatggggccactgttcatgaatggactgattgctgggatggacctggtaggacttcagaacatgagaaatatgccaggcatccccctcaccgggctggtggggtttccagctggctttgccacgatgccaacaggtgaagaggtcaaaagtaccctgagcatgctgcccatgatgctgccaggcatggctgctgtgccccagatgtttggtgttgggggactcctcagtccacccatggcaaccacctgcacttccactgctccggcgtctctatcaagcacaacgaaaagtggtacggcagtgactgaaaagactgcggaagacaagccgagtagccatgatgtgaaaacagacactttagctgaggacaagcctggtccaggtccattttctgatcagtctgaacctgcaataactactagtagtcctgtggcttttaacccatttctcatcccaggagtatctcctggactcatttacccatccatgttcctctcccctggtatgggcatggctctgccagccatgcagcaggccagacactcggaaatagtaggtctggagagccagaagaggaagaagaagaagacaaagggggacaaccccaactcccacccagagcctgctcccagctgtgaaagggagcccagcggtgatgagaactgtgccgaacccagtgcccctttgcccgcagagagagaacatggggcacaggctggggagggggcactcaaagactccaacaacgacaccaattagaacttttttcatttaagaaattattgtgacttgtaagtttcttatcccataaaggtttgttacttccctcacttcacctccataagaacctgtgtttccataagtaagattacgtacctgatttcctgtctgagaactatggtaacagatgttaatagttgcagggtctcaccacttcattagataagtgttgtctacctagtctaggaggcacagaattctcattctgttatccagttcattccagcaatcatagttaatacagtacttggtgacacgccctacccccttctcttccaagtttcccactcacttgaggaggaaaaatggcaaaagaaagctgtctagggttttaccattgaagggtggaagaacagagacaaagaggagctctttttctgtgagctgggttgcacaggaagaatgtcacagggaaccaaaaagcacagaaaaaggaagtgctggtgcatatttttgagttaaaatatttccctattttatcatgattactaagtgagtagtatagacagaagtatataactaatggttgaaaatacatatattcatttctttataaaaaacaaaaaccttaccggtagtaatataatttcccccttggtggtttttcagacacctgcagcaagaagaaatactgactgactaggcattattttctatacatccctctcaccagtgaaaagattcctcttgctgcgagaaagctttacccaccatgagttattgctgtcgacgggggtggggcaaggacccgcgctccgtagagctacacgctgcttcacaagcacacggccagcgctctgctctcacctggttcgcttacagatttctctagccattaatttgcctctctgtgtttaaagagcaccaggaccgaatggattttcacttcaggctttccttcaccaagaataaggttctttctggagcctgcaagaagacagttgcccaacactttgacacttgctagtaggtcctttgatcaagagtgtctgaggctgtcaaatgtgtgccaccctttataatacagctatgaaaagttacttctccattaatttctccattctgtttatatcttacagttggacccaaattccaaattatcagttaatcgaaactacattgcaagtcaacattattttctcttcttcctcagcttctccatgtttaggaactgagaaagaacctgcctaagcagcacttccccatgtcctttcaccttcatgttaacgtcccttctgatggtctcactcttcacaggtagttcacactctgagacttgaagtaagctcatggttcagtactctgggggcgcagggctgcgcaggacccttggaaatgttgcaagtgttactagtaccagtttcactttttgttgtcacaatttactgtattttttactttttctgttacagttttgctaatttatcagaaggtccaaaagtctgacataactatttcattttgcattatttatttatgatgcttttttcattgtcttttatacatttgggattataaattatgtaaatgttaaaatgagcatctcaaagaagtctgttaaatcatgactgaaaaaaatcaatcagatgtatcttcaaaaagtggagtcccagttttatgaatcagagatataaaccagaaattctataactgatcatagaagaagaatccagtaattgaacaaatcctatttaatgacatccttgtagcatagatggtctataatgctgaccacagatttcttagaaatgctgctctctctatttaactaacattttgttcagttttgcctccagtggaagcagaaagggttttttcagctgttaaatcctaaaaatcaatataatttatttatgtaagaaaaataactcaatcaatatatttttgaaccttttaagtactaattttctttttatcacgtagaaaaaaaaatgtatttgccctaaatccttaaaatacaaatgctataaaaattcctgtatcttgaaagccttactgcaaatgagtattatagacatccc

SEQ ID NO: 12 - DNA, 5901 bp, Homo sapiens
ctggagttcaagatttctgatgaggaggcagatgatgcagatgctgctgggagggattccccctccaacacctcccagtcagaacagcaggaatctgttgatgcagaaggcccagtggtagaaaaaattatgagcagtcgttcagtaaaaaagcagaaggaatctggagaggaggtagaaattgaggaattctatgtgaaatacaaaaacttctcttatcttcattgtcagtgggcatctatagaagatctggaaaaagataagagaattcagcaaaaaattaaacgatttaaggcaaagcagggccagaacaagttcctttcagagattgaggatgagctttttaatccagattatgtggaggttgaccggataatggactttgcacgtagcacagatgaccggggagagcctgtgactcactatctggtgaagtggtgttcacttccttatgaagacagcacgtgggagcggaggcaggacatagatcaagcaaagatcgaggagtttgagaaactaatgtccagggagccggaaacagagcgtgtggagcgacctcctgctgatgattggaagaaatcggagagttccagggagtataaaaacaataacaaactcagggaataccagttggagggagtaaactggctacttttcaattggtacaacatgcgaaactgcattttagcagatgaaatgggtttgggaaaaactatccagtccattacatttctctatgagatatatttgaaaggaatccatggcccttttttagtaattgccccattgtccacaatccccaactgggaaagggaattccgaacctggacagagttgaacgtggttgtgtatcatgggagtcaagctagtcgtcggaccattcagttgtatgaaatgtacttcaaagatccccagggtcgagtgataaaggggtcctataagtttcatgccatcatcactacatttgagatgattttgactgattgtcctgagctgcggaatattccatggcgctgtgtagtcattgatgaagcccacaggctgaagaacaggaactgcaagctgttggagggactcaagatgatggacttggaacacaaagtgctgctgacgggaaccccactccagaacactgtggaagaactcttcagcttgcttcatttcttggaaccaagtcgcttcccttcagaaaccacatttatgcaagaatttggtgatctaaaaacagaagagcaggtgcaaaaacttcaagctattctaaagccaatgatgttgagacgtctcaaagaggatgtagaaaagaacttggcccccaaagaagaaactattattgaagttgagctaacaaacattcagaagaaatattaccgagccatccttgagaagaatttcacatttctttccaaaggcggtggtcaagctaacgtacctaacctattaaacactatgatggaattgcggaagtgctgcaatcatccgtaccttatcaatggtgctgaagagaaaattttggaagagtttaaagaaacacacaatgcagagtctccagattttcagctccaggcaatgatccaggctgctggcaagctagtgctgattgacaagctgctgccaaaactgaaggctggtggccacagggtgcttatcttttcccagatggtgcgctgcttggacatactggaagactacctcattcaaagacggtacccatatgaaaggatcgacggccgagtaagaggcaacctccgccaggcagctatcgacagattctccaaacctgattctgataggtttgttttcctcctgtgtacaagggcaggaggtttaggcattaacctcactgctgctgatacctgcatcatctttgattcagactggaatccccaaaatgacctccaggctcaggctagatgtcatagaataggacagagcaaatctgtgaaaatctacaggctgattacaagaaattcctatgaaagggaaatgttcgacaaggctagtttgaaactgggcctggataaagctgtgctacagtctatgagtggaagagaaaatgctaccaatggggtacaacagctttccaagaaagaaatagaggatcttctacgaaaaggggcctatggtgcactcatggatgaggaggatgaagggtctaaattctgtgaagaagatattgatcagatcctcctacgtcgaacccacaccattaccattgagtcagaagggaaaggttccacatttgctaaggccagttttgttgcatctggaaataggacagatatttccttggatgatccaaatttctggcaaaagtgggctaagaaggctgaattggatattgatgccttaaatgggaggaacaacctggttattgatactccaagagtgagaaagcagaccaggctctacagtgcagtgaaggaagatgagctgatggagttctcagacttggaaagtgattctgaagaaaagccctgtgcaaagccacggcgtccccaggataagtcacagggctatgcaaggagtgaatgtttcagggtggagaagaatctgcttgtctatggttggggacggtggacagacattctttcccacggacgctataaacgccaactcactgagcaagatgtagaaaccatctgcagaaccatcctggtgtactgtcttaatcattacaaaggggatgagaatatcaaaagcttcatctgggatctgatcacacccacagcggatggccagactcgagccttggtcaaccattccggtttgtcagctcctgtgccaaggggaaggaagggaaagaaggtgaaagcccagagcacacagccggtggtgcaggatgccgactggctggccagctgcaacccagatgccctgttccaggaggacagctacaagaaacacctgaagcatcactgtaacaaggtcctgctgcgtgtccgcatgctgtactacctaagacaagaagtgataggagaccaggcggataagatcttagagggtgctgactcaagtgaagccgatgtgtggatccctgaacctttccatgctgaagttcctgcagattggtgggataaggaagcagacaaatccctcttaattggagtgttcaaacatggctatgagaagtacaactccatgcgagctgaccccgcgctgtgctttctggaacgagtcggtatgcctgatgccaaggccatagctgccgagcaaagaggaacagacatgctagcagatggtggtgacgggggagaatttgatagagaagatgaagacccagaatataaaccaaccagaacaccgttcaaagatgaaatagatgaatttgcaaattctccttcagaggataaggaagaatccatggaaatacatgccacaggcaagcacagtgagagtaatgctgagttaggccaactttactggcctaacacttcaaccctgactacacgtctgcgccggctcattactgcctatcagcgcagctataaaaggcaacagatgaggcaagaggccctaatgaagactgaccggcgcagacggcggcctcgagaggaagtgagagctctggaagcggaaagggaagctattatatctgagaagcggcaaaagtggacaagaagagaagaggctgatttttaccgtgtggtatccacctttggggttatttttgaccctgtgaaacagcaatttgactggaaccaatttagagcctttgccaggcttgacaaaaaatctgatgagagtttggagaaatacttcagttgttttgtggccatgtgtaggcgagtatgtcgaatgcccgtcaagccagatgatgaaccgcccgacctctcctccataattgagccgatcacagaggagcgagcctctcgaactctgtaccgcattgagctgctacggaagatccgcgagcaggttctccatcacccccagctgggagagaggcttaagctctgccagccaagcttggatctgccagagtggtgggagtgtggacggcatgaccgagacttgctggttggtgctgctaaacacggggtcagtcggacggattatcacatcctcaatgaccctgagttatccttcttggatgcacataaaaactttgctcaaaacagaggggcaggtaatacatcttccttgaacccactggcagttggatttgtccagactcctccagtcatctcatctgctcatattcaagatgagagggtactggaacaagccgaaggcaaagtggaggagcctgaaaacccagctgccaaggagaaatgtgagggcaaagaagaggaagaagaaaccgatggcagcgggaaggagagcaagcaggaatgtgaggcagaggccagctctgtgaaaaatgaactgaaaggtgttgaggtcggcgcagacactgggtccaaatctatttcagagaaaggttccgaagaggatgaagaggaaaagctggaggatgacgataagtcggaagagtcttcccagcccgaagcaggagctgtctctagagggaagaattttgatgaagaaagcaatgcttccatgagcactgctagagatgaaacccgagatggattctacatggaggacggagatccttcagtagctcagctccttcatgaaagaacatttgccttctcgttttggcctaaggatagagtaatgataaaccgcttagacaacatctgtgaagcagtgttgaaaggcaaatggccagtaaataggcgccagatgtttgatttccaaggcctcatcccaggttacacacccaccacagtggacagccccttgcagaagaggagctttgctgagctctccatggtcggccaagccagcattagtgggagtgaggacatcactacgtctcctcagttgtcaaaggaagatgccctcaacctctctgtccctcgccagcggaggaggaggaggagaaaaatcgaaattgaggccgaaagagctgccaagaggcgaaatctcatggagatggttgcccagcttcgagagtctcaggtggtctcagaaaatggacaagaaaaagttgtagatttatcaaaggcctcaagagaggcaacaagctctacctcaaatttttcatctctttcttcaaagtttatcttgcctaatgtctcaacaccagtgtctgatgcctttaagactcaaatggaactgctccaagcaggcctttcgcgcacacccacaaggcatctccttaatggctccctagtggatggagagcctcccatgaagaggaggcggggaaggaggaaaaatgtggagggacttgatctgcttttcatgagccacaaacggacgtcattgagtgcagaggatgctgaggtgaccaaagcttttgaagaagatatagagaccccaccaacaagaaacattccttctcccggacagctggacccagacacacggatccctgttatcaatcttgaagatgggactaggctggtgggggaagatgctcctaaaaataaggatttagttgaatggctgaagctgcaccctacttacactgttgatatgccaagttatgtaccaaagaatgcagatgtgctgttttcctcatttcagaaaccgaaacagaaacgacatagatgtcgaaaccctaataaattggatataaacactttgacaggagaagaaagggtgcctgttgtcaataaacgaaatgggaagaagatgggtggagctatggcgcctccaatgaaggatctacccaggtggctggaagaaaatcctgaatttgcagttgctccagactggactgatatagttaagcagtctggttttgttcctgagtcgatgtttgaccgccttctcactgggcctgtagtgcggggagagggagcgagcagaagaggaagaaggcccaaaagtgagatcgccagagcagcc

SEQ ID NO: 13 - DNA, 9110 bp, Homo sapiens
ctcgctttgtcctgttctttgtcttgttgtcttttttgttttcttttcgtgttcttcttcctctctcggttgtcccttctttgcttgtccttgtgatatttcgtttttctcttctttcttgcgcatgttcttcggtgatcctgttgtgccttctttaatctcctgattcgtcgtcctgatcccactcgtggatcccatcaactggtcaatgactatgcaccagcgcccttcccccccgggcttcatttaccatctgacagccatcttgcatcccttcacaagccttcagtgaacttatgccttctgcaccttccctatgtcacccatgaaagcaactgagttatctcatgcaaggcactcctcctccactaagtcaggccgggaagcgtctgggataccaatggaaggttggccagttattccctatatgccccattcctcagccatctcaccagccccctggtgccactgggaatcggacagaggaatatgggccccagaaacatgcagcagtctcgtccatttataggcatgtcctcggcaccaaagggaattgactgggcacatgaggccaaatggttgtcctggtgttggccttggagacccacaagcaatccaggaacgactgatacctggccaacaacatcctggtcaacagccatcttttcagcagttgccaacctgtcctccactgcagcctcacccgggcttgcaccaccagtcttcacctccacaccctcatcaccagccttgggcacagctccacccatcaccccagaacaccccgcagaaagtgcctgtgcatcagcattccccgtcggagccctttctagagaaaccagtgccggatatgactcaggttagtggaccgaatgctcagctagtgaagagtgatgattacctgccatcaatagaacagcagccacaacaaaagaagaagaaaaagaaaaacaaccacattgtagcagaggatcccagtaaaggttttggtaaagatgacttccctggtggggtagataaccaagaactaaataggaactcactggatgggtcccaagaagaaaaaaagaaaaagaaaaggtcaaaggcaaaaaaagacccgaaggaaccgaaagaacccaaggagaaaaaaaagcctaaggaacccaagaccccgaaagcccctaagattcccaaagagccaaaggaaaagaaagcaaaaactgccacgccaaaacccaaatccagcaaaaagtcaagtaataagaaacctgactcagaagcaagtgctttgaagaaaaaggtcaacaagggaaaaacagaaggttctgaaaattcagacttagacaaaacacccccaccatctcctcctcctgaagaagatgaggacccaggtgttcagaagagacggtccagcagacaggtgaagagaaagcgctacactgcccttctggagttcaagatttctgatgaggaggcagatgatgcagatgctgctgggagggattccccctccaacacctcccagtcagaacagcaggaatctgttgatgcagaaggcccagtggtagaaaaaattatgagcagtcgttcagtaaaaaagcagaaggaatctggagaggaggtagaaattgaggaattctatgtgaaatacaaaaacttctcttatcttcattgtcagtgggcatctatagaagatctggaaaaagataagagaattcagcaaaaaattaaacgatttaaggcaaagcagggccagaacaagttcctttcagagattatgtggaggttgaccggataatggactttgcacgtagcacagatgaccggggagagcctgtgactcactatctggtgaagtggtgttcacttccttatgaagacagcacgtgggagcggaggcaggacatagatcaagcaaagatcgaggagtttgagaaactaatgtccagggagccggaaacagagcgtgtggagcgacctcctgctgatgattggaagaaatcggagagttccagggagtataaaaacaataacaaactcagggaataccagttggagggagtaaactggctacttttcaattggtacaacatgcgaaactgcattttagcagatgaaatgggtttgggaaaaactatccagtccattacatttctctatgagatatatttgaaaggaatccatggcccttttttagtaattgccccattgtccacaatccccaactgggaaagggaattccgaacctggacagagttgaacgtggttgtgtatcatgggagtcaagctagtcgtcggaccattcagttgtatgaaatgtacttcaaagatccccagggtcgagtgataaaggggtcctataagtttcatgccatcatcactacatttgagatgattttgactgattgtcctgagctgcggaatattccatggcgctgtgtagtcattgatgaagcccacaggctgaagaacaggaactgcaagctgttggagggactcaagatgatggacttggaacacaaagtgctgctgacgggaaccccactccagaacactgtggaagaactcttcagcttgcttcatttcttggaaccaagtcgcttcccttcagaaaccacatttatgcaagaatttggtgatctaaaaacagaagagcaggtgcaaaaacttcaagctattctaaagccaatgatgttgagacgtctcaaagaggatgtagaaaagaacttggcccccaaagaagaaactattattgaagttgagctaacaaacattcagaagaaatattaccgagccatccttgagaagaatttcacatttctttccaaaggcggtggtcaagctaacgtacctaacctattaaacactatgatggaattgcggaagtgctgcaatcatccgtaccttatcaatggtgctgaagagaaaattttggaagagtttaaagaaacacacaatgcagagtctccagattttcagctccaggcaatgatccaggctgctggcaagctagtgctgattgacaagctgctgccaaaactgaaggctggtggccacagggtgcttatcttttcccagatggtgcgctgcttggacatactggaagactacctcattcaaagacggtacccatatgaaaggatcgacggccgagtaagaggcaacctccgccaggcagctatcgacagattctccaaacctgattctgataggtttgttttcctcctgtgtacaagggcaggaggtttaggcattaacctcactgctgctgatacctgcatcatctttgattcagactggaatccccaaaatgacctccaggctcaggctagatgtcatagaataggacagagcaaatctgtgaaaatctacaggctgattacaagaaattcctatgaaagggaaatgttcgacaaggctagtttgaaactgggcctggataaagctgtgctacagtctatgagtggaagagaaaatgctaccaatggggtacaacagctttccaagaaagaaatagaggatcttctacgaaaaggggcctatggtgcactcatggatgaggaggatgaagggtctaaattctgtgaagaagatattgatcagatcctcctacgtcgaacccacaccattaccattgagtcagaagggaaaggttccacatttgctaaggccagttttgttgcatctggaaataggacagatatttccttggatgatccaaatttctggcaaaagtgggctaagaaggctgaattggatattgatgccttaaatgggaggaacaacctggttattgatactccaagagtgagaaagcagaccaggctctacagtgcagtgaaggaagatgagctgatggagttctcagacttggaaagtgattctgaagaaaagccctgtgcaaagccacggcgtccccaggataagtcacagggctatgcaaggagtgaatgtttcagggtggagaagaatctgcttgtctatggttggggacggtggacagacattctttcccacggacgctataaacgccaactcactgagcaagatgtagaaaccatctgcagaaccatcctggtgtactgtcttaatcattacaaaggggatgagaatatcaaaagcttcatctgggatctgatcacacccacagcggatggccagactcgagccttggtcaaccattccggtttgtcagctcctgtgccaaggggaaggaagggaaagaaggtgaaagcccagagcacacagccggtggtgcaggatgccgactggctggccagctgcaacccagatgccctgttccaggaggacagctacaagaaacacctgaagcatcactgtaacaaggtcctgctgcgtgtccgcatgctgtactacctaagacaagaagtgataggagaccaggcggataagatcttagagggtgctgactcaagtgaagccgatgtgtggatccctgaacctttccatgctgaagttcctgcagattggtgggataaggaagcagacaaatccctcttaattggagtgttcaaacatggctatgagaagtacaactccatgcgagctgaccccgcgctgtgctttctggaacgagtcggtatgcctgatgccaaggccatagctgccgagcaaagaggaacagacatgctagcagatggtggtgacgggggagaatttgatagagaagatgaagacccagaatataaaccaaccagaacaccgttcaaagatgaaatagatgaatttgcaaattctccttcagaggataaggaagaatccatggaaatacatgccacaggcaagcacagtgagagtaatgctgagttaggccaactttactggcctaacacttcaaccctgactacacgtctgcgccggctcattactgcctatcagcgcagctataaaaggcaacagatgaggcaagaggccctaatgaagactgaccggcgcagacggcggcctcgagaggaagtgagagctctggaagcggaaagggaagctattatatctgagaagcggcaaaagtggacaagaagagaagaggctgatttttaccgtgtggtatccacctttggggttatttttgaccctgtgaaacagcaatttgactggaaccaatttagagcctttgccaggcttgacaaaaaatctgatgagagtttggagaaatacttcagttgttttgtggccatgtgtaggcgagtatgtcgaatgcccgtcaagccagatgatgaaccgcccgacctctcctccataattgagccgatcacagaggagcgagcctctcgaactctgtaccgcattgagctgctacggaagatccgcgagcaggttctccatcacccccagctgggagagaggcttaagctctgccagccaagcttggatctgccagagtggtgggagtgtggacggcatgaccgagacttgctggttggtgctgctaaacacggggtcagtcggacggattatcacatcctcaatgaccctgagttatccttcttggatgcacataaaaactttgctcaaaacagaggggcaggtaatacatcttccttgaacccactggcagttggatttgtccagactcctccagtcatctcatctgctcatattcaagatgagagggtactggaacaagccgaaggcaaagtggaggagcctgaaaacccagctgccaaggagaaatgtgagggcaaagaagaggaagaagaaaccgatggcagcgggaaggagagcaagcaggaatgtgaggcagaggccagctctgtgaaaaatgaactgaaaggtgttgaggtcggcgcagacactgggtccaaatctatttcagagaaaggttccgaagaggatgaagaggaaaagctggaggatgacgataagtcggaagagtcttcccagcccgaagcaggagctgtctctagagggaagaattttgatgaagaaagcaatgcttccatgagcactgctagagatgaaacccgagatggattctacatggaggacggagatccttcagtagctcagctccttcatgaaagaacatttgccttctcgttttggcctaaggatagagtaatgataaaccgcttagacaacatctgtgaagcagtgttgaaaggcaaatggccagtaaataggcgccagatgtttgatttccaaggcctcatcccaggttacacacccaccacagtggacagccccttgcagaagaggagctttgctgagctctccatggtcggccaagccagcattagtgggagtgaggacatcactacgtctcctcagttgtcaaaggaagatgccctcaacctctctgtccctcgccagcggaggaggaggaggagaaaaatcgaaattgaggccgaaagagctgccaagaggcgaaatctcatggagatggttgcccagcttcgagagtctcaggtggtctcagaaaatggacaagaaaaagttgtagatttatcaaaggcctcaagagaggcaacaagctctacctcaaatttttcatctctttcttcaaagtttatcttgcctaatgtctcaacaccagtgtctgatgcctttaagactcaaatggaactgctccaagcaggcctttcgcgcacacccacaaggcatctccttaatggctccctagtggatggagagcctcccatgaagaggaggcggggaaggaggaaaaatgtggagggacttgatctgcttttcatgagccacaaacggacgtcattgagtgcagaggatgctgaggtgaccaaagcttttgaagaagatatagagaccccaccaacaagaaacattccttctcccggacagctggacccagacacacggatccctgttatcaatcttgaagatgggactaggctggtgggggaagatgctcctaaaaataaggatttagttgaatggctgaagctgcaccctacttacactgttgatatgccaagttatgtaccaaagaatgcagatgtgctgttttcctcatttcagaaaccgaaacagaaacgacatagatgtcgaaaccctaataaattggatataaacactttgacaggagaagaaagggtgcctgttgtcaataaacgaaatgggaagaagatgggtggagctatggcgcctccaatgaaggatctacccaggtggctggaagaaaatcctgaatttgcagttgctccagactggactgatatagttaagcagtctggttttgttcctgagtcgatgtttgaccgccttctcactgggcctgtagtgcggggagagggagcgagcagaagaggaagaaggcccaaaagtgagatcgccagagcagccgcggccgccgctgctgtggcctccacgtcagggatcaaccctttgctggtgaacagcctgtttgctggaatggacctgacgagccttcagaatctccagaatctccagtcgctccagctggcaggcctcatgggcttccctccaggactggcaacagctgccaccgccggaggcgatgcgaagaaccctgctgctgtgctgcccctgatgctgccaggaatggcgggcctgcccaacgtgtttggcttgggcgggctgttgaataaccctctgtcagctgctactggaaacaccactactgcttctagtcaaggagaaccggaagacagcacttcaaaaggagaggagaaaggaaatgagaatgaagacgagaacaaagactctgagaaaagcacagatgctgtttcggctgctgactctgcgaatggatctgttggtgctgctactgccccggctggattgccctcaaacccgctagccttcaaccctttcctcctgtccacaatggccccgggcctcttctacccatccatgtttctacctccaggactggggggattgacgctgcctgggttcccagcattggcaggacttcagaatgccgtgggctccagcgaagaaaaggctgctgacaaggctgagggaggaccctttaaagatggagagacccttgaaggcagcgatgccgaggagagcctggataagactgcagagtcctccctcttagaagacgaaatagcacagggtgaagagctagactcacttgatgggggggatgaaatagaaaacaatgaaaatgatgaataaccagtaccagttccagttcaagtgtttaaaacttttgacaagtggtagtcctactgtttacactcacagttaatgttcatacctagttttataagctgttctgtaacatagtgtagcaaaaaaaaaagttcaagtcatgttatacaggtgtgtcaaaaggtatcttggtcattaagtattgtgcagtgcattatttattatccctaggagagatgaaatttgagaggtgatcatgtctttttaaggaaacttacataatgctctgctttttttttttctcttggtaccattggtattataataaagagcaatttgtaactgagtggcactaatggaagaaagtgctgctcaaaggaagtatgaagttatatatttaattttttaattttaatttttaatttttttgctgtgaaggtcaagctgaaatttaccatacatatcatacttgctcatttgtttccctttttgactgtatgggggttcccacactcgtgcatacacacacatccatacactctgacaatctccacgctagtgtgaacgcctctgtcccgaggcgcagcaataataaggcagctgttgaatgtgaagggtccctttggaaaattaacctactgggagggttcttgccagacagaactacagttccattgtctcgtggtcttgtaatgcactggtaaaaacaaaataaatagatgaataaataaagagtgagagaagagagaatcaggtaccttttttaaattaaaggactttgttactttagccacaaagctaaaacagcattacctcagctctaaactagccttgaagtttacagacatgactttgtaaatgtattgtttttctttgttgtgatgtccttttatttttttctctgaaaactgctatcatgtaagataaaatgtaaattgctgccaactgtagtaatgatgcttttaataaaagtgacccatgatatgcagagatgtaaaaaaaaaaaaaaaaaaaaaaaaaaa

SEQ ID NO: 14 - DNA, 5107 bp, Homo sapiens
gttggggacggtggacagacattctttcccacggacgctataaacgccaactcactgagcaagatgtagaaaccatctgcagaaccatcctggtgtactgtcttaatcattacaaaggggatgagaatatcaaaagcttcatctgggatctgatcacacccacagcggatggccagactcgagccttggtcaaccattccggtttgtcagctcctgtgccaaggggaaggaagggaaagaaggtgaaagcccagagcacacagccggtggtgcaggatgccgactggctggccagctgcaacccagatgccctgttccaggaggacagctacaagaaacacctgaagcatcactgtaacaaggtcctgctgcgtgtccgcatgctgtactacctaagacaagaagtgataggagaccaggcggataagatcttagagggtgctgactcaagtgaagccgatgtgtggatccctgaacctttccatgctgaagttcctgcagattggtgggataaggaagcagacaaatccctcttaattggagtgttcaaacatggctatgagaagtacaactccatgcgagctgaccccgcgctgtgctttctggaacgagtcggtatgcctgatgccaaggccatagctgccgagcaaagaggaacagacatgctagcagatggtggtgacgggggagaatttgatagagaagatgaagacccagaatataaaccaaccagaacaccgttcaaagatgaaatagatgaatttgcaaattctccttcagaggataaggaagaatccatggaaatacatgccacaggcaagcacagtgagagtaatgctgagttaggccaactttactggcctaacacttcaaccctgactacacgtctgcgccggctcattactgcctatcagcgcagctataaaaggcaacagatgaggcaagaggccctaatgaagactgaccggcgcagacggcggcctcgagaggaagtgagagctctggaagcggaaagggaagctattatatctgagaagcggcaaaagtggacaagaagagaagaggctgatttttaccgtgtggtatccacctttggggttatttttgaccctgtgaaacagcaatttgactggaaccaatttagagcctttgccaggcttgacaaaaaatctgatgagagtttggagaaatacttcagttgttttgtggccatgtgtaggcgagtatgtcgaatgcccgtcaagccagatgatgaaccgcccgacctctcctccataattgagccgatcacagaggagcgagcctctcgaactctgtaccgcattgagctgctacggaagatccgcgagcaggttctccatcacccccagctgggagagaggcttaagctctgccagccaagcttggatctgccagagtggtgggagtgtggacggcatgaccgagacttgctggttggtgctgctaaacacggggtcagtcggacggattatcacatcctcaatgaccctgagttatccttcttggatgcacataaaaactttgctcaaaacagaggggcaggtaatacatcttccttgaacccactggcagttggatttgtccagactcctccagtcatctcatctgctcatattcaagatgagagggtactggaacaagccgaaggcaaagtggaggagcctgaaaacccagctgccaaggagaaatgtgagggcaaagaagaggaagaagaaaccgatggcagcgggaaggagagcaagcaggaatgtgaggcagaggccagctctgtgaaaaatgaactgaaaggggttgaggtcggcgcagacactgggtccaaatctatttcagagaaaggttccgaagaggatgaagaggaaaagctggaggatgacgataagtcggaagagtcttcccagcccgaagcaggagctgtctctagagggaagaattttgatgaagaaagcaatgcttccatgagcactgctagagatgaaacccgagatggattctacatggaggacggagatccttcagtagctcagctccttcatgaaagaacatttgccttctcgttttggcctaaggatagagtaatgataaaccgcttagacaacatctgtgaagcagtgttgaaaggcaaatggccagtaaataggcgccagatgtttgatttccaaggcctcatcccaggttacacacccaccacagtggacagccccttgcagaagaggagctttgctgagctctccatggtcggccaagccagcattagtgggagtgaggacatcactacgtctcctcagttgtcaaagggaagatgccctcaacctctctgtccctcgccagcggaggaggaggaggagaaaaatcgaaattgaggccgaaagagctgccaagaggcgaaatctcatggagatggttgcccagcttcgagagtctcaggtggtctcagaaaatggacaagaaaaagttgtagatttatcaaaggcctcaagagaggcaacaagctctacctcaaatttttcatctctttcttcaaagtttatcttgcctaatgtctcaacaccagtgtctgatgcctttaagactcaaatggaactgctccaagcaggcctttcgcgcacacccacaaggcatctccttaatggctccctagtggatggagagcctcccatgaagaggaggcggggaaggaggaaaaatgtggagggacttgatctgcttttcatgagccacaaacggacgtcattgagtgcaggaggatgctgaggtgaccaaagcttttgaagaagatatagagaccccaccaacaagaaacattccttctcccggacagctggacccagacacacggatccctgttatcaatcttgaagatgggactaggctggtgggggaagatgctcctaaaaataaggatttagttgaatggctgaagctgcaccctacttacactgttgatatgccaaaagaatgcagatgtgctgttttcctcatttcagaaaccgaaacagaaacgacatagatgtcgaaaccctaataaattggatataaacactttgacaggagaagaaagggtgcctgttgtcaataaacgaaatgggaagaagatgggtggagctatggcgcctccaatgaaggatctacccaggtggctggaagaaaatcctgaatttgcagttgctccagactggactgatatagttaagcagtctggttttgttcctgagtcgatgtttgaccgccttctcactgggcctgtagtgcggggagagggagcgagcagaagaggaagaaggcccaaaagtgagatcgccagagcagccgcggccgccgctgctgtggcctccacgtcagggatcaaccctttgctggtgaacagcctgtttgctggaatggacctgacgagccttcagaatctccagaatctccagtcgctccagctggcaggcctcatgggcttccctccaggactggcaacagctgccaccgccggaggcgatgcgaagaaccctgctgctgtgctgcccctgatgctgccaggaatggcgggcctgcccaacgtgtttggcttgggcgggctgttgaataaccctctgtcagctgctactggaaacaccactactgcttctagtcaaggagaaccggaagacagcacttcaaaaggagaggagaaaggaaatgagaatgaagacgagaacaaagactctgagaaaagcacagatgctgtttcggctgctgactctgcgaatggatctgttggtgctgctactgccccggctggattgccctcaaacccgctagccttcaaccctttcctcctgtccacaatggccccgggcctcttctacccatccatgtttctacctccaggactggggggattgacgctgcctgggttcccagcattggcaggacttcagaatgccgtgggctccagcgaagaaaaggctgctgacaaggctgagggaggaccctttaaagatggagagacccttgaaggcagcgatgccgaggagagcctggataagactgcagagtcctccctcttagaagacgaaatagcacagggtgaagagctagactcacttgatgggggggatgaaatagaaaacaatgaaaatgatgaataaccagtaccagttccagttcaagtgtttaaaacttttgacaagtggtagtcctactgtttacactcacagttaatgttcatacctagttttataagctgttctgtaacatagtgtagcaaaaaaaaaagttcaagtcatgttatacaggtgtgtcaaaaggtatcttggtcattaagtattgtgcagtgcattatttattatccctaggagagatgaaatttgagaggtgatcatgtctttttaaggaaacttacataatgctctgctttttttttttctcttggtaccattggtattataataaagagcaatttgtaactgagtggcactaatggaagaaagtgctgctcaaaggaagtatgaagttatatatttaattttttaattttaatttttaatttttttgctgtgaaggtcaagctgaaatttaccatacatatcatacttgctcatttgtttccctttttgactgtatgggggttcccacactcgtgcatacacacacatccatacactctgacaatctccacgctagtgtgaacgcctctgtcccgaggcgcagcaataataaggcagctgttgaatgtgaagggtccctttggaaaattaacctactgggagggttcttgccagacagaactacagttccattgtctcgtggtcttgtaatgcactggtaaaaacaaaataaatagatgaataaataaagagtgagagaagagagaatcaggtaccttttttaaattaaaggactttgttactttagccacaaagctaaaacagcattacctcagctctaaactagccttgaagtttacagacatgactttgtaaatgtattgtttttctttgttgtgatgtccttttatttttttctttgaaaactgctatcatgtaagataaaatgtaaattgctgccaactgtagtaatgatgcttttaataaaagtgacccatgatatgcagagatgt

SEQ ID NO: 15 - DNA, 5879 bp, Homo sapiens
cccttctgggagttcaagatttctgatgaggaggcagatgatgcagatgctgctgggagggattccccctccaacacctcccagtcagaacagcaggaatctgttgatgcagaaggcccagtggtagaaaaaattatgagcagtcgttcagtaaaaaagcagaaggaatctggagaggaggtagaaattgaggaattctatgtgaaatacaaaaacttctcttatcttcattgtcagtgggcatctatagaagatctggaaaaagataagagaattcagcaaaaaattaaacgatttaaggcaaagcagggccagaacaagttcctttcagagattatgtggaggttgaccggataatggactttgcacgtagcacagatgaccggggagagcctgtgactcactatctggtgaagtggtgttcacttccttatgaagacagcacgtgggagcggaggcaggacatagatcaagcaaagatcgaggagtttgagaaactaatgtccagggagccggaaacagagcgtgtggagcgacctcctgctgatgattggaagaaatcggagagttccggggagtataaaaacaataacaaactcagggaataccagttggagggagtaaactggctacttttcaattggtacaacatgcgaaactgcattttagcagatgaaatgggtttgggaaaaactatccagtccattacatttctctatgagatatatttgaaaggaatccatggcccttttttagtaattgccccattgtccacaatccccaactgggaaagggaattccgaacctggacagagttgaacgtggttgtgtatcatgggagtcaagctagtcgtcggaccattcagttgtatgaaatgtacttcaaagatccccagggtcgagtgataaaggggtcctataagtttcatgccatcatcactacatttgagatgattttgactgattgtcctgagctgcggaatattccatggcgctgtgtagtcattgatgaagcccacaggctgaagaacaggaactgcaagctgttggagggactcaagatgatggacttggaacacaaagtgctgctgacgggaaccccactccagaacactgtggaagaactcttcagcttgcttcatttcttggaaccaagtcgcttcccttcagaaaccacatttatgcaagaatttggtgatctaaaaacagaagagcaggtgcaaaaacttcaagctattctaaagccaatgatgttgagacgtctcaaagaggatgtagaaaagaacttggcccccaaagaagaaactattattgaagttgagctaacaaacattcagaagaaatattaccgagccatccttgagaagaatttcacatttctttccaaaggcggtggtcaagctaacgtacctaacctattaaacactatgatggaattgcggaagtgctgcaatcatccgtaccttatcaatggtgctgaagagaaaattttggaagagtttaaagaaacacacaatgcagagtctccagattttcagctccaggcaatgatccaggctgctggcaagctagtgctgattgacaagctgctgccaaaactgaaggctggtggccacagggtgcttatcttttcccagatggtgcgctgcttggacatactggaagactacctcattcaaagacggtacccatatgaaaggatcgacggccgagtaagaggcaacctccgccaggcagctatcgacagattctccaaacctgattctgataggtttgttttcctcctgtgtacaagggcaggaggtttaggcattaacctcactgctgctgatacctgcatcatctttgattcagactggaatccccaaaatgatctccaggctcaggctagatgtcatagaataggacagagcaaatctgtgaaaatctacaggctgattacaagaaattcctatgaaagggaaatgttcgacaaggctagtttgaaactgggcctggataaagctgtgctacagtctatgagtggaagagaaaatgctaccaatggggtacaacagctttccaagaaagaaatagaggatcttctacgaaaaggggcctatggtgcactcatggatgaggaggatgaagggtctaaattctgtgaagaagatattgatcagatcctcctacgtcgaacccacaccattaccattgagtcagaagggaaaggttccacatttgctaaggccagttttgttgcatctggaaataggacagatatttccttggatgatccaaatttctggcaaaagtgggctaagaaggctgaattggatattgatgccttaaatgggaggaacaacctggttattgatactccaagagtgagaaagcagaccaggctctacagtgtagtgaaggaagatgagctgatggagttctcagacttggaaagtgattctgaagaaaagccctgtgcaaagccacggcgtccccaggataagtcacagggctatgcaaggagtgaatgtttcagggtggagaagaatctgcttgtctatggttggggacggtggacagacattctttcccacggacgctataaacgccaactcactgagcaagatgtagaaaccatctgcagaaccatcctggtgtactgtcttaatcattacaaaggggatgagaatatcaaaagcttcatctgggatctgatcacacccacagcggatggccagactcgagccttggtcaaccattccggtttgtcaactcctgtgccaaggggaaggaagggaaagaaggtgaaagcccagagcacacagccggtggtgcaggatgccgactggctggccagctgcaacccagatgccctgttccaggaggacagctacaagaaacacctgaagcatcactgtaacaaggtcctgctgcgtgtccgcatgctgtactacctaagacaagaagtgataggagaccaggcggataagatcttagagggtgctgactcaagtgaagccgatgtgtggatccctgaacctttccatgctgaagttcctgcagattggtgggataaggaagcagacaaatccctcttaattggggtgttcaaacatggctatgagaagtacaactccatgcgagccgaccccgcgctgtgctttctggaacgagtcggtatgcctgatgccaaggccatagctgccgggcaaagaggaacagacatgctagcagatggtggtgacgggggagaatttgatagagaagatgaagacccagaatataaaccaaccagaacaccgttcaaagatgaaatagatgaatttgcaaattctccttcagaggataaggaagaatccatggaaatacatgccacaggcaagcacagtgagagtaatgctgagttaggccaactttactggcctaacacttcaaccctgactacacgtctgcgccggctcattactgcctatcagcgcagctataaaaggcaacagatgaggcaagaggccctaatgaagactgaccggcgcagacggcggcctcgagaggaagtgagagctctggaagcggaaagggaagctattatatctgagaagcggcaaaagtggacaagaagagaagaggctgatttttaccgtgtggtatccacctttggggttatttttgaccctgtgaaacagcaatttgactggaaccaatttagagcctttgccaggcttgacaaaaaatctgatgagagtttggagaaatacttcagttgttttgtggccatgtgtaggcgagtatgtcgaatgcccgtcaagccagatgatgaaccgcccgacctctcctccataattgagccgatcacagaggagcgagcctctcgaactctgtaccgcattgagctgctacggaagatccgcgagcaggttctccatcacccccagctgggagagaggcttaagctctgccagccaagcttggatctgccagagtggtgggagtgtggacggcatgaccgagacttgctggttggtgctgctaaacacggggtcagtcggacggattatcacatcctcaatgaccctgagttatccttcttggatgcacataaaaactttgctcaaaacagaggggcaggtaatacatcttccttgaacccactggcagttggatttgtccagactcctccagtcatctcatctgctcatattcaagatgagagggtactggaacaagccgatggcaaagtggaggagcctgaaaacccagctgccaaggagaaatgtgagggcaaagaagaggaagaagaaaccgatggcagcgggaaggagagcaagcaggaatgtgaggcagaggccagctctgtgaaaaatgaactgaaaggtgttgaggtcggcgcagacactgggtccaaatctatttcagagaaaggttccgaagaggatgaagaggaaaagctggaggatgacgataagtcggaagagtcttcccagcccgaaggagctgtctctagagggaagaattttgatgaagaaagcaatgcttccatgagcactgctagagatgaaacccgagatggattctacatggaggacggagatccttcagtagctcagctccttcatgaaagaacatttgccttctcgttttggcctaaggatagagtaatgataaaccgcttagacaacatctgtgaagcagtgttgaaaggcaaatggccagtaaataggcgccagatgtttgatttccaaggcctcatcccaggttacacacccaccacagtggacagccccttgcagaagaggagctttgctgagctctccgtggtcggccaagccagcattagtgggagtgaggacatcactacgtctcctcagttgtcaaaggaagatgccctcaacctctctgtccctcgccagcggaggaggaggaggagaaaaatcgaaattgaggccgaaagagctgccaagaggcgaaatctcatggagatggttgcccagcttcgagagtcccaggtggtctcagaaaatggacaagaaaaagttgtagatttatcaaaggcctcaagagaggcaacaagctctacctcaaatttttcatctctttcttcaaagtttatcttgcctaatgtctcaacaccagtgtctgatgcctttaagactcaaatggaactgctccaagcaggcctttcgcgcacacccacaaggcatctccttaatggctccctagtggatggagagcctcccatgaagaggaggcggggaaggaggaaaaatgtggagggacttgatctgcttttcatgagccacaaacggacgtcattgagtgcagaggatgctgaggtgaccaaagcttttgaagaagatatagagaccccaccaacaagaaacattccttctcccggacagttggacccagacacacggatccctgttatcaatcttgaagatgggactaggctggtgggggaagatgctcctaaaaataaggatttagttgaatggctgaagctgcaccctacttacactgttgatatgccaagttatgtaccaaagaatgcagatgtgctgttttcctcatttcagaaaccgaaacagaaacgacatagatgtcgaaaccctaataaattggatataaacactttgacaggagaagaaagggtgcctgttgtcaataaacgaaatgggaagaagatgggtggagctatggcgcctccaatgaaggatctacccaggtggctggaagaaaatcctgaatttgtagttgctccagactggactgatatagttaagcagtctggttttgttcctgagtcgatgtttgaccgccttctcactgggcctgtagtgcggggagagggagcgagcagaagaggaggaaggcccaaaagtgagatcgccagagcagcc

SEQ ID NO: 16 - DNA, 9135 bp, Homo sapiens
ctcgctttgtcctgttctttgtcttgttgtcttttttgttttcttttcgtgttcttcttcctctctcggttgtcccttctttgcttgtccttgtgatatttcgtttttctcttctttcttgcgcatgttcttcggtgatcctgttgtgccttctttaatctcctgattcgtcgtcctgatcccactcgtggatcccatcaactggtcaatgactatgcaccagcgcccttcccccccgggcttcatttaccatctgacagccatcttgcatcccttcacaagccttcagtgaacttatgccttctgcaccttccctatgtcacccatgaaagcaactgagttatctcatgcaaggcactcctcctccactaagtcaggccgggaagcgtctgggataccaatggaaggttggccagttattccctatatgccccattcctcagccatctcaccagccccctggtgccactgggaatcggacagaggaatatgggccccagaaacatgcagcagtctcgtccatttataggcatgtcctcggcaccaaagggaattgactgggcacatgaggccaaatggttgtcctggtgttggccttggagacccacaagcaatccaggaacgactgatacctggccaacaacatcctggtcaacagccatcttttcagcagttgccaacctgtcctccactgcagcctcacccgggcttgcaccaccagtcttcacctccacaccctcatcaccagccttgggcacagctccacccatcaccccagaacaccccgcagaaagtgcctgtgcatcagcattccccgtcggagccctttctagagaaaccagtgccggatatgactcaggttagtggaccgaatgctcagctagtgaagagtgatgattacctgccatcaatagaacagcagccacaacaaaagaagaagaaaaagaaaaacaaccacattgtagcagaggatcccagtaaaggttttggtaaagatgacttccctggtggggtagataaccaagaactaaataggaactcactggatgggtcccaagaagaaaaaaagaaaaagaaaaggtcaaaggcaaaaaaagacccgaaggaaccgaaagaacccaaggagaaaaaaaagcctaaggaacccaagaccccgaaagcccctaagattcccaaagagccaaaggaaaagaaagcaaaaactgccacgccaaaacccaaatccagcaaaaagtcaagtaataagaaacctgactcagaagcaagtgctttgaagaaaaaggtcaacaagggaaaaacagaaggttctgaaaattcagacttagacaaaacacccccaccatctcctcctcctgaagaagatgaggacccaggtgttcagaagagacggtccagcagacaggtgaagagaaagcgctacactgcccttctggagttcaagatttctgatgaggaggcagatgatgcagatgctgctgggagggattccccctccaacacctcccagtcagaacagcaggaatctgttgatgcagaaggcccagtggtagaaaaaattatgagcagtcgttcagtaaaaaagcagaaggaatctggagaggaggtagaaattgaggaattctatgtgaaatacaaaaacttctcttatcttcattgtcagtgggcatctatagaagatctggaaaaagataagagaattcagcaaaaaattaaacgatttaaggcaaagcagggccagaacaagttcctttcagagattgaggatgagctttttaatccagattatgtggaggttgaccggataatggactttgcacgtagcacagatgaccggggagagcctgtgactcactatctggtgaagtggtgttcacttccttatgaagacagcacgtgggagcggaggcaggacatagatcaagcaaagatcgaggagtttgagaaactaatgtccagggagccggaaacagagcgtgtggagcgacctcctgctgatgattggaagaaatcggagagttccagggagtataaaaacaataacaaactcagggaataccagttggagggagtaaactggctacttttcaattggtacaacatgcgaaactgcattttagcagatgaaatgggtttgggaaaaactatccagtccattacatttctctatgagatatatttgaaaggaatccatggcccttttttagtaattgccccattgtccacaatccccaactgggaaagggaattccgaacctggacagagttgaacgtggttgtgtatcatgggagtcaagctagtcgtcggaccattcagttgtatgaaatgtacttcaaagatccccagggtcgagtgataaaggggtcctataagtttcatgccatcatcactacatttgagatgattttgactgattgtcctgagctgcggaatattccatggcgctgtgtagtcattgatgaagcccacaggctgaagaacaggaactgcaagctgttggagggactcaagatgatggacttggaacacaaagtgctgctgacgggaaccccactccagaacactgtggaagaactcttcagcttgcttcatttcttggaaccaagtcgcttcccttcagaaaccacatttatgcaagaatttggtgatctaaaaacagaagagcaggtgcaaaaacttcaagctattctaaagccaatgatgttgagacgtctcaaagaggatgtagaaaagaacttggcccccaaagaagaaactattattgaagttgagctaacaaacattcagaagaaatattaccgagccatccttgagaagaatttcacatttctttccaaaggcggtggtcaagctaacgtacctaacctattaaacactatgatggaattgcggaagtgctgcaatcatccgtaccttatcaatggtgctgaagagaaaattttggaagagtttaaagaaacacacaatgcagagtctccagattttcagctccaggcaatgatccaggctgctggcaagctagtgctgattgacaagctgctgccaaaactgaaggctggtggccacagggtgcttatcttttcccagatggtgcgctgcttggacatactggaagactacctcattcaaagacggtacccatatgaaaggatcgacggccgagtaagaggcaacctccgccaggcagctatcgacagattctccaaacctgattctgataggtttgttttcctcctgtgtacaagggcaggaggtttaggcattaacctcactgctgctgatacctgcatcatctttgattcagactggaatccccaaaatgacctccaggctcaggctagatgtcatagaataggacagagcaaatctgtgaaaatctacaggctgattacaagaaattcctatgaaagggaaatgttcgacaaggctagtttgaaactgggcctggataaagctgtgctacagtctatgagtggaagagaaaatgctaccaatggggtacaacagctttccaagaaagaaatagaggatcttctacgaaaaggggcctatggtgcactcatggatgaggaggatgaagggtctaaattctgtgaagaagatattgatcagatcctcctacgtcgaacccacaccattaccattgagtcagaagggaaaggttccacatttgctaaggccagttttgttgcatctggaaataggacagatatttccttggatgatccaaatttctggcaaaagtgggctaagaaggctgaattggatattgatgccttaaatgggaggaacaacctggttattgatactccaagagtgagaaagcagaccaggctctacagtgcagtgaaggaagatgagctgatggagttctcagacttggaaagtgattctgaagaaaagccctgtgcaaagccacggcgtccccaggataagtcacagggctatgcaaggagtgaatgtttcagggtggagaagaatctgcttgtctatggttggggacggtggacagacattctttcccacggacgctataaacgccaactcactgagcaagatgtagaaaccatctgcagaaccatcctggtgtactgtcttaatcattacaaaggggatgagaatatcaaaagcttcatctgggatctgatcacacccacagcggatggccagactcgagccttggtcaaccattccggtttgtcagctcctgtgccaaggggaaggaagggaaagaaggtgaaagcccagagcacacagccggtggtgcaggatgccgactggctggccagctgcaacccagatgccctgttccaggaggacagctacaagaaacacctgaagcatcactgtaacaaggtcctgctgcgtgtccgcatgctgtactacctaagacaagaagtgataggagaccaggcggataagatcttagagggtgctgactcaagtgaagccgatgtgtggatccctgaacctttccatgctgaagttcctgcagattggtgggataaggaagcagacaaatccctcttaattggagtgttcaaacatggctatgagaagtacaactccatgcgagctgaccccgcgctgtgctttctggaacgagtcggtatgcctgatgccaaggccatagctgccgagcaaagaggaacagacatgctagcagatggtggtgacgggggagaatttgatagagaagatgaagacccagaatataaaccaaccagaacaccgttcaaagatgaaatagatgaatttgcaaattctccttcagaggataaggaagaatccatggaaatacatgccacaggcaagcacagtgagagtaatgctgagttaggccaactttactggcctaacacttcaaccctgactacacgtctgcgccggctcattactgcctatcagcgcagctataaaaggcaacagatgaggcaagaggccctaatgaagactgaccggcgcagacggcggcctcgagaggaagtgagagctctggaagcggaaagggaagctattatatctgagaagcggcaaaagtggacaagaagagaagaggctgatttttaccgtgtggtatccacctttggggttatttttgaccctgtgaaacagcaatttgactggaaccaatttagagcctttgccaggcttgacaaaaaatctgatgagagtttggagaaatacttcagttgttttgtggccatgtgtaggcgagtatgtcgaatgcccgtcaagccagatgatgaaccgcccgacctctcctccataattgagccgatcacagaggagcgagcctctcgaactctgtaccgcattgagctgctacggaagatccgcgagcaggttctccatcacccccagctgggagagaggcttaagctctgccagccaagcttggatctgccagagtggtgggagtgtggacggcatgaccgagacttgctggttggtgctgctaaacacggggtcagtcggacggattatcacatcctcaatgaccctgagttatccttcttggatgcacataaaaactttgctcaaaacagaggggcaggtaatacatcttccttgaacccactggcagttggatttgtccagactcctccagtcatctcatctgctcatattcaagatgagagggtactggaacaagccgaaggcaaagtggaggagcctgaaaacccagctgccaaggagaaatgtgagggcaaagaagaggaagaagaaaccgatggcagcgggaaggagagcaagcaggaatgtgaggcagaggccagctctgtgaaaaatgaactgaaaggtgttgaggtcggcgcagacactgggtccaaatctatttcagagaaaggttccgaagaggatgaagaggaaaagctggaggatgacgataagtcggaagagtcttcccagcccgaagcaggagctgtctctagagggaagaattttgatgaagaaagcaatgcttccatgagcactgctagagatgaaacccgagatggattctacatggaggacggagatccttcagtagctcagctccttcatgaaagaacatttgccttctcgttttggcctaaggatagagtaatgataaaccgcttagacaacatctgtgaagcagtgttgaaaggcaaatggccagtaaataggcgccagatgtttgatttccaaggcctcatcccaggttacacacccaccacagtggacagccccttgcagaagaggagctttgctgagctctccatggtcggccaagccagcattagtgggagtgaggacatcactacgtctcctcagttgtcaaaggaagatgccctcaacctctctgtccctcgccagcggaggaggaggaggagaaaaatcgaaattgaggccgaaagagctgccaagaggcgaaatctcatggagatggttgcccagcttcgagagtctcaggtggtctcagaaaatggacaagaaaaagttgtagatttatcaaaggcctcaagagaggcaacaagctctacctcaaatttttcatctctttcttcaaagtttatcttgcctaatgtctcaacaccagtgtctgatgcctttaagactcaaatggaactgctccaagcaggcctttcgcgcacacccacaaggcatctccttaatggctccctagtggatggagagcctcccatgaagaggaggcggggaaggaggaaaaatgtggagggacttgatctgcttttcatgagccacaaacggacgtcattgagtgcagaggatgctgaggtgaccaaagcttttgaagaagatatagagaccccaccaacaagaaacattccttctcccggacagctggacccagacacacggatccctgttatcaatcttgaagatgggactaggctggtgggggaagatgctcctaaaaataaggatttagttgaatggctgaagctgcaccctacttacactgttgatatgccaagttatgtaccaaagaatgcagatgtgctgttttcctcatttcagaaaccgaaacagaaacgacatagatgtcgaaaccctaataaattggatataaacactttgacaggagaagaaagggtgcctgttgtcaataaacgaaatgggaagaagatgggtggagctatggcgcctccaatgaaggatctacccaggtggctggaagaaaatcctgaatttgcagttgctccagactggactgatatagttaagcagtctggttttgttcctgagtcgatgtttgaccgccttctcactgggcctgtagtgcggggagagggagcgagcagaagaggaagaaggcccaaaagtgagatcgccagagcagccgcggccgccgctgctgtggcctccacgtcagggatcaaccctttgctggtgaacagcctgtttgctggaatggacctgacgagccttcagaatctccagaatctccagtcgctccagctggcaggcctcatgggcttccctccaggactggcaacagctgccaccgccggaggcgatgcgaagaaccctgctgctgtgctgcccctgatgctgccaggaatggcgggcctgcccaacgtgtttggcttgggcgggctgttgaataaccctctgtcagctgctactggaaacaccactactgcttctagtcaaggagaaccggaagacagcacttcaaaaggagaggagaaaggaaatgagaatgaagacgagaacaaagactctgagaaaagcacagatgctgtttcggctgctgactctgcgaatggatctgttggtgctgctactgccccggctggattgccctcaaacccgctagccttcaaccctttcctcctgtccacaatggccccgggcctcttctacccatccatgtttctacctccaggactggggggattgacgctgcctgggttcccagcattggcaggacttcagaatgccgtgggctccagcgaagaaaaggctgctgacaaggctgagggaggaccctttaaagatggagagacccttgaaggcagcgatgccgaggagagcctggataagactgcagagtcctccctcttagaagacgaaatagcacagggtgaagagctagactcacttgatgggggggatgaaatagaaaacaatgaaaatgatgaataaccagtaccagttccagttcaagtgtttaaaacttttgacaagtggtagtcctactgtttacactcacagttaatgttcatacctagttttataagctgttctgtaacatagtgtagcaaaaaaaaaagttcaagtcatgttatacaggtgtgtcaaaaggtatcttggtcattaagtattgtgcagtgcattatttattatccctaggagagatgaaatttgagaggtgatcatgtctttttaaggaaacttacataatgctctgctttttttttttctcttggtaccattggtattataataaagagcaatttgtaactgagtggcactaatggaagaaagtgctgctcaaaggaagtatgaagttatatatttaattttttaattttaatttttaatttttttgctgtgaaggtcaagctgaaatttaccatacatatcatacttgctcatttgtttccctttttgactgtatgggggttcccacactcgtgcatacacacacatccatacactctgacaatctccacgctagtgtgaacgcctctgtcccgaggcgcagcaataataaggcagctgttgaatgtgaagggtccctttggaaaattaacctactgggagggttcttgccagacagaactacagttccattgtctcgtggtcttgtaatgcactggtaaaaacaaaataaatagatgaataaataaagagtgagagaagagagaatcaggtaccttttttaaattaaaggactttgttactttagccacaaagctaaaacagcattacctcagctctaaactagccttgaagtttacagacatgactttgtaaatgtattgtttttctttgttgtgatgtccttttatttttttctctgaaaactgctatcatgtaagataaaatgtaaattgctgccaactgtagtaatgatgcttttaataaaagtgacccatgatatgcagagatgtaaaaaaaaaaaaaaaaaaaaaaaaaaa

SEQ ID NO: 17 - DNA, 4943 bp, Homo sapiens
ccaaaggggcaggtcataccaacatgcctaatctacttaacacaatgatggagttgcgcaagtgctgcaaccacccatatctcatcaatggtgctgaagaaaaaatcctaacagaattccgtgaagcttgccatattatacctcatgactttcacctgcaggccatggttcgttcagccggcaaactggttcttattgacaagttgcttccaaagcttaaagctggtggccataaagttctgatcttctctcagatggtgcgctgcctagacatcctagaggattatttaatccagaggaggtacttatatgaacgtattgatgggcgagttagaggcaaccttcgacaggctgccattgaccgcttcagcaagcctgactcagaccgctttgtcttcttactgtgtacccgggctggtggacttggtattaatcttacagctgctgatacctgcatcatctttgattcagactggaatccacaaaatgacctgcaggcccaggcacgatgtcatcgaattgggcagagcaaagctgtgaaggtgtaccgcctcatcactcgtaattcctacgagagagagatgtttgataaggccagcctcaagttggggttggataaggctgtgcttcaatccatgagtggtcgggatggcaacattactggaatccaacagttctctaagaaggagattgaagatcttttaagaaaaggagcatatgcagccatcatggaggaagatgatgaaggctccaagttttgtgaagaggacattgaccagatcttgttaagacgaactacaaccatcaccattgaatctgaaggaaaaggttccacctttgctaaggcaagctttgttgcttctgaaaacaggacagatatttctttggatgaccccaacttttggcaaaagtgggccaaaaaggctgacctagacatggatctgctcaacagcaagaataatttggtaattgacacacctagagtacgaaaacaaacgcgccactttagcactctgaaagatgatgacctggtggaattctctgatttggaaagtgaggatgatgagcggccacgctcccgcagacatgaccgtcatcatgcctatgggcgcactgactgctttcgggtggaaaagcatctcctggtatatggttggggacgatggcgagatattttatctcatggacgcttcaagcgacgtatgactgaacgagatgtggagaccatttgtcgggccattctcgtgtactgtcttctacactaccgtggggatgaaaatattaaaggcttcatctgggacttgattagcccagctgaaaatggcaagacaaaagaattgcagaatcattcaggtctatctatccctgtgcctcgtggacgcaaaggaaaaaaagtaaagtcacaaagcacttttgatatccataaggcagattggatccggaaatataaccctgacactttgttccaagatgaaagttataagaagcacttgaaacatcagtgtaacaaggtactgttgcgggtacgaatgctatactacctgaggcaggaggttattggagaccaagcagaaaaggtgttagggggtgcgattgccagtgagattgacatatggttcccagtagtggatcaactggaggttccaacaacttggtgggacagtgaggctgacaagtcgctgctcattggagtctttaaacatggctatgagaaatataataccatgagggcagacccagccttatgtttcctagaaaaggctggccgaccagatgacaaagcaattgcagcagaacatcgagtgttggataacttctctgacatagtagaaggggttgactttgataaagattgtgaagatcctgaatataaaccactccaaggtcccccaaaggaccaagatgatgagggtgatcccttgatgatgatggatgaggagatctcagtgattgatggagatgaagcccaggtgacccaacagccaggccatttattctggcctccgggctctgccctaacagctaggcttcggcgtctagtaacagcgtatcagcgcagctacaagagagaacaaatgaagatagaggctgcagaacgtggggaccggcgaaggcggcgttgtgaagcagccttcaagctgaaagaaattgcacggcgggagaaacaacaacgatggacaaggcgtgaacaaactgatttttatcgagtggtgtctacgtttggtgtggaatatgaccctgacaccatgcagttccattgggatcgcttccgcacttttgctcgactagacaaaaagacagatgaaagccttaccaagtacttccatggctttgtggccatgtgccgccaagtatgccgccttcccccagcagctggagatgaaccccccgaccctaacctgttcattgagcccatcactgaggagagagcctcacggactctctaccgtatagaattgcttcggcgcttacgggaacaagttttatgccacccccttttggaagatcggctggcattgtgtcagcctcctggtcctgaattgcccaaatggtgggagcctgttcggcatgatggggagcttctaagaggggcagcccgccatggggtgagccaaacagactgcaacatcatgcaggacccagacttctcttttctggctgcccgtatgaattatatgcagaaccatcaagcaggagcaccagctccatccttgtcacgctgctctactccactgctgcaccagcagtatacctcacgcactgcctcaccactgcccctgcgcccagatgctcctgttgaaaagtcacccgaggagacagctacccaggtccccagtctggagagtctgactttaaagctagagcacgaggtggtggccaggagccgaccaaccccacaagactatgagatgcgagtatccccctctgatactacccctctggtttcccggagtgttccaccagtcaaactggaggatgaggatgattcggactctgagctggacttgagcaagctgtcaccatcttcttcttcttcctcatcctcatccagctccagctccagcactgatgagagtgaggatgagaaggaagagaagctaactgaccagtcccgctcaaagctctatgatgaagagagtctcctgtccctcactatgtcccaagatggattcccaaatgaagatggagaacaaatgacccctgagcttctgctactgcaggaaagacaaagagcctctgagtggcccaaggatcgtgtcctgataaaccgtattgacctcgtctgccaggctgtactctcagggaagtggccttctagccgtaggagccaggaaatggtaacaggaggaattttggggccaggcaaccacttgctagacagtccctcattgactcctggagaatatggtgactctccagtccccacaccacgaagtagtagtgcagcttccatggcagaggaggaagcatctgcagtcagcacagcggcagcccagttcaccaaacttcgccgaggcatggatgaaaaggagtttacagttcaaatcaaagatgaggaaggattgaagttaacattccagaagcacaagttgatggcgaatggagtaatgggagatggacatccactgtttcataagaagaaggggaacagaaagaagctagtagagctggaggtggagtgcatggaagagcctaatcaccttgatgtggacctggagacccggatccctgtcatcaataaggtggatggtactttgctggtgggtgaggatgcccctcgccgggctgaactggagatgtggttacagggtcatccagagtttgctgttgatccccgatttctagcgtatatggaggatcgcagaaaacagaagtggcaaagatgtaaaaaaaataataaggcagaattgaactgtttgggaatggaaccagtacagacagctaactctagaaatgggaaaaagggtcatcacactgaaacggtgttcaaccgggttttgccagggcctattgcaccagagagcagcaagaagcgggcccgtaggatgcgaccagacctttctaagatgatggccctcatgcagggtggaagcactgggtctctatctctgcataacacgttccaacacagcagtagtggcctacagtctgtgtcatctttgggtcacagcagtgccacttctgcatctttgccttttatgccatttgtgatgggtggtgcaccatcatcccctcatgtagactccagcaccatgcttcatcaccaccaccaccacccccacccccaccatcaccaccatcaccatccaggcttgagagcccctggctacccctcttcaccagtgactaccgcctctggtactaccttgcggttgccaccactgcaacctgaggaggatgacgatgaggatgaagaagatgatgatgacttatctcagggctatgatagctcagaaagggacttctcactcattgatgatcctatgatgccagcttactcagactccagtgaagatgctgatgactgaagccccagcatgggccccattgcttgggcggctgctgtattttcatttactctggcccttggactatggaaacgtgggaggggcaggggagatgtggggaagtccaggactccaggaggtgaaaaggaaaaaaaaaaaaaaatgtacctgattgctcccaattatgagaggattgggtgggcaggggaactcctaaaataatacatgaccacttcctcatttctggggaaggaaaggagactagagcagctggtgtgctcacccctccctagtcacctccattaaccacagactatgtagcgctggccctagcctctggcagagcctgttcctggccgaactgtggatacagctggagggtcaggaactgttaccttctttccccttggcattaataaatttaagttaatcctt

SEQ ID NO: 18 - DNA, 4668 bp, Homo sapiens
ctagaggattatttaatccagaggaggtacttatatgaacgtattgatgggcgagttagaggcaaccttcgacaggctgccattgaccgcttcagcaagcctgactcagaccgctttgtcttcttactgtgtacccgggctggtggacttggtattaatcttacagctgctgatacctgcatcatctttgattcagactggaatccacaaaatgacctgcaggcccaggcacgatgtcatcgaattgggcagagcaaagctgtgaaggtgtaccgcctcatcactcgtaattcctacgagagagagatgtttgataaggccagcctcaagttggggttggataaggctgtgcttcaatccatgagtggtcgggatggcaacattactggaatccaacagttctctaagaaggagattgaagatcttttaagaaaaggagcatatgcagccatcatggaggaagatgatgaaggctccaagttttgtgaagaggacattgaccagatcttgttaagacgaactacaaccatcaccattgaatctgaaggaaaaggttccacctttgctaaggcaagctttgttgcttctgaaaacaggacagatatttctttggatgaccccaacttttggcaaaagtgggccaaaaaggctgacctagacatggatctgctcaacagcaagaataatttggtaattgacacacctagagtacgaaaacaaacgcgccactttagcactctgaaagatgatgacctggtggaattctctgatttggaaagtgaggatgatgagcggccacgctcccgcagacatgaccgtcatcatgcctatgggcgcactgactgctttcgggtggaaaagcatctcctggtatatggttggggacgatggcgagatattttatctcatggacgcttcaagcgacgtatgactgaacgagatgtggagaccatttgtcgggccattctcgtgtactgtcttctacactaccgtggggatgaaaatattaaaggcttcatctgggacttgattagcccagctgaaaatggcaagacaaaagaattgcagaatcattcaggtctatctatccctgtgcctcgtggacgcaaaggaaaaaaagtaaagtcacaaagcacttttgatatccataaggcagattggatccggaaatataaccctgacactttgttccaagatgaaagttataagaagcacttgaaacatcagtgtaacaaggtactgttgcgggtacgaatgctatactacctgaggcaggaggttattggagaccaagcagaaaaggtgttagggggtgcgattgccagtgagattgacatatggttcccagtagtggatcaactggaggttccaacaacttggtgggacagtgaggctgacaagtcgctgctcattggagtctttaaacatggctatgagaaatataataccatgagggcagacccagccttatgtttcctagaaaaggctggccgaccagatgacaaagcaattgcagcagaacatcgagtgttggataacttctctgacatagtagaaggggttgactttgataaagattgtgaagatcctgaatataaaccactccaaggtcccccaaaggaccaagatgatgagggtgatcccttgatgatgatggatgaggagatctcagtgattgatggagatgaagcccaggtgacccaacagccaggccatttattctggcctccgggctctgccctaacagctaggcttcggcgtctagtaacagcgtatcagcgcagctacaagagagaacaaatgaagatagaggctgcagaacgtggggaccggcgaaggcggcgttgtgaagcagccttcaagctgaaagaaattgcacggcgggagaaacaacaacgatggacaaggcgtgaacaaactgatttttatcgagtggtgtctacgtttggtgtggaatatgaccctgacaccatgcagttccattgggatcgcttccgcacttttgctcgactagacaaaaagacagatgaaagccttaccaagtacttccatggctttgtggccatgtgccgccaagtatgccgccttcccccagcagctggagatgaaccccccgaccctaacctgttcattgagcccatcactgaggagagagcctcacggactctctaccgtatagaattgcttcggcgcttacgggaacaagttttatgccacccccttttggaagatcggctggcattgtgtcagcctcctggtcctgaattgcccaaatggtgggagcctgttcggcatgatggggagcttctaagaggggcagcccgccatggggtgagccaaacagactgcaacatcatgcaggacccagacttctcttttctggctgcccgtatgaattatatgcagaaccatcaagcaggagcaccagctccatccttgtcacgctgctctactccactgctgcaccagcagtatacctcacgcactgcctcaccactgcccctgcgcccagatgctcctgttgaaaagtcacccgaggagacagctacccaggtccccagtctggagagtctgactttaaagctagagcacgaggtggtggccaggagccgaccaaccccacaagactatgagatgcgagtatccccctctgatactacccctctggtttcccggagtgttccaccagtcaaactggaggatgaggatgattcggactctgagctggacttgagcaagctgtcaccatcttcttcttcttcctcatcctcatccagctccagctccagcactgatgagagtgaggatgagaaggaagagaagctaactgaccagtcccgctcaaagctctatgatgaagagagtctcctgtccctcactatgtcccaagatggattcccaaatgaagatggagaacaaatgacccctgagcttctgctactgcaggaaagacaaagagcctctgagtggcccaaggatcgtgtcctgataaaccgtattgacctcgtctgccaggctgtactctcagggaagtggccttctagccgtaggagccaggaaatggtaacaggaggaattttggggccaggcaaccacttgctagacagtccctcattgactcctggagaatatggtgactctccagtccccacaccacgaagtagtagtgcagcttccatggcagaggaggaagcatctgcagtcagcacagcggcagcccagttcaccaaacttcgccgaggcatggatgaaaaggagtttacagttcaaatcaaagatgaggaaggattgaagttaacattccagaagcacaagttgatggcgaatggagtaatgggagatggacatccactgtttcataagaagaaggggaacagaaagaagctagtagagctggaggtggagtgcatggaagagcctaatcaccttgatgtggacctggagacccggatccctgtcatcaataaggtggatggtactttgctggtgggtgaggatgcccctcgccgggctgaactggagatgtggttacagggtcatccagagtttgctgttgatccccgatttctagcgtatatggaggatcgcagaaaacagaagtggcaaagatgtaaaaaaaataataaggcagaattgaactgtttgggaatggaaccagtacagacagctaactctagaaatgggaaaaagggtcatcacactgaaacggtgttcaaccgggttttgccagggcctattgcaccagagagcagcaagaagcgggcccgtaggatgcgaccagacctttctaagatgatggccctcatgcagggtggaagcactgggtctctatctctgcataacacgttccaacacagcagtagtggcctacagtctgtgtcatctttgggtcacagcagtgccacttctgcatctttgccttttatgccatttgtgatgggtggtgcaccatcatcccctcatgtagactccagcaccatgcttcatcaccaccaccaccacccccacccccaccatcaccaccatcaccatccaggcttgagagcccctggctacccctcttcaccagtgactaccgcctctggtactaccttgcggttgccaccactgcaacctgaggaggatgacgatgaggatgaagaagatgatgatgacttatctcagggctatgatagctcagaaagggacttctcactcattgatgatcctatgatgccagctaactcagactccagtgaagatgctgatgactgaagccccagcatgggccccattgcttgggcggctgctgtattttcatttactctggcccttggactatggaaacgtgggaggggcaggggagatgtggggaagtccaggactccaggaggtgaaaaggaaaaaaaaaaaaaaatgtacctgattgctcccaattatgagaggattgggtgggcaggggaactcctaaaataatacatgaccacttcctcatttctggggaaggaaaggagactagagcagctggtgtgctcacccctccctagtcacctccattaaccacagactatgtagcgctggccctagcctctggcagagcctgttcctggccgaactgtggatacagctggagggtcaggaactgttaccttctttccccttggcattaataaatttaagttaatcctt

SEQ ID NO: 19 - DNA, 2692 bp, Homo sapiens
tttgttgcttctgaaaacaggacagatatttctttggatgaccccaacttttggcaaaagtgggccaaaaaggctgacctagacatggatctgctcaacagcaagaataatttggtaattgacacacctagagtacgaaaacaaacgcgccactttagcactctgaaagatgatgacctggtggaattctctgatttggaaagtgaggatgatgagcggccacgctcccgcagacatgaccgtcatcatgcctatgggcgcactgactgctttcgggtggaaaagcatctcctggtatatggttggggacgatggcgagatattttatctcatggacgcttcaagcgacgtatgactgaacgagatgtggagaccatttgtcgggccattctcgtgtactgtcttctacactaccgtggggatgaaaatattaaaggcttcatctgggacttgattagcccagctgaaaatggcaagacaaaagaattgcagaatcattcaggtctatctatccctgtgcctcgtggacgcaaaggaaaaaaagtaaagtcacaaagcacttttgatatccataaggcagattggatccggaaatataaccctgacactttgttccaagatgaaagttataagaagcacttgaaacatcagtgtaacaaggtactgttgcgggtacgaatgctatactacctgaggcaggaggttattggagaccaagcagaaaaggtgttagggggtgcgattgccagtgagattgacatatggttcccagtagtggatcaactggaggttccaacaacttggtgggacagtgaggctgacaagtcgctgctcattggagtctttaaacatggctatgagaaatataataccatgagggcagacccagccttatgtttcctagaaaaggctggccgaccagatgacaaagcaattgcagcagaacatcgagtgttggataacttctctgacatagtagaaggggttgactttgataaagattgtgaagatcctgaatataaaccactccaaggtcccccaaaggaccaagatgatgagggtgatcccttgatgatgatggatgaggagatctcagtgattgatggagatgaagcccaggtgacccaacagccaggccatttattctggcctccgggctctgccctaacagctaggcttcggcgtctagtaacagcgtatcagcgcagctacaagagagaacaaatgaagatagaggctgcagaacgtggggaccggcgaaggcggcgttgtgaagcagccttcaagctgaaagaaattgcacggcgggagaaacaacaacgatggacaaggcgtgaacaaactgatttttatcgagtggtgtctacgtttggtgtggaatatgaccctgacaccatgcagttccattgggatcgcttccgcacttttgctcgactagacaaaaagacagatgaaagccttaccaagtacttccatggctttgtggccatgtgccgccaagtatgccgccttcccccagcagctggagatgaaccccccgaccctaacctgttcattgagcccatcactgaggagagagcctcacggactctctaccgtatagaattgcttcggcgcttacgggaacaagttttatgccacccccttttggaagatcggctggcattgtgtcagcctcctggtcctgaattgcccaaatggtgggagcctgttcggcatgatggggagcttctaagaggggcagcccgccatggggtgagccaaacagactgcaacatcatgcaggacccagacttctcttttctggctgcccgtatgaattatatgcagaaccatcaagcaggagcaccagctccatccttgtcacgctgctctactccactgctgcaccagcagtatacctcacgcactgcctcaccactgcccctgcgcccagatgctcctgttgaaaagtcacccgaggagacagctacccaggtccccagtctggagagtctgactttaaagctagagcacgaggtggtggccaggagccgaccaaccccacaagactatgagatgcgagtatccccctctgatactacccctctggtttcccggagtgttccaccagtcaaactggaggatgaggatgattcggactctgagctggacttgagcaagctgtcaccatcttcttcttcttcctcatcctcatccagctccagctccagcactgatgagagtgaggatgagaaggaagagaagctaactgaccagtcccgctcaaagctctatgatgaagagagtctcctgtccctcactatgtcccaagatggattcccaaatgaagatggagaacaaatgacccctgagcttctgctactgcaggaaagacaaagagcctctgagtggcccaaggatcgtgtcctgataaaccgtattgacctcgtctgccaggctgtactctcagggaagtggccttctagccgtaggagccaggaaatggtaacaggaggaattttggggccaggcaaccacttgctagacagtccctcattgactcctggagaatatggtgactctccagtccccacaccacgaagtagtagtgcagcttccatggcagaggaggaagcatctgcagtcagcacagcggcagcccagttcaccaaacttcgccgaggcatggatgaaaaggagtttacagttc

SEQ ID NO: 20 - protein, 1559 aa, Homo sapiens
MSGSGSPSQSGSDSESEEEREKSSCDETESDYEPKNKVKSRKPQNRSKSKNGKKILGQKKRQIDSSEEDDDEEDYDNDKRSSRRQATVNVSYKEDEEMKTDSDDLLEVCGEDVPQPEEEEFETIERFMDCRIGRKGATGATTTIYAVEADGDPNAGFEKNKEPGEIQYLIKWKGWSHIHNTWETEETLKQQNVRGMKKLDNYKKKDQETKRWLKNASPEDVEYYNCQQELTDDLHKQYQIVERIIAHSNQKSAAGYPDYYCKWQGLPYSECSWEDGALISKKFQACIDEYFSRNQSKTTPFKDCKVLKQRPRFVALKKQPSYIGGHEGLELRDYQLNGLNWLAHSWCKGNSCILADEMGLGKTIQTISFLNYLFHEHQLYGPFLLVVPLSTLTSWQREIQTWASQMNAVVYLGDINSRNMIRTHEWTHHQTKRLKFNILLTTYEILLKDKAFLGGLNWAFIGVDEAHRLKNDDSLLYKTLIDFKSNHRLLITGTPLQNSLKELWSLLHFIMPEKFSSWEDFEEEHGKGREYGYASLHKELEPFLLRRVKKDVEKSLPAKVEQILRMEMSALQKQYYKWILTRNYKALSKGSKGSTSGFLNIMMELKKCCNHCYLIKPPDNNEFYNKQEALQHLIRSSGKLILLDKLLIRLRERGNRVLIFSQMVRMLDILAEYLKYRQFPFQRLDGSIKGELRKQALDHFNAEGSEDFCFLLSTRAGGLGINLASADTVVIFDSDWNPQNDLQAQARAHRIGQKKQVNIYRLVTKGSVEEDILERAKKKMVLDHLVIQRMDTTGKTVLHTGSAPSSSTPFNKEELSAILKFGAEELFKEPEGEEQEPQEMDIDEILKRAETHENEPGPLTVGDELLSQFKVANFSNMDEDDIELEPERNSKNWEEIIPEDQRRRLEEEERQKELEEIYMLPRMRNCAKQISFNGSEGRRSRSRRYSGSDSDSISEGKRPKKRGRPRTIPRENIKGFSDAEIRRFIKSYKKFGGPLERLDAIARDAELVDKSETDLRRLGELVHNGCIKALKDSSSGTERTGGRLGKVKGPTFRISGVQVNAKLVISHEEELIPLHKSIPSDPEERKQYTIPCHTKAAHFDIDWGKEDDSNLLIGIYEYGYGSWEMIKMDPDLSLTHKILPDDPDKKPQAKQLQTRADYLIKLLSRDLAKKEALSGAGSSKRRKARAKKNKAMKSIKVKEEIKSDSSPLPSEKSDEDDDKLSESKSDGRERSKKSSVSDAPVHITASGEPVPISEESEELDQKTFSICKERMRPVKAALKQLDRPEKGLSEREQLEHTRQCLIKIGDHITECLKEYTNPEQIKQWRKNLWIFVSKFTEFDARKLHKLYKHAIKKRQESQQNSDQNSNLNPHVIRNPDVERLKENTNHDDSSRDSYSSDRHLTQYHDHHKDRHQGDSYKKSDSRKRPYSSFSNGKDHRDWDHYKQDSRYYSDREKHRKLDDHRSRDHRSNLEGSLKDRSHSDHRSHSDHRLHSDHRSSSEYTHHKSSRDYRYHSDWQMDHRASSSGPRSPLDQRSYGSRSPFEHSVEHKSTPEHTWSSRKT

SEQ ID NO: 21 - protein, 1709 aa, Homo sapiens
MNGHSDEESVRNSSGESSQSDDDSGSASGSGSGSSSGSSSDGSSSQSGSSDSDSGSESGSQSESESDTSRENKVQAKPPKVDGAEFWKSSPSILAVQRSAILKKQQQQQQQQQHQASSNSGSEEDSSSSEDSDDSSSEVKRKKHKDEDWQMSGSGSPSQSGSDSESEEEREKSSCDETESDYEPKNKVKSRKPQNRSKSKNGKKILGQKKRQIDSSEEDDDEEDYDNDKRSSRRQATVNVSYKEDEEMKTDSDDLLEVCGEDVPQPEEEEFETIERFMDCRIGRKGATGATTTIYAVEADGDPNAGFEKNKEPGEIQYLIKWKGWSHIHNTWETEETLKQQNVRGMKKLDNYKKKDQETKRWLKNASPEDVEYYNCQQELTDDLHKQYQIVGRIIAHSNQKSAAGYPDYYCKWQGLPYSECSWEDGALISKKFQACIDEYFSRNQSKTTPFKDCKVLKQRPRFVALKKQPSYIGGHEGLELRDYQLNGLNWLAHSWCKGNSCILADEMGLGKTIQTISFLNYLFHEHQLYGPFLLVVPLSTLTSWQREIQTWASQMNAVVYLGDINSRNMIRTHEWTHHQTKRLKFNILLTTYEILLKDKAFLGGLNWAFIGVDEAHRLKNDDSLLYKTLIDFKSNHRLLITGTPLQNSLKELWSLLHFIMPEKFSSWEDFEEEHGKGREYGYASLHKELEPFLLRRVKKDVEKSLPAKVEQILRMEMSALQKQYYKWILTRNYKALSKGSKGSTSGFLNIMMELKKCCNHCYLIKPPDNNEFYNKQEALQHLIRSSGKLILLDKLLIRLRERGNRVLIFSQMVRMLDILAEYLKYRQFPFQRLDGSIKGELRKQALDHFNAEGSEDFCFLLSTRAGGLGINLASADTVVIFDSDWNPQNDLQAQARAHRIGQKKQVNIYRLVTKGSVEEDILERAKKKMVLDHLVIQRMDTTGKTVLHTGSAPSSSTPFNKEELSAILKFGAEELFKEPEGEEQEPQEMDIDEILKRAETHENEPGPLTVGDELLSQFKVANFSNMDEDDIELEPERNSKNWEEIIPEDQRRRLEEEERQKELEEIYMLPRMRNCAKQISFNGSEGRRSRSRRYSGSDSDSISEGKRPKKRGRPRTIPRENIKGFSDAEIRRFIKSYKKFGGPLERLDAIARDAELVDKSETDLRRLGELVHNGCIKALKDSSSGTERTGGRLGKVKGPTFRISGVQVNAKLVISHEEELIPLHKSIPSDPEERKQYTIPCHTKAAHFDIDWGKEDDSNLLIGIYEYGYGSWEMIKMDPDLSLTHKILPDDPDKKPQAKQLQTRADYLIKLLSRDLAKKEALSGAGSSKRRKARAKKNKAMKSIKVKEEIKSDSSPLPSEKSDEDDDKLSESKSDGRERSKKSSVSDAPVHITASGEPVPISEESEELDQKTFSICKERMRPVKAALKQLDRPEKGLSEREQLEHTRQCLIKIGDHITECLKEYTNPEQIKQWRKNLWIFVSKFTEFDARKLHKLYKHAIKKRQESQQNSDQNSNLNPHVIRNPDVERLKENTNHDDSSRDSYSSDRHLTQYHDHHKDRHQGDSYKKSDSRKRPYSSFSNGKDHRDWDHYKQDSRYYSDREKHRKLDDHRSRDHRSNLEGSLKDRSHSDHRSHSDHRLHSDHRSSSEYTHHKSSRDYRYHSDWQMDHRASSSGPRSPLDQRSYGSRSPFEHSVEHKSTPEHTWSSRKT

SEQ ID NO: 22 - protein, 1739 aa, Homo sapiens
MMRNKDKSQEEDSSLHSNASSHSASEEASGSDSGSQSESEQGSDPGSGHGSESNSSSESSESQSESESESAGSKSQPVLPEAKEKPASKKERIADVKKMWEEYPDVYGVRRSNRSRQEPSRFNIKEEASSGSESGSPKRRGQRQLKKQEKWKQEPSEDEQEQGTSAESEPEQKKVKARRPVPRRTVPKPRVKKQPKTQRGKRKKQDSSDEDDDDDEAPKRQTRRRAAKNVSYKEDDDFETDSDDLIEMTGEGVDEQQDNSETIEKVLDSRLGKKGATGASTTVYAIEANGDPSGDFDTEKDEGEIQYLIKWKGWSYIHSTWESEESLQQQKVKGLKKLENFKKKEDEIKQWLGKVSPEDVEYFNCQQELASELNKQYQIVERVIAVKTSKSTLGQTDFPAHSRKPAPSNEPEYLCKWMGLPYSECSWEDEALIGKKFQNCIDSFHSRNNSKTIPTRECKALKQRPRFVALKKQPAYLGGENLELRDYQLEGLNWLAHSWCKNNSVILADEMGLGKTIQTISFLSYLFHQHQLYGPFLIVVPLSTLTSWQREFEIWAPEINVVVYIGDLMSRNTIREYEWIHSQTKRLKFNALITTYEILLKDKTVLGSINWAFLGVDEAHRLKNDDSLLYKTLIDFKSNHRLLITGTPLQNSLKELWSLLHFIMPEKFEFWEDFEEDHGKGRENGYQSLHKVLEPFLLRRVKKDVEKSLPAKVEQILRVEMSALQKQYYKWILTRNYKALAKGTRGSTSGFLNIVMELKKCCNHCYLIKPPEENERENGQEILLSLIRSSGKLILLDKLLTRLRERGNRVLIFSQMVRMLDILAEYLTIKHYPFQRLDGSIKGEIRKQALDHFNADGSEDFCFLLSTRAGGLGINLASADTVVIFDSDWNPQNDLQAQARAHRIGQKKQVNIYRLVTKGTVEEEIIERAKKKMVLDHLVIQRMDTTGRTILENNSGRSNSNPFNKEELTAILKFGAEDLFKELEGEESEPQEMDIDEILRLAETRENEVSTSATDELLSQFKVANFATMEDEEELEERPHKDWDEIIPEEQRKKVEEEERQKELEEIYMLPRIRSSTKKAQTNDSDSDTESKRQAQRSSASESETEDSDDDKKPKRRGRPRSVRKDLVEGFTDAEIRRFIKAYKKFGLPLERLECLARDAELVDKSVADLKRLGELIHNSCVSAMQEYEEQLKENASEGKGPGKRRGPTIKISGVQVNVKSIIQHEEEFEMLHKSIPVDPEEKKKYCLTCRVKAAHFDVEWGVEDDSRLLLGIYEHGYGNWELIKTDPELKLTDKILPVETDKKPQGKQLQTRADYLLKLLRKGLEKKGAVTGGEEAKLKKRKPRVKKENKVPRLKEEHGIELSSPRHSDNPSEEGEVKDDGLEKSPMKKKQKKKENKENKEKQMSSRKDKEGDKERKKSKDKKEKPKSGDAKSSSKSKRSQGPVHITAGSEPVPIGEDEDDDLDQETFSICKERMRPVKKALKQLDKPDKGLNVQEQLEHTRNCLLKIGDRIAECLKAYSDQEHIKLWRRNLWIFVSKFTEFDARKLHKLYKMAHKKRSQEEEEQKKKDDVTGGKKPFRPEASGSSRDSLISQSHTSHNLHPQKPHLPASHGPQMHGHPRDNYNHPNKRHFSNADRGDWQRERKFNYGGGNNNPPWGSDRHHQYEQHWYKDHHYGDRRHMDAHRSGSYRPNNMSRKRPYDQYSSDRDHRGHRDYYDRYAKGCETPGANLCQELFLGRK

SEQ ID NO: 23 - protein, 1739 aa, Homo sapiens; identical to SEQ ID NO: 22

SEQ ID NO: 24 - protein, 1944 aa, Homo sapiens
MKAADTVILWARSKNDQLRISFPPGLCWGDRMPDKDDIRLLPSALGVKKRKRGPKKQKENKPGKPRKRKKRDSEEEFGSERDEYREKSESGGSEYGTGPGRKRRRKHREKKEKKTKRRKKGEGDGGQKQVEQKSSATLLLTWGLEDVEHVFSEEDYHTLTNYKAFSQFMRPLIAKKNPKIPMSKMMTILGAKWREFSANNPFKGSAAAVAAAAAAAAAAVAEQVSAAVSSATPIAPSGPPALPPPPAADIQPPPIRRAKTKEGKGPGHKRRSKSPRVPDGRKKLRGKKMAPLKIKLGLLGGKRKKGGSYVFQSDEGPEPEAEESDLDSGSVHSASGRPDGPVRTKKLKRGRPGRKKKKVLGCPAVAGEEEVDGYETDHQDYCEVCQQGGEIILCDTCPRAYHLVCLDPELDRAPEGKWSCPHCEKEGVQWEAKEEEEEYEEEGEEEGEKEEEDDHMEYCRVCKDGGELLCCDACISSYHIHCLNPPLPDIPNGEWLCPRCTCPVLKGRVQKILHWRWGEPPVAVPAPQQADGNPDVPPPRPLQGRSEREFFVKWVGLSYWHCSWAKELQLEIFHLVMYRNYQRKNDMDEPPPLDYGSGEDDGKSDKRKVKDPHYAEMEEKYYRFGIKPEWMTVHRIINHSVDKKGNYHYLVKWRDLPYDQSTWEEDEMNIPEYEEHKQSYWRHRELIMGEDPAQPRKYKKKKKELQGDGPPSSPTNDPTVKYETQPRFITATGGTLHMYQLEGLNWLRFSWAQGTDTILADEMGLGKTIQTIVFLYSLYKEGHTKGPFLVSAPLSTIINWEREFQMWAPKFYVVTYTGDKDSRAIIRENEFSFEDNAIKGGKKAFKMKREAQVKFHVLLTSYELITIDQAALGSIRWACLVVDEAHRLKNNQSKFFRVLNGYKIDHKLLLTGTPLQNNLEELFHLLNFLTPERFNNLEGFLEEFADISKEDQIKKLHDLLGPHMLRRLKADVFKNMPAKTELIVRVELSPMQKKYYKYILTRNFEALNSRGGGNQVSLLNIMMDLKKCCNHPYLFPVAAMESPKLPSGAYEGGALIKSSGKLMLLQKMLRKLKEQGHRVLIFSQMTKMLDLLEDFLDYEGYKYERIDGGITGALRQEAIDRFNAPGAQQFCFLLSTRAGGLGINLATADTVIIFDSDWNPHNDIQAFSRAHRIGQANKVMIYRFVTRASVEERITQVAKRKMMLTHLVVRPGLGSKAGSMSKQELDDILKFGTEELFKDENEGENKEEDSSVIHYDNEAIARLLDRNQDATEDTDVQNMNEYLSSFKVAQYVVREEDKIEEIEREIIKQEENVDPDYWEKLLRHHYEQQQEDLARNLGKGKRVRKQVNYNDAAQEDQDNQSEYSVGSEEEDEDFDERPEGRRQSKRQLRNEKDKPLPPLLARVGGNIEVLGFNTRQRKAFLNAVMRWGMPPQDAFTTQWLVRDLRGKTEKEFKAYVSLFMRHLCEPGADGSETFADGVPREGLSRQQVLTRIGVMSLVKKKVQEFEHINGRWSMPELMPDPSADSKRSSRASSPTKTSPTTPEASATNSPCTSKPATPAPSEKGEGIRTPLEKEEAENQEEKPEKNSRIGEKMETEADAPSPAPSLGERLEPRKIPLEDEVPGVPGEMEPEPGYRGDREKSEDVKGDRELRPGPRDEPRSNGRREEKTEKPRFMFNIADGGFTELHTLWQNEERAAISSGKLNEIWHRRHDYWLLAGIVLHGYARWQDIQNDAQFAIINEPFKTEANKGNFLEMKNKFLARRFKLLEQALVIEEQLRRAAYLNLSQEPAHPAMALHARFAEAECLAESHQHLSKESLAGNKPANAVLHKVLNQLEELLSDMKADVTRLPATLSRIPPIAARLQMSERSILSRLASKGTEPHPTPAYPPGPYATPPGYGAAFSAAPVGALAAAGANYSQMPAGSFITAATNGPIHRFFKKVPEIQ

SEQ ID NO: 25 - protein, 1377 aa, Homo sapiens
MEERFYRYGIKPEWMMIHRILNHSFDKKGDVHYLIKWKDLPYDQCTWEIDDIDIPYYDNLKQAYWGHRELMLGEDTRLPKRLLKKGKKLRDDKQEKPPDTPIVDPTVKFDKQPWYIDSTGGTLHPYQLEGLNWLRFSWAQGTDTILADEMGLGKTVQTIVFLYSLYKEGHSKGPYLVSAPLSTIINWEREFEMWAPDFYVVTYTGDKESRSVIRENEFSFEDNAIRSGKKVFRMKKEVQIKFHVLLTSYELITIDQAILGSIEWACLVVDEAHRLKNNQSKFFRVLNSYKIDYKLLLTGTPLQNNLEELFHLLNFLTPERFNNLEGFLEEFADISKEDQIKKLHDLLGPHMLRRLKADVFKNMPAKTELIVRVELSQMQKKYYKFILTRNFEALNSKGGGNQVSLLNIMMDLKKCCNHPYLFPVAAVEAPVLPNGSYDGSSLVKSSGKLMLLQKMLKKLRDEGHRVLIFSQMTKMLDLLEDFLEYEGYKYERIDGGITGGLRQEAIDRFNAPGAQQFCFLLSTRAGGLGINLATADTVIIYDSDWNPHNDIQAFSRAHRIGQNKKVMIYRFVTRASVEERITQVAKRKMMLTHLVVRPGLGSKSGSMTKQELDDILKFGTEELFKDDVEGMMSQGQRPVTPIPDVQSSKGGNLAASAKKKHGSTPPGDNKDVEDSSVIHYDDAAISKLLDRNQDATDDTELQNMNEYLSSFKVAQYVVREEDGVEEVEREIIKQEENVDPDYWEKLLRHHYEQQQEDLARNLGKGKRIRKQVNYNDASQEDQEWQDELSDNQSEYSIGSEDEDEDFEERPEGQSGRRQSRRQLKSDRDKPLPPLLARVGGNIEVLGFNARQRKAFLNAIMRWGMPPQDAFNSHWLVRDLRGKSEKEFRAYVSLFMRHLCEPGADGAETFADGVPREGLSRQHVLTRIGVMSLVRKKVQEFEHVNGKYSTPDLIPEGPEGKKSGEVISSDPNTPVPASPAHLLPAPLGLPDKMEAQLGYMDEKDPGAQKPRQPLEVQALPAALDRVESEDKHESPASKERAREERPEETEKAPPSPEQLPREEVLPEKEKILDKLELSLIHSRGDSSELRPDDTKAEEKEPIETQQNGDKEEDDEGKKEDKKGKFKFMFNIADGGFTELHTLWQNEERAAVSSGKIYDIWHRRHDYWLLAGIVTHGYARWQDIQNDPRYMILNEPFKSEVHKGNYLEMKNKFLARRFKLLEQALVIEEQLRRAAYLNMTQDPNHPAMALNARLAEVECLAESHQHLSKESLAGNKPANAVLHKVLNQLEELLSDMKADVTRLPSMLSRIPPVAARLQMSERSILSRLTNRAGDPTIQQGAFGSSQMYSNNFGPNFRGPGPGGIVNYNQMPLGPYVTDI

SEQ ID NO: 26 - protein, 1912 aa, Homo sapiens
MASGLGSPSPCSAGSEEEDMDALLNNSLPPPHPENEEDPEEDLSETETPKLKKKKKPKKPRDPKIPKSKRQKKERMLLCRQLGDSSGEGPEFVEEEEEVALRSDSEGSDYTPGKKKKKKLGPKKEKKSKSKRKEEEEEDDDDDDSKEPKSSAQLLEDWGMEDIDHVFSEEDYRTLTNYKAFSQFVRPLIAAKNPKIAVSKMMMVLGAKWREFSTNNPFKGSSGASVAAAAAAAVAVVESMVTATEVAPPPPPVEVPIRKAKTKEGKGPNARRKPKGSPRVPDAKKPKPKKVAPLKIKLGGFGSKRKRSSSEDDDLDVESDFDDASINSYSVSDGSTSRSSRSRKKLRTTKKKKKGEEEVTAVDGYETDHQDYCEVCQQGGEIILCDTCPRAYHMVCLDPDMEKAPEGKWSCPHCEKEGIQWEAKEDNSEGEEILEEVGGDLEEEDDHHMEFCRVCKDGGELLCCDTCPSSYHIHCLNPPLPEIPNGEWLCPRCTCPALKGKVQKILIWKWGQPPSPTPVPRPPDADPNTPSPKPLEGRPERQFFVKWQGMSYWHCSWVSELQLELHCQVMFRNYQRKNDMDEPPSGDFGGDEEKSRKRKNKDPKFAEMEERFYRYGIKPEWMMIHRILNHSVDKKGHVHYLIKWRDLPYDQASWESEDVEIQDYDLFKQSYWNHRELMRGEEGRPGKKLKKVKLRKLERPPETPTVDPTVKYERQPEYLDATGGTLHPYQMEGLNWLRFSWAQGTDTILADEMGLGKTVQTAVFLYSLYKEGHSKGPFLVSAPLSTIINWEREFEMWAPDMYVVTYVGDKDSRAIIRENEFSFEDNAIRGGKKASRMKKEASVKFHVLLTSYELITIDMAILGSIDWACLIVDEAHRLKNNQSKFFRVLNGYSLQHKLLLTGTPLQNNLEELFHLLNFLTPERFHNLEGFLEEFADIAKEDQIKKLHDMLGPHMLRRLKADVFKNMPSKTELIVRVELSPMQKKYYKYILTRNFEALNARGGGNQVSLLNVVMDLKKCCNHPYLFPVAAMEAPKMPNGMYDGSALIRASGKLLLLQKMLKNLKEGGHRVLIFSQMTKMLDLLEDFLEHEGYKYERIDGGITGNMRQEAIDRFNAPGAQQFCFLLSTRAGGLGINLATADTVIIYDSDWNPHNDIQAFSRAHRIGQNKKVMIYRFVTRASVEERITQVAKKKMMLTHLVVRPGLGSKTGSMSKQELDDILKFGTEELFKDEATDGGGDNKEGEDSSVIHYDDKAIERLLDRNQDETEDTELQGMNEYLSSFKVAQYVVREEEMGEEEEVEREIIKQEESVDPDYWEKLLRHHYEQQQEDLARNLGKGKRIRKQVNYNDGSQEDRDWQDDQSDNQSDYSVASEEGDEDFDERSEAPRRPSRKGLRNDKDKPLPPLLARVGGNIEVLGFNARQRKAFLNAIMRYGMPPQDAFTTQWLVRDLRGKSEKEFKAYVSLFMRHLCEPGADGAETFADGVPREGLSRQHVLTRIGVMSLIRKKVQEFEHVNGRWSMPELAEVEENKKMSQPGSPSPKTPTPSTPGDTQPNTPAPVPPAEDGIKIEENSLKEEESIEGEKEVKSTAPETAIECTQAPAPASEDEKVVVEPPEGEEKVEKAEVKERTEEPMETEPKGAADVEKVEEKSAIDLTPIVVEDKEEKKEEEEKKEVMLQNGETPKDLNDEKQKKNIKQRFMFNIADGGFTELHSLWQNEERAATVTKKTYEIWHRRHDYWLLAGIINHGYARWQDIQNDPRYAILNEPFKGEMNRGNFLEIKNKFLARRFKLLEQALVIEEQLRRAAYLNMSEDPSHPSMALNTRFAEVECLAESHQHLSKESMAGNKPANAVLHKVLKQLEELLSDMKADVTRLPATIARIPPVAVRLQMSERNILSRLANRAPEPTPQQVAQQQ

SEQ ID NO: 27 - protein, 1912 aa, Homo sapiens; identical to SEQ ID NO: 26

SEQ ID NO: 28 - protein, 1297 aa, Homo sapiens
KKVKTQTSSFDIQKAEWLRKYNPEQLLQDEGYKKHIKHHCNKVLLRVRMLYYLKQEVIGNECQKVFDGVDASDIDVWVPEPDHSEVPAEWWDFDADKSLLIGVFKHGYEKYNTIRADPALCFLERVGKPDEKAVAAEQRANDYMDGDVEDPEYKPAPAIFKDDIEDDVSSPGDLVIADGDGQLMEGDKVYWPTQSALTTRLRRLITAYQRTNKNRQIQQIQPTFSVPTSVMQPIYEEATLNPKMAAKIERQQRWTRREEADFYRVVSTFGVVFDPDRGQFDWTKFRAMARLHKKTDDSLEKYLYAFMSMCRRVCRLPSKEELVDPNIFIQPITEERASRTLYRIELLRKVREQALRHPQLFERLKLCHPNPDLPVWWECGPHDRDLLIGAAKHGVSRTDYHILRDPELSFMAAQRNYSQSKMAHSRTSTPLLQQYQVALSASPLTSLPRLLDAKGIILEEMKVKSENLKEEPQSSEEESMSSVETRTLIKSEPVSPKNGVLPQATGDQKSGGKCETDRRMVAARTEPLTPNPASKKPRVHKRGSESSSDSDSDSERSSCSSRSSSSSSSSSCSHSRSGSSSSSSSSCSSASSSSSSSTSSSSSSSSSSSEESDSDEEEAQKRESTTHMKAYDEESVASLSTTQDETQDSFQMNNGTPESAYILQGGYMLAASYWPKDRVMINRLDSICQTVLKGKWPSARRSYDANTVASFYTTKLLDSPGAATEYSEPSVPTPPGAGVKEEHDQSTQMSKEGGLKLTFQKQGLAQKRPFDGEDGALGQQQYLTRLRELQSASETSLVNFPKSIPVSGTSIQPTLGANGVILDNQPIVKKRRGRRKNVEGVDIFFFNRNKPPNHVSLGLTSSQISTGINPALSYTQPQGIPDTESPVPVINLKDGTRLAGDDAPKRKDLEKWLKEHPGYVEDLGAFIPRMQLHEGRPKQKRHRCRNPNKLDVNSLTGEERVQLINRRNARKVGGAFAPPLKDLCRFLKENSEYGVAPEWGDVVKQSGFLPESMYERILTGPVVREEVSRRGRRPKSGIAKATAAAAAASATSVSGNPLLANGLLPGVDLTTLQALQQNLQNLQSLQVTAGLMGMPTGLPSGGEAKNMAAMFPMLLSGMAGLPNLLGMGGLLTKPTESGTEDKKGSDSKESEGKTERTESQSSENGGENSVSSSPSASSTAALNTAAAANPLALNPLLLSNILYPGMLLTPGLNLHIPTLSQSNTFDVQNKNSDLGSSKSVEVKEEDSRIKDQEDKGGTEPSPLNENSTDEGSEKADASSGSDSTSSSSEDSDSSNED

SEQ ID NO: 29 - protein, 2131 aa, Homo sapiens
EPPEDDANIIEKILASKTVQEVHPGEPPFDLELFYVKYRNFSYLHCKWATMEELEKDPRIAQKIKRFRNKQAQMKHIFTEPDEDLFNPDYVEVDRILEVAHTKDAETGEEVTHYLVKWCSLPYEESTWELEEDVDPAKVKEFESLQVLPEIKHVERPASDSWQKLEKSREYKNSNQLREYQLEGMNWLLFNWYNRKNCILADEMGLGKTIQSITFLSEIFLRGIHGPFLIIAPLSTITNWEREFRTWTEMNAIVYHGSQISRQMIQQYEMVYRDAQGNPLSGVFKFHVVITTFEMILADCPELKKIHWSCVIIDEAHRLKNRNCKLLEGLKLMALEHKVLLTGTPLQNSVEELFSLLNFLEPSQFPSETAFLEEFGDLKTEEQVKKLQSILKPMMLRRLKDDVEKNLAPKQETIIEVELTNIQKKYYRAILEKNFSFLTKGANQHNMPNLINTMMELRKCCNHPYLINGAEEKILEDFRKTHSPDAPDFQLQAMIQAAGKLVLIDKLLPKLIAGGHKVLIFSQMVRCLDILEDYLIQRRYTYERIDGRVRGNLRQAAIDRFCKPDSDRFVFLLCTRAGGLGINLTAADTCIIFDSDWNPQNDLQAQARCHRIGQSKAVKVYRLITRNSYEREMFDKASLKLGLDKAVLQDINRKGGTNGVQQLSKMEVEDLLRKGAYGALMDEEDEGSKFCEEDIDQILQRRTHTITIQSEGKGSTFAKASFVASGNRTDISLDDPNFWQKWAKIAELDTEAKNEKESLVIDRPRVRKQTKHYNSFEEDELMEFSELDSDSDERPTRSRRLNDKARRYLRAECFRVEKNLLIFGWGRWKDILTHGRFKWHLNEKDMEMICRALLVYCVKHYKGDEKIKSFIWELITPTKDGQAQTLQNHSGLSAPVPRGRKGKKTKNQLLIPELKDADWLATCNPEVVLHDDGYKKHLKQHCNKVLLRVRMLYYLKAEILGEAAEKAFEGSPARELDVPLPDIDYMEIPVDWWDAEADKSLLIGVFKHGYERYNAMRADPALCFLEKVGMPDEKSLSAEQGVTDGTSDIPERGNTDKEDNAEDKVDGLQKQTESSSDGGDGVFSEKKDDSRAAQDGSDPDKSPWPVSSALTARLRRLVTVYQRCNRKELCRPEILGPGNQGYWVQEEMFRRTSEMDLINKEAQKRWTRREQADFYRTVSSFGVVYDQEKKTFDWTQFRIISRLDKKSDESLEQYFYSFVAMCRNVCRLPTWKDGGPPDTTIYVEPITEERAARTLYRIELLRKVREQVLKCPQLHERLQLCRPSLYLPVWWECGKHDRDLLIGTAKHGLNRTDCYIMNDPQLSFLDAYRNYAQHKRSGTQAPGNLCCLYQTNSKLYESLTYSQMSRTSESLENEPENLVRVESRDDHLSLPDVTCENFISKVQDVISINHDESLLPESLESMMYGKKVLSQEPSSFQESPSTNTESRKDVITISISKDGNCQSGGPEAEIASGPTFMGSLEAGGVAQANIKNGKHLLMSISKEGELCCSEAGQRPENIGQLEAKCLASPSLNPGNESGFVDMCSLSVCDSKRNLSSDQQLIDLLENKSLESKLILSQNHSDEEEEEEENEEENLAMAVGMGERPEVLHLTEPTTNISREKNQGFQDETKKGSLEVANQTPGLQRAFPAPAACQCHCKHMERWMHGLENDEFEIEKPKAYIPDLFKSKTNTIAMEGEPTAIPSQPFKVKHELLKEPWKESAEGQNVFPTYPLEGSELKSEDMDFENKDDYDRDGNCHSQDYPGKYSEEESKSSTSGITGDIGDELQEARAPTIAQLLQEKTLYSFSEWPKDRVIINRLDNICHVVLKGKWPSSQQYEPSGTLPTPVLTSSAGSRTSLSEPEAAEHSFSNGAALAAQIHKESFLAPVFTKDEQKHRRPYEFEVERDAKARGLEQFSATHGHTPIILNGWHGESAMDLSCSSEGSPGATSPFPVSASTPKIGAISSLQGALGMDLSGILQAGLIHPVTGQIVNGSLRRDDAATRRRRGRRKHVEGGMDLIFLKEQTLQAGILEVHEDPGQATLSTTHPEGPGPATSAPEPATAASSQAEKSIPSKSLLDWLRQQADYSLEVPGFGANFSDKPKQRRPRCKEPGKLDVSSLTGKREFLPSPRSQD

SEQ ID NO: 30 - protein, 2713 aa, Homo sapiens [sequence omitted]

SEQ ID NO: 31 - protein, 1967 aa, Homo sapiens
LEFKISDEEADDADAAGRDSPSNTSQSEQQESVDAEGPVVEKIMSSRSVKKQKESGEEVEIEEFYVKYKNFSYLHCQWASIEDLEKDKRIQQKIKRFKAKQGQNKFLSEIEDELFNPDYVEVDRIMDFARSTDDRGEPVTHYLVKWCSLPYEDSTWERRQDIDQAKIEEFEKLMSREPETERVERPPADDWKKSESSREYKNNNKLREYQLEGVNWLLFNWYNMRNCILADEMGLGKTIQSITFLYEIYLKGIHGPFLVIAPLSTIPNWEREFRTWTELNVVVYHGSQASRRTIQLYEMYFKDPQGRVIKGSYKFHAIITTFEMILTDCPELRNIPWRCVVIDEAHRLKNRNCKLLEGLKMMDLEHKVLLTGTPLQNTVEELFSLLHFLEPSRFPSETTFMQEFGDLKTEEQVQKLQAILKPMMLRRLKEDVEKNLAPKEETIIEVELTNIQKKYYRAILEKNFTFLSKGGGQANVPNLLNTMMELRKCCNHPYLINGAEEKILEEFKETHNAESPDFQLQAMIQAAGKLVLIDKLLPKLKAGGHRVLIFSQMVRCLDILEDYLIQRRYPYERIDGRVRGNLRQAAIDRFSKPDSDRFVFLLCTRAGGLGINLTAADTCIIFDSDWNPQNDLQAQARCHRIGQSKSVKIYRLITRNSYEREMFDKASLKLGLDKAVLQSMSGRENATNGVQQLSKKEIEDLLRKGAYGALMDEEDEGSKFCEEDIDQILLRRTHTITIESEGKGSTFAKASFVASGNRTDISLDDPNFWQKWAKKAELDIDALNGRNNLVIDTPRVRKQTRLYSAVKEDELMEFSDLESDSEEKPCAKPRRPQDKSQGYARSECFRVEKNLLVYGWGRWTDILSHGRYKRQLTEQDVETICRTILVYCLNHYKGDENIKSFIWDLITPTADGQTRALVNHSGLSAPVPRGRKGKKVKAQSTQPVVQDADWLASCNPDALFQEDSYKKHLKHHCNKVLLRVRMLYYLRQEVIGDQADKILEGADSSEADVWIPEPFHAEVPADWWDKEADKSLLIGVFKHGYEKYNSMRADPALCFLERVGMPDAKAIAAEQRGTDMLADGGDGGEFDREDEDPEYKPTRTPFKDEIDEFANSPSEDKEESMEIHATGKHSESNAELGQLYWPNTSTLTTRLRRLITAYQRSYKRQQMRQEALMKTDRRRRRPREEVRALEAEREAIISEKRQKWTRREEADFYRVVSTFGVIFDPVKQQFDWNQFRAFARLDKKSDESLEKYFSCFVAMCRRVCRMPVKPDDEPPDLSSIIEPITEERASRTLYRIELLRKIREQVLHHPQLGERLKLCQPSLDLPEWWECGRHDRDLLVGAAKHGVSRTDYHILNDPELSFLDAHKNFAQNRGAGNTSSLNPLAVGFVQTPPVISSAHIQDERVLEQAEGKVEEPENPAAKEKCEGKEEEEETDGSGKESKQECEAEASSVKNELKGVEVGADTGSKSISEKGSEEDEEEKLEDDDKSEESSQPEAGAVSRGKNFDEESNASMSTARDETRDGFYMEDGDPSVAQLLHERTFAFSFWPKDRVMINRLDNICEAVLKGKWPVNRRQMFDFQGLIPGYTPTTVDSPLQKRSFAELSMVGQASISGSEDITTSPQLSKEDALNLSVPRQRRRRRRKIEIEAERAAKRRNLMEMVAQLRESQVVSENGQEKVVDLSKASREATSSTSNFSSLSSKFILPNVSTPVSDAFKTQMELLQAGLSRTPTRHLLNGSLVDGEPPMKRRRGRRKNVEGLDLLFMSHKRTSLSAEDAEVTKAFEEDIETPPTRNIPSPGQLDPDTRIPVINLEDGTRLVGEDAPKNKDLVEWLKLHPTYTVDMPSYVPKNADVLFSSFQKPKQKRHRCRNPNKLDINTLTGEERVPVVNKRNGKKMGGAMAPPMKDLPRWLEENPEFAVAPDWTDIVKQSGFVPESMFDRLLTGPVVRGEGASRRGRRPKSEIARAA

SEQ ID NO: 32 - protein, 1501 aa, Homo sapiens
MPNLLNTMMELRKCCNHPYLINGAEEKILTEFREACHIIPHDFHLQAMVRSAGKLVLIDKLLPKLKAGGHKVLIFSQMVRCLDILEDYLIQRRYLYERIDGRVRGNLRQAAIDRFSKPDSDRFVFLLCTRAGGLGINLTAADTCIIFDSDWNPQNDLQAQARCHRIGQSKAVKVYRLITRNSYEREMFDKASLKLGLDKAVLQSMSGRDGNITGIQQFSKKEIEDLLRKGAYAAIMEEDDEGSKFCEEDIDQILLRRTTTITIESEGKGSTFAKASFVASENRTDISLDDPNFWQKWAKKADLDMDLLNSKNNLVIDTPRVRKQTRHFSTLKDDDLVEFSDLESEDDERPRSRRHDRHHAYGRTDCFRVEKHLLVYGWGRWRDILSHGRFKRRMTERDVETICRAILVYCLLHYRGDENIKGFIWDLISPAENGKTKELQNHSGLSIPVPRGRKGKKVKSQSTFDIHKADWIRKYNPDTLFQDESYKKHLKHQCNKVLLRVRMLYYLRQEVIGDQAEKVLGGAIASEIDIWFPVVDQLEVPTTWWDSEADKSLLIGVFKHGYEKYNTMRADPALCFLEKAGRPDDKAIAAEHRVLDNFSDIVEGVDFDKDCEDPEYKPLQGPPKDQDDEGDPLMMMDEEISVIDGDEAQVTQQPGHLFWPPGSALTARLRRLVTAYQRSYKREQMKIEAAERGDRRRRRCEAAFKLKEIARREKQQRWTRREQTDFYRVVSTFGVEYDPDTMQFHWDRFRTFARLDKKTDESLTKYFHGFVAMCRQVCRLPPAAGDEPPDPNLFIEPITEERASRTLYRIELLRRLREQVLCHPLLEDRLALCQPPGPELPKWWEPVRHDGELLRGAARHGVSQTDCNIMQDPDFSFLAARMNYMQNHQAGAPAPSLSRCSTPLLHQQYTSRTASPLPLRPDAPVEKSPEETATQVPSLESLTLKLEHEVVARSRPTPQDYEMRVSPSDTTPLVSRSVPPVKLEDEDDSDSELDLSKLSPSSSSSSSSSSSSSSTDESEDEKEEKLTDQSRSKLYDEESLLSLTMSQDGFPNEDGEQMTPELLLLQERQRASEWPKDRVLINRIDLVCQAVLSGKWPSSRRSQEMVTGGILGPGNHLLDSPSLTPGEYGDSPVPTPRSSSAASMAEEEASAVSTAAAQFTKLRRGMDEKEFTVQIKDEEGLKLTFQKHKLMANGVMGDGHPLFHKKKGNRKKLVELEVECMEEPNHLDVDLETRIPVINKVDGTLLVGEDAPRRAELEMWLQGHPEFAVDPRFLAYMEDRRKQKWQRCKKNNKAELNCLGMEPVQTANSRNGKKGHHTETVFNRVLPGPIAPESSKKRARRMRPDLSKMMALMQGGSTGSLSLHNTFQHSSSGLQSVSSLGHSSATSASLPFMPFVMGGAPSSPHVDSSTMLHHHHHHPHPHHHHHHHPGLRAPGYPSSPVTTASGTTLRLPPLQPEEDDDEDEEDDDDLSQGYDSSERDFSLIDDPMMPAYSDSSEDADD